(19) United States
(12) Patent Application Publication
(10) Pub. No.: US 2024/0141060 A1
(43) Pub. Date: May 2, 2024

(54) DOSAGE REGIMES FOR ANTI-CD73 AND ANTI-ENTPD2 ANTIBODIES AND USES THEREOF

(71) Applicant: NOVARTIS AG, Basel (CH)

(72) Inventors: Juan Gonzalez-Maffe, Basel (CH); Randi Ellen Isaacs, East Hampton, NY (US); Jaeyeon Kim, Lexington, MA (US); Lisa Nardi, Whitehouse Station, NJ (US); Javier Alberto Otero, New York, NY (US); Nehal Parikh, Chatham, NJ (US); Michael John Roy, Madison, NJ (US); Kulandayan Kasi Subramanian, Boxborough, MA (US); Tingting Zhai, Burlington, MA (US)

(21) Appl. No.: 18/263,151

(22) PCT Filed: Jan. 27, 2022
(86) PCT No.: PCT/IB2022/050703

§ 371 Date: Jul. 27, 2023

Related U.S. Application Data
(60) Provisional application No. 63/191,805, filed on May 21, 2021.
(60) Provisional application No. 63/143,132, filed on Jan. 29, 2021.

(51) Int. Cl. C07K 16/28; A61P 35/00; C07K 16/40

(52) U.S. Cl. CPC C07K 16/2896; A61P 35/00; C07K 16/2818; C07K 16/40; A61K 2039/545

(57) ABSTRACT

The invention generally relates to dosage regimes of anti-Cluster of Differentiation 73 (CD73) antibodies and/or anti-ectoenzyme ectonucleoside triphosphate diphosphohydrolase 2 (ENTPD2) antibodies, used in methods of treatment of cancer in a subject, as well as dosage regimes of anti-CD73 antibodies for use in treating cancer. The invention further generally relates to dosage regimes of combinations of agents, such as combinations comprising anti-CD73 antibodies and/or anti-ENTPD2 antibodies and at least one or more of a PD-1 inhibitor, and an adenosine A2AR antagonist.

### SEQUENCE LISTING

The instant application contains a Sequence Listing which has been submitted electronically in ASCII format and is hereby incorporated by reference in its entirety. Said ASCII copy, created on Dec. 6, 2021, is named PAT059034_SL.txt and is 435,867 bytes in size.

### INTRODUCTION

The invention generally relates to dosage regimes of anti-Cluster of Differentiation 73 (CD73) and anti-ectoenzyme ectonucleoside triphosphate diphosphohydrolase 2 (ENTPD2) antibodies, used in methods of treatment of cancer in a subject, as well as dosage regimes of anti-CD73 and anti-ENTPD2 antibodies for use in treating cancer. The invention further generally relates to dosage regimes of combinations of agents, such as combinations comprising anti-CD73 antibodies and/or anti-ENTPD2 antibodies and at least one or more of a PD-1 inhibitor, and an adenosine A2AR antagonist.

### BACKGROUND OF THE INVENTION

Cluster of Differentiation 73 (CD73), also known as ecto-5′-nucleotidase (ecto-5′NT), is a glycosyl-phosphatidylinositol (GPI)-linked cell surface enzyme found in most tissues, and particularly expressed in endothelial cells and subsets of hematopoietic cells (Resta et al., Immunol Rev 161:95-109 (1998) and Colgan et al., Prinergic Signal 2:351-60 (2006)). CD73 catalyzes the conversion of adenosine monophosphate (AMP) to adenosine. Adenosine is a signaling molecule which mediates its biological effects through several receptors, including the Adenosine A1, A2A, A2B, and A3 receptors. The A2A receptor has received particular attention due to its broad expression on immune cells. Adenosine has pleiotropic effects in the tumor microenvironment, including expansion of regulatory T cells (Tregs), inhibition of effector T cell (Teff) responses mediated by interferon (IFN)-γ, and expansion of myeloid derived suppressor cells (MDSCs). See, e.g., Allard B, et al., Curr Opin Pharmacol 29:7-16 (2016) and Allard D, et al., Immunotherapy 8:145-163 (2016).

CD73 is also expressed on cancer cells, including colon, lung, pancreas, ovary, bladder, leukemia, glioma, glioblastoma, melanoma, thyroid, esophageal, prostate, and breast (Jin et al., Cancer Res 70:2245-55 (2010) and Stagg et al., PNAS 107: 1547-52 (2010); Zhang et al., Cancer Res 70:6407-11 (2010)). High CD73 expression has been reported to correlate with poor outcome across various cancer indications, such as lung, melanoma, triple-negative breast, squamous head and neck and colorectal cancers. See, e.g., Allard B, et al., Expert Opin Ther Targets 18:863-881 (2014); Leclerc B G, et al., Clin Cancer Res 22:158-166 (2016); Ren Z H, et al., Oncotarget 7:61690-61702 (2016); Ren Z H, et al., Oncol Lett 12:556-562 (2016); and Turcotte M, et al., Cancer Res 75:4494-4503 (2015).

Cellular stress and apoptosis trigger the release of ATP into the extracellular space.

Increased ATP concentration promotes rapid inflammation, resulting in amplification of T-cell signaling, inhibiting regulatory T cells (Tregs), and promoting inflammasome activation in dendritic cells and macrophages. The ectoenzyme ectonucleoside triphosphate diphosphohydrolase 2 (ENTPD2) is part of the family of ecto-nucleosidases that hydrolyze 5′-triphosphates and is an integral membrane protein, which participates in purinergic signaling. ENTPD2 catalyzes the conversion of adenosine triphosphate (ATP) to adenosine diphosphate (ADP) and adenosine monophosphate (AMP). In turn, AMP is catalyzed by Cluster of Differentiation 73 (CD73), also known as ecto-5′-nucleotidase (ecto-5′NT), to adenosine.

In a mouse model of hepatocellular carcinoma it was shown that ENTPD2 converts extracellular ATP to AMP which prevents the differentiation of monocytic myeloid derived suppressor cells (MDSCs) to dendritic cells, therefore promoting the maintenance of MDSCs in vitro and in vivo (Chiu et al., Nat Commun. 8:517-28 (2017).

Given the ongoing need for improved strategies for targeting diseases such as cancer, new dosage regimes for regulating CD73 and/or ENTPD2 activity are highly desirable.

### SUMMARY OF THE INVENTION

Disclosed herein are anti-CD73 antibodies which can be used (alone or in combination with other therapeutic agents, procedures, or modalities, e.g., in combination with one or more of a Programmed Death 1 (PD-1) inhibitor, e.g. an anti-PD1 antibody molecule), an adenosine A2AR antagonist and an ectoenzyme ectonucleoside triphosphate diphosphohydrolase 2 (ENTPD2) inhibitor to treat or prevent disorders, such as cancerous disorders (e.g., solid tumors). The invention generally relates to dosage regimes for treating cancer using the anti-CD73 antibodies which are disclosed herein.

Accordingly in an aspect of the invention, there is provided an antibody molecule that binds to human CD73 for use in treating a cancer in a subject or in a method of treating cancer in a subject, whereby the antibody molecule is administered in a step-up dosing regime such that in the main phase the antibody molecule dose is administered at a main dose amount according to a main dosing period with a main dosing period frequency, which is preceded by an initial phase in which said antibody molecule is administered at a higher dosing period frequency than the main dosing period frequency and is administered via fractionated dose amounts of the main dose amount, wherein in the initial phase within a time period equal to the main dosing period the fractionated dose amounts summed together shall not exceed the amount of the main phase dose amount.

In one embodiment of the invention, there is provided an antibody molecule that binds to human CD73 for use in treating a cancer in a subject, whereby the antibody molecule is administered in a step-up dosing regime such that in the main phase the antibody molecule dose is administered at a main dose amount according to a main dosing period with a main dosing period frequency, which is preceded by an initial phase in which said antibody molecule is administered at a higher dosing period frequency than the main dosing period frequency and is administered via fractionated dose amounts of the main dose amount, wherein in the initial phase within a time period equal to the main dosing period the fractionated dose amounts summed together shall not exceed the amount of the main phase dose amount, wherein the antibody molecule comprises (i) a heavy chain variable region (VH) comprising a heavy chain complementarity determining region 1 (VHCDR1) amino acid sequence of SEQ ID NO: 88, a VHCDR2 amino acid sequence of SEQ ID NO: 89, and a VHCDR3 amino acid sequence of SEQ ID NO: 37; and (ii) a light chain variable region (VL) comprising a light chain complementarity determining region 1 (VLCDR1) amino acid sequence of SEQ ID NO: 48, a VLCDR2 amino acid sequence of SEQ ID NO: 49, and a VLCDR3 amino acid sequence of SEQ ID NO: 50.

In another embodiment of the invention, there is provided a method of treating cancer in a subject comprising administering to the subject an antibody molecule that binds to human CD73 in an amount effective to treat the cancer, whereby the antibody molecule is administered in a step-up dosing regime such that in the main phase the antibody molecule dose is administered at a main dose amount according to a main dosing period with a main dosing period frequency, which is preceded by an initial phase in which said antibody molecule is administered at a higher dosing period frequency than the main dosing period frequency and is administered via fractionated dose amounts of the main dose amount, wherein in the initial phase within a time period equal to the main dosing period the fractionated dose amounts summed together shall not exceed the amount of the main phase dose amount, wherein the antibody molecule comprises (i) a heavy chain variable region (VH) comprising a heavy chain complementarity determining region 1 (VHCDR1) amino acid sequence of SEQ ID NO: 88, a VHCDR2 amino acid sequence of SEQ ID NO: 89, and a VHCDR3 amino acid sequence of SEQ ID NO: 37; and (ii) a light chain variable region (VL) comprising a light chain complementarity determining region 1 (VLCDR1) amino acid sequence of SEQ ID NO: 48, a VLCDR2 amino acid sequence of SEQ ID NO: 49, and a VLCDR3 amino acid sequence of SEQ ID NO: 50.

In an embodiment of the antibody for use according to the invention or method according to the invention, the antibody molecule that binds to human CD73 comprises (i) a VH comprising a VHCDR1 amino acid sequence of SEQ ID NO: 38, a VHCDR2 amino acid sequence of SEQ ID NO: 36, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50; (ii) a VH comprising a VHCDR1 amino acid sequence of SEQ ID NO: 72, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50; (iii) a VH comprising a VHCDR1 amino acid sequence of SEQ ID NO: 38, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50; (iv) a VH comprising a VHCDR1 amino acid sequence of SEQ ID NO: 137, a VHCDR2 amino acid sequence of SEQ ID NO: 136, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50; (v) a VH comprising a VHCDR1 amino acid sequence of SEQ ID NO: 137, a VHCDR2 amino acid sequence of SEQ ID NO: 146, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50; or (vi) a VH comprising a VHCDR1 amino acid sequence of SEQ ID NO: 137, a VHCDR2 amino acid sequence of SEQ ID NO: 154, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50.

In an embodiment of the antibody for use according to the invention or method according to the invention, the antibody molecule that binds to human CD73 comprises a VH comprising a VHCDR1 amino acid sequence of SEQ ID NO: 38, a VHCDR2 amino acid sequence of SEQ ID NO: 36, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50.

In an embodiment of the antibody for use according to the invention or method according to the invention, the antibody molecule that binds to human CD73 comprises a heavy chain variable region comprising the amino acid sequence of SEQ ID NO: 44, 77, 84, 142, 151, or 159, or an amino acid sequence having at least about 85%, 90%, 95%, or 99% sequence identity to SEQ ID NO: 44, 77, 84, 142, 151, or 159.

In an embodiment of the antibody for use according to the invention or method according to the invention, the antibody molecule that binds to human CD73 comprises
(i) a heavy chain variable region comprising the amino acid sequence of SEQ ID NO: 44 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto);
(ii) a heavy chain variable region comprising the amino acid sequence of SEQ ID NO: 77 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto);
(iii) a heavy chain variable region comprising the amino acid sequence of SEQ ID NO: 84 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto);
(iv) a heavy chain variable region comprising the amino acid sequence of SEQ ID NO: 142 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto);
(v) a heavy chain variable region comprising the amino acid sequence of SEQ ID NO: 151 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto); or
(vi) a heavy chain variable region comprising the amino acid sequence of SEQ ID NO: 159 (or a sequence having at least about 85%, 90%, 95%, or 99% sequence identity thereto) and a light chain variable region comprising the amino acid sequence of SEQ ID NO: 55 (or a sequence having at least about 85%, 90%, 95%, or 99% sequence identity thereto).

In an embodiment of the antibody for use according to the invention or method according to the invention, the antibody molecule that binds to human CD73 comprises a heavy chain variable region comprising the amino acid sequence of SEQ ID NO: 44, or an amino acid sequence having at least about 85%, 90%, 95%, or 99% sequence identity to SEQ ID NO: 44.

In an embodiment of the antibody for use according to the invention or method according to the invention, the antibody molecule that binds to human CD73 comprises a light chain variable region comprising the amino acid sequence of SEQ ID NO: 55, or an amino acid sequence having at least about 85%, 90%, 95%, or 99% sequence identity to SEQ ID NO: 55.

In an embodiment of the antibody for use according to the invention or method according to the invention, the antibody molecule that binds to human CD73 comprises a heavy chain variable region comprising the amino acid sequence of SEQ ID NO: 44 and a light chain variable region comprising the amino acid sequence of SEQ ID NO: 55.

In an embodiment of the antibody for use according to the invention or method according to the invention, the antibody molecule that binds to human CD73 comprises a heavy chain comprising the amino acid sequence of SEQ ID NO: 46, 79, 86, 114, 116, or 117, or an amino acid sequence having at least about 85%, 90%, 95%, or 99% sequence identity to SEQ ID NO: 46, 79, 86, 114, 116, or 117.

In an embodiment of the antibody for use according to the invention or method according to the invention, the antibody molecule that binds to human CD73 comprises:
(i) a heavy chain comprising the amino acid sequence of SEQ ID NO: 46 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto);
(ii) a heavy chain comprising the amino acid sequence of SEQ ID NO: 114 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto);
(iii) a heavy chain comprising the amino acid sequence of SEQ ID NO: 79 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto);
(iv) a heavy chain comprising the amino acid sequence of SEQ ID NO: 116 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto);
(v) a heavy chain comprising the amino acid sequence of SEQ ID NO: 86 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto); or
(vi) a heavy chain comprising the amino acid sequence of SEQ ID NO: 117 (or a sequence having at least about 85%, 90%, 95%, or 99% sequence identity thereto) and a light chain comprising the amino acid sequence of SEQ ID NO: 57 (or a sequence having at least about 85%, 90%, 95%, or 99% sequence identity thereto).

In an embodiment of the antibody for use according to the invention or method according to the invention, the antibody molecule that binds to human CD73 comprises a heavy chain comprising the amino acid sequence of SEQ ID NO: 46, or an amino acid sequence having at least about 85%, 90%, 95%, or 99% sequence identity to SEQ ID NO: 46. In an embodiment X in SEQ ID NO: 46 is K.

In an embodiment of the antibody for use according to the invention or method according to the invention, the antibody molecule that binds to human CD73 comprises a light chain comprising the amino acid sequence of SEQ ID NO: 57, or an amino acid sequence having at least about 85%, 90%, 95%, or 99% sequence identity to SEQ ID NO: 57.

In an embodiment of the antibody for use according to the invention or method according to the invention, the antibody molecule that binds to human CD73 comprises a heavy chain comprising the amino acid sequence of SEQ ID NO: 46 and a light chain comprising the amino acid sequence of SEQ ID NO: 57. In an embodiment X in SEQ ID NO: 46 is K.

In an embodiment of the antibody for use according to the invention or method according to the invention, the antibody molecule that binds to human CD73 is antibody 373.A.

In an embodiment of the antibody for use according to the invention or method according to the invention, the antibody molecule that binds to human CD73 is a human antibody, a full length antibody, a bispecific antibody, Fab, F(ab′)2, Fv, or a single chain Fv fragment (scFv).

In an embodiment of the antibody for use according to the invention or method according to the invention, the antibody molecule that binds to human CD73 comprises a heavy chain constant region selected from IgG1, IgG2, IgG3, and IgG4, and a light chain constant region chosen from the light chain constant regions of kappa or lambda.

In an embodiment of the antibody for use according to the invention or method according to the invention, the antibody molecule that binds to human CD73 comprises a heavy chain constant region of IgG4 and a light chain constant region of kappa.

In an embodiment of the antibody for use according to the invention or method according to the invention, the antibody molecule that binds to human CD73 comprises a heavy chain constant region comprising an amino acid sequence selected from the group consisting of SEQ ID NOs: 92-103, 119, and 120, and/or a light chain constant region comprising the amino acid sequence of SEQ ID NO: 104.

In an embodiment of the antibody for use according to the invention or method according to the invention the antibody molecule that binds to human CD73 comprises
i) a human IgG4 heavy chain constant region with a mutation at position 228 according to EU numbering, or
ii) a human IgG4 heavy chain constant region with a Serine to Proline mutation at position 228 according to EU numbering, or
iii) a heavy chain constant region comprising the amino acid sequence of SEQ ID NO: 92 or 93.

In an embodiment of the antibody for use according to the invention or method according to the invention, the antibody molecule that binds to human CD73 comprises one or more (e.g., 2, 3, 4, 5, 6, 7, 8, 9, 10, 11, 12, 13, 14, 15, 16, or more, e.g., all) of the following properties:
(i) binds to human CD73 with a dissociation constant (KD) of less than about 1×10-8 M, e.g., when the antibody molecule is tested as a bivalent antibody molecule using Octet;
(ii) binds to soluble human CD73 and/or membrane-bound human CD73;
(iii) does not bind to murine CD73, e.g., as determined using Octet;
(iv) inhibits or reduces the enzymatic activity of CD73 (e.g., soluble human CD73 or membrane-bound human CD73), e.g., human CD73 mediated conversion of adenosine monophosphate (AMP) to adenosine, e.g., as measured by a malachite green (MG) phosphate assay or a modified Cell Titer Glo (CTG) assay;
(v) inhibits at least about 60%, 70%, 80%, or 90% of the enzymatic activity of membrane-bound human CD73, e.g., when the antibody molecule is tested as a bivalent antibody molecule using a modified Cell Titer Glo (CTG) assay;
(vi) increases proliferation of anti-CD3/anti-CD28 stimulated T cells, e.g., CD4+ T cells, in the presence of adenosine monophosphate (AMP), e.g., as measured by a CellTrace Violet (CTV) cell proliferation assay;
(vii) binds to the N-terminal domain of human CD73;
(viii) reduces hydrogen-deuterium exchange at one or more regions of a protein comprising the amino acid sequence of residues 27-547 of SEQ ID NO: 105 (e.g., a protein consisting of the amino acid sequence of SEQ ID NO: 171) when bound thereto, wherein the one or more regions are selected from the group consisting of residues 158-172, residues 206-215, residues 368-387, and residues 87-104 of SEQ ID NO: 105, e.g., when the antibody molecule is tested as a bivalent antibody molecule using hydrogen deuterium-exchange mass spectrometry;
(ix) when bound to a protein comprising the amino acid sequence of residues 27-547 of SEQ ID NO: 105 (e.g., a protein consisting of the amino acid sequence of SEQ ID NO: 171), induces a conformational change in residues 368-387 of SEQ ID NO: 105;
(x) contacts, e.g., directly or indirectly, at least one, two, three, or four residues within residues 158-172 of SEQ ID NO: 105;
(xi) contacts, e.g., directly or indirectly, at least one, two, three, four or five residues within residues 206-215 of SEQ ID NO: 105;
(xii) contacts, e.g., directly or indirectly, at least one, two, three, four or five residues within residues 368-387 of SEQ ID NO: 105 or 106;
(xiii) contacts, e.g., directly or indirectly, at least one, two, three, four or five residues within residues 87-104 of SEQ ID NO: 105;
(xiv) binds to a human CD73 dimer, said dimer consisting of a first CD73 monomer and a second CD73 monomer, wherein when the antibody molecule comprises a first antigen binding domain and a second antigen binding domain, the first antigen binding domain binds to the first CD73 monomer and the second antigen binding domain binds to the second CD73 monomer, e.g., when tested using size exclusion chromatography;
(xv) binds to a catalytically active closed conformation of human CD73 with lower affinity, e.g., 50%, 60%, 70%, 80%, 90%, 95%, or 99% lower affinity, than when the antibody molecule binds to a catalytically inactive open conformation of human CD73;
(xvi) locks human CD73 in a catalytically inactive open conformation; or
(xvii) prevents or reduces the conversion of human CD73 from a catalytically inactive open conformation to a catalytically active closed conformation, e.g., reduces the conversion by at least 1.5-fold, 2-fold, 5-fold, 10-fold, 20-fold, 30-fold, 40-fold, 50-fold, 60-fold, 70-fold, 80-fold, 90-fold, or 100-fold, compared to the conversion in the absence of the antibody molecule.

In an embodiment of the antibody for use according to the invention or method according to the invention, in the initial phase within a time period equal to the main dosing period, the fractionated dose amounts summed together equal the main dose amount.

In an embodiment of the antibody for use according to the invention or method according to the invention, the initial phase is used to prevent or reduce the incidence or severity of headaches and/or migraines. In an embodiment the initial phase is used to prevent the incidence and/or severity of headaches or migraines. In an embodiment the initial phase is used to prevent the incidence of headaches or migraines. In an embodiment the initial phase is used to prevent the severity of headaches or migraines. In an embodiment the initial phase is used to reduce the incidence and/or severity of headaches or migraines. In an embodiment the initial phase is used to reduce the incidence of headaches or migraines. In an embodiment the initial phase is used to reduce the severity of headaches or migraines). The antibody for use or method in such embodiments involving headaches and/or migraines is an anti-CD73 antibody.

In an embodiment of the antibody for use according to the invention or method according to the invention, the main dose amount of the antibody molecule that binds to human CD73 is 600 mg. In an embodiment the main dose frequency is Q2W.

In an embodiment of the antibody for use according to the invention or method according to the invention, in the initial phase the antibody molecule that binds to human CD73 is administered at a frequency of QW.

In an embodiment of the antibody for use according to the invention or method according to the invention, the initial phase of the dosing of the anti-CD73 antibody is two weeks.

In an embodiment of the antibody for use according to the invention or method according to the invention, in the initial phase the fractionated dose amounts that are administered within a time period equal to the main dosing period when summed together equal the main dose amount. In an embodiment these initial phase doses are administered at a lower amount followed by an intermediate amount of the main phase dose amount. The lower amount is lower than the main phase dose amount and the intermediate dose amount is a value between these two amounts. In an embodiment, in the initial phase the fractionated dose amounts are about 200 mg and about 400 mg and are administered within two weeks. In an alternative embodiment, in the initial phase the fractionated dose amounts are about 100 mg and about 500 mg and are administered within two weeks. In another alternative embodiment, in the initial phase the fractionated dose amounts administered within a time period equal to the main dosing period summed together equal the main dose amount and wherein the fractionated doses amounts are equal amounts, e.g. about 300 mg and about 300 mg. The amounts in these embodiments refer to the amount of the anti-CD73 antibody.

In an embodiment the anti-CD73 antibody is administered in a step-up dosing regime such that the anti-CD73 antibody is administered in the initial phase of two weeks once at about 200 mg in the first week and once at about 400 mg in the second week, followed by the main phase with about 600 mg administered Q2W.

In an embodiment of the antibody for use according to the invention or method according to the invention, the antibody molecule that binds to human CD73 is administered in the initial phase on day 1 at about 200 mg and on day 8 at about 400 mg, followed by the main phase beginning on day 15 with about 600 mg, and continuing thereafter with about 600 mg administered Q2W.

In an embodiment of the antibody for use according to the invention or method according to the invention, the antibody molecule that binds to human CD73 is administered by IV infusion over 1 to 2 hours at the dosing period frequency according to the respective phase.

In an embodiment of the antibody for use according to the invention or method according to the invention, the antibody molecule that binds to human CD73 is administered in combination with one or more therapeutic agents or procedures.

In an embodiment of the antibody for use according to the invention or method according to the invention, the antibody molecule that binds to human CD73 is administered in combination with one or more therapeutic agents or procedures chosen from one or more of chemotherapy, a targeted anti-cancer therapy, an oncolytic drug, a cytotoxic agent, an immune-based therapy, a cytokine, surgical procedure, a radiation procedure, an activator of a costimulatory molecule, an inhibitor of an inhibitory molecule, a vaccine, or a cell therapy. In an embodiment the one or more therapeutic agents is/are chosen from one or more of: 1) a protein kinase C (PKC) inhibitor; 2) a heat shock protein 90 (HSP90) inhibitor; 3) an inhibitor of a phosphoinositide 3-kinase (PI3K) and/or target of rapamycin (mTOR); 4) an inhibitor of cytochrome P450 (e.g., a CYP17 inhibitor or a 17alpha-Hydroxylase/C17-20 Lyase inhibitor); 5) an iron chelating agent; 6) an aromatase inhibitor; 7) an inhibitor of p53, e.g., an inhibitor of a p53/Mdm2 interaction; 8) an apoptosis inducer; 9) an angiogenesis inhibitor; 10) an aldosterone synthase inhibitor; 11) a smoothened (SMO) receptor inhibitor; 12) a prolactin receptor (PRLR) inhibitor; 13) a Wnt signaling inhibitor; 14) a CDK4/6 inhibitor; 15) a fibroblast growth factor receptor 2 (FGFR2)/fibroblast growth factor receptor 4 (FGFR4) inhibitor; 16) an inhibitor of macrophage colony-stimulating factor (M-CSF); 17) an inhibitor of one or more of c-KIT, histamine release, Flt3 (e.g., FLK2/STK1) or PKC; 18) an inhibitor of one or more of VEGFR-2 (e.g., FLK-1/KDR), PDGFRbeta, c-KIT or Raf kinase C; 19) a somatostatin agonist and/or a growth hormone release inhibitor; 20) an anaplastic lymphoma kinase (ALK) inhibitor; 21) an insulin-like growth factor 1 receptor (IGF-1R) inhibitor; 22) a P-Glycoprotein 1 inhibitor; 23) a vascular endothelial growth factor receptor (VEGFR) inhibitor; 24) a BCR-ABL kinase inhibitor; 25) an FGFR inhibitor; 26) an inhibitor of CYP11B2; 27) a HDM2 inhibitor, e.g., an inhibitor of the HDM2-p53 interaction; 28) an inhibitor of a tyrosine kinase; 29) an inhibitor of c-MET; 30) an inhibitor of JAK; 31) an inhibitor of DAC; 32) an inhibitor of 11p-hydroxylase; 33) an inhibitor of IAP; 34) an inhibitor of PIM kinase; 35) an inhibitor of Porcupine; 36) an inhibitor of BRAF, e.g., BRAF V600E or wild-type BRAF; 37) an inhibitor of HER3; 38) an inhibitor of MEK; or 39) an inhibitor of a lipid kinase.

In an embodiment of the antibody for use according to the invention or method according to the invention, the antibody molecule that binds to human CD73 is administered in combination with a triptan. In an embodiment, a triptan is administered prior to one or more doses of the antibody molecule that binds to human CD73. In an embodiment the triptan is selected from Almotriptan, Eletriptan, Frovatriptan, Naratriptan, Rizatriptan, Sumatriptan, Zolmitriptan, Lasmiditan, optionally which may be combined with an additional agent, such as Sumatriptan combined with naproxen sodium. In an embodiment the triptan is Sumatriptan or Solmitriptan.

In an embodiment of the antibody for use according to the invention or method according to the invention, the antibody molecule that binds to human CD73 is administered in combination with a PD-1 inhibitor. In an embodiment the PD-1 inhibitor is selected from the group consisting of Spartalizumab, Nivolumab, Pembrolizumab, Pidilizumab, MEDI0680, REGN2810, TSR-042, PF-06801591 and AMP-224. In an embodiment the PD-1 inhibitor is selected from the group consisting of Tislelizumab, Spartalizumab, Nivolumab, Pembrolizumab, Pidilizumab, MEDI0680, REGN2810, TSR-042, PF-06801591 and AMP-224. In an embodiment the PD-1 inhibitor is Spartalizumab. In an embodiment the PD-1 inhibitor is Tislelizumab. In an embodiment the PD-1 inhibitor is an anti-PD-1 antibody molecule, wherein the anti-PD-1 antibody molecule is administered at a dose of about 300 mg Q3W or at a dose of about 400 mg Q4W. In an embodiment the PD-1 inhibitor is an anti-PD-1 antibody molecule, wherein the anti-PD-1 antibody molecule is administered at a dose of about 400 mg Q4W. In an embodiment the PD-1 inhibitor is Spartalizumab and is administered at a dose of about 400 mg Q4W. In an embodiment the PD-1 inhibitor is Tislelizumab and is administered at a dose of about 300 mg Q4W.

In an embodiment of the antibody for use according to the invention or method according to the invention, the antibody molecule that binds to human CD73 is administered in combination with an adenosine A2AR antagonist. In an embodiment the
(i) the adenosine A2AR antagonist is selected from the group consisting of PBF509, CP1444, AZD4635, Vipadenant, GBV-2034, and AB928; or
(ii) the adenosine A2AR antagonist is selected from the group consisting of 5-bromo-2,6-di-(1H-pyrazol-1-yl)pyrimidine-4-amine; (S)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine; (R)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine, or racemate thereof; 7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine; and 6-(2-chloro-6-methylpyridin-4-yl)-5-(4-fluorophenyl)-1,2,4-triazin-3-amine;

In an embodiment of the antibody for use according to the invention or method according to the invention, the antibody molecule that binds to human CD73 is administered in combination with an adenosine A2AR antagonist that is PBF509. PBF509 is also known as NIR178.

In an embodiment of the antibody for use according to the invention or method according to the invention, the adenosine A2AR antagonist is administered at a dose of about 80 mg, about 160 mg, 240 mg or about 320 mg. In an embodiment of the antibody for use according to the invention or method according to the invention, the adenosine A2AR antagonist is administered at a dose of about 160 mg twice a day (BID) or about 240 mg twice a day (BID). In an embodiment of the antibody for use according to the invention or method according to the invention, the antibody molecule that binds to human CD73 is administered in combination with an adenosine A2AR antagonist that is PBF509 at a dose of about 240 mg twice a day (BID).

In an embodiment of the antibody for use according to the invention or method according to the invention, the antibody molecule that binds to human CD73 is administered in combination with an anti-human ENTPD2 antibody. In an embodiment the anti-human ENTPD2 antibody is administered in combination with an anti-human ENTPD2 antibody with a sequence given in Table 9. In an embodiment the anti-human ENTPD2 antibody comprises a VH comprising a VHCDR1 amino acid sequence of SEQ ID NO: 401, a VHCDR2 amino acid sequence of SEQ ID NO: 402, and a VHCDR3 amino acid sequence of SEQ ID NO: 403; and a VL comprising a VLCDR1 amino acid sequence of SEQ ID NO: 414, a VLCDR2 amino acid sequence of SEQ ID NO: 415, and a VLCDR3 amino acid sequence of SEQ ID NO: 416. In an embodiment the anti-human ENTPD2 antibody comprises a VH with a sequence of SEQ ID NO: 410 and VL with a sequence of SEQ ID NO: 421. In an embodiment the anti-human ENTPD2 antibody comprises a heavy chain with a sequence of SEQ ID NO: 412 and a light chain with a sequence of SEQ ID NO: 423.

In an embodiment of the antibody for use according to the invention or method according to the invention, the antibody molecule that binds to human CD73 is administered in the initial phase at about 200 mg QW, then about 400 mg QW, followed by the main phase at about 600 mg Q2W. In an embodiment of the antibody for use according to the invention or method according to the invention, the antibody molecule that binds to human CD73 is administered in the initial phase at about 200 mg QW, then about 400 mg QW, followed by the main phase at about 600 mg Q2W, in combination with Spartalizumab administered at about 400 mg Q4W and PBF509 administered at about 240 mg BID.

In an embodiment of the antibody for use according to the invention or method according to the invention, the cancer is chosen from lung cancer (e.g., non-small cell lung cancer), pancreas cancer (e.g., pancreatic ductal adenocarcinoma), breast cancer (e.g., triple-negative breast cancer), melanoma, head and neck cancer (e.g., squamous head and neck cancer), colorectal cancer (e.g., microsatellite stable (MSS) colorectal cancer), ovarian cancer, metastatic castration resistant prostate cancer or renal cancer (e.g., renal cell carcinoma). In an embodiment of the antibody for use according to the invention or method according to the invention, the cancer is chosen from non-small cell lung cancer, pancreatic ductal adenocarcinoma, triple-negative breast cancer, microsatellite stable (MSS) colorectal cancer, metastatic castration resistant prostate cancer, ovarian cancer or renal cell carcinoma.

In an embodiment of the antibody for use according to the invention or method according to the invention, the antibody molecule that binds to human CD73 is in the form of a pharmaceutical composition comprising the anti-CD73 antibody molecule as defined herein and a pharmaceutically acceptable carrier, excipient or stabilizer.

Disclosed herein are anti-ENTPD2 antibodies which can be used alone or in combination with other therapeutic agents, procedures, or modalities, e.g., in combination with one or more of a Programmed Death 1 (PD-1) inhibitor (e.g. an anti-PD1 antibody molecule), an adenosine A2AR antagonist and a CD73 inhibitor (e.g., an anti-CD73 antibody molecule), to treat or prevent disorders, such as cancerous disorders (e.g., solid tumors). The invention generally relates to dosage regimes for treating cancer using the anti-ENTPD2 antibodies which are disclosed herein.

In one embodiment of the invention, there is provided an antibody molecule that binds to human ENTPD2 for use in treating a cancer in a subject, whereby the antibody molecule is administered in a step-up dosing regime such that in the main phase the antibody molecule dose is administered at a main dose amount according to a main dosing period with a main dosing period frequency, which is preceded by an initial phase in which said antibody molecule is administered at an equal or higher dosing period frequency than the main dosing period frequency and is administered via fractionated dose amounts of the main dose amount, wherein in the initial phase within a time period equal to the main dosing period the fractionated dose amounts summed together shall not exceed the amount of the main phase dose amount.

In another embodiment of the invention, there is provided a method of treating cancer in a subject comprising administering to the subject an antibody molecule that binds to human ENTPD2 in an amount effective to treat the cancer, whereby the antibody molecule is administered in a step-up dosing regime such that in the main phase the antibody molecule dose is administered at a main dose amount according to a main dosing period with a main dosing period frequency, which is preceded by an initial phase in which said antibody molecule is administered at an equal or higher dosing period frequency than the main dosing period frequency and is administered via fractionated dose amounts of the main dose amount, wherein in the initial phase within a time period equal to the main dosing period the fractionated dose amounts summed together shall not exceed the amount of the main phase dose amount.

In an embodiment of the anti-ENTPD2 antibody for use according to the invention or method of treatment involving the anti-ENTPD2 antibody according to the invention, the antibody molecule comprises:
(i) a heavy chain variable region (VH) comprising a heavy chain complementarity determining region 1 (VHCDR1) amino acid sequence of SEQ ID NO: 401, a VHCDR2 amino acid sequence of SEQ ID NO: 402, and a VHCDR3 amino acid sequence of SEQ ID NO: 403; and
(ii) a light chain variable region (VL) comprising a light chain complementarity determining region 1 (VLCDR1) amino acid sequence of SEQ ID NO: 414, a VLCDR2 amino acid sequence of SEQ ID NO: 415, and a VLCDR3 amino acid sequence of SEQ ID NO: 416.

In an embodiment of the anti-ENTPD2 antibody for use according to the invention or method of treatment involving the anti-ENTPD2 antibody according to the invention, the antibody molecule comprises: a heavy chain variable region (VH) comprising SEQ ID NO: 410 or a sequence at least about 95% or more identical thereto, and a light chain variable region (VL) comprising SEQ ID NO: 421 or a sequence at least about 95% or more identical thereto.

In an embodiment of the anti-ENTPD2 antibody for use according to the invention or method of treatment involving the anti-ENTPD2 antibody according to the invention, the antibody molecule comprises: a heavy chain comprising SEQ ID NO: 412 or a sequence at least about 95% or more identical thereto, and a light chain comprising SEQ ID NO: 423 or a sequence at least about 95% or more identical thereto.

In an embodiment of the antibody for use according to the invention or method according to the invention, the initial phase is used to prevent or reduce the incidence or severity of cytokine release syndrome (CRS). The antibody for use or method in such an embodiment is an anti-ENTPD2 antibody.

The following embodiments relate to the dosage regime of the anti-ENTPD2 antibody. In an embodiment of the antibody for use according to the invention or method according to the invention, the main dose amount is 300 mg, 600 mg, 1200 mg, or 2400 mg, and/or the main dose frequency is Q2W. In an embodiment of the antibody for use according to the invention or method according to the invention, in the initial phase the antibody molecule is administered at a frequency of QW or Q2W. In an embodiment of the antibody for use according to the invention or method according to the invention, in the initial phase the fractionated dose amount is 100 mg. In an embodiment of the antibody for use according to the invention or method according to the invention, in the initial phase the antibody molecule is administered once or twice within two weeks. In an embodiment of the antibody for use according to the invention or method according to the invention, the antibody molecule is administered in the initial phase on day 1 at about 100 mg, followed by the main phase beginning on day 15 with about 300 mg, and continuing thereafter with about 300 mg administered Q2W. In an embodiment of the antibody for use according to the invention or method according to the invention, the antibody molecule is administered in the initial phase on day 1 at about 100 mg and on day 8 at about 100 mg, followed by the main phase beginning on day 15 with about 300 mg, and continuing thereafter with about 300 mg administered Q2W. In an embodiment of the antibody for use according to the invention or method according to the invention, the antibody molecule is administered to the subject intravenously as a 1 hr infusion (up to 2 hours if clinically indicated).

In an embodiment of the antibody for use according to the invention or method according to the invention, the initial phase of the dosing of the anti-ENTPD2 antibody is two weeks.

In an embodiment the main dose frequency of the anti-ENTPD2 antibody is Q2W.

In an embodiment of the antibody for use according to the invention or method according to the invention, the antibody molecule is administered in combination with one or more therapeutic agents or procedures.

The following embodiments relate to the dosage regime of the anti-ENTPD2 antibody and combination agents administered together with the anti-ENTPD2 antibody. In an embodiment of the antibody for use according to the invention or method according to the invention, the antibody molecule is administered in combination with a PD-1 inhibitor. In one embodiment, the PD-1 inhibitor is selected from the group consisting of Tislelizumab, Spartalizumab, Nivolumab, Pembrolizumab, Pidilizumab, MEDI0680, REGN2810, TSR-042, PF-06801591, and AMP-224. In one embodiment, the PD-1 inhibitor is an anti-PD-1 antibody molecule, wherein the anti-PD-1 antibody molecule is administered at a dose of about 400 mg Q4W.

The following embodiments relate to the dosage regime of the anti-ENTPD2 antibody and combination agents administered together with the anti-ENTPD2 antibody. In an embodiment of the antibody for use according to the invention or method according to the invention, the antibody molecule is administered in combination with an adenosine A2AR antagonist. In one embodiment, (i) the adenosine A2AR antagonist is selected from the group consisting of PBF509, CP1444, AZD4635, Vipadenant, GBV-2034, and AB928; or (ii) the adenosine A2AR antagonist is selected from the group consisting of 5-bromo-2,6-di-(1H-pyrazol-1-yl)pyrimidine-4-amine; (S)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine; (R)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine, or racemate thereof; 7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine; and 6-(2-chloro-6-methylpyridin-4-yl)-5-(4-fluorophenyl)-1,2,4-triazin-3-amine; preferably wherein the adenosine A2AR antagonist is PBF509. In one embodiment, the adenosine A2AR antagonist is administered at a dose of about 80 mg, about 160 mg, 240 mg or about 320 mg, preferably wherein the adenosine A2AR antagonist is administered at a dose of about 160 mg twice a day (BID).

The following embodiments relate to the dosage regime of the anti-ENTPD2 antibody and combination agents administered together with the anti-ENTPD2 antibody. In an embodiment of the antibody for use according to the invention or method according to the invention, the antibody molecule is administered in combination with an anti-human CD73 antibody. In one embodiment, the anti-human CD73 antibody comprises: (i) a heavy chain variable region (VH) comprising a heavy chain complementarity determining region 1 (VHCDR1) amino acid sequence of SEQ ID NO: 88, a VHCDR2 amino acid sequence of SEQ ID NO: 89, and a VHCDR3 amino acid sequence of SEQ ID NO: 37; and (ii) a light chain variable region (VL) comprising a light chain complementarity determining region 1 (VLCDR1) amino acid sequence of SEQ ID NO: 48, a VLCDR2 amino acid sequence of SEQ ID NO: 49, and a VLCDR3 amino acid sequence of SEQ ID NO: 50. In one embodiment, the anti-human CD73 antibody comprises: a heavy chain variable region comprising the amino acid sequence of SEQ ID NO: 44, or an amino acid sequence having at least about 85%, 90%, 95%, or 99% sequence identity to SEQ ID NO: 44 and/or a light chain variable region comprising the amino acid sequence of SEQ ID NO: 55, or an amino acid sequence having at least about 85%, 90%, 95%, or 99% sequence identity to SEQ ID NO: 55. In one embodiment, the anti-human CD73 antibody comprises: a heavy chain comprising the amino acid sequence of SEQ ID NO: 46, or an amino acid sequence having at least about 85%, 90%, 95%, or 99% sequence identity to SEQ ID NO: 46, wherein X in SEQ ID NO: 46 is K and/or a light chain comprising the amino acid sequence of SEQ ID NO: 57, or an amino acid sequence having at least about 85%, 90%, 95%, or 99% sequence identity to SEQ ID NO: 57.

In an embodiment of the antibody for use according to the invention or method according to the invention, the cancer is MSS colorectal cancer (CRC), cholangiocarcinoma (intrahepatic or extrahepatic), pancreatic cancer, esophageal cancer, esophageal gastric junction (EGJ) cancer, or gastric cancer.

In an embodiment of the antibody for use according to the invention or method according to the invention, the antibody molecule that binds to human ENTPD2 is in the form of a pharmaceutical composition comprising the antibody molecule as defined in claim 33 and a pharmaceutically acceptable carrier, excipient or stabilizer.

In an embodiment of the antibody for use according to the invention or method according to the invention, the antibody molecule that binds to human ENTPD2 is in the form of a pharmaceutical composition comprising the anti-ENTPD2 antibody as disclosed herein and a pharmaceutically acceptable carrier, excipient or stabilizer.

In an aspect of the invention, both the anti-CD73 antibody and the anti-ENTPD2 antibody are administered in step-up dosing regimes as described herein. In an embodiment both the anti-CD73 antibody and the anti-ENTPD2 antibody are administered in step-up dosing regimes such that
a) in the main phase of the dosing of the anti-CD73 antibody molecule the dose is administered at a main dose amount of the anti-CD73 antibody according to a main dosing period and a main dosing period frequency of the anti-CD73 antibody, which is preceded by an initial phase in which said anti-CD73 antibody molecule is administered at a higher dosing period frequency than the main dosing period frequency of the anti-CD73 antibody and is administered via fractionated dose amounts of the main dose amount of the anti-CD73 antibody, wherein in the initial phase within a time period equal to the main dosing period of the anti-CD73 antibody the fractionated dose amounts of the anti-CD73 antibody summed together shall not exceed the amount of the main phase dose amount of the anti-CD73 antibody; and
b) in the main phase of the dosing of the anti-ENTPD2 antibody molecule the dose is administered at a main dose amount of the anti-ENTPD2 antibody according to a main dosing period and a main dosing period frequency of the anti-ENTPD2 antibody, which is preceded by an initial phase in which said anti-ENTPD2 antibody molecule is administered at a higher dosing period frequency than the main dosing period frequency of the anti-ENTPD2 antibody and is administered via fractionated dose amounts of the main dose amount of the anti-ENTPD2 antibody, wherein in the initial phase within a time period equal to the main dosing period of the anti-ENTPD2 antibody the fractionated dose amounts of the anti-ENTPD2 antibody summed together shall not exceed the amount of the main phase dose amount of the anti-ENTPD2 antibody.

In an embodiment both the anti-CD73 antibody and the anti-ENTPD2 antibody are administered in step-up dosing regimes such that
a) the anti-CD73 antibody is administered in the initial phase of two weeks once at about 200 mg in the first week and once at about 400 mg in the second week, followed by the main phase with about 600 mg administered Q2W thereafter and
b) the anti-ENTPD2 antibody is administered in the initial phase of two weeks either i) once at about 100 mg or ii) 100 mg in the first week and once at about 100 mg in the second week, followed by the main phase with about 300 mg administered Q2W.

### BRIEF DESCRIPTION OF THE TABLES

Table 1 provides amino acid and nucleotide sequences for exemplary anti-CD73 antibodies.

Table 2 provides consensus CDR sequences for exemplary anti-CD73 antibodies.

Table 3 provides amino acid sequences of human IgG heavy chains and human kappa light chain.

Table 4 provides exemplary sequences of CD73.

Tables 5 and 6 provide amino acid and/or nucleotide sequences of exemplary anti-PD-1 antibody molecules.

Tables 7 and 8 provide amino acid and/or nucleotide sequences of exemplary anti-PD-L1 antibody molecules.

Table 9 provides amino acid and nucleotide sequences for exemplary anti-ENTPD2 antibodies.

Table 10 provides nomenclatures for two lineages of anti-CD73 antibodies.

Table 11 provides affinities of anti-CD73 antibodies.

Table 12 provides affinities of anti-CD73 Fabs.

Table 13 provides provisional dose levels for anti-CD73 antibody 373.A.

Table 14 provides provisional dose levels for anti-CD73 antibody 373.A in combination with PBF509.

Table 15 provides provisional dose levels for anti-CD73 antibody 373.A in combination with Spartalizumab.

Table 16 provides provisional dose levels for PBF509 in combination with anti-CD73 antibody 373.A and Spartalizumab.

Table 17 provides corresponding germline sequences of anti-CD73 antibodies.

Table 18 provides a list of the Investigational drugs.

Table 19 describes the starting dose and the provisional dose levels for anti-ENTPD2 mAb1 Schedule 1.

Table 20 describes the provisional dose levels for anti-ENTPD2 mAb1 Schedule 2 and Schedule 3.

Table 21 describes the provisional dose levels for anti-ENTPD2 mAb1 in combination with Spartalizumab.

Table 22 describes the provisional dose levels for anti-ENTPD2 mAb1 in combination with NIR178.

Table 23 describes the provisional dose levels for anti-ENTPD2 mAb1 in combination with an anti-CD73 antibody.

### DETAILED DESCRIPTION

The term “CD73” as used herein refers to “Cluster of Differentiation 73,” also known as 5′-nucleotidase (5′-NT) or ecto-5′-nucleotidase. The term “CD73” includes mutants, fragments, variants, isoforms, and homologs of full-length wild-type CD73. In one embodiment, the protein CD73 is encoded by the NT5E gene. Exemplary CD73 sequences are available at the Uniprot database under accession numbers Q6NZX3 and P21589. Exemplary immature CD73 amino acid sequences are provided as SEQ ID NOs: 105-107. A “CD73 monomer” refers to a polypeptide comprising an extracellular domain of CD73. In one embodiment, a CD73 monomer is a full-length CD73. A “CD73 dimer” refers to two polypeptides (e.g., two non-covalently associated polypeptides) consisting of two CD73 monomers (e.g., two identical CD73 monomers) interacting with each other to form a stable dimer, e.g., a dimer formed via protein-protein interactions between the C-terminal domains of the CD73 monomers. In one embodiment, the CD73 dimer is a naturally-occurring CD73 dimer.

Without wishing to be bound by theory, human CD73 has two domains. A conserved N-terminal domain (corresponding to approximately residues 29-310 of SEQ ID NO: 105) and a conserved C-terminal domain (corresponding to approximately residues 343-513 of SEQ ID NO: 105), which are linked by a single alpha-helix (corresponding to approximately residues 318-336 of SEQ ID NO: 105). The active site is detected primarily in the closed conformation and is formed between C- and N-terminal domains. For enzyme catalysis, a domain motion of −100° of the N-terminal domain with respect to the C-terminal domain can enable substrate binding and release, which occurs in the open (catalytic inactive) conformation. Human CD73 forms a dimer through protein-protein interactions between C-terminal domains. The buried surface area as well as the molecular interactions at the dimer interface are significantly different between active and inactive conformations of the enzyme. See, e.g., Knapp K, et al., Structure 20:2161-73 (2012), incorporated herein by reference in its entirety.

Disclosed herein are antibody molecules that bind to CD73 with high affinity and specificity. In one embodiment, disclosed herein are human antibodies that bind to CD73. In one embodiment, disclosed herein are antibody molecules that are capable of inhibiting or reducing the enzymatic activity of CD73, e.g., human CD73, e.g., soluble human CD73 or membrane-bound human CD73. In one embodiment, disclosed herein are antibody molecules that are capable of inhibiting or reducing CD73-mediated conversion of adenosine monophosphate (AMP) to adenosine. The anti-CD73 antibody molecules disclosed herein can be used to treat, prevent and/or diagnose cancerous or malignant disorders, e.g., solid and liquid tumors, e.g., lung cancer (e.g., non-small cell lung cancer), pancreas cancer (e.g., pancreatic ductal adenocarcinoma), breast cancer (e.g., triple-negative breast cancer), melanoma, head and neck cancer (e.g., squamous head and neck cancer), colorectal cancer (e.g., microsatellite stable (MSS) colorectal cancer), ovarian cancer, metastatic castration resistant prostate cancer or renal cancer (e.g., renal cell carcinoma). In an embodiment of the invention the cancer to be treated is non-small cell lung cancer, pancreatic ductal adenocarcinoma, triple-negative breast cancer, microsatellite stable (MSS) colorectal cancer, metastatic castration resistant prostate cancer, ovarian cancer or renal cell carcinoma.

As used herein, ectonucleoside triphosphate diphosphohydrolase 2 (ENTPD2) (also known as CD39 Antigen-Like 1, CD39-like-1, CD39L1, Ecto-ATP Diphosphohydrolase 2, ecto-ATPase, Ecto-ATPase 2, Ecto-ATPDase 2, NTPDase-2, NTPDase 2) refers to the type 2 enzyme of the ecto-nucleoside triphosphate diphosphohydrolase family (E-NTPDase), which are a family of ecto-nucleosidases that hydrolyze 5′-triphosphates. The ENTPD2 enzyme is encoded by the gene ENTPD2. The human ENTPD2 gene is mapped to chromosomal location 9q34.3, and the genomic sequence of human ENTPD2 gene can be found in GenBank at NC_000009.12. The mRNA and protein sequences of the ENTPD2 human transcript variants can be found in GenBank with the following Accession Nos:
Isoform1: NM_203468.2 (mRNA)→NP_982293.1 (protein with 495 aa);
Isoform2: NM_001246.3 (mRNA)→NP_001237.1 (protein with 472 aa);

```
Ectonucleoside triphosphate diphosphohydrolase 2 isoform 1
[Homo sapiens, NP_982293.1]
(SEQ ID NO: 923)
MAGKVRSLLPPLLLAAAGLAGLLLLCVPTRDVREPPALKYGIVLDAGSSH
TSMFIYKWPADKENDTGIVGQHSSCDVPGGGISSYADNPSGASQSLVGCL
EQALQDVPKERHAGTPLYLGATAGMRLLNLTNPEASTSVLMAVTHTLTQY
PFDFRGARILSGQEEGVFGWVTANYLLENFIKYGWVGRWFRPRKGTLGAM
DLGGASTQITFETTSPAEDRASEVQLHLYGQHYRVYTHSFLCYGRDQVLQ
RLLASALQTHGFHPCWPRGFSTQVLLGDVYQSPCTMAQRPQNFNSSARVS
LSGSSDPHLCRDLVSGLFSFSSCPFSRCSFNGVFQPPVAGNFVAFSAFFY
TVDFLRTSMGLPVATLQQLEAAAVNVCNQTWAQLQARVPGQRARLADYCA
GAMFVQQLLSRGYGFDERAFGGVIFQKKAADTAVGWALGYMLNLTNLIPA
DPPGLRKGTDFSSWVVLLLLFASALLAALVLLLRQVHSAKLPSTI
Homo sapiens ectonucleoside triphosphate diphosphohydrolase 2
(ENTPD2), transcript variant 1, mRNA [NM_203468.2]
(SEQ ID NO: 924)
      1 ggctccccgc actctccggg tccacgcatc gtcctcccgc gcgcccgccc gcccatggcc
    61 gggaaggtgc ggtcactgct gccgccgctg ctgctggccg ccgcgggcct cgccggcctc
  121 ctactgctgt gcgtccccac ccgcgacgtc cgggagccgc ccgccctcaa gtatggcatc
  181 gtcctggacg ctggttcttc acacacgtcc atgtttatct acaagtggcc ggcagacaag
  241 gagaacgaca caggcattgt gggccagcac agctcctgtg atgttccagg tgggggcatc
  301 tccagctatg cagacaaccc ttctggggcc agccagagtc ttgttggatg cctcgaacag
  361 gcgcttcagg atgtgcccaa agagagacac gcgggcacac ccctctacct gggagccaca
  421 gcgggtatgc gcctgctcaa cctgaccaat ccagaggcct cgaccagtgt gctcatggca
  481 gtgactcaca cactgaccca gtaccccttt gacttccggg gtgcacgcat cctctcgggc
  541 caggaagagg gggtgtttgg ctgggtgact gccaactacc tgctggagaa cttcatcaag
  601 tacggctggg tgggccggtg gttccggcca cggaagggga cactgggggc catggacctg
  661 gggggtgcct ctacccagat cacttttgag acaaccagtc cagctgagga cagagccagc
  721 gaggtccagc tgcatctcta cggccagcac taccgagtct acacccacag cttcctctgc
  781 tatggccgtg accaggtcct ccagaggctg ctggccagcg ccctccagac ccacggcttc
  841 cacccctgct ggccgagggg cttttccacc caagtgctgc tcggggatgt gtaccagtca
  901 ccatgcacca tggcccagcg gccccagaac ttcaacagca gtgccagggt cagcctgtca
  961 gggagcagtg acccccacct ctgccgagat ctggtttctg ggctcttcag cttctcctcc
1021 tgccccttct cccgatgctc tttcaatggg gtcttccagc ccccagtggc tgggaacttt
1081 gtggccttct ctgccttctt ctacactgtg gactttttgc ggacttcgat ggggctgccc
1141 gtggccaccc tgcagcagct ggaggcagcc gcagtgaatg tctgcaacca gacctgggct
1201 cagctgcaag ctcgggtgcc agggcaacgg gcccgcctgg ccgactactg cgccggggcc
1261 atgttcgtgc agcagctgct gagtcgcggc tacggcttcg acgagcgcgc cttcggcggc
1321 gtgatcttcc agaagaaggc cgcggacact gcagtgggct gggcgctcgg ctacatgctg
1381 aacctgacca acctgatccc cgccgacccg ccggggctgc gcaagggcac agacttcagc
1441 tcctgggtcg tcctcctgct gctcttcgcc tccgcgctcc tggctgcgct tgtcctgctg
1501 ctgcgtcagg tgcactccgc caagctgcca agcaccattt aggggccgac gggggcagct
1561 gccccatccc tcccccaacc cctgtatccc caccccgtac tcccacccct cccacaaccc
1621 ctgtacctcc cacccctgta tccccacccc tecacccacc cctctcccaa cctctetccc
1681 cgcccctgta tcctgcattc ctccacccac cctctatccc ccaccgctcc accccaccac
1741 tgtcttctcc atccttccac cccaccctca gcgtctctgc ccctaaggca gcccaggaaa
1801 taggaactga gactctggta cccacaggag cctgggtggg caaagagcgc tcaatccagc
1861 tccttgaacc cctccagccc gcttcagcct gggcatcact gcaggccccg tgctcctcct
1921 cctcctcctc agggctgggt ctccagagag tggggccttg gtcctgagaa tcagccctta
1981 gaggctcctt ctgtgtagtc tgggtctgta ctggggaggg tcacagccca cgggctggca
2041 gccagcccag cacctacttg taaaaatttt gtaataaaaa gtttttccta gagacgtgaa
2101 aaaaaaaaaa aaaaaaaaaa aaaaaaaaaa aaaaaaaaaa aaaaaaaaaa aaaaaa
Ectonucleoside triphosphate diphosphohydrolase 2 isoform 2
[Homo sapiens, NP_001237.1]
(SEQ ID NO: 925)
MAGKVRSLLPPLLLAAAGLAGLLLLCVPTRDVREPPALKYGIVLDAGSSH
TSMFIYKWPADKENDTGIVGQHSSCDVPGGGISSYADNPSGASQSLVGCL
EQALQDVPKERHAGTPLYLGATAGMRLLNLTNPEASTSVLMAVTHTLTQY
PFDFRGARILSGQEEGVFGWVTANYLLENFIKYGWVGRWFRPRKGTLGAM
DLGGASTQITFETTSPAEDRASEVQLHLYGQHYRVYTHSFLCYGRDQVLQ
RLLASALQTHGFHPCWPRGFSTQVLLGDVYQSPCTMAQRPQNFNSSARVS
LSGSSDPHLCRDLVSGLFSFSSCPFSRCSFNGVFQPPVAGNFVAFSAFFY
TVDFLRTSMGLPVATLQQLEAAAVNVCNQTWAQQLLSRGYGFDERAFGGV
IFQKKAADTAVGWALGYMLNLTNLIPADPPGLRKGTDFSSWVVLLLLFAS
ALLAALVLLLRQVHSAKLPSTI
Homo sapiens ectonucleoside triphosphate diphosphohydrolase 2
(ENTPD2), transcript variant 2, mRNA [NM_001246.3]
(SEQ ID NO: 926)
      1 ggctccccgc actctccggg tccacgcate gtcctcccgc gcgcccgccc gcccatggcc
    61 gggaaggtgc ggtcactgct gccgccgctg ctgctggccg ccgcgggcct cgccggcctc
  121 ctactgetgt gcgtccccac ccgcgacgtc cgggagccgc ccgccctcaa gtatggcatc
  181 gtcctggacg ctggttcttc acacacgtcc atgtttatct acaagtggcc ggcagacaag
  241 gagaacgaca caggcattgt gggccagcac agctcctgtg atgttccagg tgggggcatc
  301 tccagctatg cagacaaccc ttctggggcc agccagagtc ttgttggatg cctcgaacag
  361 gcgcttcagg atgtgcccaa agagagacac gcgggcacac ccctctacct gggagccaca
  421 gcgggtatgc gcctgctcaa cctgaccaat ccagaggcct cgaccagtgt gctcatggca
  481 gtgactcaca cactgaccca gtaccccttt gacttccggg gtgcacgcat cctctcgggc
  541 caggaagagg gggtgtttgg ctgggtgact gccaactacc tgctggagaa cttcatcaag
  601 tacggctggg tgggccggtg gttccggcca cggaagggga cactgggggc catggacctg
  661 gggggtgcct ctacccagat cacttttgag acaaccagtc cagctgagga cagagccagc
  721 gaggtccagc tgcatctcta cggccagcac taccgagtct acacccacag cttcctctgc
  781 tatggccgtg accaggtcct ccagaggctg ctggccagcg ccctccagac ccacggcttc
  841 cacccctgct ggccgagggg cttttccacc caagtgctgc tcggggatgt gtaccagtca
  901 ccatgcacca tggcccagcg gccccagaac ttcaacagca gtgccagggt cagcctgtca
  961 gggagcagtg acccccacct ctgccgagat ctggtttctg ggctcttcag cttctcctcc
1021 tgccccttct cccgatgctc tttcaatggg gtcttccage ccccagtggc tgggaacttt
1081 gtggccttct ctgccttctt ctacactgtg gactttttgc ggacttcgat ggggctgccc
1141 gtggccaccc tgcagcagct ggaggcagcc gcagtgaatg tctgcaacca gacctgggct
1201 cagcagctgc tgagtcgcgg ctacggcttc gacgagegcg ccttcggcgg cgtgatcttc
1261 cagaagaagg ccgcggacac tgcagtgggc tgggcgctcg gctacatgct gaacctgacc
1321 aacctgatcc ccgccgaccc gccggggctg cgcaagggca cagacttcag ctcctgggtc
1381 gtcctcctgc tgctcttcgc ctccgcgctc ctggctgcgc ttgtcctgct gctgcgtcag
1441 gtgcactccg ccaagctgcc aagcaccatt taggggccga cgggggcagc tgccccatcc
1501 ctcccccaac ccctgtatcc ccaccccgta ctcccacccc tcccacaacc cctgtacctc
1561 ccacccctgt atccccaccc ctccacccac ccctctccca acctctctcc ccgcccctgt
1621 atcctgcatt cctccaccca ccctctatcc cccaccgctc caccccacca ctgtcttctc
1681 catccttcca ccccaccctc agcgtctctg cccctaaggc agcccaggaa ataggaactg
1741 agactctggt acccacagga gcctgggtgg gcaaagagcg ctcaatccag ctccttgaac
1801 ccctccagcc cgcttcagcc tgggcatcac tgcaggcccc gtgctcctcc tectectect
1861 cagggctggg tctccagaga gtggggcctt ggtcctgaga atcagccctt agaggctcct
1921 tctgtgtagt ctgggtctgt actggggagg gtcacagccc acgggctggc agccagccca
1981 gcacctactt gtaaaaattt tgtaataaaa agtttttcct agagacgtga aaaaaaaaaa
2041 aaaaaaaaaa aaaaaaaaaa aaaaaaaaaa aaaaaaaaaa aaaaaaa
```

As used herein, human ENTPD2 protein also encompasses proteins that have over its full length at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with any one of the ENTPD2 isoforms. The sequences of murine, Cynomolgus monkey, and other animal ENTPD2 proteins are known in the art.

Additional terms are defined below and throughout the application.

As used herein, the articles “a” and “an” refer to one or to more than one (e.g., to at least one) of the grammatical object of the article. As used herein, “plurality” means two or more.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The compositions and methods disclosed herein encompass polypeptides and nucleic acids having the sequences specified, or sequences substantially identical or similar thereto, e.g., sequences having at least about 85%, 90%, 95%, 97% or 99% sequence identity to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%, 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, e.g., at least 40%, 50%, 60%, e.g., at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a some embodiments, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In certain embodiments, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. One suitable set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid as described herein. BLAST protein searches can be performed with the XBLAST program, score=50, wordlength=3 to obtain amino acid sequences homologous to protein molecules described herein. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are suitable conditions and the ones that should be used unless otherwise specified.

It is understood that the molecules used in the dosage regimes of the invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide,” “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs.

The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a nonnatural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

### Antibody Molecules

In one embodiment of the dosage regimes according to the invention, the antibody molecule binds to a mammalian, e.g., human, CD73. For example, the antibody molecule binds to an epitope, e.g., linear or conformational epitope, e.g., an epitope as described herein, on CD73.

In another embodiment of the dosage regimes according to the invention, the antibody molecule binds to a mammalian, e.g., human, ENTPD2. For example, the antibody molecule binds to an epitope, e.g., linear or conformational epitope, e.g., an epitope as described herein, on ENTPD2.

As used herein, the term “antibody molecule” refers to a protein, e.g., an immunoglobulin chain or fragment thereof, comprising at least one immunoglobulin variable domain sequence. The term “antibody molecule” includes, for example, a monoclonal antibody (including a full length antibody which has an immunoglobulin Fc region). In an embodiment, an antibody molecule comprises a full length antibody, or a full length immunoglobulin chain. In an embodiment, an antibody molecule comprises an antigen binding or functional fragment of a full length antibody, or a full length immunoglobulin chain.

As used herein, an antibody molecule “binds to” an antigen as such binding is understood by one skilled in the art. In one embodiment, an antibody binds to an antigen with a dissociation constant (K_{D}) of about 1×10⁻³ M or less, 1×10⁻⁴ M or less, or 1×10⁻⁵ M or less.

In an embodiment, an antibody molecule is a monospecific antibody molecule and binds a single epitope, e.g., a monospecific antibody molecule having a plurality of immunoglobulin variable domain sequences, each of which binds the same epitope.

In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., different proteins (or different subunits of a multimeric protein). In an embodiment, a multispecific antibody molecule comprises a third, fourth or fifth immunoglobulin variable domain. In an embodiment, a multispecific antibody molecule is a bispecific antibody molecule, a trispecific antibody molecule, or tetraspecific antibody molecule.

In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., different proteins (or different subunits of a multimeric protein). In an embodiment, a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody molecule comprises a half antibody having binding specificity for a first epitope and a half antibody having binding specificity for a second epitope. In an embodiment, a bispecific antibody molecule comprises a half antibody, or fragment thereof, having binding specificity for a first epitope and a half antibody, or fragment thereof, having binding specificity for a second epitope. In an embodiment, a bispecific antibody molecule comprises a scFv, or fragment thereof, have binding specificity for a first epitope and a scFv, or fragment thereof, have binding specificity for a second epitope.

In an embodiment, an antibody molecule comprises a diabody, and a single-chain molecule, as well as an antigen-binding fragment of an antibody (e.g., Fab, F(ab′)₂, and Fv).

For example, an antibody molecule can include a heavy (H) chain variable domain sequence (abbreviated herein as VH), and a light (L) chain variable domain sequence (abbreviated herein as VL). In an embodiment, an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. A preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments may be obtained using any suitable method, including conventional techniques known to those with skill in the art, and the fragments can be screened for utility in the same manner as are intact antibodies.

The term “antibody” includes intact molecules as well as functional fragments thereof. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

The antibodies disclosed herein can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. In an embodiment, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region” and “CDR” as used herein refer to the sequences of amino acids within antibody variable regions which confer antigen specificity and binding affinity. In some embodiments, there are three CDRs in each heavy chain variable region (HCDR1, HCDR2, and HCDR3) and three CDRs in each light chain variable region (LCDR1, LCDR2, and LCDR3).

The precise amino acid sequence boundaries of a given CDR can be determined using any of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme). As used herein, the CDRs defined according to the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

Under all definitions, each VH and VL typically includes three CDRs and four FRs, arranged from amino-terminus to carboxy-terminus in the following order: FR1, CDR1, FR2, CDR2, FR3, CDR3, FR4.

Generally, unless specifically indicated, the anti-CD73 and anti-ENTPD2 antibody molecules can include any combination of one or more Kabat CDRs, Chothia CDRs, combination of Kabat and Chothia CDRs, IMGT CDRs, and/or an alternative definition, e.g., described in Table 1 and Table 9.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding site” refers to the part of an antibody molecule that comprises determinants that form an interface that binds to a CD73 polypeptide or an ENTPD2 polypeptide, or an epitope thereof. With respect to proteins (or protein mimetics), the antigen-binding site typically includes one or more loops (of at least, e.g., four amino acids or amino acid mimics) that form an interface that binds to a CD73 polypeptide or an ENTPD2 polypeptide. Typically, the antigen-binding site of an antibody molecule includes at least one or two CDRs and/or hypervariable loops, or more typically at least three, four, five or six CDRs and/or hypervariable loops.

As used herein, the term “Eu numbering” refers to the Eu numbering convention for the constant regions of an antibody, as described in Edelman, G. M. et al., Proc. Natl. Acad. USA, 63, 78-85 (1969) and Kabat et al., in “Sequences of Proteins of Immunological Interest”, U.S. Dept. Health and Human Services, 5th edition, 1991.

The terms “compete” or “cross-compete” are used interchangeably herein to refer to the ability of an antibody molecule to interfere with binding of an anti-CD73 or an anti-ENTPD2 antibody molecule, e.g., an anti-CD73 or an anti-ENTPD2 antibody molecule provided herein, to a target, e.g., human CD73 or human ENTPD2. The interference with binding can be direct or indirect (e.g., through an allosteric modulation of the antibody molecule or the target). The extent to which an antibody molecule is able to interfere with the binding of another antibody molecule to the target, and therefore whether it can be said to compete, can be determined using a competition binding assay, for example, a flow cytometry assay, an ELISA or BIACORE assay. In some embodiments, a competition binding assay is a quantitative competition assay. In some embodiments, a first anti-CD73 or an anti-ENTPD2 antibody molecule is said to compete for binding to the target with a second anti-CD73 or an anti-ENTPD2 antibody molecule when the binding of the first antibody molecule to the target is reduced by 10% or more, e.g., 20% or more, 30% or more, 40% or more, 50% or more, 55% or more, 60% or more, 65% or more, 70% or more, 75% or more, 80% or more, 85% or more, 90% or more, 95% or more, 98% or more, 99% or more in a competition binding assay (e.g., a competition assay described herein).

As used herein, the term “epitope” refers to the moieties of an antigen (e.g., human CD73 or human ENTPD2) that specifically interact with an antibody molecule. Such moieties, also referred to herein as epitopic determinants, typically comprise, or are part of, elements such as amino acid side chains or sugar side chains. An epitopic determinant can be defined by methods known in the art or disclosed herein, e.g., by crystallography or by hydrogen-deuterium exchange. At least one or some of the moieties on the antibody molecule that specifically interact with an epitopic determinant are typically located in a CDR(s). Typically, an epitope has a specific three dimensional structural characteristics. Typically, an epitope has specific charge characteristics. Some epitopes are linear epitopes while others are conformational epitopes.

In an embodiment, an epitopic determinant is a moiety on the antigen, e.g., such as amino acid side chain or sugar side chain, or part thereof, which, when the antigen and antibody molecule are co-crystallized, is within a predetermined distance, e.g., within 5 Angstroms, of a moiety on the antibody molecule, referred to herein as a “crystallographic epitopic determinant.” The crystallographic epitopic determinants of an epitope are collectively referred to as the “crystallographic epitope.”

A first antibody molecule binds the same epitope as a second antibody molecule (e.g., a reference antibody molecule, e.g., an antibody molecule disclosed herein) if the first antibody interacts with the same epitopic determinants on the antigen as does the second or reference antibody, e.g., when interaction is measured in the same way for both the antibody and the second or reference antibody. Epitopes that overlap share at least one epitopic determinant. A first antibody molecule binds an overlapping epitope with a second antibody molecule (e.g., a reference antibody molecule, e.g., an antibody disclosed herein) when both antibody molecules interact with a common epitopic determinant. A first and a second antibody molecule (e.g., a reference antibody molecule, e.g., an antibody molecule disclosed herein) bind substantially overlapping epitopes if at least half of the epitopic determinants of the second or reference antibody are found as epitopic determinants in the epitope of the first antibody. A first and a second antibody molecule (e.g., a reference antibody molecule, e.g., an antibody molecule disclosed herein) bind substantially the same epitope if the first antibody molecule binds at least half of the core epitopic determinants of the epitope of the second or reference antibody, wherein the core epitopic determinants are defined by, e.g., crystallography or hydrogen-deuterium exchange.

As used herein, an antibody molecule “reduces hydrogen-deuterium exchange” in an antigen fragment when the hydrogen-deuterium exchange in the antigen fragment in the presence of the antibody molecule is lower than the hydrogen-deuterium exchange in the antigen fragment in the absence of the antibody molecule, as measured in a hydrogen-deuterium exchange assay.

As used herein, a reduction in “the average hydrogen-deuterium exchange” is determined by the level of normalized hydrogen-deuterium exchange (Da per residue) in an antigen fragment in the absence of an antibody minus the level of normalized hydrogen-deuterium exchange (Da per residue) in the antigen fragment in the presence of the antibody.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

The antibody molecule can be a polyclonal or a monoclonal antibody. In other embodiments, the antibody can be recombinantly produced, e.g., produced by yeast display, phage display, or by combinatorial methods. Alternatively, such antibodies may be selected from synthetic yeast-based antibody presentation systems, such as those described in, e.g., Y. Xu et al, Addressing polyspecificity of antibodies selected from an in vitro yeast presentation system: a FACS-based, high-throughput selection and analytical tool. PEDS 26.10, 663-70 (2013); WO2009036379; WO2010105256; and WO2012009568, herein incorporated by reference in their entireties.

In one embodiment, the antibody is a fully human antibody (e.g., an antibody produced by yeast display, an antibody produced by phage display, or an antibody made in a mouse which has been genetically engineered to produce an antibody from a human immunoglobulin sequence), or a non-human antibody, e.g., a rodent (mouse or rat), goat, primate (e.g., monkey), or camel antibody. Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody can be one in which the variable region, or a portion thereof, e.g., the CDRs, are generated in a non-human organism, e.g., a rat or mouse. Chimeric, CDR-grafted, and humanized antibodies are within the antibodies useful in the dosage regimes of the invention. Antibodies generated in a non-human organism, e.g., a rat or mouse, and then modified, e.g., in the variable framework or constant region, to decrease antigenicity in a human are within the antibodies useful in the dosage regimes of the invention.

Antibodies can be produced by any suitable recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immunoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to CD73 or ENTPD2. In some embodiments, the donor is a rodent antibody, e.g., a rat or mouse antibody, and the recipient is a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, e.g., 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art (see e.g., Morrison, S. L., 1985, Science 229:1202-1207, by Oi et al., 1986, Bio Techniques 4:214, and by Queen et al. U.S. Pat. Nos. 5,585,089, 5,693,761 and 5,693,762, the contents of all of which are hereby incorporated by reference).

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference.

Also within the scope of the antibodies useful in the dosage regime of the invention are humanized antibodies in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFV) may be engineered (see, for example, Colcher, D. et al. (1999) Ann NY Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In some embodiments the antibody has effector function and can fix complement. In other embodiments the antibody does not recruit effector cells or fix complement. In certain embodiments, the antibody has reduced or no ability to bind an Fc receptor. For example, it may be an isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Amino acid mutations which stabilize antibody structure, such as S228P (Eu numbering) in human IgG4, are also contemplated. Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules used in the dosage regimes of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody molecule may be derivatized (or labeled) include fluorescent compounds, various enzymes, prosthetic groups, luminescent materials, bioluminescent materials, fluorescent emitting metal atoms, e.g., europium (Eu), and other anthanides, and radioactive materials (described below). Exemplary fluorescent detectable agents include fluorescein, fluorescein isothiocyanate, rhodamine, 5dimethylamine-1-napthalenesulfonyl chloride, phycoerythrin and the like. An antibody may also be derivatized with detectable enzymes, such as alkaline phosphatase, horseradish peroxidase, β-galactosidase, acetylcholinesterase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody molecule may also be derivatized with a prosthetic group (e.g., streptavidin/biotin and avidin/biotin). For example, an antibody may be derivatized with biotin, and detected through indirect measurement of avidin or streptavidin binding. Examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of bioluminescent materials include luciferase, luciferin, and aequorin.

Labeled antibody molecule can be used, for example, diagnostically and/or experimentally in a number of contexts, including (i) to isolate a predetermined antigen by standard techniques, such as affinity chromatography or immunoprecipitation; (ii) to detect a predetermined antigen (e.g., in a cellular lysate or cell supernatant) in order to evaluate the abundance and pattern of expression of the protein; (iii) to monitor protein levels in tissue as part of a clinical testing procedure, e.g., to determine the efficacy of a given treatment regimen.

An antibody molecule may be conjugated to another molecular entity, typically a label or a therapeutic (e.g., immunomodulatory, immunostimulatory, cytotoxic, or cytostatic) agent or moiety. Radioactive isotopes can be used in diagnostic or therapeutic applications. Radioactive isotopes that can be coupled to the anti-CD73 antibodies or anti-ENTPD2 antibodies include, but are not limited to α-, β-, or γ-emitters, or β- and γ-emitters. Such radioactive isotopes include, but are not limited to iodine (¹³¹I or ¹²⁵I), yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹ mTc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga). Radioisotopes useful as therapeutic agents include yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), and rhodium (¹⁸⁸Rh). Radioisotopes useful as labels, e.g., for use in diagnostics, include iodine (¹³¹I or ¹²⁵I), indium (¹¹¹In), technetium (⁹⁹mTc), phosphorus (³²P), carbon (¹⁴C), and tritium (³H), or one or more of the therapeutic isotopes listed above.

As is discussed above, the antibody molecule can be conjugated to a therapeutic agent. Therapeutically active radioisotopes have already been mentioned. Examples of other therapeutic agents include taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846, 545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclinies (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

In certain embodiments, the antibody molecule is a multi-specific (e.g., a bispecific or a trispecific) antibody molecule. Protocols for generating bispecific or heterodimeric antibody molecules are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhydryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH-CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also disclosed creating bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. Additional exemplary multispecific and bispecific molecules and methods of making the same are found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US2002004587A1, US2002076406A1, US2002103345A1, US2003207346A1, US2003211078A1, US2004219643A1, US2004220388A1, US2004242847A1, US2005003403A1, US2005004352A1, US2005069552A1, US2005079170A1, US2005100543A1, US2005136049A1, US2005136051A1, US2005163782A1, US2005266425A1, US2006083747A1, US2006120960A1, US2006204493A1, US2006263367A1, US2007004909A1, US2007087381A1, US2007128150A1, US2007141049A1, US2007154901A1, US2007274985A1, US2008050370A1, US2008069820A1, US2008152645A1, US2008171855A1, US2008241884A1, US2008254512A1, US2008260738A1, US2009130106A1, US2009148905A1, US2009155275A1, US2009162359A1, US2009162360A1, US2009175851A1, US2009175867A1, US2009232811 A1, US2009234105A1, US2009263392A1, US2009274649A1, EP346087A2, WO0006605A2, WO02072635A2, WO04081051A1, WO06020258A2, WO2007044887A2, WO2007095338A2, WO2007137760A2, WO2008119353A1, WO2009021754A2, WO2009068630A1, WO9103493A1, WO9323537A1, WO9409131A1, WO9412625A2, WO9509917A1, WO9637621A2, WO9964460A1. The contents of the above-referenced applications are incorporated herein by reference in their entireties.

In other embodiments, the anti-CD73 antibody molecule or anti-ENTPD2 antibody molecule (e.g., a monospecific, bispecific, or multispecific antibody molecule) is covalently linked, e.g., fused, to another partner e.g., a protein e.g., one, two or more cytokines, e.g., as a fusion molecule for example a fusion protein.

A “fusion protein” and a “fusion polypeptide” refer to a polypeptide having at least two portions covalently linked together, where each of the portions is a polypeptide having a different property. The property may be a biological property, such as activity in vitro or in vivo.

The property can also be simple chemical or physical property, such as binding to a target molecule, catalysis of a reaction, etc. The two portions can be linked directly by a single peptide bond or through a peptide linker, but are in reading frame with each other.

Disclosed herein is an isolated nucleic acid molecule encoding the above antibody molecule, vectors and host cells thereof. The nucleic acid molecule includes but is not limited to RNA, genomic DNA and cDNA.

Exemplary anti-CD73 Antibody Molecules

In one embodiment of the dosage regimes of the invention, the anti-CD73 antibody is an anti-CD73 antibody molecule as described in WO2018237157, published on 27 Dec. 2018, entitled “Antibody Molecules to CD73 and Uses Thereof,” which is incorporated by reference in its entirety.

In some embodiments, the anti-CD73 antibody molecule comprises at least one antigen-binding region, e.g., a variable region or an antigen-binding fragment thereof, from an antibody described herein, e.g., an antibody chosen from 918, 350, 356, 358, 930, 373, 374, 376, 377, 379, 363, 366, 407, 893, 939, 430, or 398; or as described in Table 1; or encoded by a nucleotide sequence in Table 1; or a sequence substantially identical (e.g., having at least about 80%, 85%, 90%, 92%, 95%, 97%, 98%, or 99% sequence identity) to any of the aforesaid sequences.

In some embodiments, the anti-CD73 antibody molecule comprises at least one or two heavy chain variable regions from an antibody described herein, e.g., an antibody chosen from 918, 350, 356, 358, 930, 373, 374, 376, 377, 379, 363, 366, 407, 893, 939, 430, or 398; or as described in Table 1; or encoded by a nucleotide sequence in Table 1; or a sequence substantially identical (e.g., having at least about 80%, 85%, 90%, 92%, 95%, 97%, 98%, or 99% sequence identity) to any of the aforesaid sequences.

In certain embodiments, the anti-CD73 antibody molecule comprises at least one or two light chain variable regions from an antibody described herein, e.g., an antibody chosen from 918, 350, 356, 358, 930, 373, 374, 376, 377, 379, 363, 366, 407, 893, 939, 430, or 398; or as described in Table 1; or encoded by a nucleotide sequence in Table 1; or a sequence substantially identical (e.g., having at least about 80%, 85%, 90%, 92%, 95%, 97%, 98%, or 99% sequence identity) to any of the aforesaid sequences.

In one embodiment, the anti-CD73 antibody molecule includes a heavy chain constant region of an IgG4, e.g., a human IgG4. In another embodiment, the human IgG4 includes a substitution (e.g., a Ser to Pro substitution) at position 228 according to Eu numbering. In still another embodiment, the anti-CD73 antibody molecule includes a heavy chain constant region of an IgG1, e.g., a human IgG1. In one embodiment, the human IgG1 includes a substitution (e.g., an Asn to Ala substitution) at position 297 according to Eu numbering. In one embodiment, the human IgG1 includes a substitution (e.g., an Asp to Ala substitution) at position 265 according to Eu numbering, a substitution (e.g., a Pro to Ala substitution) at position 329 according to Eu numbering, or both. In one embodiment, the human IgG1 includes a substitution (e.g., a Leu to Ala substitution) at position 234 according to Eu numbering, a substitution (e.g., a Leu to Ala substitution) at position 235 according to Eu numbering, or both. In one embodiment, the heavy chain constant region comprises an amino acid sequence set forth in Table 3, or a sequence substantially identical (e.g., having at least about 80%, 85%, 90%, 92%, 95%, 97%, 98%, or 99% sequence identity) thereto.

In yet another embodiment, the anti-CD73 antibody molecule includes a kappa light chain constant region, e.g., a human kappa light chain constant region. In one embodiment, the light chain constant region comprises an amino acid sequence set forth in Table 3, or a sequence substantially identical (e.g., having at least about 80%, 85%, 90%, 92%, 95%, 97%, 98%, or 99% sequence identity) thereto.

In another embodiment, the anti-CD73 antibody molecule includes a heavy chain constant region of an IgG4, e.g., a human IgG4, and a kappa light chain constant region, e.g., a human kappa light chain constant region, e.g., a heavy and light chain constant region comprising an amino acid sequence set forth in Table 3, or a sequence substantially identical (e.g., having at least about 80%, 85%, 90%, 92%, 95%, 97%, 98%, or 99% sequence identity) thereto. In yet another embodiment, the anti-CD73 antibody molecule includes a heavy chain constant region of an IgG1, e.g., a human IgG1, and a kappa light chain constant region, e.g., a human kappa light chain constant region, e.g., a heavy and light chain constant region comprising an amino acid sequence set forth in Table 3, or a sequence substantially identical (e.g., having at least about 80%, 85%, 90%, 92%, 95%, 97%, 98%, or 99% sequence identity) thereto. In one embodiment, the human IgG1 includes a substitution at position 297 according to Eu numbering (e.g., an Asn to Ala substitution). In one embodiment, the human IgG1 includes a substitution at position 265 according to Eu numbering, a substitution at position 329 according to Eu numbering, or both (e.g., an Asp to Ala substitution at position 265 and/or a Pro to Ala substitution at position 329). In one embodiment, the human IgG1 includes a substitution at position 234 according to Eu numbering, a substitution at position 235 according to Eu numbering, or both (e.g., a Leu to Ala substitution at position 234 and/or a Leu to Ala substitution at position 235).

In another embodiment, the anti-CD73 antibody molecule includes a heavy chain variable region and a constant region, a light chain variable region and a constant region, or both, comprising the amino acid sequence of 918, 350, 356, 358, 930, 373, 374, 376, 377, 379, 363, 366, 407, 893, 939, 430, or 398; or as described in Table 1; or encoded by a nucleotide sequence in Table 1; or a sequence substantially identical (e.g., having at least about 80%, 85%, 90%, 92%, 95%, 97%, 98%, or 99% sequence identity) to any of the aforesaid sequences.

In some embodiments, the anti-CD73 antibody molecule includes at least one, two, or three complementarity determining regions (CDRs) from a heavy chain variable region of an antibody described herein, e.g., an antibody chosen from 918, 350, 356, 358, 930, 373, 374, 376, 377, 379, 363, 366, 407, 893, 939, 430, or 398; or as described in Table 1, or encoded by a nucleotide sequence in Table 1; or a sequence substantially identical (e.g., having at least about 80%, 85%, 90%, 92%, 95%, 97%, 98%, or 99% sequence identity) to any of the aforesaid sequences.

In some embodiments, the anti-CD73 antibody molecule includes at least one, two, or three complementarity determining regions (CDRs) from a light chain variable region of an antibody described herein, e.g., an antibody chosen from 918, 350, 356, 358, 930, 373, 374, 376, 377, 379, 363, 366, 407, 893, 939, 430, or 398; or as described in Table 1, or encoded by a nucleotide sequence in Table 1; or a sequence substantially identical (e.g., having at least about 80%, 85%, 90%, 92%, 95%, 97%, 98%, or 99% sequence identity) to any of the aforesaid sequences.

In certain embodiments, the anti-CD73 antibody molecule includes all six CDRs from an antibody described herein, e.g., an antibody chosen from 918, 350, 356, 358, 930, 373, 374, 376, 377, 379, 363, 366, 407, 893, 939, 430, or 398; or as described in Table 1; or encoded by a nucleotide sequence in Table 1, or closely related CDRs, e.g., CDRs which are identical or which have at least one amino acid alteration, but not more than two, three or four alterations (e.g., substitutions, deletions, or insertions, e.g., conservative substitutions). In certain embodiments, the anti-CD73 antibody molecule may include any CDR described herein. In certain embodiments, the anti-CD73 antibody molecule includes a substitution in a heavy chain CDR, e.g., one or more substitutions in a CDR1, CDR2 and/or CDR3 of the heavy chain.

In some embodiments, the anti-CD73 antibody molecule includes all six CDRs according to Kabat et al. (e.g., all six CDRs according to the Kabat definition as set out in Table 1) from the heavy and light chain variable regions of an antibody described herein, e.g., an antibody chosen from 918, 350, 356, 358, 930, 373, 374, 376, 377, 379, 363, 366, 407, 893, 939, 430, or 398; or as described in Table 1; or encoded by a nucleotide sequence in Table 1; or a sequence substantially identical (e.g., having at least about 80%, 85%, 90%, 92%, 95%, 97%, 98%, or 99% sequence identity) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Table 1. In one embodiment, the anti-CD73 antibody molecule may include any CDR described herein.

In some embodiments, the anti-CD73 antibody molecule includes all six hypervariable loops (e.g., all six hypervariable loops according to the Chothia definition as set out in Table 1) of an antibody described herein, e.g., an antibody chosen from 918, 350, 356, 358, 930, 373, 374, 376, 377, 379, 363, 366, 407, 893, 939, 430, or 398; or closely related hypervariable loops, e.g., hypervariable loops which are identical or which have at least one amino acid alteration, but not more than two, three or four alterations (e.g., substitutions, deletions, or insertions, e.g., conservative substitutions); or which have at least one amino acid alteration, but not more than two, three or four alterations (e.g., substitutions, deletions, or insertions, e.g., conservative substitutions) relative to all six hypervariable loops according to Chothia et al. shown in Table 1. In one embodiment, the anti-CD73 antibody molecule may include any hypervariable loop described herein.

In certain embodiments, the anti-CD73 antibody molecule includes a combination of CDRs or hypervariable loops defined according to the Kabat et al. and Chothia et al.

In some embodiments, the anti-CD73 antibody molecule includes all six CDRs according to the IMGT definition (e.g., all six CDRs according to the IMGT definition as set out in Table 1) from the heavy and light chain variable regions of an antibody described herein, e.g., an antibody chosen from 918, 350, 356, 358, 930, 373, 374, 376, 377, 379, 363, 366, 407, 893, 939, 430, or 398; or as described in Table 1; or encoded by a nucleotide sequence in Table 1; or a sequence substantially identical (e.g., having at least about 80%, 85%, 90%, 92%, 95%, 97%, 98%, or 99% sequence identity) to any of the aforesaid sequences; or which have at least one amino acid alteration, but not more than two, three or four alterations (e.g., substitutions, deletions, or insertions, e.g., conservative substitutions) relative to all six CDRs according to the IMGT definition shown in Table 1. In one embodiment, the anti-CD73 antibody molecule may include any CDR described herein.

In some embodiments, the heavy or light chain variable domain, or both, of the anti-CD73 antibody molecule includes an amino acid sequence, which is substantially identical to an amino acid disclosed herein, e.g., having at least about 80%, 85%, 90%, 92%, 95%, 97%, 98%, or 99% sequence identity to a variable region of an antibody described herein, e.g., an antibody chosen from 918, 350, 356, 358, 930, 373, 374, 376, 377, 379, 363, 366, 407, 893, 939, 430, or 398; or as described in Table 1; or encoded by a nucleotide sequence in Table 1; or which differs at least 1 or 5 residues, but less than 40, 30, 20, or 10 residues, from a variable region of an antibody described herein.

In certain embodiments, the heavy or light chain variable region, or both, of the anti-CD73 antibody molecule includes an amino acid sequence encoded by a nucleic acid sequence described herein or a nucleic acid that hybridizes to a nucleic acid sequence described herein (e.g., a nucleic acid sequence as shown in Table 1) or its complement, e.g., under low stringency, medium stringency, or high stringency, or other hybridization condition described herein.

In some embodiments, the antibody molecule has a variable region that is identical in sequence, or which differs by 1, 2, 3, or 4 amino acids from a variable region described herein (e.g., an FR region disclosed herein).

In one embodiment, the anti-CD73 antibody molecule comprises a heavy chain variable region (VH) comprising a VHCDR3 amino acid sequence of GGLYGSGSYLSDFDL (SEQ ID NO: 37). In one embodiment, the anti-CD73 antibody molecule comprises a heavy chain variable region (VH) comprising a VHCDR3 amino acid sequence of ESQESPYNNWFDP (SEQ ID NO: 3).

In one embodiment, the anti-CD73 antibody molecule comprises a heavy chain variable region (VH) comprising a VHCDR1 amino acid sequence of SEQ ID NO: 88, a VHCDR2 amino acid sequence of SEQ ID NO: 89, and a VHCDR3 amino acid sequence of SEQ ID NO: 37; and a light chain variable region (VL) comprising a VLCDR1 amino acid sequence of SEQ ID NO: 48, a VLCDR2 amino acid sequence of SEQ ID NO: 49, and a VLCDR3 amino acid sequence of SEQ ID NO: 50, each disclosed in Table 2. In one embodiment, the anti-CD73 antibody molecule comprises a heavy chain variable region (VH) comprising a VHCDR1 amino acid sequence of SEQ ID NO: 90, a VHCDR2 amino acid sequence of SEQ ID NO: 91, and a VHCDR3 amino acid sequence of SEQ ID NO: 3; and a light chain variable region (VL) comprising a VLCDR1 amino acid sequence of SEQ ID NO: 14, a VLCDR2 amino acid sequence of SEQ ID NO: 15, and a VLCDR3 amino acid sequence of SEQ ID NO: 16, each disclosed in Table 2.

In one embodiment, the anti-CD73 antibody molecule comprises a VH comprising a VHCDR1 amino acid sequence of SEQ ID NO: 38, a VHCDR2 amino acid sequence of SEQ ID NO: 36, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 72, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 38, a VHCDR2 amino acid sequence of SEQ ID NO: 71, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 137, a VHCDR2 amino acid sequence of SEQ ID NO: 136, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 137, a VHCDR2 amino acid sequence of SEQ ID NO: 146, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 137, a VHCDR2 amino acid sequence of SEQ ID NO: 154, and a VHCDR3 amino acid sequence of SEQ ID NO: 37; and a VL comprising a VLCDR1 amino acid sequence of SEQ ID NO: 48, a VLCDR2 amino acid sequence of SEQ ID NO: 49, and a VLCDR3 amino acid sequence of SEQ ID NO: 50. In one embodiment, the anti-CD73 antibody molecule comprises a VH comprising a VHCDR1 amino acid sequence of SEQ ID NO: 61, a VHCDR2 amino acid sequence of SEQ ID NO: 60, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16. In one embodiment, the anti-CD73 antibody molecule comprises a VH comprising a VHCDR1 amino acid sequence of SEQ ID NO: 4, a VHCDR2 amino acid sequence of SEQ ID NO: 26, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16. In one embodiment, the anti-CD73 antibody molecule comprises a VH comprising a VHCDR1 amino acid sequence of SEQ ID NO: 4, a VHCDR2 amino acid sequence of SEQ ID NO: 2, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16. In one embodiment, the anti-CD73 antibody molecule comprises a VH comprising a VHCDR1 amino acid sequence of SEQ ID NO: 163, a VHCDR2 amino acid sequence of SEQ ID NO: 162, and a VHCDR3 amino acid sequence of SEQ ID NO: 3; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 16.

In one embodiment, the anti-CD73 antibody molecule comprises a VH comprising a VHCDR1 amino acid sequence of SEQ ID NO: 39, a VHCDR2 amino acid sequence of SEQ ID NO: 40, and a VHCDR3 amino acid sequence of SEQ ID NO: 37; and a VL comprising a VLCDR1 amino acid sequence of SEQ ID NO: 51, a VLCDR2 amino acid sequence of SEQ ID NO: 52, and a VLCDR3 amino acid sequence of SEQ ID NO: 53. In one embodiment, the anti-CD73 antibody molecule comprises a VH comprising a VHCDR1 amino acid sequence of SEQ ID NO: 73, a VHCDR2 amino acid sequence of SEQ ID NO: 74, and a VHCDR3 amino acid sequence of SEQ ID NO: 37; and a VL comprising a VLCDR1 amino acid sequence of SEQ ID NO: 51, a VLCDR2 amino acid sequence of SEQ ID NO: 52, and a VLCDR3 amino acid sequence of SEQ ID NO: 53. In one embodiment, the anti-CD73 antibody molecule comprises a VH comprising a VHCDR1 amino acid sequence of SEQ ID NO: 82, a VHCDR2 amino acid sequence of SEQ ID NO: 74, and a VHCDR3 amino acid sequence of SEQ ID NO: 37; and a VL comprising a VLCDR1 amino acid sequence of SEQ ID NO: 51, a VLCDR2 amino acid sequence of SEQ ID NO: 52, and a VLCDR3 amino acid sequence of SEQ ID NO: 53. In one embodiment, the anti-CD73 antibody molecule comprises a VH comprising a VHCDR1 amino acid sequence of SEQ ID NO: 138, a VHCDR2 amino acid sequence of SEQ ID NO: 139, and a VHCDR3 amino acid sequence of SEQ ID NO: 37; and a VL comprising a VLCDR1 amino acid sequence of SEQ ID NO: 51, a VLCDR2 amino acid sequence of SEQ ID NO: 52, and a VLCDR3 amino acid sequence of SEQ ID NO: 53. In one embodiment, the anti-CD73 antibody molecule comprises a VH comprising a VHCDR1 amino acid sequence of SEQ ID NO: 147, a VHCDR2 amino acid sequence of SEQ ID NO: 148, and a VHCDR3 amino acid sequence of SEQ ID NO: 37; and a VL comprising a VLCDR1 amino acid sequence of SEQ ID NO: 51, a VLCDR2 amino acid sequence of SEQ ID NO: 52, and a VLCDR3 amino acid sequence of SEQ ID NO: 53. In one embodiment, the anti-CD73 antibody molecule comprises a VH comprising a VHCDR1 amino acid sequence of SEQ ID NO: 155, a VHCDR2 amino acid sequence of SEQ ID NO: 156, and a VHCDR3 amino acid sequence of SEQ ID NO: 37; and a VL comprising a VLCDR1 amino acid sequence of SEQ ID NO: 51, a VLCDR2 amino acid sequence of SEQ ID NO: 52, and a VLCDR3 amino acid sequence of SEQ ID NO: 53. In one embodiment, the anti-CD73 antibody molecule comprises a VH comprising a VHCDR1 amino acid sequence of SEQ ID NO: 62, a VHCDR2 amino acid sequence of SEQ ID NO: 63, and a VHCDR3 amino acid sequence of SEQ ID NO: 3; and a VL comprising a VLCDR1 amino acid sequence of SEQ ID NO: 17, a VLCDR2 amino acid sequence of SEQ ID NO: 18, and a VLCDR3 amino acid sequence of SEQ ID NO: 19. In one embodiment, the anti-CD73 antibody molecule comprises a VH comprising a VHCDR1 amino acid sequence of SEQ ID NO: 27, a VHCDR2 amino acid sequence of SEQ ID NO: 28, and a VHCDR3 amino acid sequence of SEQ ID NO: 3; and a VL comprising a VLCDR1 amino acid sequence of SEQ ID NO: 17, a VLCDR2 amino acid sequence of SEQ ID NO: 18, and a VLCDR3 amino acid sequence of SEQ ID NO: 19. In one embodiment, the anti-CD73 antibody molecule comprises a VH comprising a VHCDR1 amino acid sequence of SEQ ID NO: 5, a VHCDR2 amino acid sequence of SEQ ID NO: 6, and a VHCDR3 amino acid sequence of SEQ ID NO: 3; and a VL comprising a VLCDR1 amino acid sequence of SEQ ID NO: 17, a VLCDR2 amino acid sequence of SEQ ID NO: 18, and a VLCDR3 amino acid sequence of SEQ ID NO: 19. In one embodiment, the anti-CD73 antibody molecule comprises a VH comprising a VHCDR1 amino acid sequence of SEQ ID NO: 164, a VHCDR2 amino acid sequence of SEQ ID NO: 165, and a VHCDR3 amino acid sequence of SEQ ID NO: 3; and a VL comprising a VLCDR1 amino acid sequence of SEQ ID NO: 17, a VLCDR2 amino acid sequence of SEQ ID NO: 18, and a VLCDR3 amino acid sequence of SEQ ID NO: 19.

In other embodiments, the aforesaid antibodies comprise a heavy chain variable region comprising an amino acid sequence having at least about 85% (e.g., at least 90%, 95%, 97%, 98%, 99%, or 100%) sequence identity to any of SEQ ID NOs: 44, 77, 84, 142, 151, or 159. In other embodiments, the aforesaid antibodies comprise a heavy chain variable region comprising an amino acid sequence having at least about 85% (e.g., at least 90%, 95%, 97%, 98%, 99%, or 100%) sequence identity to any of SEQ ID NOs: 66, 31, 10, or 168.

In other embodiments, the aforesaid antibodies comprise a light chain variable region comprising an amino acid sequence having at least about 85% (e.g., at least 90%, 95%, 97%, 98%, 99%, or 100%) sequence identity to any of SEQ ID NOs: 55 or 21.

In other embodiments, the aforesaid antibodies comprise a heavy chain comprising an amino acid sequence having at least about 85% (e.g., at least 90%, 95%, 97%, 98%, 99%, or 100%) sequence identity to any of SEQ ID NOs: 46, 79, 86, 114, 116, or 117. In other embodiments, the aforesaid antibodies comprise a heavy chain comprising an amino acid sequence having at least about 85% (e.g., at least 90%, 95%, 97%, 98%, 99%, or 100%) sequence identity to any of SEQ ID NOs: 68, 33, 12, 115, 113, or 112.

In other embodiments, the aforesaid antibodies comprise a light chain comprising an amino acid sequence having at least about 85% (e.g., at least 90%, 95%, 97%, 98%, 99%, or 100%) sequence identity to any of SEQ ID NOs: 57 or 23.

In other embodiments, the antibody molecule comprises a heavy chain variable region comprising an amino acid sequence having at least about 85% (e.g., at least 90%, 95%, 97%, 98%, 99%, or 100%) sequence identity to SEQ ID NO: 44; and a light chain variable region comprising an amino acid sequence having at least about 85% (e.g., at least 90%, 95%, 97%, 98%, 99%, or 100%) sequence identity to SEQ ID NO: 55. In other embodiments, the antibody molecule comprises a heavy chain variable region comprising an amino acid sequence having at least about 85% (e.g., at least 90%, 95%, 97%, 98%, 99%, or 100%) sequence identity to SEQ ID NO: 77; and a light chain variable region comprising an amino acid sequence having at least about 85% (e.g., at least 90%, 95%, 97%, 98%, 99%, or 100%) sequence identity to SEQ ID NO: 55. In other embodiments, the antibody molecule comprises a heavy chain variable region comprising an amino acid sequence having at least about 85% (e.g., at least 90%, 95%, 97%, 98%, 99%, or 100%) sequence identity to SEQ ID NO: 84; and a light chain variable region comprising an amino acid sequence having at least about 85% (e.g., at least 90%, 95%, 97%, 98%, 99%, or 100%) sequence identity to SEQ ID NO: 55. In other embodiments, the antibody molecule comprises a heavy chain variable region comprising an amino acid sequence having at least about 85% (e.g., at least 90%, 95%, 97%, 98%, 99%, or 100%) sequence identity to SEQ ID NO: 142; and a light chain variable region comprising an amino acid sequence having at least about 85% (e.g., at least 90%, 95%, 97%, 98%, 99%, or 100%) sequence identity to SEQ ID NO: 55. In other embodiments, the antibody molecule comprises a heavy chain variable region comprising an amino acid sequence having at least about 85% (e.g., at least 90%, 95%, 97%, 98%, 99%, or 100%) sequence identity to SEQ ID NO: 151; and a light chain variable region comprising an amino acid sequence having at least about 85% (e.g., at least 90%, 95%, 97%, 98%, 99%, or 100%) sequence identity to SEQ ID NO: 55. In other embodiments, the antibody molecule comprises a heavy chain variable region comprising an amino acid sequence having at least about 85% (e.g., at least 90%, 95%, 97%, 98%, 99%, or 100%) sequence identity to SEQ ID NO: 159; and a light chain variable region comprising an amino acid sequence having at least about 85% (e.g., at least 90%, 95%, 97%, 98%, 99%, or 100%) sequence identity to SEQ ID NO: 55. In other embodiments, the antibody molecule comprises a heavy chain variable region comprising an amino acid sequence having at least about 85% (e.g., at least 90%, 95%, 97%, 98%, 99%, or 100%) sequence identity to SEQ ID NO: 66; and a light chain variable region comprising an amino acid sequence having at least about 85% (e.g., at least 90%, 95%, 97%, 98%, 99%, or 100%) sequence identity to SEQ ID NO: 21. In other embodiments, the antibody molecule comprises a heavy chain variable region comprising an amino acid sequence having at least about 85% (e.g., at least 90%, 95%, 97%, 98%, 99%, or 100%) sequence identity to SEQ ID NO: 31; and a light chain variable region comprising an amino acid sequence having at least about 85% (e.g., at least 90%, 95%, 97%, 98%, 99%, or 100%) sequence identity to SEQ ID NO: 21. In other embodiments, the antibody molecule comprises a heavy chain variable region comprising an amino acid sequence having at least about 85% (e.g., at least 90%, 95%, 97%, 98%, 99%, or 100%) sequence identity to SEQ ID NO: 10; and a light chain variable region comprising an amino acid sequence having at least about 85% (e.g., at least 90%, 95%, 97%, 98%, 99%, or 100%) sequence identity to SEQ ID NO: 21. In other embodiments, the antibody molecule comprises a heavy chain variable region comprising an amino acid sequence having at least about 85% (e.g., at least 90%, 95%, 97%, 98%, 99%, or 100%) sequence identity to SEQ ID NO: 168; and a light chain variable region comprising an amino acid sequence having at least about 85% (e.g., at least 90%, 95%, 97%, 98%, 99%, or 100%) sequence identity to SEQ ID NO: 21.

In other embodiments, the antibody molecule comprises a heavy chain comprising an amino acid sequence having at least about 85% (e.g., at least 90%, 95%, 97%, 98%, 99%, or 100%) sequence identity to SEQ ID NO: 46; and a light chain comprising an amino acid sequence having at least about 85% (e.g., at least 90%, 95%, 97%, 98%, 99%, or 100%) sequence identity to SEQ ID NO: 57. In other embodiments, the antibody molecule comprises a heavy chain comprising an amino acid sequence having at least about 85% (e.g., at least 90%, 95%, 97%, 98%, 99%, or 100%) sequence identity to SEQ ID NO: 79; and a light chain comprising an amino acid sequence having at least about 85% (e.g., at least 90%, 95%, 97%, 98%, 99%, or 100%) sequence identity to SEQ ID NO: 57. In other embodiments, the antibody molecule comprises a heavy chain comprising an amino acid sequence having at least about 85% (e.g., at least 90%, 95%, 97%, 98%, 99%, or 100%) sequence identity to SEQ ID NO: 86; and a light chain comprising an amino acid sequence having at least about 85% (e.g., at least 90%, 95%, 97%, 98%, 99%, or 100%) sequence identity to SEQ ID NO: 57. In other embodiments, the antibody molecule comprises a heavy chain comprising an amino acid sequence having at least about 85% (e.g., at least 90%, 95%, 97%, 98%, 99%, or 100%) sequence identity to SEQ ID NO: 114; and a light chain comprising an amino acid sequence having at least about 85% (e.g., at least 90%, 95%, 97%, 98%, 99%, or 100%) sequence identity to SEQ ID NO: 57. In other embodiments, the antibody molecule comprises a heavy chain comprising an amino acid sequence having at least about 85% (e.g., at least 90%, 95%, 97%, 98%, 99%, or 100%) sequence identity to SEQ ID NO: 116; and a light chain comprising an amino acid sequence having at least about 85% (e.g., at least 90%, 95%, 97%, 98%, 99%, or 100%) sequence identity to SEQ ID NO: 57. In other embodiments, the antibody molecule comprises a heavy chain comprising an amino acid sequence having at least about 85% (e.g., at least 90%, 95%, 97%, 98%, 99%, or 100%) sequence identity to SEQ ID NO: 117; and a light chain comprising an amino acid sequence having at least about 85% (e.g., at least 90%, 95%, 97%, 98%, 99%, or 100%) sequence identity to SEQ ID NO: 57. In other embodiments, the antibody molecule comprises a heavy chain comprising an amino acid sequence having at least about 85% (e.g., at least 90%, 95%, 97%, 98%, 99%, or 100%) sequence identity to SEQ ID NO: 68; and a light chain comprising an amino acid sequence having at least about 85% (e.g., at least 90%, 95%, 97%, 98%, 99%, or 100%) sequence identity to SEQ ID NO: 23. In other embodiments, the antibody molecule comprises a heavy chain comprising an amino acid sequence having at least about 85% (e.g., at least 90%, 95%, 97%, 98%, 99%, or 100%) sequence identity to SEQ ID NO: 33; and a light chain comprising an amino acid sequence having at least about 85% (e.g., at least 90%, 95%, 97%, 98%, 99%, or 100%) sequence identity to SEQ ID NO: 23. In other embodiments, the antibody molecule comprises a heavy chain comprising an amino acid sequence having at least about 85% (e.g., at least 90%, 95%, 97%, 98%, 99%, or 100%) sequence identity to SEQ ID NO: 12; and a light chain comprising an amino acid sequence having at least about 85% (e.g., at least 90%, 95%, 97%, 98%, 99%, or 100%) sequence identity to SEQ ID NO: 23. In other embodiments, the antibody molecule comprises a heavy chain comprising an amino acid sequence having at least about 85% (e.g., at least 90%, 95%, 97%, 98%, 99%, or 100%) sequence identity to SEQ ID NO: 115; and a light chain comprising an amino acid sequence having at least about 85% (e.g., at least 90%, 95%, 97%, 98%, 99%, or 100%) sequence identity to SEQ ID NO: 23. In other embodiments, the antibody molecule comprises a heavy chain comprising an amino acid sequence having at least about 85% (e.g., at least 90%, 95%, 97%, 98%, 99%, or 100%) sequence identity to SEQ ID NO: 113; and a light chain comprising an amino acid sequence having at least about 85% (e.g., at least 90%, 95%, 97%, 98%, 99%, or 100%) sequence identity to SEQ ID NO: 23. In other embodiments, the antibody molecule comprises a heavy chain comprising an amino acid sequence having at least about 85% (e.g., at least 90%, 95%, 97%, 98%, 99%, or 100%) sequence identity to SEQ ID NO: 112; and a light chain comprising an amino acid sequence having at least about 85% (e.g., at least 90%, 95%, 97%, 98%, 99%, or 100%) sequence identity to SEQ ID NO: 23.

In other embodiments, the aforesaid antibody molecules are chosen from a full antibody, a bispecific antibody, Fab, F(ab′)2, Fv, or a single chain Fv fragment (scFv).

In other embodiments, the aforesaid antibody molecules comprise a heavy chain constant region selected from IgG1, IgG2, IgG3, and IgG4.

In other embodiments, the aforesaid antibody molecules comprise a light chain constant region chosen from the light chain constant regions of kappa or lambda.

In some embodiments, the anti-CD73 antibody molecule comprises a heavy chain variable region, a light chain variable region, a heavy chain constant region, and/or a light chain constant region disclosed in Table 1. In some embodiments, the anti-CD73 antibody molecule comprises a heavy chain constant region, and/or a light chain constant region disclosed in Table 3. In some embodiments, the anti-CD73 antibody molecule comprises a heavy chain constant region comprising an amino acid sequence selected from the group consisting of SEQ ID NOs: 92-103, 119, and 120. In some embodiments, the anti-CD73 antibody molecule comprises a light chain constant region comprising the amino acid sequence of SEQ ID NO: 104.

Exemplary sequences of anti-CD73 antibodies are described in Tables 1 and 2 below.

TABLE 1
Amino acid and nucleotide sequences for exemplary anti-CD73 antibodies

| | | |
|---|---|---|
| 358 | | |
| SEQ ID NO: 1 | HCDR1 | GGSISGRYWS |
| (Combined) | | |
| SEQ ID NO: 2 | HCDR2 | YIYGTGSTNYNPSLKS |
| (Combined) | | |
| SEQ ID NO: 3 | HCDR3 | ESQESPYNNWFDP |
| (Combined) | | |
| SEQ ID NO: 4 (Kabat) | HCDR1 | GRYWS |
| SEQ ID NO: 2 (Kabat) | HCDR2 | YIYGTGSTNYNPSLKS |
| SEQ ID NO: 3 (Kabat) | HCDR3 | ESQESPYNNWFDP |
| SEQ ID NO: 5 (Chothia) | HCDR1 | GGSISGR |
| SEQ ID NO: 6 (Chothia) | HCDR2 | YGTGS |
| SEQ ID NO: 3 (Chothia) | HCDR3 | ESQESPYNNWFDP |
| SEQ ID NO: 7 (IMGT) | HCDR1 | GGSISGRY |
| SEQ ID NO: 8 (IMGT) | HCDR2 | IYGTGST |
| SEQ ID NO: 9 (IMGT) | HCDR3 | ARESQESPYNNWFDP |
| SEQ ID NO: 199 | HCDR1 | GSISGRYWS |
| (Alternative) | | |
| SEQ ID NO: 2 | HCDR2 | YIYGTGSTNYNPSLKS |
| (Alternative) | | |
| SEQ ID NO: 9 | HCDR3 | ARESQESPYNNWFDP |
| (Alternative) | | |
| SEQ ID NO: 10 | VH | QVQLQESGPGLVKPSETLSLTCTVSGGSISGRYWSWI |
| | | RQPPGKGLEWIGYIYGTGSTNYNPSLKSRVTISVDTSK |
| | | NQFSLKLSSVTAADTAVYYCARESQESPYNNWFDPW |
| | | GQGTLVTVSS |
| SEQ ID NO: 11 | DNA VH | CAAGTGCAGCTGCAGGAATCTGGCCCTGGCCTGGT |
| | | CAAGCCCTCCGAGACACTGTCCCTGACCTGCACCGT |
| | | GTCCGGCGGCTCCATCTCCGGCCGGTACTGGTCTT |
| | | GGATCCGGCAGCCTCCCGGCAAGGGCCTGGAATGG |
| | | ATCGGCTACATCTACGGCACCGGCTCCACCAACTAC |
| | | AACCCCAGCCTGAAGTCCAGAGTGACCATCTCCGTG |
| | | GACACCTCCAAGAACCAGTTCTCCCTGAAGCTGTCC |
| | | TCCGTGACCGCCGCTGACACCGCCGTGTACTACTGC |
| | | GCCAGAGAGTCCCAGGAATCCCCTTACAACAATTGG |
| | | TTCGACCCCTGGGGCCAGGGCACCCTGGTCACCGT |
| | | GTCCTCT |
| SEQ ID NO: 12 | 358.A | QVQLQESGPGLVKPSETLSLTCTVSGGSISGRYWSWI |
| | Heavy | RQPPGKGLEWIGYIYGTGSTNYNPSLKSRVTISVDTSK |
| | Chain | NQFSLKLSSVTAADTAVYYCARESQESPYNNWFDPW |
| | | GQGTLVTVSSASTKGPSVFPLAPCSRSTSESTAALGCL |
| | | VKDYFPEPVTVSWNSGALTSGVHTFPAVLQSSGLYSL |
| | | SSVVTVPSSSLGTKTYTCNVDHKPSNTKVDKRVESKY |
| | | GPPCPPCPAPEFLGGPSVFLFPPKPKDTLMISRTPEVT |
| | | CVVVDVSQEDPEVQFNWYVDGVEVHNAKTKPREEQF |
| | | NSTYRVVSVLTVLHQDWLNGKEYKCKVSNKGLPSSIEK |
| | | TISKAKGQPREPQVYTLPPSQEEMTKNQVSLTCLVKGF |
| | | YPSDIAVEWESNGQPENNYKTTPPVLDSDGSFFLYSRL |
| | | TVDKSRWQEGNVFSCSVMHEALHNHYTQKSLSLSLGX, |
| | | wherein X is K or absent. |
| SEQ ID NO: 13 | 358.A | CAAGTGCAGCTGCAGGAATCTGGCCCTGGCCTGGT |
| | DNA | CAAGCCCTCCGAGACACTGTCCCTGACCTGCACCGT |
| | Heavy | GTCCGGCGGCTCCATCTCCGGCCGGTACTGGTCTT |
| | Chain | GGATCCGGCAGCCTCCCGGCAAGGGCCTGGAATGG |
| | | ATCGGCTACATCTACGGCACCGGCTCCACCAACTAC |
| | | AACCCCAGCCTGAAGTCCAGAGTGACCATCTCCGTG |
| | | GACACCTCCAAGAACCAGTTCTCCCTGAAGCTGTCC |
| | | TCCGTGACCGCCGCTGACACCGCCGTGTACTACTGC |
| | | GCCAGAGAGTCCCAGGAATCCCCTTACAACAATTGG |
| | | TTCGACCCCTGGGGCCAGGGCACCCTGGTCACCGT |
| | | GTCCTCTGCTTCCACCAAGGGCCCCTCCGTGTTCCC |
| | | TCTGGCCCCTTGCTCCCGGTCCACCTCCGAGTCTAC |
| | | CGCCGCTCTGGGCTGCCTCGTGAAGGACTACTTCCC |
| | | CGAGCCCGTGACCGTGTCCTGGAACTCTGGCGCCC |
| | | TGACCTCCGGCGTGCACACCTTCCCTGCCGTGCTGC |
| | | AGTCCTCCGGCCTGTACTCCCTGTCCAGCGTCGTGA |
| | | CCGTGCCCTCCTCCAGCCTGGGCACCAAGACCTACA |
| | | CCTGTAACGTGGACCACAAGCCCTCCAACACCAAAG |
| | | TGGACAAGCGGGTGGAATCTAAGTACGGCCCTCCCT |
| | | GCCCTCCTTGCCCTGCCCCTGAGTTCCTGGGCGGA |
| | | CCTTCCGTGTTCCTGTTCCCTCCAAAGCCCAAGGAC |
| | | ACCCTGATGATCTCCCGGACCCCTGAAGTGACCTGC |
| | | GTGGTGGTGGACGTGTCCCAGGAAGATCCCGAAGT |
| | | CCAGTTCAATTGGTACGTGGACGGCGTGGAAGTGCA |
| | | CAACGCCAAGACCAAGCCCAGAGAGGAACAGTTCAA |
| | | CTCCACCTACCGGGTGGTGTCCGTGCTGACCGTGCT |
| | | GCACCAGGACTGGCTGAACGGCAAAGAGTACAAGT |
| | | GCAAAGTGTCCAACAAGGGCCTGCCCTCCAGCATCG |
| | | AAAAGACCATCTCCAAGGCCAAGGGCCAGCCCCGC |
| | | GAGCCCCAAGTGTACACCCTGCCTCCCAGCCAGGA |
| | | AGAGATGACCAAGAATCAAGTGTCCCTGACTTGTCT |
| | | GGTCAAGGGCTTCTACCCCTCCGATATCGCCGTGGA |
| | | GTGGGAGTCCAACGGCCAGCCCGAGAACAACTACA |
| | | AGACCACCCCTCCCGTGCTGGACTCCGACGGCTCC |
| | | TTCTTCCTGTACTCTCGGCTGACCGTGGACAAGTCC |
| | | CGGTGGCAGGAAGGCAACGTCTTCTCCTGCTCCGT |
| | | GATGCACGAGGCCCTGCACAACCACTACACCCAGAA |
| | | GTCCCTGTCCCTGTCTCTGGGCX₁X₂X₃, |
| | | wherein X₁ is A, X₂ is A, and X₃ is G; |
| | | or X₁ is absent, X₂ isabsent, and X₃ |
| | | is absent. |
| SEQ ID NO: 112 | 358.B | QVQLQESGPGLVKPSETLSLTCTVSGGSISGRYWSWI |
| | Heavy | RQPPGKGLEWIGYIYGTGSTNYNPSLKSRVTISVDTSK |
| | Chain | NQFSLKLSSVTAADTAVYYCARESQESPYNNWFDPW |
| | | GQGTLVTVSSASTKGPSVFPLAPCSRSTSESTAALGCL |
| | | VKDYFPEPVTVSWNSGALTSGVHTFPAVLQSSGLYSL |
| | | SSVVTVPSSSLGTKTYTCNVDHKPSNTKVDKRVESKY |
| | | GPPCPPCPAPEFEGGPSVFLFPPKPKDTLMISRTPEVT |
| | | CVVVDVSQEDPEVQFNWYVDGVEVHNAKTKPREEQF |
| | | NSTYRVVSVLTVLHQDWLNGKEYKCKVSNKGLPSSIEK |
| | | TISKAKGQPREPQVYTLPPSQEEMTKNQVSLTCLVKGF |
| | | YPSDIAVEWESNGQPENNYKTTPPVLDSDGSFFLYSRL |
| | | TVDKSRWQEGNVFSCSVMHEALHNHYTQKSLSLSLGX. |
| | | wherein X is K or absent |
| SEQ ID NO: 14 | LCDR1 | RASQGISSWLA |
| (Combined) | | |
| SEQ ID NO: 15 | LCDR2 | AASSLQS |
| (Combined) | | |
| SEQ ID NO: 16 | LCDR3 | QQGNSFPRT |
| (Combined) | | |
| SEQ ID NO: 14 (Kabat) | LCDR1 | RASQGISSWLA |
| SEQ ID NO: 15 (Kabat) | LCDR2 | AASSLQS |
| SEQ ID NO: 16 (Kabat) | LCDR3 | QQGNSFPRT |
| SEQ ID NO: 17 (Chothia) | LCDR1 | SQGISSW |
| SEQ ID NO: 18 (Chothia) | LCDR2 | AAS |
| SEQ ID NO: 19 (Chothia) | LCDR3 | GNSFPR |
| SEQ ID NO: 20 (IMGT) | LCDR1 | QGISSW |
| SEQ ID NO: 18 (IMGT) | LCDR2 | AAS |
| SEQ ID NO: 16 (IMGT) | LCDR3 | QQGNSFPRT |
| SEQ ID NO: 14 | LCDR1 | RASQGISSWLA |
| (Alternative) | | |
| SEQ ID NO: 15 | LCDR2 | AASSLQS |
| (Alternative) | | |
| SEQ ID NO: 16 | LCDR3 | QQGNSFPRT |
| (Alternative) | | |
| SEQ ID NO: 21 | VL | DIQMTQSPSSVSASVGDRVTITCRASQGISSWLAWYQ |
| | | QKPGKAPKLLIYAASSLQSGVPSRFSGSGSGTDFTLTIS |
| | | SLQPEDFATYYCQQGNSFPRTFGGGTKVEIK |
| SEQ ID NO: 22 | DNA VL | GACATCCAGATGACCCAGAGCCCCTCCTCCGTGTCC |
| | | GCCTCCGTGGGCGACAGAGTGACCATCACCTGTCG |
| | | GGCCTCCCAGGGCATCTCCAGCTGGCTGGCCTGGT |
| | | ATCAGCAGAAGCCCGGCAAGGCCCCTAAGCTGCTG |
| | | ATCTACGCCGCCTCCAGCCTGCAGTCCGGCGTGCC |
| | | CTCCAGATTCTCCGGCTCTGGCTCCGGCACCGACTT |
| | | CACCCTGACCATCAGCTCCCTGCAGCCCGAGGACTT |
| | | CGCCACCTACTACTGCCAGCAGGGCAACTCCTTCCC |
| | | TCGGACCTTCGGCGGAGGCACCAAAGTGGAAATCAA |
| | | G |
| SEQ ID NO: 23 | Light | DIQMTQSPSSVSASVGDRVTITCRASQGISSWLAWYQ |
| | Chain | QKPGKAPKLLIYAASSLQSGVPSRFSGSGSGTDFTLTIS |
| | | SLQPEDFATYYCQQGNSFPRTFGGGTKVEIKRTVAAP |
| | | SVFIFPPSDEQLKSGTASVVCLLNNFYPREAKVQWKVD |
| | | NALQSGNSQESVTEQDSKDSTYSLSSTLTLSKADYEKH |
| | | KVYACEVTHQGLSSPVTKSFNRGEC |
| SEQ ID NO: 24 | DNA | GACATCCAGATGACCCAGAGCCCCTCCTCCGTGTCC |
| | Light | GCCTCCGTGGGCGACAGAGTGACCATCACCTGTCG |
| | Chain | GGCCTCCCAGGGCATCTCCAGCTGGCTGGCCTGGT |
| | | ATCAGCAGAAGCCCGGCAAGGCCCCTAAGCTGCTG |
| | | ATCTACGCCGCCTCCAGCCTGCAGTCCGGCGTGCC |
| | | CTCCAGATTCTCCGGCTCTGGCTCCGGCACCGACTT |
| | | CACCCTGACCATCAGCTCCCTGCAGCCCGAGGACTT |
| | | CGCCACCTACTACTGCCAGCAGGGCAACTCCTTCCC |
| | | TCGGACCTTCGGCGGAGGCACCAAAGTGGAAATCAA |
| | | GCGTACGGTGGCCGCTCCCTCCGTGTTCATCTTCCC |
| | | ACCCTCCGACGAGCAGCTGAAGTCCGGCACCGCCT |
| | | CCGTCGTGTGCCTGCTGAACAACTTCTACCCTCGCG |
| | | AGGCCAAAGTGCAGTGGAAAGTGGACAACGCCCTG |
| | | CAGTCCGGCAACTCCCAGGAATCCGTCACCGAGCA |
| | | GGACTCCAAGGACAGCACCTACTCCCTGTCCTCCAC |
| | | CCTGACCCTGTCCAAGGCCGACTACGAGAAGCACAA |
| | | AGTGTACGCCTGCGAAGTGACCCACCAGGGCCTGT |
| | | CCAGCCCCGTGACCAAGTCCTTCAACCGGGGCGAG |
| | | TGC |
| 356 | | |
| SEQ ID NO: 25 | HCDR1 | GGSIEGRYWS |
| (Combined) | | |
| SEQ ID NO: 26 | HCDR2 | YIYGSGSTKYNPSLKS |
| (Combined) | | |
| SEQ ID NO: 3 | HCDR3 | ESQESPYNNWFDP |
| (Combined) | | |
| SEQ ID NO: 4 (Kabat) | HCDR1 | GRYWS |
| SEQ ID NO: 26 (Kabat) | HCDR2 | YIYGSGSTKYNPSLKS |
| SEQ ID NO: 3 (Kabat) | HCDR3 | ESQESPYNNWFDP |
| SEQ ID NO: 27 (Chothia) | HCDR1 | GGSIEGR |
| SEQ ID NO: 28 (Chothia) | HCDR2 | YGSGS |
| SEQ ID NO: 3 (Chothia) | HCDR3 | ESQESPYNNWFDP |
| SEQ ID NO: 29 (IMGT) | HCDR1 | GGSIEGRY |
| SEQ ID NO: 30 (IMGT) | HCDR2 | IYGSGST |
| SEQ ID NO: 9 (IMGT) | HCDR3 | ARESQESPYNNWFDP |
| SEQ ID NO: 198 | HCDR1 | GSIEGRYWS |
| (Alternative) | | |
| SEQ ID NO: 26 | HCDR2 | YIYGSGSTKYNPSLKS |
| (Alternative) | | |
| SEQ ID NO: 9 | HCDR3 | ARESQESPYNNWFDP |
| (Alternative) | | |
| SEQ ID NO: 31 | VH | QVQLQESGPGLVKPSETLSLTCTVSGGSIEGRYWSWI |
| | | RQPPGKGLEWIGYIYGSGSTKYNPSLKSRVTISVDTSK |
| | | NQFSLKLSSVTAADTAVYYCARESQESPYNNWFDPW |
| | | GQGTLVTVSS |
| SEQ ID NO: 32 | DNA VH | CAAGTGCAGCTGCAGGAATCTGGCCCTGGCCTGGT |
| | | CAAGCCCTCCGAGACACTGTCCCTGACCTGCACCGT |
| | | GTCCGGCGGCTCTATCGAGGGCCGGTACTGGTCCT |
| | | GGATCCGGCAGCCTCCTGGCAAGGGCCTGGAATGG |
| | | ATCGGCTACATCTACGGCTCCGGCTCCACCAAGTAC |
| | | AACCCCAGCCTGAAGTCCAGAGTGACCATCTCCGTG |
| | | GACACCTCCAAGAACCAGTTCTCCCTGAAGCTGTCC |
| | | TCCGTGACCGCCGCTGACACCGCCGTGTACTACTGC |
| | | GCCAGAGAGTCCCAGGAATCCCCTTACAACAATTGG |
| | | TTCGACCCCTGGGGCCAGGGCACCCTGGTCACCGT |
| | | GTCCTCT |
| SEQ ID NO: 33 | 356.A | QVQLQESGPGLVKPSETLSLTCTVSGGSIEGRYWSWI |
| | Heavy | RQPPGKGLEWIGYIYGSGSTKYNPSLKSRVTISVDTSK |
| | Chain | NQFSLKLSSVTAADTAVYYCARESQESPYNNWFDPW |
| | | GQGTLVTVSSASTKGPSVFPLAPCSRSTSESTAALGCL |
| | | VKDYFPEPVTVSWNSGALTSGVHTFPAVLQSSGLYSL |
| | | SSVVTVPSSSLGTKTYTCNVDHKPSNTKVDKRVESKY |
| | | GPPCPPCPAPEFLGGPSVFLFPPKPKDTLMISRTPEVT |
| | | CVVVDVSQEDPEVQFNWYVDGVEVHNAKTKPREEQF |
| | | NSTYRVVSVLTVLHQDWLNGKEYKCKVSNKGLPSSIEK |
| | | TISKAKGQPREPQVYTLPPSQEEMTKNQVSLTCLVKGF |
| | | YPSDIAVEWESNGQPENNYKTTPPVLDSDGSFFLYSRL |
| | | TVDKSRWQEGNVFSCSVMHEALHNHYTQKSLSLSLGX, |
| | | wherein X is K or absent |
| SEQ ID NO: 34 | 356.A | CAAGTGCAGCTGCAGGAATCTGGCCCTGGCCTGGT |
| | DNA | CAAGCCCTCCGAGACACTGTCCCTGACCTGCACCGT |
| | Heavy | GTCCGGCGGCTCTATCGAGGGCCGGTACTGGTCCT |
| | Chain | GGATCCGGCAGCCTCCTGGCAAGGGCCTGGAATGG |
| | | ATCGGCTACATCTACGGCTCCGGCTCCACCAAGTAC |
| | | AACCCCAGCCTGAAGTCCAGAGTGACCATCTCCGTG |
| | | GACACCTCCAAGAACCAGTTCTCCCTGAAGCTGTCC |
| | | TCCGTGACCGCCGCTGACACCGCCGTGTACTACTGC |
| | | GCCAGAGAGTCCCAGGAATCCCCTTACAACAATTGG |
| | | TTCGACCCCTGGGGCCAGGGCACCCTGGTCACCGT |
| | | GTCCTCTGCCTCCACCAAGGGCCCCTCCGTGTTCCC |
| | | TCTGGCCCCTTGCTCCCGGTCCACCTCCGAGTCTAC |
| | | CGCCGCTCTGGGCTGCCTCGTGAAGGACTACTTCCC |
| | | CGAGCCCGTGACCGTGTCCTGGAACTCTGGCGCCC |
| | | TGACCTCCGGCGTGCACACCTTCCCTGCCGTGCTGC |
| | | AGTCCTCCGGCCTGTACTCCCTGTCCAGCGTCGTGA |
| | | CCGTGCCCTCCTCCAGCCTGGGCACCAAGACCTACA |
| | | CCTGTAACGTGGACCACAAGCCCTCCAACACCAAAG |
| | | TGGACAAGCGGGTGGAATCTAAGTACGGCCCTCCCT |
| | | GCCCTCCTTGCCCTGCCCCTGAGTTCCTGGGCGGA |
| | | CCTTCCGTGTTCCTGTTCCCTCCAAAGCCCAAGGAC |
| | | ACCCTGATGATCTCCCGGACCCCTGAAGTGACCTGC |
| | | GTGGTGGTGGACGTGTCCCAGGAAGATCCCGAAGT |
| | | CCAGTTCAATTGGTACGTGGACGGCGTGGAAGTGCA |
| | | CAACGCCAAGACCAAGCCCAGAGAGGAACAGTTCAA |
| | | CTCCACCTACCGGGTGGTGTCCGTGCTGACCGTGCT |
| | | GCACCAGGACTGGCTGAACGGCAAAGAGTACAAGT |
| | | GCAAAGTGTCCAACAAGGGCCTGCCCTCCAGCATCG |
| | | AAAAGACCATCTCCAAGGCCAAGGGCCAGCCCCGC |
| | | GAGCCCCAAGTGTACACCCTGCCTCCCAGCCAGGA |
| | | AGAGATGACCAAGAATCAAGTGTCCCTGACTTGTCT |
| | | GGTCAAGGGCTTCTACCCCTCCGATATCGCCGTGGA |
| | | GTGGGAGTCCAACGGCCAGCCCGAGAACAACTACA |
| | | AGACCACCCCTCCCGTGCTGGACTCCGACGGCTCC |
| | | TTCTTCCTGTACTCTCGGCTGACCGTGGACAAGTCC |
| | | CGGTGGCAGGAAGGCAACGTCTTCTCCTGCTCCGT |
| | | GATGCACGAGGCCCTGCACAACCACTACACCCAGAA |
| | | GTCCCTGTCCCTGTCTCTGGGC X₁X₂X₃, |
| | | wherein X₁ is A, X₂ is A, and X₃ is |
| | | G; or X₁ is absent, X₂ is absent, and |
| | | X₃ is absent. |
| SEQ ID NO: 113 | 356.B | QVQLQESGPGLVKPSETLSLTCTVSGGSIEGRYWSWI |
| | Heavy | RQPPGKGLEWIGYIYGSGSTKYNPSLKSRVTISVDTSK |
| | chain | NQFSLKLSSVTAADTAVYYCARESQESPYNNWFDPW |
| | | GQGTLVTVSSASTKGPSVFPLAPCSRSTSESTAALGCL |
| | | VKDYFPEPVTVSWNSGALTSGVHTFPAVLQSSGLYSL |
| | | SSVVTVPSSSLGTKTYTCNVDHKPSNTKVDKRVESKY |
| | | GPPCPPCPAPEFEGGPSVFLFPPKPKDTLMISRTPEVT |
| | | CVVVDVSQEDPEVQFNWYVDGVEVHNAKTKPREEQF |
| | | NSTYRVVSVLTVLHQDWLNGKEYKCKVSNKGLPSSIEK |
| | | TISKAKGQPREPQVYTLPPSQEEMTKNQVSLTCLVKGF |
| | | YPSDIAVEWESNGQPENNYKTTPPVLDSDGSFFLYSRL |
| | | TVDKSRWQEGNVFSCSVMHEALHNHYTQKSLSLSLGX, |
| | | wherein X is K or absent |
| SEQ ID NO: 14 | LCDR1 | RASQGISSWLA |
| (Combined) | | |
| SEQ ID NO: 15 | LCDR2 | AASSLQS |
| (Combined) | | |
| SEQ ID NO: 16 | LCDR3 | QQGNSFPRT |
| (Combined) | | |
| SEQ ID NO: 14 (Kabat) | LCDR1 | RASQGISSWLA |
| SEQ ID NO: 15 (Kabat) | LCDR2 | AASSLQS |
| SEQ ID NO: 16 (Kabat) | LCDR3 | QQGNSFPRT |
| SEQ ID NO: 17 (Chothia) | LCDR1 | SQGISSW |
| SEQ ID NO: 18 (Chothia) | LCDR2 | AAS |
| SEQ ID NO: 19 (Chothia) | LCDR3 | GNSFPR |
| SEQ ID NO: 20 (IMGT) | LCDR1 | QGISSW |
| SEQ ID NO: 18 (IMGT) | LCDR2 | AAS |
| SEQ ID NO: 16 (IMGT) | LCDR3 | QQGNSFPRT |
| SEQ ID NO: 14 | LCDR1 | RASQGISSWLA |
| (Alternative) | | |
| SEQ ID NO: 15 | LCDR2 | AASSLQS |
| (Alternative) | | |
| SEQ ID NO: 16 | LCDR3 | QQGNSFPRT |
| (Alternative) | | |
| SEQ ID NO: 21 | VL | DIQMTQSPSSVSASVGDRVTITCRASQGISSWLAWYQ |
| | | QKPGKAPKLLIYAASSLQSGVPSRFSGSGSGTDFTLTIS |
| | | SLQPEDFATYYCQQGNSFPRTFGGGTKVEIK |
| SEQ ID NO: 22 | DNA VL | GACATCCAGATGACCCAGAGCCCCTCCTCCGTGTCC |
| | | GCCTCCGTGGGCGACAGAGTGACCATCACCTGTCG |
| | | GGCCTCCCAGGGCATCTCCAGCTGGCTGGCCTGGT |
| | | ATCAGCAGAAGCCCGGCAAGGCCCCTAAGCTGCTG |
| | | ATCTACGCCGCCTCCAGCCTGCAGTCCGGCGTGCC |
| | | CTCCAGATTCTCCGGCTCTGGCTCCGGCACCGACTT |
| | | CACCCTGACCATCAGCTCCCTGCAGCCCGAGGACTT |
| | | CGCCACCTACTACTGCCAGCAGGGCAACTCCTTCCC |
| | | TCGGACCTTCGGCGGAGGCACCAAAGTGGAAATCAA |
| | | G |
| SEQ ID NO: 23 | Light | DIQMTQSPSSVSASVGDRVTITCRASQGISSWLAWYQ |
| | Chain | QKPGKAPKLLIYAASSLQSGVPSRFSGSGSGTDFTLTIS |
| | DNA | SLQPEDFATYYCQQGNSFPRTFGGGTKVEIKRTVAAP |
| | | SVFIFPPSDEQLKSGTASVVCLLNNFYPREAKVQWKVD |
| | | NALQSGNSQESVTEQDSKDSTYSLSSTLTLSKADYEKH |
| | | KVYACEVTHQGLSSPVTKSFNRGEC |
| SEQ ID NO: 24 | Light | GACATCCAGATGACCCAGAGCCCCTCCTCCGTGTCC |
| | Chain | GCCTCCGTGGGCGACAGAGTGACCATCACCTGTCG |
| | | GGCCTCCCAGGGCATCTCCAGCTGGCTGGCCTGGT |
| | | ATCAGCAGAAGCCCGGCAAGGCCCCTAAGCTGCTG |
| | | ATCTACGCCGCCTCCAGCCTGCAGTCCGGCGTGCC |
| | | CTCCAGATTCTCCGGCTCTGGCTCCGGCACCGACTT |
| | | CACCCTGACCATCAGCTCCCTGCAGCCCGAGGACTT |
| | | CGCCACCTACTACTGCCAGCAGGGCAACTCCTTCCC |
| | | TCGGACCTTCGGCGGAGGCACCAAAGTGGAAATCAA |
| | | GCGTACGGTGGCCGCTCCCTCCGTGTTCATCTTCCC |
| | | ACCCTCCGACGAGCAGCTGAAGTCCGGCACCGCCT |
| | | CCGTCGTGTGCCTGCTGAACAACTTCTACCCTCGCG |
| | | AGGCCAAAGTGCAGTGGAAAGTGGACAACGCCCTG |
| | | CAGTCCGGCAACTCCCAGGAATCCGTCACCGAGCA |
| | | GGACTCCAAGGACAGCACCTACTCCCTGTCCTCCAC |
| | | CCTGACCCTGTCCAAGGCCGACTACGAGAAGCACAA |
| | | AGTGTACGCCTGCGAAGTGACCCACCAGGGCCTGT |
| | | CCAGCCCCGTGACCAAGTCCTTCAACCGGGGCGAG |
| | | TGC |
| 373 | | |
| SEQ ID NO: 35 | HCDR1 | GFTFHRYAMS |
| (Combined) | | |
| SEQ ID NO: 36 | HCDR2 | AISGSGMNTYYADSVKG |
| (Combined) | | |
| SEQ ID NO: 37 | HCDR3 | GGLYGSGSYLSDFDL |
| (Combined) | | |
| SEQ ID NO: 38 (Kabat) | HCDR1 | RYAMS |
| SEQ ID NO: 36 (Kabat) | HCDR2 | AISGSGMNTYYADSVKG |
| SEQ ID NO: 37 (Kabat) | HCDR3 | GGLYGSGSYLSDFDL |
| SEQ ID NO: 39 (Chothia) | HCDR1 | GFTFHRY |
| SEQ ID NO: 40 (Chothia) | HCDR2 | SGSGMN |
| SEQ ID NO: 37 (Chothia) | HCDR3 | GGLYGSGSYLSDFDL |
| SEQ ID NO: 41 (IMGT) | HCDR1 | GFTFHRYA |
| SEQ ID NO: 42 (IMGT) | HCDR2 | ISGSGMNT |
| SEQ ID NO: 43 (IMGT) | HCDR3 | ARGGLYGSGSYLSDFDL |
| SEQ ID NO: 190 | HCDR1 | FTFHRYAMS |
| (Alternative) | | |
| SEQ ID NO: 36 | HCDR2 | AISGSGMNTYYADSVKG |
| (Alternative) | | |
| SEQ ID NO: 43 | HCDR3 | ARGGLYGSGSYLSDFDL |
| (Alternative) | | |
| SEQ ID NO: 44 | VH | EVQLLESGGGLVQPGGSLRLSCAASGFTFHRYAMSW |
| | | VRQAPGKGLEWVSAISGSGMNTYYADSVKGRFTISRD |
| | | NSKNTLYLQMNSLRAEDTAVYYCARGGLYGSGSYLSD |
| | | FDLWGRGTLVTVSS |
| SEQ ID NO: 45 | DNA VH | GAAGTGCAGCTGCTGGAATCTGGCGGCGGACTGGT |
| | | GCAGCCTGGCGGCTCCCTGAGACTGTCTTGCGCCG |
| | | CCTCCGGCTTCACCTTCCACAGATACGCCATGTCCT |
| | | GGGTCCGACAGGCCCCTGGCAAGGGCCTGGAGTGG |
| | | GTGTCCGCCATCTCCGGCTCCGGCATGAACACCTAC |
| | | TACGCCGACTCCGTGAAGGGCCGGTTCACCATCTCC |
| | | CGGGACAACTCCAAGAACACCCTGTACCTGCAGATG |
| | | AACTCCCTGCGGGCCGAGGACACCGCCGTGTACTA |
| | | CTGTGCCAGAGGCGGCCTGTACGGCTCCGGCTCCT |
| | | ACCTGTCCGACTTCGACCTGTGGGGCAGAGGCACC |
| | | CTGGTCACCGTGTCCTCC |
| SEQ ID NO: 46 | 373.A | EVQLLESGGGLVQPGGSLRLSCAASGFTFHRYAMSW |
| | Heavy | VRQAPGKGLEWVSAISGSGMNTYYADSVKGRFTISRD |
| | Chain | NSKNTLYLQMNSLRAEDTAVYYCARGGLYGSGSYLSD |
| | | FDLWGRGTLVTVSSASTKGPSVFPLAPCSRSTSESTAA |
| | | LGCLVKDYFPEPVTVSWNSGALTSGVHTFPAVLQSSG |
| | | LYSLSSVVTVPSSSLGTKTYTCNVDHKPSNTKVDKRVE |
| | | SKYGPPCPPCPAPEFLGGPSVFLFPPKPKDTLMISRTP |
| | | EVTCVVVDVSQEDPEVQFNWYVDGVEVHNAKTKPRE |
| | | EQFNSTYRVVSVLTVLHQDWLNGKEYKCKVSNKGLPS |
| | | SIEKTISKAKGQPREPQVYTLPPSQEEMTKNQVSLTCL |
| | | VKGFYPSDIAVEWESNGQPENNYKTTPPVLDSDGSFF |
| | | LYSRLTVDKSRWQEGNVFSCSVMHEALHNHYTQKSLS |
| | | LSLGX, |
| | | wherein X is K or absent |
| SEQ ID NO: 47 | 373.A | GAAGTGCAGCTGCTGGAATCTGGCGGCGGACTGGT |
| | DNA | GCAGCCTGGCGGCTCCCTGAGACTGTCTTGCGCCG |
| | Heavy | CCTCCGGCTTCACCTTCCACAGATACGCCATGTCCT |
| | Chain | GGGTCCGACAGGCCCCTGGCAAGGGCCTGGAGTGG |
| | | GTGTCCGCCATCTCCGGCTCCGGCATGAACACCTAC |
| | | TACGCCGACTCCGTGAAGGGCCGGTTCACCATCTCC |
| | | CGGGACAACTCCAAGAACACCCTGTACCTGCAGATG |
| | | AACTCCCTGCGGGCCGAGGACACCGCCGTGTACTA |
| | | CTGTGCCAGAGGCGGCCTGTACGGCTCCGGCTCCT |
| | | ACCTGTCCGACTTCGACCTGTGGGGCAGAGGCACC |
| | | CTGGTCACCGTGTCCTCCGCCTCCACAAAGGGCCC |
| | | CTCCGTGTTCCCTCTGGCCCCTTGCTCCCGGTCCAC |
| | | CTCCGAGTCTACCGCCGCTCTGGGCTGCCTCGTGAA |
| | | GGACTACTTCCCCGAGCCCGTGACCGTGTCCTGGAA |
| | | CTCTGGCGCCCTGACCTCCGGCGTGCACACCTTCC |
| | | CTGCCGTGCTGCAGTCCTCCGGCCTGTACTCCCTGT |
| | | CCAGCGTCGTGACCGTGCCCTCCTCCAGCCTGGGC |
| | | ACCAAGACCTACACCTGTAACGTGGACCACAAGCCC |
| | | TCCAACACCAAAGTGGACAAGCGGGTGGAATCTAAG |
| | | TACGGCCCTCCCTGCCCTCCTTGCCCTGCCCCTGAG |
| | | TTCCTGGGCGGACCTTCCGTGTTCCTGTTCCCTCCA |
| | | AAGCCCAAGGACACCCTGATGATCTCCCGGACCCCT |
| | | GAAGTGACCTGCGTGGTGGTGGACGTGTCCCAGGA |
| | | AGATCCCGAAGTCCAGTTCAATTGGTACGTGGACGG |
| | | CGTGGAAGTGCACAACGCCAAGACCAAGCCCAGAG |
| | | AGGAACAGTTCAACTCCACCTACCGGGTGGTGTCCG |
| | | TGCTGACCGTGCTGCACCAGGACTGGCTGAACGGC |
| | | AAAGAGTACAAGTGCAAAGTGTCCAACAAGGGCCTG |
| | | CCCTCCAGCATCGAAAAGACCATCTCCAAGGCCAAG |
| | | GGCCAGCCCCGCGAGCCCCAAGTGTACACCCTGCC |
| | | TCCCAGCCAGGAAGAGATGACCAAGAATCAAGTGTC |
| | | CCTGACTTGTCTGGTCAAGGGCTTCTACCCCTCCGA |
| | | TATCGCCGTGGAGTGGGAGTCCAACGGCCAGCCCG |
| | | AGAACAACTACAAGACCACCCCTCCCGTGCTGGACT |
| | | CCGACGGCTCCTTCTTCCTGTACTCTCGGCTGACCG |
| | | TGGACAAGTCCCGGTGGCAGGAAGGCAACGTCTTCT |
| | | CCTGCTCCGTGATGCACGAGGCCCTGCACAACCACT |
| | | ACACCCAGAAGTCCCTGTCCCTGTCTCTGGGC |
| | | X₁X₂X₃, |
| | | wherein X₁ is A, X₂ is A, and X₃ is |
| | | G; or X₁ is absent, X₂ is absent, |
| | | and X₃ is absent. |
| SEQ ID NO: 114 | 373.B | EVQLLESGGGLVQPGGSLRLSCAASGFTFHRYAMSW |
| | Heavy | VRQAPGKGLEWVSAISGSGMNTYYADSVKGRFTISRD |
| | chain | NSKNTLYLQMNSLRAEDTAVYYCARGGLYGSGSYLSD |
| | | FDLWGRGTLVTVSSASTKGPSVFPLAPCSRSTSESTAA |
| | | LGCLVKDYFPEPVTVSWNSGALTSGVHTFPAVLQSSG |
| | | LYSLSSVVTVPSSSLGTKTYTCNVDHKPSNTKVDKRVE |
| | | SKYGPPCPPCPAPEFEGGPSVFLFPPKPKDTLMISRTP |
| | | EVTCVVVDVSQEDPEVQFNWYVDGVEVHNAKTKPRE |
| | | EQFNSTYRVVSVLTVLHQDWLNGKEYKCKVSNKGLPS |
| | | SIEKTISKAKGQPREPQVYTLPPSQEEMTKNQVSLTCL |
| | | VKGFYPSDIAVEWESNGQPENNYKTTPPVLDSDGSFF |
| | | LYSRLTVDKSRWQEGNVFSCSVMHEALHNHYTQKSLS |
| | | LSLGX, |
| | | wherein X is K or absent |
| SEQ ID NO: 48 | LCDR1 | RASQSVGSNLA |
| (Combined) | | |
| SEQ ID NO: 49 | LCDR2 | GASTRAT |
| (Combined) | | |
| SEQ ID NO: 50 | LCDR3 | QQHNAFPYT |
| (Combined) | | |
| SEQ ID NO: 48 (Kabat) | LCDR1 | RASQSVGSNLA |
| SEQ ID NO: 49 (Kabat) | LCDR2 | GASTRAT |
| SEQ ID NO: 50 (Kabat) | LCDR3 | QQHNAFPYT |
| SEQ ID NO: 51 (Chothia) | LCDR1 | SQSVGSN |
| SEQ ID NO: 52 (Chothia) | LCDR2 | GAS |
| SEQ ID NO: 53 (Chothia) | LCDR3 | HNAFPY |
| SEQ ID NO: 54 (IMGT) | LCDR1 | QSVGSN |
| SEQ ID NO: 52 (IMGT) | LCDR2 | GAS |
| SEQ ID NO: 50 (IMGT) | LCDR3 | QQHNAFPYT |
| SEQ ID NO: 48 | LCDR1 | RASQSVGSNLA |
| (Alternative) | | |
| SEQ ID NO: 49 | LCDR2 | GASTRAT |
| (Alternative) | | |
| SEQ ID NO: 50 | LCDR3 | QQHNAFPYT |
| (Alternative) | | |
| SEQ ID NO: 55 | VL | EIVLTQSPATLSVSPGERATLSCRASQSVGSNLAWYQ |
| | | QKPGQAPRLLIYGASTRATGIPARFSGSGSGTEFTLTIS |
| | | SLQSEDFAVYYCQQHNAFPYTFGGGTKVEIK |
| SEQ ID NO: 56 | DNA VL | GAGATCGTGCTGACACAGTCCCCTGCCACCCTGTCT |
| | | GTGTCTCCCGGCGAGAGAGCCACCCTGAGCTGCAG |
| | | AGCCTCCCAGTCCGTGGGCTCCAACCTGGCCTGGT |
| | | ATCAGCAGAAGCCCGGCCAGGCCCCTCGGCTGCTG |
| | | ATCTACGGCGCCTCTACCAGAGCCACCGGCATCCCT |
| | | GCCAGATTCTCCGGCTCTGGCTCCGGCACCGAGTTC |
| | | ACCCTGACCATCTCCAGCCTGCAGTCCGAGGACTTC |
| | | GCCGTGTACTACTGCCAGCAGCACAACGCCTTCCCT |
| | | TACACCTTCGGCGGAGGCACCAAAGTGGAAATCAAG |
| SEQ ID NO: 57 | Light | EIVLTQSPATLSVSPGERATLSCRASQSVGSNLAWYQ |
| | Chain | QKPGQAPRLLIYGASTRATGIPARFSGSGSGTEFTLTIS |
| | | SLQSEDFAVYYCQQHNAFPYTFGGGTKVEIKRTVAAPS |
| | | VFIFPPSDEQLKSGTASVVCLLNNFYPREAKVQWKVDN |
| | | ALQSGNSQESVTEQDSKDSTYSLSSTLTLSKADYEKHK |
| | | VYACEVTHQGLSSPVTKSFNRGEC |
| SEQ ID NO: 58 | DNA | GAGATCGTGCTGACACAGTCCCCTGCCACCCTGTCT |
| | Light | GTGTCTCCCGGCGAGAGAGCCACCCTGAGCTGCAG |
| | Chain | AGCCTCCCAGTCCGTGGGCTCCAACCTGGCCTGGT |
| | | ATCAGCAGAAGCCCGGCCAGGCCCCTCGGCTGCTG |
| | | ATCTACGGCGCCTCTACCAGAGCCACCGGCATCCCT |
| | | GCCAGATTCTCCGGCTCTGGCTCCGGCACCGAGTTC |
| | | ACCCTGACCATCTCCAGCCTGCAGTCCGAGGACTTC |
| | | GCCGTGTACTACTGCCAGCAGCACAACGCCTTCCCT |
| | | TACACCTTCGGCGGAGGCACCAAAGTGGAAATCAAG |
| | | CGTACGGTGGCCGCTCCCTCCGTGTTCATCTTCCCA |
| | | CCCTCCGACGAGCAGCTGAAGTCCGGCACCGCCTC |
| | | CGTCGTGTGCCTGCTGAACAACTTCTACCCTCGCGA |
| | | GGCCAAAGTGCAGTGGAAAGTGGACAACGCCCTGC |
| | | AGTCCGGCAACTCCCAGGAATCCGTCACCGAGCAG |
| | | GACTCCAAGGACAGCACCTACTCCCTGTCCTCCACC |
| | | CTGACCCTGTCCAAGGCCGACTACGAGAAGCACAAA |
| | | GTGTACGCCTGCGAAGTGACCCACCAGGGCCTGTC |
| | | CAGCCCCGTGACCAAGTCCTTCAACCGGGGCGAGT |
| | | GC |
| 350 | | |
| SEQ ID NO: 59 | HCDR1 | GGSIERYYWS |
| (Combined) | | |
| SEQ ID NO: 60 | HCDR2 | YIYGRGSTNYNPSLKS |
| (Combined) | | |
| SEQ ID NO: 3 | HCDR3 | ESQESPYNNWFDP |
| (Combined) | | |
| SEQ ID NO: 61 (Kabat) | HCDR1 | RYYWS |
| SEQ ID NO: 60 (Kabat) | HCDR2 | YIYGRGSTNYNPSLKS |
| SEQ ID NO: 3 (Kabat) | HCDR3 | ESQESPYNNWFDP |
| SEQ ID NO: 62 (Chothia) | HCDR1 | GGSIERY |
| SEQ ID NO: 63 (Chothia) | HCDR2 | YGRGS |
| SEQ ID NO: 3 (Chothia) | HCDR3 | ESQESPYNNWFDP |
| SEQ ID NO: 64 (IMGT) | HCDR1 | GGSIERYY |
| SEQ ID NO: 65 (IMGT) | HCDR2 | IYGRGST |
| SEQ ID NO: 9 (IMGT) | HCDR3 | ARESQESPYNNWFDP |
| SEQ ID NO: 197 | HCDR1 | GSIERYYWS |
| (Alternative) | | |
| SEQ ID NO: 60 | HCDR2 | YIYGRGSTNYNPSLKS |
| (Alternative) | | |
| SEQ ID NO: 9 | HCDR3 | ARESQESPYNNWFDP |
| (Alternative) | | |
| SEQ ID NO: 66 | VH | QVQLQESGPGLVKPSETLSLTCTVSGGSIERYYWSWIR |
| | | QPPGKGLEWIGYIYGRGSTNYNPSLKSRVTISVDTSKN |
| | | QFSLKLSSVTAADTAVYYCARESQESPYNNWFDPWG |
| | | QGTLVTVSS |
| SEQ ID NO: 67 | DNA VH | CAAGTGCAGCTGCAGGAATCTGGCCCTGGCCTGGT |
| | | CAAGCCCTCCGAGACACTGTCCCTGACCTGCACCGT |
| | | GTCCGGCGGCTCCATCGAGCGGTACTACTGGTCCT |
| | | GGATCCGGCAGCCTCCCGGCAAGGGCCTGGAATGG |
| | | ATCGGCTACATCTACGGCAGAGGCTCCACCAACTAC |
| | | AACCCCAGCCTGAAGTCCAGAGTGACCATCTCCGTG |
| | | GACACCTCCAAGAACCAGTTCTCCCTGAAGCTGTCC |
| | | TCCGTGACCGCCGCTGACACCGCCGTGTACTACTGC |
| | | GCCAGAGAGTCCCAGGAATCCCCTTACAACAATTGG |
| | | TTCGACCCCTGGGGCCAGGGCACCCTGGTCACCGT |
| | | GTCCTCT |
| SEQ ID NO: 68 | 350.A | QVQLQESGPGLVKPSETLSLTCTVSGGSIERYYWSWIR |
| | Heavy | QPPGKGLEWIGYIYGRGSTNYNPSLKSRVTISVDTSKN |
| | Chain | QFSLKLSSVTAADTAVYYCARESQESPYNNWFDPWG |
| | | QGTLVTVSSASTKGPSVFPLAPCSRSTSESTAALGCLV |
| | | KDYFPEPVTVSWNSGALTSGVHTFPAVLQSSGLYSLS |
| | | SVVTVPSSSLGTKTYTCNVDHKPSNTKVDKRVESKYG |
| | | PPCPPCPAPEFLGGPSVFLFPPKPKDTLMISRTPEVTC |
| | | VVVDVSQEDPEVQFNWYVDGVEVHNAKTKPREEQFN |
| | | STYRVVSVLTVLHQDWLNGKEYKCKVSNKGLPSSIEKT |
| | | ISKAKGQPREPQVYTLPPSQEEMTKNQVSLTCLVKGFY |
| | | PSDIAVEWESNGQPENNYKTTPPVLDSDGSFFLYSRLT |
| | | VDKSRWQEGNVFSCSVMHEALHNHYTQKSLSLSLGX, |
| | | wherein X is K or absent |
| SEQ ID NO: 69 | 350.A | CAAGTGCAGCTGCAGGAATCTGGCCCTGGCCTGGT |
| | DNA | CAAGCCCTCCGAGACACTGTCCCTGACCTGCACCGT |
| | Heavy | GTCCGGCGGCTCCATCGAGCGGTACTACTGGTCCT |
| | Chain | GGATCCGGCAGCCTCCCGGCAAGGGCCTGGAATGG |
| | | ATCGGCTACATCTACGGCAGAGGCTCCACCAACTAC |
| | | AACCCCAGCCTGAAGTCCAGAGTGACCATCTCCGTG |
| | | GACACCTCCAAGAACCAGTTCTCCCTGAAGCTGTCC |
| | | TCCGTGACCGCCGCTGACACCGCCGTGTACTACTGC |
| | | GCCAGAGAGTCCCAGGAATCCCCTTACAACAATTGG |
| | | TTCGACCCCTGGGGCCAGGGCACCCTGGTCACCGT |
| | | GTCCTCTGCCTCCACCAAGGGCCCCTCCGTGTTCCC |
| | | TCTGGCCCCTTGCTCCCGGTCCACCTCCGAGTCTAC |
| | | CGCCGCTCTGGGCTGCCTCGTGAAGGACTACTTCCC |
| | | CGAGCCCGTGACCGTGTCCTGGAACTCTGGCGCCC |
| | | TGACCTCCGGCGTGCACACCTTCCCTGCCGTGCTGC |
| | | AGTCCTCCGGCCTGTACTCCCTGTCCAGCGTCGTGA |
| | | CCGTGCCCTCCTCCAGCCTGGGCACCAAGACCTACA |
| | | CCTGTAACGTGGACCACAAGCCCTCCAACACCAAAG |
| | | TGGACAAGCGGGTGGAATCTAAGTACGGCCCTCCCT |
| | | GCCCTCCTTGCCCTGCCCCTGAGTTCCTGGGCGGA |
| | | CCTTCCGTGTTCCTGTTCCCTCCAAAGCCCAAGGAC |
| | | ACCCTGATGATCTCCCGGACCCCTGAAGTGACCTGC |
| | | GTGGTGGTGGACGTGTCCCAGGAAGATCCCGAAGT |
| | | CCAGTTCAATTGGTACGTGGACGGCGTGGAAGTGCA |
| | | CAACGCCAAGACCAAGCCCAGAGAGGAACAGTTCAA |
| | | CTCCACCTACCGGGTGGTGTCCGTGCTGACCGTGCT |
| | | GCACCAGGACTGGCTGAACGGCAAAGAGTACAAGT |
| | | GCAAAGTGTCCAACAAGGGCCTGCCCTCCAGCATCG |
| | | AAAAGACCATCTCCAAGGCCAAGGGCCAGCCCCGC |
| | | GAGCCCCAAGTGTACACCCTGCCTCCCAGCCAGGA |
| | | AGAGATGACCAAGAATCAAGTGTCCCTGACTTGTCT |
| | | GGTCAAGGGCTTCTACCCCTCCGATATCGCCGTGGA |
| | | GTGGGAGTCCAACGGCCAGCCCGAGAACAACTACA |
| | | AGACCACCCCTCCCGTGCTGGACTCCGACGGCTCC |
| | | TTCTTCCTGTACTCTCGGCTGACCGTGGACAAGTCC |
| | | CGGTGGCAGGAAGGCAACGTCTTCTCCTGCTCCGT |
| | | GATGCACGAGGCCCTGCACAACCACTACACCCAGAA |
| | | GTCCCTGTCCCTGTCTCTGGGC X₁X₂X₃, |
| | | wherein X₁ is A, X₂ is A, and X₃ is |
| | | G; or X₁ is absent, X₂ is absent, and |
| | | X₃ is absent. |
| SEQ ID NO: 115 | 350.B | QVQLQESGPGLVKPSETLSLTCTVSGGSIERYYWSWIR |
| | Heavy | QPPGKGLEWIGYIYGRGSTNYNPSLKSRVTISVDTSKN |
| | chain | QFSLKLSSVTAADTAVYYCARESQESPYNNWFDPWG |
| | | QGTLVTVSSASTKGPSVFPLAPCSRSTSESTAALGCLV |
| | | KDYFPEPVTVSWNSGALTSGVHTFPAVLQSSGLYSLS |
| | | SWVTVPSSSLGTKTYTCNVDHKPSNTKVDKRVESKYG |
| | | PPCPPCPAPEFEGGPSVFLFPPKPKDTLMISRTPEVTC |
| | | VVVDVSQEDPEVQFNWYVDGVEVHNAKTKPREEQFN |
| | | STYRVVSVLTVLHQDWLNGKEYKCKVSNKGLPSSIEKT |
| | | ISKAKGQPREPQVYTLPPSQEEMTKNQVSLTCLVKGFY |
| | | PSDIAVEWESNGQPENNYKTTPPVLDSDGSFFLYSRLT |
| | | VDKSRWQEGNVFSCSVMHEALHNHYTQKSLSLSLGX, |
| | | wherein X is K or absent |
| SEQ ID NO: 331 | 350 Fab | QVQLQESGPGLVKPSETLSLTCTVSGGSIERYYWSWIR |
| | heavy | QPPGKGLEWIGYIYGRGSTNYNPSLKSRVTISVDTSKN |
| | chain | QFSLKLSSVTAADTAVYYCARESQESPYNNWFDPWG |
| | | QGTLVTVSSASTKGPSVFPLAPCSRSTSESTAALGCLV |
| | | KDYFPEPVTVSWNSGALTSGVHTFPAVLQSSGLYSLS |
| | | SWVTVPSSSLGTKTYTCNVDHKPSNTKVDKRVESKYG |
| | | P |
| SEQ ID NO: 14 | LCDR1 | RASQGISSWLA |
| (Combined) | | |
| SEQ ID NO: 15 | LCDR2 | AASSLQS |
| (Combined) | | |
| SEQ ID NO: 16 | LCDR3 | QQGNSFPRT |
| (Combined) | | |
| SEQ ID NO: 14 (Kabat) | LCDR1 | RASQGISSWLA |
| SEQ ID NO: 15 (Kabat) | LCDR2 | AASSLQS |
| SEQ ID NO: 16 (Kabat) | LCDR3 | QQGNSFPRT |
| SEQ ID NO: 17 (Chothia) | LCDR1 | SQGISSW |
| SEQ ID NO: 18 (Chothia) | LCDR2 | AAS |
| SEQ ID NO: 19 (Chothia) | LCDR3 | GNSFPR |
| SEQ ID NO: 20 (IMGT) | LCDR1 | QGISSW |
| SEQ ID NO: 18 (IMGT) | LCDR2 | AAS |
| SEQ ID NO: 16 (IMGT) | LCDR3 | QQGNSFPRT |
| SEQ ID NO: 14 | LCDR1 | RASQGISSWLA |
| (Alternative) | | |
| SEQ ID NO: 15 | LCDR2 | AASSLQS |
| (Alternative) | | |
| SEQ ID NO: 16 | LCDR3 | QQGNSFPRT |
| (Alternative) | | |
| SEQ ID NO: 21 | VL | DIQMTQSPSSVSASVGDRVTITCRASQGISSWLAWYQ |
| | | QKPGKAPKLLIYAASSLQSGVPSRFSGSGSGTDFTLTIS |
| | | SLQPEDFATYYCQQGNSFPRTFGGGTKVEIK |
| SEQ ID NO: 22 | DNA VL | GACATCCAGATGACCCAGAGCCCCTCCTCCGTGTCC |
| | | GCCTCCGTGGGCGACAGAGTGACCATCACCTGTCG |
| | | GGCCTCCCAGGGCATCTCCAGCTGGCTGGCCTGGT |
| | | ATCAGCAGAAGCCCGGCAAGGCCCCTAAGCTGCTG |
| | | ATCTACGCCGCCTCCAGCCTGCAGTCCGGCGTGCC |
| | | CTCCAGATTCTCCGGCTCTGGCTCCGGCACCGACTT |
| | | CACCCTGACCATCAGCTCCCTGCAGCCCGAGGACTT |
| | | CGCCACCTACTACTGCCAGCAGGGCAACTCCTTCCC |
| | | TCGGACCTTCGGCGGAGGCACCAAAGTGGAAATCAA |
| | | G |
| SEQ ID NO: 23 | Light | DIQMTQSPSSVSASVGDRVTITCRASQGISSWLAWYQ |
| | Chain | QKPGKAPKLLIYAASSLQSGVPSRFSGSGSGTDFTLTIS |
| | | SLQPEDFATYYCQQGNSFPRTFGGGTKVEIKRTVAAP |
| | | SVFIFPPSDEQLKSGTASVVCLLNNFYPREAKVQWKVD |
| | | NALQSGNSQESVTEQDSKDSTYSLSSTLTLSKADYEKH |
| | | KVYACEVTHQGLSSPVTKSFNRGEC |
| SEQ ID NO: 24 | DNA | GACATCCAGATGACCCAGAGCCCCTCCTCCGTGTCC |
| | Light | GCCTCCGTGGGCGACAGAGTGACCATCACCTGTCG |
| | Chain | GGCCTCCCAGGGCATCTCCAGCTGGCTGGCCTGGT |
| | | ATCAGCAGAAGCCCGGCAAGGCCCCTAAGCTGCTG |
| | | ATCTACGCCGCCTCCAGCCTGCAGTCCGGCGTGCC |
| | | CTCCAGATTCTCCGGCTCTGGCTCCGGCACCGACTT |
| | | CACCCTGACCATCAGCTCCCTGCAGCCCGAGGACTT |
| | | CGCCACCTACTACTGCCAGCAGGGCAACTCCTTCCC |
| | | TCGGACCTTCGGCGGAGGCACCAAAGTGGAAATCAA |
| | | GCGTACGGTGGCCGCTCCCTCCGTGTTCATCTTCCC |
| | | ACCCTCCGACGAGCAGCTGAAGTCCGGCACCGCCT |
| | | CCGTCGTGTGCCTGCTGAACAACTTCTACCCTCGCG |
| | | AGGCCAAAGTGCAGTGGAAAGTGGACAACGCCCTG |
| | | CAGTCCGGCAACTCCCAGGAATCCGTCACCGAGCA |
| | | GGACTCCAAGGACAGCACCTACTCCCTGTCCTCCAC |
| | | CCTGACCCTGTCCAAGGCCGACTACGAGAAGCACAA |
| | | AGTGTACGCCTGCGAAGTGACCCACCAGGGCCTGT |
| | | CCAGCCCCGTGACCAAGTCCTTCAACCGGGGCGAG |
| | | TGC |
| 374 | | |
| SEQ ID NO: 70 | HCDR1 | GFTFSYNAMS |
| (Combined) | | |
| SEQ ID NO: 71 | HCDR2 | SISGTGGSTYYADSVKG |
| (Combined) | | |
| SEQ ID NO: 37 | HCDR3 | GGLYGSGSYLSDFDL |
| (Combined) | | |
| SEQ ID NO: 72 (Kabat) | HCDR1 | YNAMS |
| SEQ ID NO: 71 (Kabat) | HCDR2 | SISGTGGSTYYADSVKG |
| SEQ ID NO: 37 (Kabat) | HCDR3 | GGLYGSGSYLSDFDL |
| SEQ ID NO: 73 (Chothia) | HCDR1 | GFTFSYN |
| SEQ ID NO: 74 (Chothia) | HCDR2 | SGTGGS |
| SEQ ID NO: 37 (Chothia) | HCDR3 | GGLYGSGSYLSDFDL |
| SEQ ID NO: 75 (IMGT) | HCDR1 | GFTFSYNA |
| SEQ ID NO: 76 (IMGT) | HCDR2 | ISGTGGST |
| SEQ ID NO: 43 (IMGT) | HCDR3 | ARGGLYGSGSYLSDFDL |
| SEQ ID NO: 191 | HCDR1 | FTFSYNAMS |
| (Alternative) | | |
| SEQ ID NO: 71 | HCDR2 | SISGTGGSTYYADSVKG |
| (Alternative) | | |
| SEQ ID NO: 43 | HCDR3 | ARGGLYGSGSYLSDFDL |
| (Alternative) | | |
| SEQ ID NO: 77 | VH | EVQLLESGGGLVQPGGSLRLSCAASGFTFSYNAMSWV |
| | | RQAPGKGLEWVSSISGTGGSTYYADSVKGRFTISRDN |
| | | SKNTLYLQMNSLRAEDTAVYYCARGGLYGSGSYLSDF |
| | | DLWGRGTLVTVSS |
| SEQ ID NO: 78 | DNA VH | GAAGTGCAGCTGCTGGAATCTGGCGGCGGACTGGT |
| | | GCAGCCTGGCGGCTCCCTGAGACTGTCTTGCGCCG |
| | | CCTCCGGCTTCACCTTCTCCTACAACGCCATGTCCT |
| | | GGGTCCGACAGGCCCCTGGCAAGGGCCTGGAGTGG |
| | | GTGTCCTCCATCTCCGGCACCGGCGGCTCCACCTAC |
| | | TACGCCGACTCTGTGAAGGGCCGGTTCACCATCTCC |
| | | CGGGACAACTCCAAGAACACCCTGTACCTGCAGATG |
| | | AACTCCCTGCGGGCCGAGGACACCGCCGTGTACTA |
| | | CTGTGCCAGAGGCGGCCTGTACGGCTCCGGCTCCT |
| | | ACCTGTCCGACTTCGACCTGTGGGGCAGAGGCACC |
| | | CTGGTCACCGTGTCCTCC |
| SEQ ID NO: 79 | 374.A | EVQLLESGGGLVQPGGSLRLSCAASGFTFSYNAMSWV |
| | Heavy | RQAPGKGLEWVSSISGTGGSTYYADSVKGRFTISRDN |
| | Chain | SKNTLYLQMNSLRAEDTAVYYCARGGLYGSGSYLSDF |
| | | DLWGRGTLVTVSSASTKGPSVFPLAPCSRSTSESTAAL |
| | | GCLVKDYFPEPVTVSWNSGALTSGVHTFPAVLQSSGL |
| | | YSLSSVVTVPSSSLGTKTYTCNVDHKPSNTKVDKRVES |
| | | KYGPPCPPCPAPEFLGGPSVFLFPPKPKDTLMISRTPE |
| | | VTCVVVDVSQEDPEVQFNWYVDGVEVHNAKTKPREE |
| | | QFNSTYRVVSVLTVLHQDWLNGKEYKCKVSNKGLPSSI |
| | | EKTISKAKGQPREPQVYTLPPSQEEMTKNQVSLTCLVK |
| | | GFYPSDIAVEWESNGQPENNYKTTPPVLDSDGSFFLY |
| | | SRLTVDKSRWQEGNVFSCSVMHEALHNHYTQKSLSLS |
| | | LGX, |
| | | wherein X is K or absent |
| SEQ ID NO: 80 | 374.A | GAAGTGCAGCTGCTGGAATCTGGCGGCGGACTGGT |
| | DNA | GCAGCCTGGCGGCTCCCTGAGACTGTCTTGCGCCG |
| | Heavy | CCTCCGGCTTCACCTTCTCCTACAACGCCATGTCCT |
| | Chain | GGGTCCGACAGGCCCCTGGCAAGGGCCTGGAGTGG |
| | | GTGTCCTCCATCTCCGGCACCGGCGGCTCCACCTAC |
| | | TACGCCGACTCTGTGAAGGGCCGGTTCACCATCTCC |
| | | CGGGACAACTCCAAGAACACCCTGTACCTGCAGATG |
| | | AACTCCCTGCGGGCCGAGGACACCGCCGTGTACTA |
| | | CTGTGCCAGAGGCGGCCTGTACGGCTCCGGCTCCT |
| | | ACCTGTCCGACTTCGACCTGTGGGGCAGAGGCACC |
| | | CTGGTCACCGTGTCCTCCGCCTCCACAAAGGGCCC |
| | | CTCCGTGTTCCCTCTGGCCCCTTGCTCCCGGTCCAC |
| | | CTCCGAGTCTACCGCCGCTCTGGGCTGCCTCGTGAA |
| | | GGACTACTTCCCCGAGCCCGTGACCGTGTCCTGGAA |
| | | CTCTGGCGCCCTGACCTCCGGCGTGCACACCTTCC |
| | | CTGCCGTGCTGCAGTCCTCCGGCCTGTACTCCCTGT |
| | | CCAGCGTCGTGACCGTGCCCTCCTCCAGCCTGGGC |
| | | ACCAAGACCTACACCTGTAACGTGGACCACAAGCCC |
| | | TCCAACACCAAAGTGGACAAGCGGGTGGAATCTAAG |
| | | TACGGCCCTCCCTGCCCTCCTTGCCCTGCCCCTGAG |
| | | TTCCTGGGCGGACCTTCCGTGTTCCTGTTCCCTCCA |
| | | AAGCCCAAGGACACCCTGATGATCTCCCGGACCCCT |
| | | GAAGTGACCTGCGTGGTGGTGGACGTGTCCCAGGA |
| | | AGATCCCGAAGTCCAGTTCAATTGGTACGTGGACGG |
| | | CGTGGAAGTGCACAACGCCAAGACCAAGCCCAGAG |
| | | AGGAACAGTTCAACTCCACCTACCGGGTGGTGTCCG |
| | | TGCTGACCGTGCTGCACCAGGACTGGCTGAACGGC |
| | | AAAGAGTACAAGTGCAAAGTGTCCAACAAGGGCCTG |
| | | CCCTCCAGCATCGAAAAGACCATCTCCAAGGCCAAG |
| | | GGCCAGCCCCGCGAGCCCCAAGTGTACACCCTGCC |
| | | TCCCAGCCAGGAAGAGATGACCAAGAATCAAGTGTC |
| | | CCTGACTTGTCTGGTCAAGGGCTTCTACCCCTCCGA |
| | | TATCGCCGTGGAGTGGGAGTCCAACGGCCAGCCCG |
| | | AGAACAACTACAAGACCACCCCTCCCGTGCTGGACT |
| | | CCGACGGCTCCTTCTTCCTGTACTCTCGGCTGACCG |
| | | TGGACAAGTCCCGGTGGCAGGAAGGCAACGTCTTCT |
| | | CCTGCTCCGTGATGCACGAGGCCCTGCACAACCACT |
| | | ACACCCAGAAGTCCCTGTCCCTGTCTCTGGGC |
| | | X₁X₂X₃, |
| | | wherein X₁ is A, X₂ is A, and X₃ is |
| | | G; or X₁ is absent, X₂ is absent, |
| | | and X₃ is absent. |
| SEQ ID NO: 116 | 374.B | EVQLLESGGGLVQPGGSLRLSCAASGFTFSYNAMSWV |
| | Heavy | RQAPGKGLEWVSSISGTGGSTYYADSVKGRFTISRDN |
| | chain | SKNTLYLQMNSLRAEDTAVYYCARGGLYGSGSYLSDF |
| | | DLWGRGTLVTVSSASTKGPSVFPLAPCSRSTSESTAAL |
| | | GCLVKDYFPEPVTVSWNSGALTSGVHTFPAVLQSSGL |
| | | YSLSSWVTVPSSSLGTKTYTCNVDHKPSNTKVDKRVES |
| | | KYGPPCPPCPAPEFEGGPSVFLFPPKPKDTLMISRTPE |
| | | VTCVVVDVSQEDPEVQFNWYVDGVEVHNAKTKPREE |
| | | QFNSTYRVVSVLTVLHQDWLNGKEYKCKVSNKGLPSSI |
| | | EKTISKAKGQPREPQVYTLPPSQEEMTKNQVSLTCLVK |
| | | GFYPSDIAVEWESNGQPENNYKTTPPVLDSDGSFFLY |
| | | SRLTVDKSRWQEGNVFSCSVMHEALHNHYTQKSLSLS |
| | | LGX, |
| | | wherein X is K or absent |
| SEQ ID NO: 48 | LCDR1 | RASQSVGSNLA |
| (Combined) | | |
| SEQ ID NO: 49 | LCDR2 | GASTRAT |
| (Combined) | | |
| SEQ ID NO: 50 | LCDR3 | QQHNAFPYT |
| (Combined) | | |
| SEQ ID NO: 48 (Kabat) | LCDR1 | RASQSVGSNLA |
| SEQ ID NO: 49 (Kabat) | LCDR2 | GASTRAT |
| SEQ ID NO: 50 (Kabat) | LCDR3 | QQHNAFPYT |
| SEQ ID NO: 51 (Chothia) | LCDR1 | SQSVGSN |
| SEQ ID NO: 52 (Chothia) | LCDR2 | GAS |
| SEQ ID NO: 53 (Chothia) | LCDR3 | HNAFPY |
| SEQ ID NO: 54 (IMGT) | LCDR1 | QSVGSN |
| SEQ ID NO: 52 (IMGT) | LCDR2 | GAS |
| SEQ ID NO: 50 (IMGT) | LCDR3 | QQHNAFPYT |
| SEQ ID NO: 48 | LCDR1 | RASQSVGSNLA |
| (Alternative) | | |
| SEQ ID NO: 49 | LCDR2 | GASTRAT |
| (Alternative) | | |
| SEQ ID NO: 50 | LCDR3 | QQHNAFPYT |
| (Alternative) | | |
| SEQ ID NO: 55 | VL | EIVLTQSPATLSVSPGERATLSCRASQSVGSNLAWYQ |
| | | QKPGQAPRLLIYGASTRATGIPARFSGSGSGTEFTLTIS |
| | | SLQSEDFAVYYCQQHNAFPYTFGGGTKVEIK |
| SEQ ID NO: 56 | DNA VL | GAGATCGTGCTGACACAGTCCCCTGCCACCCTGTCT |
| | | GTGTCTCCCGGCGAGAGAGCCACCCTGAGCTGCAG |
| | | AGCCTCCCAGTCCGTGGGCTCCAACCTGGCCTGGT |
| | | ATCAGCAGAAGCCCGGCCAGGCCCCTCGGCTGCTG |
| | | ATCTACGGCGCCTCTACCAGAGCCACCGGCATCCCT |
| | | GCCAGATTCTCCGGCTCTGGCTCCGGCACCGAGTTC |
| | | ACCCTGACCATCTCCAGCCTGCAGTCCGAGGACTTC |
| | | GCCGTGTACTACTGCCAGCAGCACAACGCCTTCCCT |
| | | TACACCTTCGGCGGAGGCACCAAAGTGGAAATCAAG |
| SEQ ID NO: 57 | Light | EIVLTQSPATLSVSPGERATLSCRASQSVGSNLAWYQ |
| | Chain | QKPGQAPRLLIYGASTRATGIPARFSGSGSGTEFTLTIS |
| | | SLQSEDFAVYYCQQHNAFPYTFGGGTKVEIKRTVAAPS |
| | | VFIFPPSDEQLKSGTASVVCLLNNFYPREAKVQWKVDN |
| | | ALQSGNSQESVTEQDSKDSTYSLSSTLTLSKADYEKHK |
| | | VYACEVTHQGLSSPVTKSFNRGEC |
| SEQ ID NO: 58 | DNA | GAGATCGTGCTGACACAGTCCCCTGCCACCCTGTCT |
| | Light | GTGTCTCCCGGCGAGAGAGCCACCCTGAGCTGCAG |
| | Chain | AGCCTCCCAGTCCGTGGGCTCCAACCTGGCCTGGT |
| | | ATCAGCAGAAGCCCGGCCAGGCCCCTCGGCTGCTG |
| | | ATCTACGGCGCCTCTACCAGAGCCACCGGCATCCCT |
| | | GCCAGATTCTCCGGCTCTGGCTCCGGCACCGAGTTC |
| | | ACCCTGACCATCTCCAGCCTGCAGTCCGAGGACTTC |
| | | GCCGTGTACTACTGCCAGCAGCACAACGCCTTCCCT |
| | | TACACCTTCGGCGGAGGCACCAAAGTGGAAATCAAG |
| | | CGTACGGTGGCCGCTCCCTCCGTGTTCATCTTCCCA |
| | | CCCTCCGACGAGCAGCTGAAGTCCGGCACCGCCTC |
| | | CGTCGTGTGCCTGCTGAACAACTTCTACCCTCGCGA |
| | | GGCCAAAGTGCAGTGGAAAGTGGACAACGCCCTGC |
| | | AGTCCGGCAACTCCCAGGAATCCGTCACCGAGCAG |
| | | GACTCCAAGGACAGCACCTACTCCCTGTCCTCCACC |
| | | CTGACCCTGTCCAAGGCCGACTACGAGAAGCACAAA |
| | | GTGTACGCCTGCGAAGTGACCCACCAGGGCCTGTC |
| | | CAGCCCCGTGACCAAGTCCTTCAACCGGGGCGAGT |
| | | GC |
| 379 | | |
| SEQ ID NO: 81 | HCDR1 | GFTFSRYAMS |
| (Combined) | | |
| SEQ ID NO: 71 | HCDR2 | SISGTGGSTYYADSVKG |
| (Combined) | | |
| SEQ ID NO: 37 | HCDR3 | GGLYGSGSYLSDFDL |
| (Combined) | | |
| SEQ ID NO: 38 (Kabat) | HCDR1 | RYAMS |
| SEQ ID NO: 71 (Kabat) | HCDR2 | SISGTGGSTYYADSVKG |
| SEQ ID NO: 37 (Kabat) | HCDR3 | GGLYGSGSYLSDFDL |
| SEQ ID NO: 82 (Chothia) | HCDR1 | GFTFSRY |
| SEQ ID NO: 74 (Chothia) | HCDR2 | SGTGGS |
| SEQ ID NO: 37 (Chothia) | HCDR3 | GGLYGSGSYLSDFDL |
| SEQ ID NO: 83 (IMGT) | HCDR1 | GFTFSRYA |
| SEQ ID NO: 76 (IMGT) | HCDR2 | ISGTGGST |
| SEQ ID NO: 43 (IMGT) | HCDR3 | ARGGLYGSGSYLSDFDL |
| SEQ ID NO: 192 | HCDR1 | FTFSRYAMS |
| (Alternative) | | |
| SEQ ID NO: 71 | HCDR2 | SISGTGGSTYYADSVKG |
| (Alternative) | | |
| SEQ ID NO: 43 | HCDR3 | ARGGLYGSGSYLSDFDL |
| (Alternative) | | |
| SEQ ID NO: 84 | VH | EVQLLESGGGLVQPGGSLRLSCAASGFTFSRYAMSWV |
| | | RQAPGKGLEWVSSISGTGGSTYYADSVKGRFTISRDN |
| | | SKNTLYLQMNSLRAEDTAVYYCARGGLYGSGSYLSDF |
| | | DLWGRGTLVTVSS |
| SEQ ID NO: 85 | DNA VH | GAAGTGCAGCTGCTGGAATCTGGCGGCGGACTGGT |
| | | GCAGCCTGGCGGCTCCCTGAGACTGTCTTGCGCCG |
| | | CCTCCGGCTTCACCTTCTCCAGATACGCCATGTCCT |
| | | GGGTCCGACAGGCCCCTGGCAAGGGCCTGGAGTGG |
| | | GTGTCCTCCATCTCCGGCACCGGCGGCTCCACCTAC |
| | | TACGCCGACTCTGTGAAGGGCCGGTTCACCATCTCC |
| | | CGGGACAACTCCAAGAACACCCTGTACCTGCAGATG |
| | | AACTCCCTGCGGGCCGAGGACACCGCCGTGTACTA |
| | | CTGTGCCAGAGGCGGCCTGTACGGCTCCGGCTCCT |
| | | ACCTGTCCGACTTCGACCTGTGGGGCAGAGGCACC |
| | | CTGGTCACCGTGTCCTCC |
| SEQ ID NO: 86 | 379.A | EVQLLESGGGLVQPGGSLRLSCAASGFTFSRYAMSWV |
| | Heavy | RQAPGKGLEWVSSISGTGGSTYYADSVKGRFTISRDN |
| | Chain | SKNTLYLQMNSLRAEDTAVYYCARGGLYGSGSYLSDF |
| | | DLWGRGTLVTVSSASTKGPSVFPLAPCSRSTSESTAAL |
| | | GCLVKDYFPEPVTVSWNSGALTSGVHTFPAVLQSSGL |
| | | YSLSSVVTVPSSSLGTKTYTCNVDHKPSNTKVDKRVES |
| | | KYGPPCPPCPAPEFLGGPSVFLFPPKPKDTLMISRTPE |
| | | VTCVVVDVSQEDPEVQFNWYVDGVEVHNAKTKPREE |
| | | QFNSTYRVVSVLTVLHQDWLNGKEYKCKVSNKGLPSSI |
| | | EKTISKAKGQPREPQVYTLPPSQEEMTKNQVSLTCLVK |
| | | GFYPSDIAVEWESNGQPENNYKTTPPVLDSDGSFFLY |
| | | SRLTVDKSRWQEGNVFSCSVMHEALHNHYTQKSLSLS |
| | | LGX, |
| | | wherein X is K or absent |
| SEQ ID NO: 87 | 379.A | GAAGTGCAGCTGCTGGAATCTGGCGGCGGACTGGT |
| | DNA | GCAGCCTGGCGGCTCCCTGAGACTGTCTTGCGCCG |
| | Heavy | CCTCCGGCTTCACCTTCTCCAGATACGCCATGTCCT |
| | Chain | GGGTCCGACAGGCCCCTGGCAAGGGCCTGGAGTGG |
| | | GTGTCCTCCATCTCCGGCACCGGCGGCTCCACCTAC |
| | | TACGCCGACTCTGTGAAGGGCCGGTTCACCATCTCC |
| | | CGGGACAACTCCAAGAACACCCTGTACCTGCAGATG |
| | | AACTCCCTGCGGGCCGAGGACACCGCCGTGTACTA |
| | | CTGTGCCAGAGGCGGCCTGTACGGCTCCGGCTCCT |
| | | ACCTGTCCGACTTCGACCTGTGGGGCAGAGGCACC |
| | | CTGGTCACCGTGTCCTCCGCCTCCACAAAGGGCCC |
| | | CTCCGTGTTCCCTCTGGCCCCTTGCTCCCGGTCCAC |
| | | CTCCGAGTCTACCGCCGCTCTGGGCTGCCTCGTGAA |
| | | GGACTACTTCCCCGAGCCCGTGACCGTGTCCTGGAA |
| | | CTCTGGCGCCCTGACCTCCGGCGTGCACACCTTCC |
| | | CTGCCGTGCTGCAGTCCTCCGGCCTGTACTCCCTGT |
| | | CCAGCGTCGTGACCGTGCCCTCCTCCAGCCTGGGC |
| | | ACCAAGACCTACACCTGTAACGTGGACCACAAGCCC |
| | | TCCAACACCAAAGTGGACAAGCGGGTGGAATCTAAG |
| | | TACGGCCCTCCCTGCCCTCCTTGCCCTGCCCCTGAG |
| | | TTCCTGGGCGGACCTTCCGTGTTCCTGTTCCCTCCA |
| | | AAGCCCAAGGACACCCTGATGATCTCCCGGACCCCT |
| | | GAAGTGACCTGCGTGGTGGTGGACGTGTCCCAGGA |
| | | AGATCCCGAAGTCCAGTTCAATTGGTACGTGGACGG |
| | | CGTGGAAGTGCACAACGCCAAGACCAAGCCCAGAG |
| | | AGGAACAGTTCAACTCCACCTACCGGGTGGTGTCCG |
| | | TGCTGACCGTGCTGCACCAGGACTGGCTGAACGGC |
| | | AAAGAGTACAAGTGCAAAGTGTCCAACAAGGGCCTG |
| | | CCCTCCAGCATCGAAAAGACCATCTCCAAGGCCAAG |
| | | GGCCAGCCCCGCGAGCCCCAAGTGTACACCCTGCC |
| | | TCCCAGCCAGGAAGAGATGACCAAGAATCAAGTGTC |
| | | CCTGACTTGTCTGGTCAAGGGCTTCTACCCCTCCGA |
| | | TATCGCCGTGGAGTGGGAGTCCAACGGCCAGCCCG |
| | | AGAACAACTACAAGACCACCCCTCCCGTGCTGGACT |
| | | CCGACGGCTCCTTCTTCCTGTACTCTCGGCTGACCG |
| | | TGGACAAGTCCCGGTGGCAGGAAGGCAACGTCTTCT |
| | | CCTGCTCCGTGATGCACGAGGCCCTGCACAACCACT |
| | | ACACCCAGAAGTCCCTGTCCCTGTCTCTGGGC |
| | | X₁X₂X₃, |
| | | wherein X₁ is A, X₂ is A, and X₃ is |
| | | G; or X₁ is absent, X₂ is absent, |
| | | and X₃ is absent. |
| SEQ ID NO: 117 | 379.B | EVQLLESGGGLVQPGGSLRLSCAASGFTFSRYAMSWV |
| | Heavy | RQAPGKGLEWVSSISGTGGSTYYADSVKGRFTISRDN |
| | chain | SKNTLYLQMNSLRAEDTAVYYCARGGLYGSGSYLSDF |
| | | DLWGRGTLVTVSSASTKGPSVFPLAPCSRSTSESTAAL |
| | | GCLVKDYFPEPVTVSWNSGALTSGVHTFPAVLQSSGL |
| | | YSLSSVVTVPSSSLGTKTYTCNVDHKPSNTKVDKRVES |
| | | KYGPPCPPCPAPEFEGGPSVFLFPPKPKDTLMISRTPE |
| | | VTCVVVDVSQEDPEVQFNWYVDGVEVHNAKTKPREE |
| | | QFNSTYRVVSVLTVLHQDWLNGKEYKCKVSNKGLPSSI |
| | | EKTISKAKGQPREPQVYTLPPSQEEMTKNQVSLTCLVK |
| | | GFYPSDIAVEWESNGQPENNYKTTPPVLDSDGSFFLY |
| | | SRLTVDKSRWQEGNVFSCSVMHEALHNHYTQKSLSLS |
| | | LGX, |
| | | wherein X is K or absent |
| SEQ ID NO: 48 | LCDR1 | RASQSVGSNLA |
| (Combined) | | |
| SEQ ID NO: 49 | LCDR2 | GASTRAT |
| (Combined) | | |
| SEQ ID NO: 50 | LCDR3 | QQHNAFPYT |
| (Combined) | | |
| SEQ ID NO: 48 (Kabat) | LCDR1 | RASQSVGSNLA |
| SEQ ID NO: 49 (Kabat) | LCDR2 | GASTRAT |
| SEQ ID NO: 50 (Kabat) | LCDR3 | QQHNAFPYT |
| SEQ ID NO: 51 (Chothia) | LCDR1 | SQSVGSN |
| SEQ ID NO: 52 (Chothia) | LCDR2 | GAS |
| SEQ ID NO: 53 (Chothia) | LCDR3 | HNAFPY |
| SEQ ID NO: 54 (IMGT) | LCDR1 | QSVGSN |
| SEQ ID NO: 52 (IMGT) | LCDR2 | GAS |
| SEQ ID NO: 50 (IMGT) | LCDR3 | QQHNAFPYT |
| SEQ ID NO: 48 | LCDR1 | RASQSVGSNLA |
| (Alternative) | | |
| SEQ ID NO: 49 | LCDR2 | GASTRAT |
| (Alternative) | | |
| SEQ ID NO: 50 | LCDR3 | QQHNAFPYT |
| (Alternative) | | |
| SEQ ID NO: 55 | VL | EIVLTQSPATLSVSPGERATLSCRASQSVGSNLAWYQ |
| | | QKPGQAPRLLIYGASTRATGIPARFSGSGSGTEFTLTIS |
| | | SLQSEDFAVYYCQQHNAFPYTFGGGTKVEIK |
| SEQ ID NO: 56 | DNA VL | GAGATCGTGCTGACACAGTCCCCTGCCACCCTGTCT |
| | | GTGTCTCCCGGCGAGAGAGCCACCCTGAGCTGCAG |
| | | AGCCTCCCAGTCCGTGGGCTCCAACCTGGCCTGGT |
| | | ATCAGCAGAAGCCCGGCCAGGCCCCTCGGCTGCTG |
| | | ATCTACGGCGCCTCTACCAGAGCCACCGGCATCCCT |
| | | GCCAGATTCTCCGGCTCTGGCTCCGGCACCGAGTTC |
| | | ACCCTGACCATCTCCAGCCTGCAGTCCGAGGACTTC |
| | | GCCGTGTACTACTGCCAGCAGCACAACGCCTTCCCT |
| | | TACACCTTCGGCGGAGGCACCAAAGTGGAAATCAAG |
| SEQ ID NO: 57 | Light | EIVLTQSPATLSVSPGERATLSCRASQSVGSNLAWYQ |
| | Chain | QKPGQAPRLLIYGASTRATGIPARFSGSGSGTEFTLTIS |
| | | SLQSEDFAVYYCQQHNAFPYTFGGGTKVEIKRTVAAPS |
| | | VFIFPPSDEQLKSGTASVVCLLNNFYPREAKVQWKVDN |
| | | ALQSGNSQESVTEQDSKDSTYSLSSTLTLSKADYEKHK |
| | | VYACEVTHQGLSSPVTKSFNRGEC |
| SEQ ID NO: 58 | DNA | GAGATCGTGCTGACACAGTCCCCTGCCACCCTGTCT |
| | Light | GTGTCTCCCGGCGAGAGAGCCACCCTGAGCTGCAG |
| | Chain | AGCCTCCCAGTCCGTGGGCTCCAACCTGGCCTGGT |
| | | ATCAGCAGAAGCCCGGCCAGGCCCCTCGGCTGCTG |
| | | ATCTACGGCGCCTCTACCAGAGCCACCGGCATCCCT |
| | | GCCAGATTCTCCGGCTCTGGCTCCGGCACCGAGTTC |
| | | ACCCTGACCATCTCCAGCCTGCAGTCCGAGGACTTC |
| | | GCCGTGTACTACTGCCAGCAGCACAACGCCTTCCCT |
| | | TACACCTTCGGCGGAGGCACCAAAGTGGAAATCAAG |
| | | CGTACGGTGGCCGCTCCCTCCGTGTTCATCTTCCCA |
| | | CCCTCCGACGAGCAGCTGAAGTCCGGCACCGCCTC |
| | | CGTCGTGTGCCTGCTGAACAACTTCTACCCTCGCGA |
| | | GGCCAAAGTGCAGTGGAAAGTGGACAACGCCCTGC |
| | | AGTCCGGCAACTCCCAGGAATCCGTCACCGAGCAG |
| | | GACTCCAAGGACAGCACCTACTCCCTGTCCTCCACC |
| | | CTGACCCTGTCCAAGGCCGACTACGAGAAGCACAAA |
| | | GTGTACGCCTGCGAAGTGACCCACCAGGGCCTGTC |
| | | CAGCCCCGTGACCAAGTCCTTCAACCGGGGCGAGT |
| | | GC |
| 376 | | |
| SEQ ID NO: 135 | HCDR1 | GFTFRSYAMS |
| (Combined) | | |
| SEQ ID NO: 136 | HCDR2 | AITGSGGLTYYADSVKG |
| (Combined) | | |
| SEQ ID NO: 37 | HCDR3 | GGLYGSGSYLSDFDL |
| (Combined) | | |
| SEQ ID NO: 137 (Kabat) | HCDR1 | SYAMS |
| SEQ ID NO: 136 (Kabat) | HCDR2 | AITGSGGLTYYADSVKG |
| SEQ ID NO: 37 (Kabat) | HCDR3 | GGLYGSGSYLSDFDL |
| SEQ ID NO: 138 | HCDR1 | GFTFRSY |
| (Chothia) | | |
| SEQ ID NO: 139 | HCDR2 | TGSGGL |
| (Chothia) | | |
| SEQ ID NO: 37 (Chothia) | HCDR3 | GGLYGSGSYLSDFDL |
| SEQ ID NO: 140 (IMGT) | HCDR1 | GFTFRSYA |
| SEQ ID NO: 141 (IMGT) | HCDR2 | ITGSGGLT |
| SEQ ID NO: 43 (IMGT) | HCDR3 | ARGGLYGSGSYLSDFDL |
| SEQ ID NO: 193 | HCDR1 | FTFRSYAMS |
| (Alternative) | | |
| SEQ ID NO: 136 | HCDR2 | AITGSGGLTYYADSVKG |
| (Alternative) | | |
| SEQ ID NO: 43 | HCDR3 | ARGGLYGSGSYLSDFDL |
| (Alternative) | | |
| SEQ ID NO: 142 | VH | EVQLLESGGGLVQPGGSLRLSCAASGFTFRSYAMSWV |
| | | RQAPGKGLEWVSAITGSGGLTYYADSVKGRFTISRDN |
| | | SKNTLYLQMNSLRAEDTAVYYCARGGLYGSGSYLSDF |
| | | DLWGRGTLVTVSS |
| SEQ ID NO: 143 | DNA VH | GAGGTGCAGCTGTTGGAGTCTGGGGGAGGCTTGGT |
| | | ACAGCCTGGGGGGTCCCTGAGACTCTCCTGTGCAG |
| | | CCTCTGGATTCACCTTTCGTAGCTATGCCATGAGCT |
| | | GGGTCCGCCAGGCTCCAGGGAAGGGGCTGGAGTG |
| | | GGTCTCAGCTATTACGGGAAGTGGTGGTTTGACATA |
| | | CTACGCAGACTCCGTGAAGGGCCGGTTCACCATCTC |
| | | CAGAGACAATTCCAAGAACACGCTGTATCTGCAAAT |
| | | GAACAGCCTGAGAGCCGAGGACACGGCGGTGTACT |
| | | ACTGCGCCAGAGGTGGATTGTACGGAAGCGGAAGC |
| | | TACTTGAGTGACTTCGACCTATGGGGGAGAGGTACC |
| | | TTGGTCACCGTCTCCTCA |
| SEQ ID NO: 48 | LCDR1 | RASQSVGSNLA |
| (Combined) | | |
| SEQ ID NO: 49 | LCDR2 | GASTRAT |
| (Combined) | | |
| SEQ ID NO: 50 | LCDR3 | QQHNAFPYT |
| (Combined) | | |
| SEQ ID NO: 48 (Kabat) | LCDR1 | RASQSVGSNLA |
| SEQ ID NO: 49 (Kabat) | LCDR2 | GASTRAT |
| SEQ ID NO: 50 (Kabat) | LCDR3 | QQHNAFPYT |
| SEQ ID NO: 51 (Chothia) | LCDR1 | SQSVGSN |
| SEQ ID NO: 52 (Chothia) | LCDR2 | GAS |
| SEQ ID NO: 53 (Chothia) | LCDR3 | HNAFPY |
| SEQ ID NO: 54 (IMGT) | LCDR1 | QSVGSN |
| SEQ ID NO: 52 (IMGT) | LCDR2 | GAS |
| SEQ ID NO: 50 (IMGT) | LCDR3 | QQHNAFPYT |
| SEQ ID NO: 48 | LCDR1 | RASQSVGSNLA |
| (Alternative) | | |
| SEQ ID NO: 49 | LCDR2 | GASTRAT |
| (Alternative) | | |
| SEQ ID NO: 50 | LCDR3 | QQHNAFPYT |
| (Alternative) | | |
| SEQ ID NO: 55 | VL | EIVLTQSPATLSVSPGERATLSCRASQSVGSNLAWYQ |
| | | QKPGQAPRLLIYGASTRATGIPARFSGSGSGTEFTLTIS |
| | | SLQSEDFAVYYCQQHNAFPYTFGGGTKVEIK |
| SEQ ID NO: 144 | DNA VL | GAAATAGTGTTGACGCAGTCTCCAGCCACCCTGTCT |
| | | GTGTCTCCAGGGGAAAGAGCCACCCTCTCCTGCAG |
| | | GGCCAGTCAGAGTGTTGGCAGCAACTTAGCCTGGTA |
| | | CCAGCAGAAACCTGGCCAGGCTCCCAGGCTCCTCAT |
| | | CTATGGTGCATCCACCAGGGCCACTGGTATCCCAGC |
| | | CAGGTTCAGTGGCAGTGGGTCTGGGACAGAGTTCA |
| | | CTCTCACCATCAGCAGCCTGCAGTCTGAAGATTTTG |
| | | CAGTTTATTACTGTCAGCAGCACAATGCCTTCCCTTA |
| | | CACTTTTGGCGGAGGGACCAAGGTTGAGATCAAA |
| 377 | | |
| SEQ ID NO: 145 | HCDR1 | GFTFKSYAMS |
| (Combined) | | |
| SEQ ID NO: 146 | HCDR2 | AISGSGSYTYYADSVKG |
| (Combined) | | |
| SEQ ID NO: 37 | HCDR3 | GGLYGSGSYLSDFDL |
| (Combined) | | |
| SEQ ID NO: 137 (Kabat) | HCDR1 | SYAMS |
| SEQ ID NO: 146 (Kabat) | HCDR2 | AISGSGSYTYYADSVKG |
| SEQ ID NO: 37 (Kabat) | HCDR3 | GGLYGSGSYLSDFDL |
| SEQ ID NO: 147 | HCDR1 | GFTFKSY |
| (Chothia) | | |
| SEQ ID NO: 148 | HCDR2 | SGSGSY |
| (Chothia) | | |
| SEQ ID NO: 37 (Chothia) | HCDR3 | GGLYGSGSYLSDFDL |
| SEQ ID NO: 149 (IMGT) | HCDR1 | GFTFKSYA |
| SEQ ID NO: 150 (IMGT) | HCDR2 | ISGSGSYT |
| SEQ ID NO: 43 (IMGT) | HCDR3 | ARGGLYGSGSYLSDFDL |
| SEQ ID NO: 194 | HCDR1 | FTFKSYAMS |
| (Alternative) | | |
| SEQ ID NO: 146 | HCDR2 | AISGSGSYTYYADSVKG |
| (Alternative) | | |
| SEQ ID NO: 43 | HCDR3 | ARGGLYGSGSYLSDFDL |
| (Alternative) | | |
| SEQ ID NO: 151 | VH | EVQLLESGGGLVQPGGSLRLSCAASGFTFKSYAMSWV |
| | | RQAPGKGLEWVSAISGSGSYTYYADSVKGRFTISRDN |
| | | SKNTLYLQMNSLRAEDTAVYYCARGGLYGSGSYLSDF |
| | | DLWGRGTLVTVSS |
| SEQ ID NO: 152 | DNA VH | GAGGTGCAGCTGTTGGAGTCTGGGGGAGGCTTGGT |
| | | ACAGCCTGGGGGGTCCCTGAGACTCTCCTGTGCAG |
| | | CCTCTGGATTCACCTTTAAGAGCTATGCCATGAGTTG |
| | | GGTCCGCCAGGCTCCAGGGAAGGGGCTGGAGTGG |
| | | GTCTCAGCTATTAGTGGAAGTGGTTCGTATACATACT |
| | | ACGCAGACTCCGTGAAGGGCCGGTTCACCATCTCCA |
| | | GAGACAATTCCAAGAACACGCTGTATCTGCAAATGA |
| | | ACAGCCTGAGAGCCGAGGACACGGCGGTGTACTAC |
| | | TGCGCCAGAGGTGGATTGTACGGAAGCGGAAGCTA |
| | | CTTGAGTGACTTCGACCTATGGGGGAGAGGTACCTT |
| | | GGTCACCGTCTCCTCA |
| SEQ ID NO: 48 | LCDR1 | RASQSVGSNLA |
| (Combined) | | |
| SEQ ID NO: 49 | LCDR2 | GASTRAT |
| (Combined) | | |
| SEQ ID NO: 50 | LCDR3 | QQHNAFPYT |
| (Combined) | | |
| SEQ ID NO: 48 (Kabat) | LCDR1 | RASQSVGSNLA |
| SEQ ID NO: 49 (Kabat) | LCDR2 | GASTRAT |
| SEQ ID NO: 50 (Kabat) | LCDR3 | QQHNAFPYT |
| SEQ ID NO: 51 (Chothia) | LCDR1 | SQSVGSN |
| SEQ ID NO: 52 (Chothia) | LCDR2 | GAS |
| SEQ ID NO: 53 (Chothia) | LCDR3 | HNAFPY |
| SEQ ID NO: 54 (IMGT) | LCDR1 | QSVGSN |
| SEQ ID NO: 52 (IMGT) | LCDR2 | GAS |
| SEQ ID NO: 50 (IMGT) | LCDR3 | QQHNAFPYT |
| SEQ ID NO: 48 | LCDR1 | RASQSVGSNLA |
| (Alternative) | | |
| SEQ ID NO: 49 | LCDR2 | GASTRAT |
| (Alternative) | | |
| SEQ ID NO: 50 | LCDR3 | QQHNAFPYT |
| (Alternative) | | |
| SEQ ID NO: 55 | VL | EIVLTQSPATLSVSPGERATLSCRASQSVGSNLAWYQ |
| | | QKPGQAPRLLIYGASTRATGIPARFSGSGSGTEFTLTIS |
| | | SLQSEDFAVYYCQQHNAFPYTFGGGTKVEIK |
| SEQ ID NO: 144 | DNA VL | GAAATAGTGTTGACGCAGTCTCCAGCCACCCTGTCT |
| | | GTGTCTCCAGGGGAAAGAGCCACCCTCTCCTGCAG |
| | | GGCCAGTCAGAGTGTTGGCAGCAACTTAGCCTGGTA |
| | | CCAGCAGAAACCTGGCCAGGCTCCCAGGCTCCTCAT |
| | | CTATGGTGCATCCACCAGGGCCACTGGTATCCCAGC |
| | | CAGGTTCAGTGGCAGTGGGTCTGGGACAGAGTTCA |
| | | CTCTCACCATCAGCAGCCTGCAGTCTGAAGATTTTG |
| | | CAGTTTATTACTGTCAGCAGCACAATGCCTTCCCTTA |
| | | CACTTTTGGCGGAGGGACCAAGGTTGAGATCAAA |
| 930 | | |
| SEQ ID NO: 153 | HCDR1 | GFTFSSYAMS |
| (Combined) | | |
| SEQ ID NO: 154 | HCDR2 | AISGSGGSTYYADSVKG |
| (Combined) | | |
| SEQ ID NO: 37 | HCDR3 | GGLYGSGSYLSDFDL |
| (Combined) | | |
| SEQ ID NO: 137 (Kabat) | HCDR1 | SYAMS |
| SEQ ID NO: 154 (Kabat) | HCDR2 | AISGSGGSTYYADSVKG |
| SEQ ID NO: 37 (Kabat) | HCDR3 | GGLYGSGSYLSDFDL |
| SEQ ID NO: 155 | HCDR1 | GFTFSSY |
| (Chothia) | | |
| SEQ ID NO: 156 | HCDR2 | SGSGGS |
| (Chothia) | | |
| SEQ ID NO: 37 (Chothia) | HCDR3 | GGLYGSGSYLSDFDL |
| SEQ ID NO: 157 (IMGT) | HCDR1 | GFTFSSYA |
| SEQ ID NO: 158 (IMGT) | HCDR2 | ISGSGGST |
| SEQ ID NO: 43 (IMGT) | HCDR3 | ARGGLYGSGSYLSDFDL |
| SEQ ID NO: 195 | HCDR1 | FTFSSYAMS |
| (Alternative) | | |
| SEQ ID NO: 154 | HCDR2 | AISGSGGSTYYADSVKG |
| (Alternative) | | |
| SEQ ID NO: 43 | HCDR3 | ARGGLYGSGSYLSDFDL |
| (Alternative) | | |
| SEQ ID NO: 159 | VH | EVQLLESGGGLVQPGGSLRLSCAASGFTFSSYAMSWV |
| | | RQAPGKGLEWVSAISGSGGSTYYADSVKGRFTISRDN |
| | | SKNTLYLQMNSLRAEDTAVYYCARGGLYGSGSYLSDF |
| | | DLWGRGTLVTVSS |
| SEQ ID NO: 160 | DNA VH | GAGGTGCAGCTGTTGGAGTCTGGGGGAGGCTTGGT |
| | | ACAGCCTGGGGGGTCCCTGAGACTCTCCTGTGCAG |
| | | CCTCTGGATTCACCTTTAGCAGCTATGCCATGAGCT |
| | | GGGTCCGCCAGGCTCCAGGGAAGGGGCTGGAGTG |
| | | GGTCTCAGCTATTAGTGGTAGTGGTGGTAGCACATA |
| | | CTACGCAGACTCCGTGAAGGGCCGGTTCACCATCTC |
| | | CAGAGACAATTCCAAGAACACGCTGTATCTGCAAAT |
| | | GAACAGCCTGAGAGCCGAGGACACGGCGGTGTACT |
| | | ACTGCGCCAGAGGTGGATTGTACGGAAGCGGAAGC |
| | | TACTTGAGTGACTTCGACCTATGGGGGAGAGGTACC |
| | | TTGGTCACCGTCTCCTCA |
| SEQ ID NO: 48 | LCDR1 | RASQSVGSNLA |
| (Combined) | | |
| SEQ ID NO: 49 | LCDR2 | GASTRAT |
| (Combined) | | |
| SEQ ID NO: 50 | LCDR3 | QQHNAFPYT |
| (Combined) | | |
| SEQ ID NO: 48 (Kabat) | LCDR1 | RASQSVGSNLA |
| SEQ ID NO: 49 (Kabat) | LCDR2 | GASTRAT |
| SEQ ID NO: 50 (Kabat) | LCDR3 | QQHNAFPYT |
| SEQ ID NO: 51 (Chothia) | LCDR1 | SQSVGSN |
| SEQ ID NO: 52 (Chothia) | LCDR2 | GAS |
| SEQ ID NO: 53 (Chothia) | LCDR3 | HNAFPY |
| SEQ ID NO: 54 (IMGT) | LCDR1 | QSVGSN |
| SEQ ID NO: 52 (IMGT) | LCDR2 | GAS |
| SEQ ID NO: 50 (IMGT) | LCDR3 | QQHNAFPYT |
| SEQ ID NO: 48 | LCDR1 | RASQSVGSNLA |
| (Alternative) | | |
| SEQ ID NO: 49 | LCDR2 | GASTRAT |
| (Alternative) | | |
| SEQ ID NO: 50 | LCDR3 | QQHNAFPYT |
| (Alternative) | | |
| SEQ ID NO: 55 | VL | EIVLTQSPATLSVSPGERATLSCRASQSVGSNLAWYQ |
| | | QKPGQAPRLLIYGASTRATGIPARFSGSGSGTEFTLTIS |
| | | SLQSEDFAVYYCQQHNAFPYTFGGGTKVEIK |
| SEQ ID NO: 144 | DNA VL | GAAATAGTGTTGACGCAGTCTCCAGCCACCCTGTCT |
| | | GTGTCTCCAGGGGAAAGAGCCACCCTCTCCTGCAG |
| | | GGCCAGTCAGAGTGTTGGCAGCAACTTAGCCTGGTA |
| | | CCAGCAGAAACCTGGCCAGGCTCCCAGGCTCCTCAT |
| | | CTATGGTGCATCCACCAGGGCCACTGGTATCCCAGC |
| | | CAGGTTCAGTGGCAGTGGGTCTGGGACAGAGTTCA |
| | | CTCTCACCATCAGCAGCCTGCAGTCTGAAGATTTTG |
| | | CAGTTTATTACTGTCAGCAGCACAATGCCTTCCCTTA |
| | | CACTTTTGGCGGAGGGACCAAGGTTGAGATCAAA |
| 918 | | |
| SEQ ID NO: 161 | HCDR1 | GGSISSYYWS |
| (Combined) | | |
| SEQ ID NO: 162 | HCDR2 | YIYSSGSTNYNPSLKS |
| (Combined) | | |
| SEQ ID NO: 3 | HCDR3 | ESQESPYNNWFDP |
| (Combined) | | |
| SEQ ID NO: 163 (Kabat) | HCDR1 | SYYWS |
| SEQ ID NO: 162 (Kabat) | HCDR2 | YIYSSGSTNYNPSLKS |
| SEQ ID NO: 3 (Kabat) | HCDR3 | ESQESPYNNWFDP |
| SEQ ID NO: 164 | HCDR1 | GGSISSY |
| (Chothia) | | |
| SEQ ID NO: 165 | HCDR2 | YSSGS |
| (Chothia) | | |
| SEQ ID NO: 3 (Chothia) | HCDR3 | ESQESPYNNWFDP |
| SEQ ID NO: 166 (IMGT) | HCDR1 | GGSISSYY |
| SEQ ID NO: 167 (IMGT) | HCDR2 | IYSSGST |
| SEQ ID NO: 9 (IMGT) | HCDR3 | ARESQESPYNNWFDP |
| SEQ ID NO: 200 | HCDR1 | GSISSYYWS |
| (Alternative) | | |
| SEQ ID NO: 162 | HCDR2 | YIYSSGSTNYNPSLKS |
| (Alternative) | | |
| SEQ ID NO: 9 | HCDR3 | ARESQESPYNNWFDP |
| (Alternative) | | |
| SEQ ID NO: 168 | VH | QVQLQESGPGLVKPSETLSLTCTVSGGSISSYYWSWIR |
| | | QPPGKGLEWIGYIYSSGSTNYNPSLKSRVTISVDTSKN |
| | | QFSLKLSSVTAADTAVYYCARESQESPYNNWFDPWG |
| | | QGTLVTVSS |
| SEQ ID NO: 169 | DNA VH | CAGGTGCAGCTGCAGGAGTCGGGCCCAGGACTGGT |
| | | GAAGCCTTCGGAGACCCTGTCCCTCACCTGCACTGT |
| | | CTCTGGTGGCTCCATCAGTAGTTACTACTGGAGCTG |
| | | GATCCGGCAGCCCCCAGGGAAGGGACTGGAGTGGA |
| | | TTGGGTATATCTATAGTAGTGGGAGCACCAACTACAA |
| | | CCCCTCCCTCAAGAGTCGAGTCACCATATCAGTAGA |
| | | CACGTCCAAGAACCAGTTCTCCCTGAAGCTGAGTTC |
| | | TGTGACCGCCGCAGACACGGCGGTGTACTACTGCG |
| | | CCAGAGAATCTCAGGAGAGTCCATACAACAATTGGT |
| | | TCGACCCATGGGGACAGGGTACATTGGTCACCGTCT |
| | | CCTCA |
| SEQ ID NO: 14 | LCDR1 | RASQGISSWLA |
| (Combined) | | |
| SEQ ID NO: 15 | LCDR2 | AASSLQS |
| (Combined) | | |
| SEQ ID NO: 16 | LCDR3 | QQGNSFPRT |
| (Combined) | | |
| SEQ ID NO: 14 (Kabat) | LCDR1 | RASQGISSWLA |
| SEQ ID NO: 15 (Kabat) | LCDR2 | AASSLQS |
| SEQ ID NO: 16 (Kabat) | LCDR3 | QQGNSFPRT |
| SEQ ID NO: 17 (Chothia) | LCDR1 | SQGISSW |
| SEQ ID NO: 18 (Chothia) | LCDR2 | AAS |
| SEQ ID NO: 19 (Chothia) | LCDR3 | GNSFPR |
| SEQ ID NO: 20 (IMGT) | LCDR1 | QGISSW |
| SEQ ID NO: 18 (IMGT) | LCDR2 | AAS |
| SEQ ID NO: 16 (IMGT) | LCDR3 | QQGNSFPRT |
| SEQ ID NO: 14 | LCDR1 | RASQGISSWLA |
| (Alternative) | | |
| SEQ ID NO: 15 | LCDR2 | AASSLQS |
| (Alternative) | | |
| SEQ ID NO: 16 | LCDR3 | QQGNSFPRT |
| (Alternative) | | |
| SEQ ID NO: 21 | VL | DIQMTQSPSSVSASVGDRVTITCRASQGISSWLAWYQ |
| | | QKPGKAPKLLIYAASSLQSGVPSRFSGSGSGTDFTLTIS |
| | | SLQPEDFATYYCQQGNSFPRTFGGGTKVEIK |
| SEQ ID NO: 170 | DNA VL | GACATCCAGATGACCCAGTCTCCATCTTCCGTGTCT |
| | | GCATCTGTAGGAGACAGAGTCACCATCACTTGTCGG |
| | | GCGAGTCAGGGTATTAGCAGCTGGTTAGCCTGGTAT |
| | | CAGCAGAAACCAGGGAAAGCCCCTAAGCTCCTGATC |
| | | TATGCTGCATCCAGTTTGCAAAGTGGGGTCCCATCA |
| | | AGGTTCAGCGGCAGTGGATCTGGGACAGATTTCACT |
| | | CTCACCATCAGCAGCCTGCAGCCTGAAGATTTTGCA |
| | | ACTTATTACTGTCAGCAGGGAAACAGTTTCCCTAGGA |
| | | CTTTTGGCGGAGGGACCAAGGTTGAGATCAAA |
| 363 | | |
| SEQ ID NO: 81 | HCDR1 | GFTFSRYAMS |
| (Combined) | | |
| SEQ ID NO: 201 | HCDR2 | AISGTGISTYYADSVKG |
| (Combined) | | |
| SEQ ID NO: 37 | HCDR3 | GGLYGSGSYLSDFDL |
| (Combined) | | |
| SEQ ID NO: 38 (Kabat) | HCDR1 | RYAMS |
| SEQ ID NO: 201 (Kabat) | HCDR2 | AISGTGISTYYADSVKG |
| SEQ ID NO: 37 (Kabat) | HCDR3 | GGLYGSGSYLSDFDL |
| SEQ ID NO: 82 (Chothia) | HCDR1 | GFTFSRY |
| SEQ ID NO: 202 | HCDR2 | SGTGIS |
| (Chothia) | | |
| SEQ ID NO: 37 (Chothia) | HCDR3 | GGLYGSGSYLSDFDL |
| SEQ ID NO: 83 (IMGT) | HCDR1 | GFTFSRYA |
| SEQ ID NO: 203 (IMGT) | HCDR2 | ISGTGIST |
| SEQ ID NO: 43 (IMGT) | HCDR3 | ARGGLYGSGSYLSDFDL |
| SEQ ID NO: 192 | HCDR1 | FTFSRYAMS |
| (Alternative) | | |
| SEQ ID NO: 201 | HCDR2 | AISGTGISTYYADSVKG |
| (Alternative) | | |
| SEQ ID NO: 43 | HCDR3 | ARGGLYGSGSYLSDFDL |
| (Alternative) | | |
| SEQ ID NO: 204 | VH | EVQLLESGGGLVQPGGSLRLSCAASGFTFSRYAMSWV |
| | | RQAPGKGLEWVSAISGTGISTYYADSVKGRFTISRDNS |
| | | KNTLYLQMNSLRAEDTAVYYCARGGLYGSGSYLSDFD |
| | | LWGRGTLVTVSS |
| SEQ ID NO: 205 | LCDR1 | RASQSISSWLA |
| (Combined) | | |
| SEQ ID NO: 206 | LCDR2 | DASSLES |
| (Combined) | | |
| SEQ ID NO: 207 | LCDR3 | QQSNTFYT |
| (Combined) | | |
| SEQ ID NO: 205 (Kabat) | LCDR1 | RASQSISSWLA |
| SEQ ID NO: 206 (Kabat) | LCDR2 | DASSLES |
| SEQ ID NO: 207 (Kabat) | LCDR3 | QQSNTFYT |
| SEQ ID NO: 208 | LCDR1 | SQSISSW |
| (Chothia) | | |
| SEQ ID NO: 209 | LCDR2 | DAS |
| (Chothia) | | |
| SEQ ID NO: 228 | LCDR3 | SNTFY |
| (Chothia) | | |
| SEQ ID NO: 229 (IMGT) | LCDR1 | QSISSW |
| SEQ ID NO: 209 (IMGT) | LCDR2 | DAS |
| SEQ ID NO: 207 (IMGT) | LCDR3 | QQSNTFYT |
| SEQ ID NO: 205 | LCDR1 | RASQSISSWLA |
| (Alternative) | | |
| SEQ ID NO: 206 | LCDR2 | DASSLES |
| (Alternative) | | |
| SEQ ID NO: 207 | LCDR3 | QQSNTFYT |
| (Alternative) | | |
| SEQ ID NO: 230 | VL | DIQMTQSPSTLSASVGDRVTITCRASQSISSWLAWYQQ |
| | | KPGKAPKLLIYDASSLESGVPSRFSGSGSGTEFTLTISS |
| | | LQPDDFATYYCQQSNTFYTFGGGTKVEIK |
| 366 | HCDR1 | GFTFSRYAMS |
| SEQ ID NO: 81 | | |
| (Combined) | | |
| SEQ ID NO: 231 | HCDR2 | AISGTGLSTYYADSVKG |
| (Combined) | | |
| SEQ ID NO: 37 | HCDR3 | GGLYGSGSYLSDFDL |
| (Combined) | | |
| SEQ ID NO: 38 (Kabat) | HCDR1 | RYAMS |
| SEQ ID NO: 231 (Kabat) | HCDR2 | AISGTGLSTYYADSVKG |
| SEQ ID NO: 37 (Kabat) | HCDR3 | GGLYGSGSYLSDFDL |
| SEQ ID NO: 82 (Chothia) | HCDR1 | GFTFSRY |
| SEQ ID NO: 232 | HCDR2 | SGTGLS |
| (Chothia) | | |
| SEQ ID NO: 37 (Chothia) | HCDR3 | GGLYGSGSYLSDFDL |
| SEQ ID NO: 83 (IMGT) | HCDR1 | GFTFSRYA |
| SEQ ID NO: 233 (IMGT) | HCDR2 | ISGTGLST |
| SEQ ID NO: 43 (IMGT) | HCDR3 | ARGGLYGSGSYLSDFDL |
| SEQ ID NO: 192 | HCDR1 | FTFSRYAMS |
| (Alternative) | | |
| SEQ ID NO: 231 | HCDR2 | AISGTGLSTYYADSVKG |
| (Alternative) | | |
| SEQ ID NO: 43 | HCDR3 | ARGGLYGSGSYLSDFDL |
| (Alternative) | | |
| SEQ ID NO: 234 | VH | EVQLLESGGGLVQPGGSLRLSCAASGFTFSRYAMSWV |
| | | RQAPGKGLEWVSAISGTGLSTYYADSVKGRFTISRDNS |
| | | KNTLYLQMNSLRAEDTAVYYCARGGLYGSGSYLSDFD |
| | | LWGRGTLVTVSS |
| SEQ ID NO: 205 | LCDR1 | RASQSISSWLA |
| (Combined) | | |
| SEQ ID NO: 206 | LCDR2 | DASSLES |
| (Combined) | | |
| SEQ ID NO: 207 | LCDR3 | QQSNTFYT |
| (Combined) | | |
| SEQ ID NO: 205 (Kabat) | LCDR1 | RASQSISSWLA |
| SEQ ID NO: 206 (Kabat) | LCDR2 | DASSLES |
| SEQ ID NO: 207 (Kabat) | LCDR3 | QQSNTFYT |
| SEQ ID NO: 208 | LCDR1 | SQSISSW |
| (Chothia) | | |
| SEQ ID NO: 209 | LCDR2 | DAS |
| (Chothia) | | |
| SEQ ID NO: 228 | LCDR3 | SNTFY |
| (Chothia) | | |
| SEQ ID NO: 229 (IMGT) | LCDR1 | QSISSW |
| SEQ ID NO: 209 (IMGT) | LCDR2 | DAS |
| SEQ ID NO: 207 (IMGT) | LCDR3 | QQSNTFYT |
| SEQ ID NO: 205 | LCDR1 | RASQSISSWLA |
| (Alternative) | | |
| SEQ ID NO: 206 | LCDR2 | DASSLES |
| (Alternative) | | |
| SEQ ID NO: 207 | LCDR3 | QQSNTFYT |
| (Alternative) | | |
| SEQ ID NO: 230 | VL | DIQMTQSPSTLSASVGDRVTITCRASQSISSWLAWYQQ |
| | | KPGKAPKLLIYDASSLESGVPSRFSGSGSGTEFTLTISS |
| | | LQPDDFATYYCQQSNTFYTFGGGTKVEIK |
| 407 | | |
| SEQ ID NO: 235 | HCDR1 | GYTFTYYWMH |
| (Combined) | | |
| SEQ ID NO: 236 | HCDR2 | SINPNSGSTNYAQKFQG |
| (Combined) | | |
| SEQ ID NO: 237 | HCDR3 | DTGGDKSPLTYYYYGMDV |
| (Combined) | | |
| SEQ ID NO: 238 (Kabat) | HCDR1 | YYWMH |
| SEQ ID NO: 236 (Kabat) | HCDR2 | SINPNSGSTNYAQKFQG |
| SEQ ID NO: 237 (Kabat) | HCDR3 | DTGGDKSPLTYYYYGMDV |
| SEQ ID NO: 239 | HCDR1 | GYTFTYY |
| (Chothia) | | |
| SEQ ID NO: 240 | HCDR2 | NPNSGS |
| (Chothia) | | |
| SEQ ID NO: 237 | HCDR3 | DTGGDKSPLTYYYYGMDV |
| (Chothia) | | |
| SEQ ID NO: 241 (IMGT) | HCDR1 | GYTFTYYW |
| SEQ ID NO: 242 (IMGT) | HCDR2 | INPNSGST |
| SEQ ID NO: 243 (IMGT) | HCDR3 | ARDTGGDKSPLTYYYYGMDV |
| SEQ ID NO: 244 | HCDR1 | YTFTYYWMH |
| (Alternative) | | |
| SEQ ID NO: 236 | HCDR2 | SINPNSGSTNYAQKFQG |
| (Alternative) | | |
| SEQ ID NO: 243 | HCDR3 | ARDTGGDKSPLTYYYYGMDV |
| (Alternative) | | |
| SEQ ID NO: 245 | VH | QVQLVQSGAEVKKPGASVKVSCKASGYTFTYYWMHW |
| | | VRQAPGQGLEWMGSINPNSGSTNYAQKFQGRVTMTR |
| | | DTSISTAYMELSRLRSDDTAVYYCARDTGGDKSPLTYY |
| | | YYGMDVWGQGTTVTVSS |
| SEQ ID NO: 246 | LCDR1 | RASQGISRWLA |
| (Combined) | | |
| SEQ ID NO: 15 | LCDR2 | AASSLQS |
| (Combined) | | |
| SEQ ID NO: 247 | LCDR3 | QQAIALPPFT |
| (Combined) | | |
| SEQ ID NO: 246 (Kabat) | LCDR1 | RASQGISRWLA |
| SEQ ID NO: 15 (Kabat) | LCDR2 | AASSLQS |
| SEQ ID NO: 247 (Kabat) | LCDR3 | QQAIALPPFT |
| SEQ ID NO: 248 | LCDR1 | SQGISRW |
| (Chothia) | | |
| SEQ ID NO: 18 (Chothia) \| | LCDR2 | AAS |
| SEQ ID NO: 249 | LCDR3 | AIALPPF |
| (Chothia) | | |
| SEQ ID NO: 250 (IMGT) | LCDR1 | QGISRW |
| SEQ ID NO: 18 (IMGT) | LCDR2 | AAS |
| SEQ ID NO: 247 (IMGT) | LCDR3 | QQAIALPPFT |
| SEQ ID NO: 246 | LCDR1 | RASQGISRWLA |
| (Alternative) | | |
| SEQ ID NO: 15 | LCDR2 | AASSLQS |
| (Alternative) | | |
| SEQ ID NO: 247 | LCDR3 | QQAIALPPFT |
| (Alternative) | | |
| SEQ ID NO: 251 | VL | DIQMTQSPSSVSASVGDRVTITCRASQGISRWLAWYQ |
| | | QKPGKAPKLLIYAASSLQSGVPSRFSGSGSGTDFTLTIS |
| | | SLQPEDFATYYCQQAIALPPFTFGGGTKVEIK |
| 893 | | |
| SEQ ID NO: 252 | HCDR1 | GFTFSSYWMS |
| (Combined) | | |
| SEQ ID NO: 253 | HCDR2 | NIKQDGSEKYYVDSVKG |
| (Combined) | | |
| SEQ ID NO: 254 | HCDR3 | APEYYSTTTRLYYYYGMDV |
| (Combined) | | |
| SEQ ID NO: 255 (Kabat) | HCDR1 | SYWMS |
| SEQ ID NO: 253 (Kabat) | HCDR2 | NIKQDGSEKYYVDSVKG |
| SEQ ID NO: 254 (Kabat) | HCDR3 | APEYYSTTTRLYYYYGMDV |
| SEQ ID NO: 155 | HCDR1 | GFTFSSY |
| (Chothia) | | |
| SEQ ID NO: 256 | HCDR2 | KQDGSE |
| (Chothia) | | |
| SEQ ID NO: 254 | HCDR3 | APEYYSTTTRLYYYYGMDV |
| (Chothia) | | |
| SEQ ID NO: 257 (IMGT) | HCDR1 | GFTFSSYW |
| SEQ ID NO: 258 (IMGT) | HCDR2 | IKQDGSEK |
| SEQ ID NO: 259 (IMGT) | HCDR3 | ARAPEYYSTTTRLYYYYGMDV |
| SEQ ID NO: 260 | HCDR1 | FTFSSYWMS |
| (Alternative) | | |
| SEQ ID NO: 253 | HCDR2 | NIKQDGSEKYYVDSVKG |
| (Alternative) | | |
| SEQ ID NO: 259 | HCDR3 | ARAPEYYSTTTRLYYYYGMDV |
| (Alternative) | | |
| SEQ ID NO: 261 | VH | EVQLVESGGGLVQPGGSLRLSCAASGFTFSSYWMSW |
| | | VRQAPGKGLEWVANIKQDGSEKYYVDSVKGRFTISRD |
| | | NAKNSLYLQMNSLRAEDTAVYYCARAPEYYSTTTRLYY |
| | | YYGMDVWGQGTTVTVSS |
| SEQ ID NO: 262 | LCDR1 | KSSQSVLYSSNNKNYLA |
| (Combined) | | |
| SEQ ID NO: 263 | LCDR2 | WASTRES |
| (Combined) | | |
| SEQ ID NO: 264 | LCDR3 | QQYDAHPFT |
| (Combined) | | |
| SEQ ID NO: 262 (Kabat) | LCDR1 | KSSQSVLYSSNNKNYLA |
| SEQ ID NO: 263 (Kabat) | LCDR2 | WASTRES |
| SEQ ID NO: 264 (Kabat) | LCDR3 | QQYDAHPFT |
| SEQ ID NO: 265 | LCDR1 | SQSVLYSSNNKNY |
| (Chothia) | | |
| SEQ ID NO: 266 | LCDR2 | WAS |
| (Chothia) | | |
| SEQ ID NO: 267 | LCDR3 | YDAHPF |
| (Chothia) | | |
| SEQ ID NO: 268 (IMGT) | LCDR1 | QSVLYSSNNKNY |
| SEQ ID NO: 266 (IMGT) | LCDR2 | WAS |
| SEQ ID NO: 264 (IMGT) | LCDR3 | QQYDAHPFT |
| SEQ ID NO: 262 | LCDR1 | KSSQSVLYSSNNKNYLA |
| (Alternative) | | |
| SEQ ID NO: 263 | LCDR2 | WASTRES |
| (Alternative) | | |
| SEQ ID NO: 264 | LCDR3 | QQYDAHPFT |
| (Alternative) | | |
| SEQ ID NO: 269 | VL | DIVMTQSPDSLAVSLGERATINCKSSQSVLYSSNNKNY |
| | | LAWYQQKPGQPPKLLIYWASTRESGVPDRFSGSGSGT |
| | | DFTLTISSLQAEDVAVYYCQQYDAHPFTFGGGTKVEIK |
| 939 | | |
| SEQ ID NO: 287 | HCDR1 | GGTFSSYAIS |
| (Combined) | | |
| SEQ ID NO: 288 | HCDR2 | GIIPIFGTANYAQKFQG |
| (Combined) | | |
| SEQ ID NO: 289 | HCDR3 | HYYDYWSGYYTNTGIY |
| (Combined) | | |
| SEQ ID NO: 290 (Kabat) | HCDR1 | SYAIS |
| SEQ ID NO: 288 (Kabat) | HCDR2 | GIIPIFGTANYAQKFQG |
| SEQ ID NO: 289 (Kabat) | HCDR3 | HYYDYWSGYYTNTGIY |
| SEQ ID NO: 291 | HCDR1 | GGTFSSY |
| (Chothia) | | |
| SEQ ID NO: 292 | HCDR2 | IPIFGT |
| (Chothia) | | |
| SEQ ID NO: 289 | HCDR3 | HYYDYWSGYYTNTGIY |
| (Chothia) | | |
| SEQ ID NO: 293 (IMGT) | HCDR1 | GGTFSSYA |
| SEQ ID NO: 294 (IMGT) | HCDR2 | IIPIFGTA |
| SEQ ID NO: 295 (IMGT) | HCDR3 | ARHYYDYWSGYYTNTGIY |
| SEQ ID NO: 296 | HCDR1 | GTFSSYAIS |
| (Alternative) | | |
| SEQ ID NO: 288 | HCDR2 | GIIPIFGTANYAQKFQG |
| (Alternative) | | |
| SEQ ID NO: 295 | HCDR3 | ARHYYDYWSGYYTNTGIY |
| (Alternative) | | |
| SEQ ID NO: 297 | VH | QVQLVQSGAEVKKPGSSVKVSCKASGGTFSSYAISWV |
| | | RQAPGQGLEWMGGIIPIFGTANYAQKFQGRVTITADES |
| | | TSTAYMELSSLRSEDTAVYYCARHYYDYWSGYYTNTG |
| | | IYWGQGTLVTVSS |
| SEQ ID NO: 298 | LCDR1 | RASQSVSSNLA |
| (Combined) | | |
| SEQ ID NO: 49 | LCDR2 | GASTRAT |
| (Combined) | | |
| SEQ ID NO: 299 | LCDR3 | QQSGALPIT |
| (Combined) | | |
| SEQ ID NO: 298 (Kabat) | LCDR1 | RASQSVSSNLA |
| SEQ ID NO: 49 (Kabat) | LCDR2 | GASTRAT |
| SEQ ID NO: 299 (Kabat) | LCDR3 | QQSGALPIT |
| SEQ ID NO: 300 | LCDR1 | SQSVSSN |
| (Chothia) | | |
| SEQ ID NO: 52 (Chothia) | LCDR2 | GAS |
| SEQ ID NO: 301 | LCDR3 | SGALPI |
| (Chothia) | | |
| SEQ ID NO: 302 (IMGT) | LCDR1 | QSVSSN |
| SEQ ID NO: 52 (IMGT) | LCDR2 | GAS |
| SEQ ID NO: 299 (IMGT) | LCDR3 | QQSGALPIT |
| SEQ ID NO: 298 | LCDR1 | RASQSVSSNLA |
| (Alternative) | | |
| SEQ ID NO: 49 | LCDR2 | GASTRAT |
| (Alternative) | | |
| SEQ ID NO: 299 | LCDR3 | QQSGALPIT |
| (Alternative) | | |
| SEQ ID NO: 303 | VL | EIVMTQSPATLSVSPGERATLSCRASQSVSSNLAWYQ |
| | | QKPGQAPRLLIYGASTRATGIPARFSGSGSGTEFTLTIS |
| | | SLQSEDFAVYYCQQSGALPITFGGGTKVEIK |
| 430 | | |
| SEQ ID NO: 304 | HCDR1 | GGSFLRYYWS |
| (Combined) | | |
| SEQ ID NO: 305 | HCDR2 | EIDHSGSTNYNPSLKS |
| (Combined) | | |
| SEQ ID NO: 306 | HCDR3 | GQNYYGSGSADGFDP |
| (Combined) | | |
| SEQ ID NO: 61 (Kabat) | HCDR1 | RYYWS |
| SEQ ID NO: 305 (Kabat) | HCDR2 | EIDHSGSTNYNPSLKS |
| SEQ ID NO: 306 (Kabat) | HCDR3 | GQNYYGSGSADGFDP |
| SEQ ID NO: 307 | HCDR1 | GGSFLRY |
| (Chothia) | | |
| SEQ ID NO: 308 | HCDR2 | DHSGS |
| (Chothia) | | |
| SEQ ID NO: 306 | HCDR3 | GQNYYGSGSADGFDP |
| (Chothia) | | |
| SEQ ID NO: 309 (IMGT) | HCDR1 | GGSFLRYY |
| SEQ ID NO: 310 (IMGT) | HCDR2 | IDHSGST |
| SEQ ID NO: 311 (IMGT) | HCDR3 | ARGQNYYGSGSADGFDP |
| SEQ ID NO: 312 | HCDR1 | GSFLRYYWS |
| (Alternative) | | |
| SEQ ID NO: 305 | HCDR2 | EIDHSGSTNYNPSLKS |
| (Alternative) | | |
| SEQ ID NO: 311 | HCDR3 | ARGQNYYGSGSADGFDP |
| (Alternative) | | |
| SEQ ID NO: 313 | VH | QVQLQQWGAGLLKPSETLSLTCAVYGGSFLRYYWSWI |
| | | RQPPGKGLEWIGEIDHSGSTNYNPSLKSRVTISVDTSK |
| | | NQFSLKLSSVTAADTAVYYCARGQNYYGSGSADGFDP |
| | | WGQGTLVTVSS |
| SEQ ID NO: 14 | LCDR1 | RASQGISSWLA |
| (Combined) | | |
| SEQ ID NO: 15 | LCDR2 | AASSLQS |
| (Combined) | | |
| SEQ ID NO: 314 | LCDR3 | QQANSFPPT |
| (Combined) | | |
| SEQ ID NO: 14 (Kabat) | LCDR1 | RASQGISSWLA |
| SEQ ID NO: 15 (Kabat) | LCDR2 | AASSLQS |
| SEQ ID NO: 314 (Kabat) | LCDR3 | QQANSFPPT |
| SEQ ID NO: 17 (Chothia) | LCDR1 | SQGISSW |
| SEQ ID NO: 18 (Chothia) | LCDR2 | AAS |
| SEQ ID NO: 315 | LCDR3 | ANSFPP |
| (Chothia) | | |
| SEQ ID NO: 20 (IMGT) | LCDR1 | QGISSW |
| SEQ ID NO: 18 (IMGT) | LCDR2 | AAS |
| SEQ ID NO: 314 (IMGT) | LCDR3 | QQANSFPPT |
| SEQ ID NO: 14 | LCDR1 | RASQGISSWLA |
| (Alternative) | | |
| SEQ ID NO: 15 | LCDR2 | AASSLQS |
| (Alternative) | | |
| SEQ ID NO: 314 | LCDR3 | QQANSFPPT |
| (Alternative) | | |
| SEQ ID NO: 316 | VL | DIQMTQSPSSVSASVGDRVTITCRASQGISSWLAWYQ |
| | | QKPGKAPKLLIYAASSLQSGVPSRFSGSGSGTDFTLTIS |
| | | SLQPEDFATYYCQQANSFPPTFGGGTKVEIK |
| 398 | | |
| SEQ ID NO: 317 | HCDR1 | GGSFSGYYWS |
| (Combined) | | |
| SEQ ID NO: 318 | HCDR2 | EIDASGSTRYNPSLKS |
| (Combined) | | |
| SEQ ID NO: 319 | HCDR3 | PLYDAYLDV |
| (Combined) | | |
| SEQ ID NO: 320 (Kabat) | HCDR1 | GYYWS |
| SEQ ID NO: 318 (Kabat) | HCDR2 | EIDASGSTRYNPSLKS |
| SEQ ID NO: 319 (Kabat) | HCDR3 | PLYDAYLDV |
| SEQ ID NO: 321 | HCDR1 | GGSFSGY |
| (Chothia) | | |
| SEQ ID NO: 322 | HCDR2 | DASGS |
| (Chothia) | | |
| SEQ ID NO: 319 | HCDR3 | PLYDAYLDV |
| (Chothia) | | |
| SEQ ID NO: 323 (IMGT) | HCDR1 | GGSFSGYY |
| SEQ ID NO: 324 (IMGT) | HCDR2 | IDASGST |
| SEQ ID NO: 325 (IMGT) | HCDR3 | ARPLYDAYLDV |
| SEQ ID NO: 326 | HCDR1 | GSFSGYYWS |
| (Alternative) | | |
| SEQ ID NO: 318 | HCDR2 | EIDASGSTRYNPSLKS |
| (Alternative) | | |
| SEQ ID NO: 325 | HCDR3 | ARPLYDAYLDV |
| (Alternative) | | |
| SEQ ID NO: 327 | VH | QVQLQQWGAGLLKPSETLSLTCAVYGGSFSGYYWSWI |
| | | RQPPGKGLEWIGEIDASGSTRYNPSLKSRVTISVDTSK |
| | | NQFSLKLSSVTAADTAVYYCARPLYDAYLDVWGQGTM |
| | | VTVSS |
| SEQ ID NO: 14 | LCDR1 | RASQGISSWLA |
| (Combined) | | |
| SEQ ID NO: 15 | LCDR2 | AASSLQS |
| (Combined) | | |
| SEQ ID NO: 328 | LCDR3 | QQAPIYPIT |
| (Combined) | | |
| SEQ ID NO: 14 (Kabat) | LCDR1 | RASQGISSWLA |
| SEQ ID NO: 15 (Kabat) | LCDR2 | AASSLQS |
| SEQ ID NO: 328 (Kabat) | LCDR3 | QQAPIYPIT |
| SEQ ID NO: 17 (Chothia) | LCDR1 | SQGISSW |
| SEQ ID NO: 18 (Chothia) | LCDR2 | AAS |
| SEQ ID NO: 329 | LCDR3 | APIYPI |
| (Chothia) | | |
| SEQ ID NO: 20 (IMGT) | LCDR1 | QGISSW |
| SEQ ID NO: 18 (IMGT) | LCDR2 | AAS |
| SEQ ID NO: 328 (IMGT) | LCDR3 | QQAPIYPIT |
| SEQ ID NO: 14 | LCDR1 | RASQGISSWLA |
| (Alternative) | | |
| SEQ ID NO: 15 | LCDR2 | AASSLQS |
| (Alternative) | | |
| SEQ ID NO: 328 | LCDR3 | QQAPIYPIT |
| (Alternative) | | |
| SEQ ID NO: 330 | VL | DIQMTQSPSSVSASVGDRVTITCRASQGISSWLAWYQ |
| | | QKPGKAPKLLIYAASSLQSGVPSRFSGSGSGTDFTLTIS |
| | | SLQPEDFATYYCQQAPIYPITFGGGTKVEIK |

TABLE 2
Consensus CDR sequences for exemplary anti-CD73 antibodies

| | | |
|---|---|---|
| Lineage 3 | | |
| SEQ ID NO: 126 | HCDR1 | GFTFX₁X₂X₃AMS, wherein |
| (Combined) | | X₁ is H, S, R, or K; |
| | | X₂ is R, Y, or S; and |
| | | X₃ is Y or N |
| SEQ ID NO: 89 | HCDR2 | X₁IX₂GX₃GX₄X₅TYYADSVKG, wherein |
| (Combined) | | X₁ is A or S; |
| | | X₂ is S or T; |
| | | X₃ is S or T; |
| | | X₄ is M, G, or S; and |
| | | X₅ is N, S, L, or Y |
| SEQ ID NO: 37 | HCDR3 | GGLYGSGSYLSDFDL |
| (Combined) | | |
| SEQ ID NO: 48 | LCDR1 | RASQSVGSNLA |
| (Combined) | | |
| SEQ ID NO: 49 | LCDR2 | GASTRAT |
| (Combined) | | |
| SEQ ID NO: 50 | LCDR3 | QQHNAFPYT |
| (Combined) | | |
| SEQ ID NO: 88 (Kabat) | HCDR1 | X₁X₂AMS, wherein |
| | | X₁ is R, Y, or S; and |
| | | X₂ is Y or N |
| SEQ ID NO: 89 (Kabat) | HCDR2 | X₁IX₂GX₃GX₄X₅TYYADSVKG, wherein |
| | | X₁ is A or S; |
| | | X₂ is S or T; X₃ is S or T; X₄ is M, G, or S; and |
| | | X₅ is N, S, L, or Y |
| SEQ ID NO: 37 | HCDR3 | GGLYGSGSYLSDFDL |
| (Kabat) | | |
| SEQ ID NO: 48 (Kabat) | LCDR1 | RASQSVGSNLA |
| SEQ ID NO: 49 (Kabat) | LCDR2 | GASTRAT |
| SEQ ID NO: 50 (Kabat) | LCDR3 | QQHNAFPYT |
| SEQ ID NO: 122 | HCDR1 | GFTFX₁X₂X₃, wherein |
| (Chothia) | | X₁ is H, S, R, or K; |
| | | X₂ is R, Y, or S; and |
| | | X₃ is Y or N |
| SEQ ID NO: 123 | HCDR2 | X₁GX₂GX₃X₄, wherein |
| (Chothia) | | X₁ is S or T; |
| | | X₂ is S or T; |
| | | X₃ is M, G, or S; and |
| | | X₄ is N, S, L, or Y |
| SEQ ID NO: 37 (Chothia) | HCDR3 | GGLYGSGSYLSDFDL |
| SEQ ID NO: 51 (Chothia) | LCDR1 | SQSVGSN |
| SEQ ID NO: 52 (Chothia) | LCDR2 | GAS |
| SEQ ID NO: 53 (Chothia) | LCDR3 | HNAFPY |
| SEQ ID NO: 128 (IMGT) | HCDR1 | GFTFX₁X₂X₃A, wherein |
| | | X₁ is H, S, R, or K; |
| | | X₂ is R, Y, or S; and |
| | | X₃ is Y or N |
| SEQ ID NO: 129 (IMGT) | HCDR2 | IX₁GX₂GX₃X₄T, wherein |
| | | X₁ is S or T; |
| | | X₂ is S or T; |
| | | X₃ is M, G, or S; and |
| | | X₄ is N, S, L, or Y |
| SEQ ID NO: 43 (IMGT) | HCDR3 | ARGGLYGSGSYLSDFDL |
| SEQ ID NO: 54 (IMGT) | LCDR1 | QSVGSN |
| SEQ ID NO: 52 (IMGT) | LCDR2 | GAS |
| SEQ ID NO: 50 (IMGT) | LCDR3 | QQHNAFPYT |
| SEQ ID NO: 189 | HCDR1 | FTFX₁X₂X₃AMS, wherein |
| (Alternative) | | X₁ is H, S, R, or K; |
| | | X₂ is R, Y, or S; and |
| | | X₃ is Y or N |
| SEQ ID NO: 89 | HCDR2 | X₁IX₂GX₃GX₄X₅TYYADSVKG, wherein |
| (Alternative) | | X₁ is A or S; |
| | | X₂ is S or T; |
| | | X₃ is S or T; |
| | | X₄ is M, G, or S; and |
| | | X₅ is N, S, L, or Y |
| SEQ ID NO: 43 | HCDR3 | ARGGLYGSGSYLSDFDL |
| (Alternative) | | |
| SEQ ID NO: 48 | LCDR1 | RASQSVGSNLA |
| (Alternative) | | |
| SEQ ID NO: 49 | LCDR2 | GASTRAT |
| (Alternative) | | |
| SEQ ID NO: 50 | LCDR3 | QQHNAFPYT |
| (Alternative) | | |
| Lineage 1 | | |
| SEQ ID NO: 127 | HCDR1 | GGSIX₁X₂X₃YWS, wherein |
| (Combined) | | X₁ is E or S; |
| | | X₂ is R, G, or S; and |
| | | X₃ is Y or R |
| SEQ ID NO: 91 | HCDR2 | YIYX₁X₂GSTX₃YNPSLKS, wherein |
| (Combined) | | X₁ is G or S; |
| | | X₂ is R, S, or T; and |
| | | X₃ is N or K |
| SEQ ID NO: 3 | HCDR3 | ESQESPYNNWFDP |
| (Combined) | | |
| SEQ ID NO: 14 | LCDR1 | RASQGISSWLA |
| (Combined) | | |
| SEQ ID NO: 15 | LCDR2 | AASSLQS |
| (Combined) | | |
| SEQ ID NO: 16 | LCDR3 | QQGNSFPRT |
| (Combined) | | |
| SEQ ID NO: 90 (Kabat) | HCDR1 | X₁X₂YWS, wherein |
| | | X₁ is R, G, or S; and |
| | | X₂ is Y or R |
| SEQ ID NO: 91 (Kabat) | | YIYX₁X₂GSTX₃YNPSLKS, wherein |
| | | X₁ is G or S; |
| | | X₂ is R, S, or T; and |
| | HCDR1 | X₃ is N or K |
| SEQ ID NO: 3 (Kabat) | HCDR3 | ESQESPYNNWFDP |
| SEQ ID NO: 14 (Kabat) | LCDR1 | RASQGISSWLA |
| SEQ ID NO: 15 (Kabat) | LCDR2 | AASSLQS |
| SEQ ID NO: 16 (Kabat) | LCDR3 | QQGNSFPRT |
| SEQ ID NO: 124 | HCDR1 | GGSIX₁X₂X₃, wherein |
| (Chothia) | | X₁ is E or S; |
| | | X₂ is R, G, or S; and |
| | | X₃ is Y or R |
| SEQ ID NO: 125 | HCDR2 | YX₁X₂GS, wherein |
| (Chothia) | | X₁ is G or S; and |
| | | X₂ is R, S, or T |
| SEQ ID NO: 3 (Chothia) | HCDR3 | ESQESPYNNWFDP |
| SEQ ID NO: 17 (Chothia) | LCDR1 | SQGISSW |
| SEQ ID NO: 18 (Chothia) | LCDR2 | AAS |
| SEQ ID NO: 19 (Chothia) | LCDR3 | GNSFPR |
| SEQ ID NO: 130 (IMGT) | HCDR1 | GGSIX₁X₂X₃Y, wherein |
| | | X₁ is E or S; |
| | | X₂ is R, G, or S; and |
| | | X₃ is Y or R |
| SEQ ID NO: 131 (IMGT) | HCDR2 | IYX₁X₂GST, wherein |
| | | X₁ is G or S; and |
| | | X₂ is R, S, or T |
| SEQ ID NO: 9 (IMGT) | HCDR3 | ARESQESPYNNWFDP |
| SEQ ID NO: 20 (IMGT) | LCDR1 | QGISSW |
| SEQ ID NO: 18 (IMGT) | LCDR2 | AAS |
| SEQ ID NO: 16 (IMGT) | LCDR3 | QQGNSFPRT |
| SEQ ID NO: 196 | HCDR1 | GSIX₁X₂X₃YWS, wherein |
| (Alternative) | | X₁ is E or S; |
| | | X₂ is R, G, or S; and |
| | | X₃ is Y or R |
| SEQ ID NO: 91 | HCDR2 | YIYX₁X₂GSTX₃YNPSLKS, wherein |
| (Alternative) | | X₁ is G or S; |
| | | X₂ is R, S, or T; and |
| | | X₃ is N or K |
| SEQ ID NO: 9 | HCDR3 | ARESQESPYNNWFDP |
| (Alternative) | | |
| SEQ ID NO: 14 | LCDR1 | RASQGISSWLA |
| (Alternative) | | |
| SEQ ID NO: 15 | LCDR2 | AASSLQS |
| (Alternative) | | |
| SEQ ID NO: 16 | LCDR3 | QQGNSFPRT |
| (Alternative) | | |

TABLE 17
Corresponding germline sequences of anti-CD73 antibodies

| Antibody | VH germline | VL germline |
|---|---|---|
| 918 | VH4-59 | VK1-12 |
| 350 | VH4-59 | VK1-12 |
| 356 | VH4-59 | VK1-12 |
| 358 | VH4-59 | VK1-12 |
| 930 | VH3-23 | VK3-15 |
| 373 | VH3-23 | VK3-15 |
| 374 | VH3-23 | VK3-15 |
| 376 | VH3-23 | VK3-15 |
| 377 | VH3-23 | VK3-15 |
| 379 | VH3-23 | VK3-15 |
| 363 | VH3-23 | VK1-05 |
| 366 | VH3-23 | VK1-05 |
| 407 | VH1-02 | VK1-12 |
| 893 | VH3-07 | VK4-01 |
| 939 | VH1-69 | VK3-15 |
| 430 | VH4-34 | VK1-12 |
| 398 | VH4-34 | VK1-12 |

TABLE 3
Constant region amino acid sequences of human IgG heavy chains and human kappa
light chain

| SEQ | | |
|---|---|---|
| ID | | |
| NO | Description | Sequence |
| 92 | IgG4 (S228P) | ASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTK |
| | | VDKRVESKYGPPCPPCPAPEFLGGPSVFLFPPKPKDTLMISRTPEVT |
| | | CVVVDVSQEDPEVQFNWYVDGVEVHNAKTKPREEQFNSTYRVVSV |
| | | LTVLHQDWLNGKEYKCKVSNKGLPSSIEKTISKAKGQPREPQVYTLP |
| | | PSQEEMTKNQVSLTCLVKGFYPSDIAVEWESNGQPENNYKTTPPVL |
| | | DSDGSFFLYSRLTVDKSRWQEGNVFSCSVMHEALHNHYTQKSLSL |
| | | SLGK |
| 93 | IgG4 (S228P) | ASTKGPSVFPLAPCSRSTSESTAALGCLVKDYFPEPVTVSWNSGAL |
| | lacking C- | TSGVHTFPAVLQSSGLYSLSSVVTVPSSSLGTKTYTCNVDHKPSNTK |
| | terminal lysine | VDKRVESKYGPPCPPCPAPEFLGGPSVFLFPPKPKDTLMISRTPEVT |
| | | CVVVDVSQEDPEVQFNWYVDGVEVHNAKTKPREEQFNSTYRVVSV |
| | | LTVLHQDWLNGKEYKCKVSNKGLPSSIEKTISKAKGQPREPQVYTLP |
| | | PSQEEMTKNQVSLTCLVKGFYPSDIAVEWESNGQPENNYKTTPPVL |
| | | DSDGSFFLYSRLTVDKSRWQEGNVFSCSVMHEALHNHYTQKSLSL |
| | | SLG |
| 94 | IgG4 | ASTKGPSVFPLAPCSRSTSESTAALGCLVKDYFPEPVTVSWNSGAL |
| | (S228P/L235E) | TSGVHTFPAVLQSSGLYSLSSVVTVPSSSLGTKTYTCNVDHKPSNTK |
| | | VDKRVESKYGPPCPPCPAPEFEGGPSVFLFPPKPKDTLMISRTPEVT |
| | | CVVVDVSQEDPEVQFNWYVDGVEVHNAKTKPREEQFNSTYRVVSV |
| | | LTVLHQDWLNGKEYKCKVSNKGLPSSIEKTISKAKGQPREPQVYTLP |
| | | PSQEEMTKNQVSLTCLVKGFYPSDIAVEWESNGQPENNYKTTPPVL |
| | | DSDGSFFLYSRLTVDKSRWQEGNVFSCSVMHEALHNHYTQKSLSL |
| | | SLGK |
| 95 | IgG4 | ASTKGPSVFPLAPCSRSTSESTAALGCLVKDYFPEPVTVSWNSGAL |
| | (S228P/L235E) | TSGVHTFPAVLQSSGLYSLSSVVTVPSSSLGTKTYTCNVDHKPSNTK |
| | lacking C- | VDKRVESKYGPPCPPCPAPEFEGGPSVFLFPPKPKDTLMISRTPEVT |
| | terminal lysine | CVVVDVSQEDPEVQFNWYVDGVEVHNAKTKPREEQFNSTYRVVSV |
| | | LTVLHQDWLNGKEYKCKVSNKGLPSSIEKTISKAKGQPREPQVYTLP |
| | | PSQEEMTKNQVSLTCLVKGFYPSDIAVEWESNGQPENNYKTTPPVL |
| | | DSDGSFFLYSRLTVDKSRWQEGNVFSCSVMHEALHNHYTQKSLSL |
| | | SLG |
| 119 | IgG1 wild type | ASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTK |
| | | VDKKVEPKSCDKTHTCPPCPAPELLGGPSVFLFPPKPKDTLMISRTP |
| | | EVTCVVVDVSHEDPEVKFNWYVDGVEVHNAKTKPREEQYNSTYRV |
| | | VSVLTVLHQDWLNGKEYKCKVSNKALPAPIEKTISKAKGQPREPQVY |
| | | TLPPSRDELTKNQVSLTCLVKGFYPSDIAVEWESNGQPENNYKTTP |
| | | PVLDSDGSFFLYSKLTVDKSRWQQGNVFSCSVMHEALHNHYTQKS |
| | | LSLSPGK |
| 120 | IgG1 wild type | ASTKGPSVFPLAPSSKSTSGGTAALGCLVKDYFPEPVTVSWNSGAL |
| | lacking C- | TSGVHTFPAVLQSSGLYSLSSVVTVPSSSLGTQTYICNVNHKPSNTK |
| | terminal lysine | VDKKVEPKSCDKTHTCPPCPAPELLGGPSVFLFPPKPKDTLMISRTP |
| | | EVTCVVVDVSHEDPEVKFNWYVDGVEVHNAKTKPREEQYNSTYRV |
| | | VSVLTVLHQDWLNGKEYKCKVSNKALPAPIEKTISKAKGQPREPQVY |
| | | TLPPSRDELTKNQVSLTCLVKGFYPSDIAVEWESNGQPENNYKTTP |
| | | PVLDSDGSFFLYSKLTVDKSRWQQGNVFSCSVMHEALHNHYTQKS |
| | | LSLSPG |
| 6 | IgG1 wild type | ASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISRTP |
| | | EVTCVVVDVSHEDPEVKFNWYVDGVEVHNAKTKPREEQYNSTYRV |
| | | VSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTP |
| | | PVLDSDGSFFLYSKLTVDKSRWQQGNVFSCSVMHEALHNHYTQKS |
| | | LSLSPGK |
| 97 | IgG1 wild type | ASTKGPSVFPLAPSSKSTSGGTAALGCLVKDYFPEPVTVSWNSGAL |
| | lacking C- | TSGVHTFPAVLQSSGLYSLSSVVTVPSSSLGTQTYICNVNHKPSNTK |
| | terminal lysine | VDKRVEPKSCDKTHTCPPCPAPELLGGPSVFLFPPKPKDTLMISRTP |
| | | EVTCVVVDVSHEDPEVKFNWYVDGVEVHNAKTKPREEQYNSTYRV |
| | | VSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTP |
| | | PVLDSDGSFFLYSKLTVDKSRWQQGNVFSCSVMHEALHNHYTQKS |
| | | LSLSPG |
| 98 | IgG1 (N297A) | ASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISRTP |
| | | EVTCVVVDVSHEDPEVKFNWYVDGVEVHNAKTKPREEQYASTYRV |
| | | VSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTP |
| | | PVLDSDGSFFLYSKLTVDKSRWQQGNVFSCSVMHEALHNHYTQKS |
| | | LSLSPGK |
| 99 | IgG1 (N297A) | ASTKGPSVFPLAPSSKSTSGGTAALGCLVKDYFPEPVTVSWNSGAL |
| | lacking C- | TSGVHTFPAVLQSSGLYSLSSVVTVPSSSLGTQTYICNVNHKPSNTK |
| | terminal lysine | VDKRVEPKSCDKTHTCPPCPAPELLGGPSVFLFPPKPKDTLMISRTP |
| | | EVTCVVVDVSHEDPEVKFNWYVDGVEVHNAKTKPREEQYASTYRV |
| | | VSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTP |
| | | PVLDSDGSFFLYSKLTVDKSRWQQGNVFSCSVMHEALHNHYTQKS |
| | | LSLSPG |
| 100 | IgG1 | ASTKGPSVFPLAPSSKSTSGGTAALGCLVKDYFPEPVTVSWNSGAL |
| | (D265A/P329A) | TSGVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISRTP |
| | | EVTCVVVAVSHEDPEVKFNWYVDGVEVHNAKTKPREEQYNSTYRV |
| | | VSVLTVLHQDWLNGKEYKCKVSNKALAAPIEKTISKAKGQPREPQVY |
| | | TLPPSREEMTKNQVSLTCLVKGFYPSDIAVEWESNGQPENNYKTTP |
| | | PVLDSDGSFFLYSKLTVDKSRWQQGNVFSCSVMHEALHNHYTQKS |
| | | LSLSPGK |
| 101 | IgG1 | ASTKGPSVFPLAPSSKSTSGGTAALGCLVKDYFPEPVTVSWNSGAL |
| | (D265A/P329A) | TSGVHTFPAVLQSSGLYSLSSVVTVPSSSLGTQTYICNVNHKPSNTK |
| | lacking C- | VDKRVEPKSCDKTHTCPPCPAPELLGGPSVFLFPPKPKDTLMISRTP |
| | terminal lysine | EVTCVVVAVSHEDPEVKFNWYVDGVEVHNAKTKPREEQYNSTYRV |
| | | VSVLTVLHQDWLNGKEYKCKVSNKALAAPIEKTISKAKGQPREPQVY |
| | | TLPPSREEMTKNQVSLTCLVKGFYPSDIAVEWESNGQPENNYKTTP |
| | | PVLDSDGSFFLYSKLTVDKSRWQQGNVFSCSVMHEALHNHYTQKS |
| | | LSLSPG |
| 102 | IgG1 | ASTKGPSVFPLAPSSKSTSGGTAALGCLVKDYFPEPVTVSWNSGAL |
| | (L234A/L235A) | TSGVHTFPAVLQSSGLYSLSSVVTVPSSSLGTQTYICNVNHKPSNTK |
| | | VDKRVEPKSCDKTHTCPPCPAPEAAGGPSVFLFPPKPKDTLMISRT |
| | | PEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTT |
| | | PPVLDSDGSFFLYSKLTVDKSRWQQGNVFSCSVMHEALHNHYTQK |
| | | SLSLSPGK |
| 103 | IgG1 | ASTKGPSVFPLAPSSKSTSGGTAALGCLVKDYFPEPVTVSWNSGAL |
| | (L234A/L235A) | TSGVHTFPAVLQSSGLYSLSSVVTVPSSSLGTQTYICNVNHKPSNTK |
| | lacking C- | VDKRVEPKSCDKTHTCPPCPAPEAAGGPSVFLFPPKPKDTLMISRT |
| | terminal lysine | PEVTCVVVDVSHEDPEVKFNWYVDGVEVHNAKTKPREEQYNSTYR |
| | | VVSVLTVLHQDWLNGKEYKCKVSNKALPAPIEKTISKAKGQPREPQV |
| | | YTLPPSREEMTKNQVSLTCLVKGFYPSDIAVEWESNGQPENNYKTT |
| | | PPVLDSDGSFFLYSKLTVDKSRWQQGNVFSCSVMHEALHNHYTQK |
| | | SLSLSPG |
| 104 | Human kappa | RTVAAPSVFIFPPSDEQLKSGTASVVCLLNNFYPREAKVQWKVDNA |
| | constant | LQSGNSQESVTEQDSKDSTYSLSSTLTLSKADYEKHKVYACEVTHQ |
| | region | GLSSPVTKSFNRGEC |

TABLE 4
Exemplary sequences of CD73

| SEQ ID | | |
|---|---|---|
| NO | Description | Sequence |
| 105 | Human CD73 | MCPRAARAPATLLLALGAVLWPAAGAWELTILHTNDVHSR |
| | (Q6NZX3) | LEQTSEDSSKCVNASRCMGGVARLFTKVQQIRRAEPNVLL |
| | | LDAGDQYQGTIWFTVYKGAEVAHFMNALRYDAMALGNHE |
| | | FDNGVEGLIEPLLKEAKFPILSANIKAKGPLASQISGLYLPYK |
| | | VLPVGDEVVGIVGYTSKETPFLSNPGTNLVFEDEITALQPE |
| | | VDKLKTLNVNKIIALGHSGFEMDKLIAQKVRGVDVVVGGHS |
| | | NTFLYTGNPPSKEVPAGKYPFIVTSDDGRKVPVVQAYAFG |
| | | KYLGYLKIEFDERGNVISSHGNPILLNSSIPEDPSIKADINKW |
| | | RIKLDNYSTQELGKTIVYLDGSSQSCRFRECNMGNLICDAM |
| | | INNNLRHADETFWNHVSMCILNGGGIRSPIDERNNGTITWE |
| | | NLAAVLPFGGTFDLVQLKGSTLKKAFEHSVHRYGQSTGEF |
| | | LQVGGIHVVYDLSRKPGDRVVKLDVLCTKCRVPSYDPLKM |
| | | DEVYKVILPNFLANGGDGFQMIKDELLRHDSGDQDINVVST |
| | | YISKMKVIYPAVEGRIKFSTGSHCHGSFSLIFLSLWAVIFVLY |
| | | Q |
| 106 | Human CD73 isoform | MCPRAARAPATLLLALGAVLWPAAGAWELTILHTNDVHSR |
| | (P21589-1) | LEQTSEDSSKCVNASRCMGGVARLFTKVQQIRRAEPNVLL |
| | | LDAGDQYQGTIWFTVYKGAEVAHFMNALRYDAMALGNHE |
| | | FDNGVEGLIEPLLKEAKFPILSANIKAKGPLASQISGLYLPYK |
| | | VLPVGDEVVGIVGYTSKETPFLSNPGTNLVFEDEITALQPE |
| | | VDKLKTLNVNKIIALGHSGFEMDKLIAQKVRGVDVVVGGHS |
| | | NTFLYTGNPPSKEVPAGKYPFIVTSDDGRKVPVVQAYAFG |
| | | KYLGYLKIEFDERGNVISSHGNPILLNSSIPEDPSIKADINKW |
| | | RIKLDNYSTQELGKTIVYLDGSSQSCRFRECNMGNLICDAM |
| | | INNNLRHTDEMFWNHVSMCILNGGGIRSPIDERNNGTITWE |
| | | NLAAVLPFGGTFDLVQLKGSTLKKAFEHSVHRYGQSTGEF |
| | | LQVGGIHVVYDLSRKPGDRVVKLDVLCTKCRVPSYDPLKM |
| | | DEVYKVILPNFLANGGDGFQMIKDELLRHDSGDQDINVVST |
| | | YISKMKVIYPAVEGRIKFSTGSHCHGSFSLIFLSLWAVIFVLY |
| | | Q |
| 107 | Human CD73 isoform | MCPRAARAPATLLLALGAVLWPAAGAWELTILHTNDVHSR |
| | (P21589-2) | LEQTSEDSSKCVNASRCMGGVARLFTKVQQIRRAEPNVLL |
| | | LDAGDQYQGTIWFTVYKGAEVAHFMNALRYDAMALGNHE |
| | | FDNGVEGLIEPLLKEAKFPILSANIKAKGPLASQISGLYLPYK |
| | | VLPVGDEVVGIVGYTSKETPFLSNPGTNLVFEDEITALQPE |
| | | VDKLKTLNVNKIIALGHSGFEMDKLIAQKVRGVDVVVGGHS |
| | | NTFLYTGNPPSKEVPAGKYPFIVTSDDGRKVPVVQAYAFG |
| | | KYLGYLKIEFDERGNVISSHGNPILLNSSIPEDPSIKADINKW |
| | | RIKLDNYSTQELGKTIVYLDGSSQSCRFRECNMGNLICDAM |
| | | INNNLRHTDEMFWNHVSMCILNGGGIRSPIDERNNGIHVVY |
| | | DLSRKPGDRVVKLDVLCTKCRVPSYDPLKMDEVYKVILPNF |
| | | LANGGDGFQMIKDELLRHDSGDQDINVVSTYISKMKVIYPA |
| | | VEGRIKFSTGSHCHGSFSLIFLSLWAVIFVLYQ |
| 108 | CD73 fragment | YLPYKVLPVGDEVVG |
| | (residue range 158- | |
| | 172 of SEQ ID NO: | |
| | 105) | |
| 109 | CD73 fragment | KLKTLNVNKI |
| | (residue range 206- | |
| | 215 of SEQ ID NO: | |
| | 105) | |
| 110 | CD73 fragment | MINNNLRHADETFWNHVSMC |
| | (residue range 368- | |
| | 387 of SEQ ID NO: | |
| | 105) | |
| 121 | CD73 fragment | MINNNLRHTDEMFWNHVSMC |
| | (residue range 368- | |
| | 387 of SEQ ID NO: | |
| | 106) | |
| 111 | CD73 fragment | YQGTIWFTVYKGAEVAHF |
| | (residue range 87-104 | |
| | of SEQ ID NO: 105) | |
| 118 | CD73 fragment | RGNVISSHGNPIL |
| | (residue range 297- | |
| | 309 of SEQ ID NO: | |
| | 105) | |
| 134 | CD73 fragment | WELTILHTNDVHSRLEQTSEDSSKCVNASRCMGGVARLFT |
| | (residue range 27-547 | KVQQIRRAEPNVLLLDAGDQYQGTIWFTVYKGAEVAHFMN |
| | of SEQ ID NO: 105) | ALRYDAMALGNHEFDNGVEGLIEPLLKEAKFPILSANIKAKG |
| | | PLASQISGLYLPYKVLPVGDEVVGIVGYTSKETPFLSNPGT |
| | | NLVFEDEITALQPEVDKLKTLNVNKIIALGHSGFEMDKLIAQ |
| | | KVRGVDVVVGGHSNTFLYTGNPPSKEVPAGKYPFIVTSDD |
| | | GRKVPVVQAYAFGKYLGYLKIEFDERGNVISSHGNPILLNS |
| | | SIPEDPSIKADINKWRIKLDNYSTQELGKTIVYLDGSSQSCR |
| | | FRECNMGNLICDAMINNNLRHADETFWNHVSMCILNGGGI |
| | | RSPIDERNNGTITWENLAAVLPFGGTFDLVQLKGSTLKKAF |
| | | EHSVHRYGQSTGEFLQVGGIHVVYDLSRKPGDRVVKLDVL |
| | | CTKCRVPSYDPLKMDEVYKVILPNFLANGGDGFQMIKDELL |
| | | RHDSGDQDINVVSTYISKMKVIYPAVEGRIK |
| 171 | CD73 fragment | WELTILHTNDVHSRLEQTSEDSSKCVNASRCMGGVARLFT |
| | (residue range 27-547 | KVQQIRRAEPNVLLLDAGDQYQGTIWFTVYKGAEVAHFMN |
| | of SEQ ID NO: 105) | ALRYDAMALGNHEFDNGVEGLIEPLLKEAKFPILSANIKAKG |
| | fused to a C-terminal | PLASQISGLYLPYKVLPVGDEVVGIVGYTSKETPFLSNPGT |
| | 6-His tag (SEQ ID | NLVFEDEITALQPEVDKLKTLNVNKIIALGHSGFEMDKLIAQ |
| | NO: 922) | KVRGVDVVVGGHSNTFLYTGNPPSKEVPAGKYPFIVTSDD |
| | | GRKVPVVQAYAFGKYLGYLKIEFDERGNVISSHGNPILLNS |
| | | SIPEDPSIKADINKWRIKLDNYSTQELGKTIVYLDGSSQSCR |
| | | FRECNMGNLICDAMINNNLRHADETFWNHVSMCILNGGGI |
| | | RSPIDERNNGTITWENLAAVLPFGGTFDLVQLKGSTLKKAF |
| | | EHSVHRYGQSTGEFLQVGGIHVVYDLSRKPGDRVVKLDVL |
| | | CTKCRVPSYDPLKMDEVYKVILPNFLANGGDGFQMIKDELL |
| | | RHDSGDQDINVVSTYISKMKVIYPAVEGRIKHHHHHH |
| 332 | CD73 fragment | MCPRAARAPATLLLALGAVLWPAAGAWELTILHTNDVHSR |
| | (residue range 27-547 | LEQTSEDSSKCVNASRCMGGVARLFTKVQQIRRAEPNVLL |
| | of SEQ ID NO: 105) | LDAGDQYQGTIWFTVYKGAEVAHFMNALRYDAMALGNHE |
| | fused to a C-terminal | FDNGVEGLIEPLLKEAKFPILSANIKAKGPLASQISGLYLPYK |
| | 6-His tag (SEQ ID | VLPVGDEVVGIVGYTSKETPFLSNPGTNLVFEDEITALQPE |
| | NO: 922), including a | VDKLKTLNVNKIIALGHSGFEMDKLIAQKVRGVDVVVGGHS |
| | signal peptide | NTFLYTGNPPSKEVPAGKYPFIVTSDDGRKVPVVQAYAFG |
| | | KYLGYLKIEFDERGNVISSHGNPILLNSSIPEDPSIKADINKW |
| | | RIKLDNYSTQELGKTIVYLDGSSQSCRFRECNMGNLICDAM |
| | | INNNLRHADETFWNHVSMCILNGGGIRSPIDERNNGTITWE |
| | | NLAAVLPFGGTFDLVQLKGSTLKKAFEHSVHRYGQSTGEF |
| | | LQVGGIHVVYDLSRKPGDRVVKLDVLCTKCRVPSYDPLKM |
| | | DEVYKVILPNFLANGGDGFQMIKDELLRHDSGDQDINVVST |
| | | YISKMKVIYPAVEGRIKHHHHHH |

In other embodiments, the aforesaid antibody molecules are capable of binding to human CD73 with a dissociation constant (K_{D}) of less than about 1×10⁻⁴ M, 1×10⁻⁵ M, 1×10⁻⁶ M, 1×10⁻⁷ M, 1×10⁻⁸ M, 1×10⁻⁹ M, e.g., as measured by Biacore, Octet, flow cytometry, or ELISA.

In some embodiments, the antibody molecule binds to a mammalian, e.g., human or cynomolgus, CD73. For example, the antibody molecule binds to an epitope, e.g., linear or conformational epitope, (e.g., an epitope as described herein), on CD73. In certain aspects, it is advantageous to identify an antibody that binds with high affinity to the human and cynomolgus homologs of a protein of interest. This desirable cross-reactivity allows the same antibody (or two antibodies with the same CDRs or variable regions) to be tested in an animal model and then administered to human patients as a therapeutic.

In some embodiments, disclosed herein is an isolated antibody molecule that competes for binding to human CD73 with the aforesaid anti-CD73 antibody molecules.

In some embodiments, disclosed herein is an isolated antibody molecule that binds to the same epitope as, substantially the same epitope as, an epitope that overlaps with, or an epitope that substantially overlaps with, the epitope of the aforesaid anti-CD73 antibody molecules.

In another aspect, disclosed herein is an isolated nucleic acid encoding any of the aforesaid antibody molecules, vectors and host cells thereof. The nucleic acid molecule includes but is not limited to RNA, genomic DNA and cDNA.

In some embodiments, the isolated nucleic acid encodes the antibody heavy chain variable region, light chain variable region, heavy chain, and/or light chain of any of the aforesaid antibody molecules.

In some embodiments, the isolated nucleic acid encodes a heavy chain variable region, wherein the nucleic acid comprises the nucleotide sequence of SEQ ID NO: 45, 78, 85, 143, 152, 160, 67, 32, 11, or 169, or a nucleotide sequence having at least about 85%, 90%, 95%, or 99% sequence identity to SEQ ID NO: 45, 78, 85, 143, 152, 160, 67, 32, 11, or 169.

In some embodiments, the isolated nucleic acid encodes a heavy chain, wherein the nucleic acid comprises the nucleotide sequence of SEQ ID NO: 47, 80, 87, 69, 34, or 13, or a nucleotide sequence having at least about 85%, 90%, 95%, or 99% sequence identity to SEQ ID NO: 47, 80, 87, 69, 34, or 13.

In some embodiments, the isolated nucleic acid encodes a light chain variable region, wherein the nucleic acid comprises the nucleotide sequence of SEQ ID NO: 56, 144, 22, or 170, or a nucleotide sequence having at least about 85%, 90%, 95%, or 99% sequence identity to SEQ ID NO: 56, 144, 22, or 170.

In some embodiments, the isolated nucleic acid encodes a light chain, wherein the nucleic acid comprises the nucleotide sequence of SEQ ID NO: 58 or 24, or a nucleotide sequence having at least about 85%, 90%, 95%, or 99% sequence identity to SEQ ID NO: 58 or 24.

Exemplary Antibody Molecules that Specifically Bind to Human ENTPD2

In one embodiment of the dosage regimes of the invention, the anti-ENTPD2 antibody is an anti-ENTPD2 antibody molecule as described in WO2019229658, published on 5 Dec. 2019, entitled “ENTPD2 ANTIBODIES, COMBINATION THERAPIES, AND METHODS OF USING THE ANTIBODIES AND COMBINATION THERAPIES,” which is incorporated by reference in its entirety.

In one aspect, provided herein are antibodies or antigen binding fragments thereof, e.g., monoclonal antibodies or antigen binding fragments thereof, that specifically bind to ENTPD2 protein (“ENTPD2 antibodies or antigen binding fragments” or “anti-ENTPD2 antibodies or antigen binding fragments”). In some embodiments, provided herein are antibodies or antigen binding fragments thereof, e.g., monoclonal antibodies or antigen binding fragments thereof, that specifically bind to human ENTPD2 protein (“human ENTPD2 antibodies or antigen binding fragments” or “anti-human ENTPD2 antibodies or antigen binding fragments”). In some embodiments, the anti-ENTPD2 antibodies or antigen-binding fragments thereof (e.g., anti-human ENTPD2 antibodies or antigen binding fragments) provided herein include a heavy chain CDR1 (HCDR1), a heavy chain CDR2 (HCDR2), a heavy chain CDR3 (HCDR3), and a light chain CDR1 (LCDR1), a light chain CDR2 (LCDR2), and a light chain CDR3 (LCDR3). In some embodiments, the anti-ENTPD2 antibodies or antigen binding fragments (e.g., anti-human ENTPD2 antibodies or antigen binding fragments) provided herein include a heavy chain variable region (VH) comprising CDR1, CDR2, and CDR3 and a light chain variable region (VL) comprising CDR1, CDR2, and CDR3. In some embodiments, the anti-ENTPD2 antibodies or antigen-binding fragments (e.g., anti-human ENTPD2 antibodies or antigen binding fragments) provided herein include a full length heavy chain sequence (HC) and a full length light chain sequence (LC).

Table 9 lists the sequence of ENTPD2 antibodies that specifically bind to human ENTPD2 protein.

TABLE 9
Sequences of exemplary anti-ENTPD2 antibodies

| | | |
|---|---|---|
| ANTI-HUMAN | | |
| ENTPD2 MAB1 | | |
| SEQ ID NO: 401 | HCDR1 (KABAT) | DYNMD |
| SEQ ID NO: 402 | HCDR2 (KABAT) | DINPKYDISTYNQQFKG |
| SEQ ID NO: 403 | HCDR3 (KABAT) | RGFFLYYGINYYYFDV |
| SEQ ID NO: 404 | HCDR1 (CHOTHIA) | GYTFTDY |
| SEQ ID NO: 405 | HCDR2 (CHOTHIA) | NPKYDI |
| SEQ ID NO: 403 | HCDR3 (CHOTHIA) | RGFFLYYGINYYYFDV |
| SEQ ID NO: 406 | HCDR1 | GYTFTDYNMD |
| | (COMBINED) | |
| SEQ ID NO: 402 | HCDR2 | DINPKYDISTYNQQFKG |
| | (COMBINED) | |
| SEQ ID NO: 403 | HCDR3 | RGFFLYYGINYYYFDV |
| | (COMBINED) | |
| SEQ ID NO: 407 | HCDR1 (IMGT) | GYTFTDYN |
| SEQ ID NO: 408 | HCDR2 (IMGT) | INPKYDIS |
| SEQ ID NO: 409 | HCDR3 (IMGT) | ARRGFFLYYGINYYYFDV |
| SEQ ID NO: 410 | VH | EVQLVQSGAEVKKPGESLKISCKASGYTFTDYNMD |
| | | WVRQMPGKGLEWMGDINPKYDISTYNQQFKGQVTI |
| | | SADKSIRTAYLQWSSLKASDTAMYYCARRGFFLYY |
| | | GINYYYFDVWGQGTLVTVSS |
| SEQ ID NO: 411 | VH DNA | GAAGTGCAGCTGGTGCAGTCTGGCGCCGAAGTG |
| | | AAGAAGCCTGGCGAGTCCCTGAAGATCTCCTGCA |
| | | AGGCCTCCGGCTACACCTTCACCGACTACAACAT |
| | | GGACTGGGTGCGACAGATGCCCGGCAAGGGCCT |
| | | GGAATGGATGGGCGACATCAACCCTAAGTACGAC |
| | | ATCTCCACCTACAACCAGCAGTTCAAGGGCCAAG |
| | | TGACCATCTCCGCCGACAAGTCCATCCGGACCGC |
| | | CTACCTGCAGTGGTCCTCCCTGAAGGCCTCTGAC |
| | | ACCGCCATGTACTACTGCGCCAGACGGGGCTTCT |
| | | TCCTGTACTACGGCATCAACTACTACTACTTCGAC |
| | | GTGTGGGGCCAGGGCACCCTCGTGACAGTGTCA |
| | | TCT |
| SEQ ID NO: 412 | HC | EVQLVQSGAEVKKPGESLKISCKASGYTFTDYNMD |
| | | WVRQMPGKGLEWMGDINPKYDISTYNQQFKGQVTI |
| | | SADKSIRTAYLQWSSLKASDTAMYYCARRGFFLYY |
| | | GINYYYFDVWGQGTLVTVSSASTKGPSVFPLAPSSK |
| | | STSGGTAALGCLVKDYFPEPVTVSWNSGALTSGVH |
| | | TFPAVLQSSGLYSLSSVVTVPSSSLGTQTYICNVNH |
| | | KPSNTKVDKRVEPKSCDKTHTCPPCPAPELLGGPS |
| | | VFLFPPKPKDTLMISRTPEVTCVVVDVSHEDPEVKF |
| | | NWYVDGVEVHNAKTKPREEQYNSTYRVVSVLTVLH |
| | | QDWLNGKEYKCKVSNKALPAPIEKTISKAKGQPREP |
| | | QVYTLPPSREEMTKNQVSLTCLVKGFYPSDIAVEWE |
| | | SNGQPENNYKTTPPVLDSDGSFFLYSKLTVDKSRW |
| | | QQGNVFSCSVMHEALHNHYTQKSLSLSPGK |
| SEQ ID NO: 413 | HC DNA | GAAGTGCAGCTGGTGCAGTCTGGCGCCGAAGTG |
| | | AAGAAGCCTGGCGAGTCCCTGAAGATCTCCTGCA |
| | | AGGCCTCCGGCTACACCTTCACCGACTACAACAT |
| | | GGACTGGGTGCGACAGATGCCCGGCAAGGGCCT |
| | | GGAATGGATGGGCGACATCAACCCTAAGTACGAC |
| | | ATCTCCACCTACAACCAGCAGTTCAAGGGCCAAG |
| | | TGACCATCTCCGCCGACAAGTCCATCCGGACCGC |
| | | CTACCTGCAGTGGTCCTCCCTGAAGGCCTCTGAC |
| | | ACCGCCATGTACTACTGCGCCAGACGGGGCTTCT |
| | | TCCTGTACTACGGCATCAACTACTACTACTTCGAC |
| | | GTGTGGGGCCAGGGCACCCTCGTGACAGTGTCA |
| | | TCTGCTAGCACCAAGGGCCCAAGTGTGTTTCCCC |
| | | TGGCCCCCAGCAGCAAGTCTACTTCCGGCGGAAC |
| | | TGCTGCCCTGGGTTGCCTGGTGAAGGACTACTTC |
| | | CCCGAGCCCGTGACAGTGTCCTGGAACTCTGGG |
| | | GCTCTGACTTCCGGCGTGCACACCTTCCCCGCCG |
| | | TGCTGCAGAGCAGCGGCCTGTACAGCCTGAGCA |
| | | GCGTGGTGACAGTGCCCTCCAGCTCTCTGGGAAC |
| | | CCAGACCTATATCTGCAACGTGAACCACAAGCCC |
| | | AGCAACACCAAGGTGGACAAGAGAGTGGAGCCC |
| | | AAGAGCTGCGACAAGACCCACACCTGCCCCCCCT |
| | | GCCCAGCTCCAGAACTGCTGGGAGGGCCTTCCG |
| | | TGTTCCTGTTCCCCCCCAAGCCCAAGGACACCCT |
| | | GATGATCAGCAGGACCCCCGAGGTGACCTGCGT |
| | | GGTGGTGGACGTGTCCCACGAGGACCCAGAGGT |
| | | GAAGTTCAACTGGTACGTGGACGGCGTGGAGGT |
| | | GCACAACGCCAAGACCAAGCCCAGAGAGGAGCA |
| | | GTACAACAGCACCTACAGGGTGGTGTCCGTGCTG |
| | | ACCGTGCTGCACCAGGACTGGCTGAACGGCAAA |
| | | GAATACAAGTGCAAAGTCTCCAACAAGGCCCTGC |
| | | CAGCCCCAATCGAAAAGACAATCAGCAAGGCCAA |
| | | GGGCCAGCCACGGGAGCCCCAGGTGTACACCCT |
| | | GCCCCCCAGCCGGGAGGAGATGACCAAGAACCA |
| | | GGTGTCCCTGACCTGTCTGGTGAAGGGCTTCTAC |
| | | CCCAGCGATATCGCCGTGGAGTGGGAGAGCAAC |
| | | GGCCAGCCCGAGAACAACTACAAGACCACCCCC |
| | | CCAGTGCTGGACAGCGACGGCAGCTTCTTCCTGT |
| | | ACAGCAAGCTGACCGTGGACAAGTCCAGGTGGC |
| | | AGCAGGGCAACGTGTTCAGCTGCAGCGTGATGCA |
| | | CGAGGCCCTGCACAACCACTACACCCAGAAGTCC |
| | | CTGAGCCTGAGCCCCGGCAAG |
| SEQ ID NO: 414 | LCDR1 (KABAT) | SASSSVSYIH |
| SEQ ID NO: 415 | LCDR2 (KABAT) | STSNLAS |
| SEQ ID NO: 416 | LCDR3 (KABAT) | HQWSSYPWT |
| SEQ ID NO: 417 | LCDR1 (CHOTHIA) | SSSVSY |
| SEQ ID NO: 418 | LCDR2 (CHOTHIA) | STS |
| SEQ ID NO: 419 | LCDR3 (CHOTHIA) | WSSYPW |
| SEQ ID NO: 414 | LCDR1 | SASSSVSYIH |
| | (COMBINED) | |
| SEQ ID NO: 415 | LCDR2 | STSNLAS |
| | (COMBINED) | |
| SEQ ID NO: 416 | LCDR3 | HQWSSYPWT |
| | (COMBINED) | |
| SEQ ID NO: 420 | LCDR1 (IMGT) | SSVSY |
| SEQ ID NO: 418 | LCDR2 (IMGT) | STS |
| SEQ ID NO: 416 | LCDR3 (IMGT) | HQWSSYPWT |
| SEQ ID NO: 421 | VL | DIQLTQSPSSLSASVGDRVTITCSASSSVSYIHWFQ |
| | | QKPGKAPKLLIYSTSNLASGVPSRFSGSGSGTFFTL |
| | | TISSLQPEDFATYFCHQWSSYPWTFGQGTKVEIK |
| SEQ ID NO: 422 | VL DNA | GATATCCAGCTGACCCAGTCCCCTTCCAGCCTGT |
| | | CTGCCTCTGTGGGCGACAGAGTGACAATTACCTG |
| | | CTCCGCCTCCTCCTCCGTGTCCTACATCCACTGG |
| | | TTCCAGCAGAAGCCCGGCAAGGCCCCCAAGCTG |
| | | CTGATCTACTCCACCTCCAACCTGGCCTCCGGCG |
| | | TGCCCTCTAGATTCTCCGGCTCTGGCTCTGGCAC |
| | | CTTTTTTACCCTGACCATCTCCAGCCTGCAGCCC |
| | | GAGGACTTCGCCACCTACTTTTGCCACCAGTGGT |
| | | CCAGCTACCCCTGGACCTTTGGCCAGGGCACCAA |
| | | GGTGGAAATCAAG |
| SEQ ID NO: 423 | LC | DIQLTQSPSSLSASVGDRVTITCSASSSVSYIHWFQ |
| | | QKPGKAPKLLIYSTSNLASGVPSRFSGSGSGTFFTL |
| | | TISSLQPEDFATYFCHQWSSYPWTFGQGTKVEIKRT |
| | | VAAPSVFIFPPSDEQLKSGTASVVCLLNNFYPREAK |
| | | VQWKVDNALQSGNSQESVTEQDSKDSTYSLSSTLT |
| | | LSKADYEKHKVYACEVTHQGLSSPVTKSFNRGEC |
| SEQ ID NO: 424 | LC DNA | GATATCCAGCTGACCCAGTCCCCTTCCAGCCTGT |
| | | CTGCCTCTGTGGGCGACAGAGTGACAATTACCTG |
| | | CTCCGCCTCCTCCTCCGTGTCCTACATCCACTGG |
| | | TTCCAGCAGAAGCCCGGCAAGGCCCCCAAGCTG |
| | | CTGATCTACTCCACCTCCAACCTGGCCTCCGGCG |
| | | TGCCCTCTAGATTCTCCGGCTCTGGCTCTGGCAC |
| | | CTTTTTTACCCTGACCATCTCCAGCCTGCAGCCC |
| | | GAGGACTTCGCCACCTACTTTTGCCACCAGTGGT |
| | | CCAGCTACCCCTGGACCTTTGGCCAGGGCACCAA |
| | | GGTGGAAATCAAGCGTACGGTGGCCGCTCCCAG |
| | | CGTGTTCATCTTCCCCCCCAGCGACGAGCAGCTG |
| | | AAGAGTGGCACCGCCAGCGTGGTGTGCCTGCTG |
| | | AACAACTTCTACCCCCGGGAGGCCAAGGTGCAGT |
| | | GGAAGGTGGACAACGCCCTGCAGAGCGGCAACA |
| | | GCCAGGAGAGCGTCACCGAGCAGGACAGCAAGG |
| | | ACTCCACCTACAGCCTGAGCAGCACCCTGACCCT |
| | | GAGCAAGGCCGACTACGAGAAGCATAAGGTGTAC |
| | | GCCTGCGAGGTGACCCACCAGGGCCTGTCCAGC |
| | | CCCGTGACCAAGAGCTTCAACAGGGGCGAGTGC |
| ANTI-HUMAN | | |
| ENTPD2 MAB2 | | |
| SEQ ID NO: 401 | HCDR1 (KABAT) | DYNMD |
| SEQ ID NO: 402 | HCDR2 (KABAT) | DINPKYDISTYNQQFKG |
| SEQ ID NO: 403 | HCDR3 (KABAT) | RGFFLYYGINYYYFDV |
| SEQ ID NO: 404 | HCDR1 (CHOTHIA) | GYTFTDY |
| SEQ ID NO: 405 | HCDR2 (CHOTHIA) | NPKYDI |
| SEQ ID NO: 403 | HCDR3 (CHOTHIA) | RGFFLYYGINYYYFDV |
| SEQ ID NO: 406 | HCDR1 | GYTFTDYNMD |
| | (COMBINED) | |
| SEQ ID NO: 402 | HCDR2 | DINPKYDISTYNQQFKG |
| | (COMBINED) | |
| SEQ ID NO: 403 | HCDR3 | RGFFLYYGINYYYFDV |
| | (COMBINED) | |
| SEQ ID NO: 407 | HCDR1 (IMGT) | GYTFTDYN |
| SEQ ID NO: 408 | HCDR2 (IMGT) | INPKYDIS |
| SEQ ID NO: 409 | HCDR3 (IMGT) | ARRGFFLYYGINYYYFDV |
| SEQ ID NO: 425 | VH | QVQLVQSGAEVVKPGASVKISCKASGYTFTDYNMD |
| | | WVKQAPGQRLEWIGDINPKYDISTYNQQFKGKATIT |
| | | VDKSASTAYMELSSLRSEDTAVYYCARRGFFLYYGI |
| | | NYYYFDVWGQGTLVTVSS |
| SEQ ID NO: 426 | VH DNA | CAGGTGCAGCTGGTGCAGTCTGGCGCCGAGGTG |
| | | GTGAAACCTGGCGCCTCCGTGAAGATCTCCTGCA |
| | | AGGCCTCCGGCTACACCTTCACCGACTACAACAT |
| | | GGACTGGGTGAAACAGGCCCCTGGCCAGCGGCT |
| | | GGAATGGATCGGCGACATCAACCCTAAGTACGAC |
| | | ATCTCCACCTACAACCAGCAGTTCAAGGGCAAGG |
| | | CCACCATCACCGTGGACAAGTCCGCCTCCACCGC |
| | | CTACATGGAACTGTCCTCCCTGCGGAGCGAGGAC |
| | | ACCGCCGTGTACTACTGCGCCAGACGGGGCTTCT |
| | | TCCTGTACTACGGCATCAACTACTACTACTTCGAC |
| | | GTGTGGGGCCAGGGCACCCTGGTGACAGTGTCC |
| | | TCC |
| SEQ ID NO: 427 | HC | QVQLVQSGAEVVKPGASVKISCKASGYTFTDYNMD |
| | | WVKQAPGQRLEWIGDINPKYDISTYNQQFKGKATIT |
| | | VDKSASTAYMELSSLRSEDTAVYYCARRGFFLYYGI |
| | | NYYYFDVWGQGTLVTVSSASTKGPSVFPLAPSSKS |
| | | TSGGTAALGCLVKDYFPEPVTVSWNSGALTSGVHT |
| | | FPAVLQSSGLYSLSSVVTVPSSSLGTQTYICNVNHK |
| | | PSNTKVDKRVEPKSCDKTHTCPPCPAPELLGGPSV |
| | | FLFPPKPKDTLMISRTPEVTCVVVDVSHEDPEVKFN |
| | | WYVDGVEVHNAKTKPREEQYNSTYRVVSVLTVLHQ |
| | | DWLNGKEYKCKVSNKALPAPIEKTISKAKGQPREPQ |
| | | VYTLPPSREEMTKNQVSLTCLVKGFYPSDIAVEWES |
| | | NGQPENNYKTTPPVLDSDGSFFLYSKLTVDKSRWQ |
| | | QGNVFSCSVMHEALHNHYTQKSLSLSPGK |
| SEQ ID NO: 428 | HC DNA | CAGGTGCAGCTGGTGCAGTCTGGCGCCGAGGTG |
| | | GTGAAACCTGGCGCCTCCGTGAAGATCTCCTGCA |
| | | AGGCCTCCGGCTACACCTTCACCGACTACAACAT |
| | | GGACTGGGTGAAACAGGCCCCTGGCCAGCGGCT |
| | | GGAATGGATCGGCGACATCAACCCTAAGTACGAC |
| | | ATCTCCACCTACAACCAGCAGTTCAAGGGCAAGG |
| | | CCACCATCACCGTGGACAAGTCCGCCTCCACCGC |
| | | CTACATGGAACTGTCCTCCCTGCGGAGCGAGGAC |
| | | ACCGCCGTGTACTACTGCGCCAGACGGGGCTTCT |
| | | TCCTGTACTACGGCATCAACTACTACTACTTCGAC |
| | | GTGTGGGGCCAGGGCACCCTGGTGACAGTGTCC |
| | | TCCGCTAGCACCAAGGGCCCAAGTGTGTTTCCCC |
| | | TGGCCCCCAGCAGCAAGTCTACTTCCGGCGGAAC |
| | | TGCTGCCCTGGGTTGCCTGGTGAAGGACTACTTC |
| | | CCCGAGCCCGTGACAGTGTCCTGGAACTCTGGG |
| | | GCTCTGACTTCCGGCGTGCACACCTTCCCCGCCG |
| | | TGCTGCAGAGCAGCGGCCTGTACAGCCTGAGCA |
| | | GCGTGGTGACAGTGCCCTCCAGCTCTCTGGGAAC |
| | | CCAGACCTATATCTGCAACGTGAACCACAAGCCC |
| | | AGCAACACCAAGGTGGACAAGAGAGTGGAGCCC |
| | | AAGAGCTGCGACAAGACCCACACCTGCCCCCCCT |
| | | GCCCAGCTCCAGAACTGCTGGGAGGGCCTTCCG |
| | | TGTTCCTGTTCCCCCCCAAGCCCAAGGACACCCT |
| | | GATGATCAGCAGGACCCCCGAGGTGACCTGCGT |
| | | GGTGGTGGACGTGTCCCACGAGGACCCAGAGGT |
| | | GAAGTTCAACTGGTACGTGGACGGCGTGGAGGT |
| | | GCACAACGCCAAGACCAAGCCCAGAGAGGAGCA |
| | | GTACAACAGCACCTACAGGGTGGTGTCCGTGCTG |
| | | ACCGTGCTGCACCAGGACTGGCTGAACGGCAAA |
| | | GAATACAAGTGCAAAGTCTCCAACAAGGCCCTGC |
| | | CAGCCCCAATCGAAAAGACAATCAGCAAGGCCAA |
| | | GGGCCAGCCACGGGAGCCCCAGGTGTACACCCT |
| | | GCCCCCCAGCCGGGAGGAGATGACCAAGAACCA |
| | | GGTGTCCCTGACCTGTCTGGTGAAGGGCTTCTAC |
| | | CCCAGCGATATCGCCGTGGAGTGGGAGAGCAAC |
| | | GGCCAGCCCGAGAACAACTACAAGACCACCCCC |
| | | CCAGTGCTGGACAGCGACGGCAGCTTCTTCCTGT |
| | | ACAGCAAGCTGACCGTGGACAAGTCCAGGTGGC |
| | | AGCAGGGCAACGTGTTCAGCTGCAGCGTGATGCA |
| | | CGAGGCCCTGCACAACCACTACACCCAGAAGTCC |
| | | CTGAGCCTGAGCCCCGGCAAG |
| SEQ ID NO: 414 | LCDR1 (KABAT) | SASSSVSYIH |
| SEQ ID NO: 415 | LCDR2 (KABAT) | STSNLAS |
| SEQ ID NO: 416 | LCDR3 (KABAT) | HQWSSYPWT |
| SEQ ID NO: 417 | LCDR1 (CHOTHIA) | SSSVSY |
| SEQ ID NO: 418 | LCDR2 (CHOTHIA) | STS |
| SEQ ID NO: 419 | LCDR3 (CHOTHIA) | WSSYPW |
| SEQ ID NO: 414 | LCDR1 | SASSSVSYIH |
| | (COMBINED) | |
| SEQ ID NO: 415 | LCDR2 | STSNLAS |
| | (COMBINED) | |
| SEQ ID NO: 416 | LCDR3 | HQWSSYPWT |
| | (COMBINED) | |
| SEQ ID NO: 420 | LCDR1 (IMGT) | SSVSY |
| SEQ ID NO: 418 | LCDR2 (IMGT) | STS |
| SEQ ID NO: 416 | LCDR3 (IMGT) | HQWSSYPWT |
| SEQ ID NO: 429 | VL | EIVLTQSPATLSASPGERITLSCSASSSVSYIHWYQQ |
| | | KPGQAPRLLIYSTSNLASGIPARFSGSGSGTFYTLTI |
| | | SSVEPEDAAVYYCHQWSSYPWTFGGGTKLEIK |
| SEQ ID NO: 430 | VL DNA | GAGATCGTGCTGACCCAGTCCCCTGCCACCCTGT |
| | | CTGCCAGCCCTGGCGAGCGGATCACCCTGTCCT |
| | | GCTCCGCCTCCTCCTCCGTGTCCTACATCCACTG |
| | | GTATCAGCAGAAGCCCGGCCAGGCCCCTCGGCT |
| | | GCTGATCTACTCCACCTCCAACCTGGCCTCCGGC |
| | | ATCCCTGCCAGATTCTCCGGCTCTGGCTCCGGCA |
| | | CCTTTTACACCCTGACCATCTCCAGCGTGGAACC |
| | | CGAGGACGCCGCCGTGTACTACTGCCACCAGTG |
| | | GTCCAGCTACCCCTGGACCTTCGGCGGAGGCAC |
| | | CAAGCTGGAAATCAAG |
| SEQ ID NO: 431 | LC | EIVLTQSPATLSASPGERITLSCSASSSVSYIHWYQQ |
| | | KPGQAPRLLIYSTSNLASGIPARFSGSGSGTFYTLTI |
| | | SSVEPEDAAVYYCHQWSSYPWTFGGGTKLEIKRTV |
| | | AAPSVFIFPPSDEQLKSGTASVVCLLNNFYPREAKV |
| | | QWKVDNALQSGNSQESVTEQDSKDSTYSLSSTLTL |
| | | SKADYEKHKVYACEVTHQGLSSPVTKSFNRGEC |
| SEQ ID NO: 432 | LC DNA | GAGATCGTGCTGACCCAGTCCCCTGCCACCCTGT |
| | | CTGCCAGCCCTGGCGAGCGGATCACCCTGTCCT |
| | | GCTCCGCCTCCTCCTCCGTGTCCTACATCCACTG |
| | | GTATCAGCAGAAGCCCGGCCAGGCCCCTCGGCT |
| | | GCTGATCTACTCCACCTCCAACCTGGCCTCCGGC |
| | | ATCCCTGCCAGATTCTCCGGCTCTGGCTCCGGCA |
| | | CCTTTTACACCCTGACCATCTCCAGCGTGGAACC |
| | | CGAGGACGCCGCCGTGTACTACTGCCACCAGTG |
| | | GTCCAGCTACCCCTGGACCTTCGGCGGAGGCAC |
| | | CAAGCTGGAAATCAAGCGTACGGTGGCCGCTCCC |
| | | AGCGTGTTCATCTTCCCCCCCAGCGACGAGCAGC |
| | | TGAAGAGTGGCACCGCCAGCGTGGTGTGCCTGC |
| | | TGAACAACTTCTACCCCCGGGAGGCCAAGGTGCA |
| | | GTGGAAGGTGGACAACGCCCTGCAGAGCGGCAA |
| | | CAGCCAGGAGAGCGTCACCGAGCAGGACAGCAA |
| | | GGACTCCACCTACAGCCTGAGCAGCACCCTGACC |
| | | CTGAGCAAGGCCGACTACGAGAAGCATAAGGTGT |
| | | ACGCCTGCGAGGTGACCCACCAGGGCCTGTCCA |
| | | GCCCCGTGACCAAGAGCTTCAACAGGGGCGAGT |
| | | GC |
| ANTI-HUMAN | | |
| ENTPD2 MAB3 | | |
| SEQ ID NO: 401 | HCDR1 (KABAT) | DYNMD |
| SEQ ID NO: 402 | HCDR2 (KABAT) | DINPKYDISTYNQQFKG |
| SEQ ID NO: 403 | HCDR3 (KABAT) | RGFFLYYGINYYYFDV |
| SEQ ID NO: 404 | HCDR1 (CHOTHIA) | GYTFTDY |
| SEQ ID NO: 405 | HCDR2 (CHOTHIA) | NPKYDI |
| SEQ ID NO: 403 | HCDR3 (CHOTHIA) | RGFFLYYGINYYYFDV |
| SEQ ID NO: 406 | HCDR1 | GYTFTDYNMD |
| | (COMBINED) | |
| SEQ ID NO: 402 | HCDR2 | DINPKYDISTYNQQFKG |
| | (COMBINED) | |
| SEQ ID NO: 403 | HCDR3 | RGFFLYYGINYYYFDV |
| | (COMBINED) | |
| SEQ ID NO: 407 | HCDR1 (IMGT) | GYTFTDYN |
| SEQ ID NO: 408 | HCDR2 (IMGT) | INPKYDIS |
| SEQ ID NO: 409 | HCDR3 (IMGT) | ARRGFFLYYGINYYYFDV |
| SEQ ID NO: 433 | VH | QVQLVQSGAEVVKPGASVKISCKASGYTFTDYNMD |
| | | WVKQAPGQRLEWIGDINPKYDISTYNQQFKGKATIT |
| | | VDTSASTAYMELSSLRSEDTAVYYCARRGFFLYYGI |
| | | NYYYFDVWGQGTLVTVSS |
| SEQ ID NO: 434 | VH DNA | CAGGTGCAGCTGGTGCAGTCTGGCGCCGAGGTG |
| | | GTGAAACCTGGCGCCTCCGTGAAGATCTCCTGCA |
| | | AGGCCTCCGGCTACACCTTCACCGACTACAACAT |
| | | GGACTGGGTGAAACAGGCCCCTGGCCAGCGGCT |
| | | GGAATGGATCGGCGACATCAACCCTAAGTACGAC |
| | | ATCTCCACCTACAACCAGCAGTTCAAGGGCAAGG |
| | | CCACCATCACCGTGGACACCTCCGCCTCCACCGC |
| | | CTACATGGAACTGTCCTCCCTGCGGAGCGAGGAC |
| | | ACCGCCGTGTACTACTGCGCCAGACGGGGCTTCT |
| | | TCCTGTACTACGGCATCAACTACTACTACTTCGAC |
| | | GTGTGGGGCCAGGGCACCCTGGTGACAGTGTCC |
| | | TCC |
| SEQ ID NO: 435 | HC | QVQLVQSGAEVVKPGASVKISCKASGYTFTDYNMD |
| | | WVKQAPGQRLEWIGDINPKYDISTYNQQFKGKATIT |
| | | VDTSASTAYMELSSLRSEDTAVYYCARRGFFLYYGI |
| | | NYYYFDVWGQGTLVTVSSASTKGPSVFPLAPSSKS |
| | | TSGGTAALGCLVKDYFPEPVTVSWNSGALTSGVHT |
| | | FPAVLQSSGLYSLSSVVTVPSSSLGTQTYICNVNHK |
| | | PSNTKVDKRVEPKSCDKTHTCPPCPAPELLGGPSV |
| | | FLFPPKPKDTLMISRTPEVTCVVVDVSHEDPEVKFN |
| | | WYVDGVEVHNAKTKPREEQYNSTYRVVSVLTVLHQ |
| | | DWLNGKEYKCKVSNKALPAPIEKTISKAKGQPREPQ |
| | | VYTLPPSREEMTKNQVSLTCLVKGFYPSDIAVEWES |
| | | NGQPENNYKTTPPVLDSDGSFFLYSKLTVDKSRWQ |
| | | QGNVFSCSVMHEALHNHYTQKSLSLSPGK |
| SEQ ID NO: 436 | HC DNA | CAGGTGCAGCTGGTGCAGTCTGGCGCCGAGGTG |
| | | GTGAAACCTGGCGCCTCCGTGAAGATCTCCTGCA |
| | | AGGCCTCCGGCTACACCTTCACCGACTACAACAT |
| | | GGACTGGGTGAAACAGGCCCCTGGCCAGCGGCT |
| | | GGAATGGATCGGCGACATCAACCCTAAGTACGAC |
| | | ATCTCCACCTACAACCAGCAGTTCAAGGGCAAGG |
| | | CCACCATCACCGTGGACACCTCCGCCTCCACCGC |
| | | CTACATGGAACTGTCCTCCCTGCGGAGCGAGGAC |
| | | ACCGCCGTGTACTACTGCGCCAGACGGGGCTTCT |
| | | TCCTGTACTACGGCATCAACTACTACTACTTCGAC |
| | | GTGTGGGGCCAGGGCACCCTGGTGACAGTGTCC |
| | | TCCGCTAGCACCAAGGGCCCAAGTGTGTTTCCCC |
| | | TGGCCCCCAGCAGCAAGTCTACTTCCGGCGGAAC |
| | | TGCTGCCCTGGGTTGCCTGGTGAAGGACTACTTC |
| | | CCCGAGCCCGTGACAGTGTCCTGGAACTCTGGG |
| | | GCTCTGACTTCCGGCGTGCACACCTTCCCCGCCG |
| | | TGCTGCAGAGCAGCGGCCTGTACAGCCTGAGCA |
| | | GCGTGGTGACAGTGCCCTCCAGCTCTCTGGGAAC |
| | | CCAGACCTATATCTGCAACGTGAACCACAAGCCC |
| | | AGCAACACCAAGGTGGACAAGAGAGTGGAGCCC |
| | | AAGAGCTGCGACAAGACCCACACCTGCCCCCCCT |
| | | GCCCAGCTCCAGAACTGCTGGGAGGGCCTTCCG |
| | | TGTTCCTGTTCCCCCCCAAGCCCAAGGACACCCT |
| | | GATGATCAGCAGGACCCCCGAGGTGACCTGCGT |
| | | GGTGGTGGACGTGTCCCACGAGGACCCAGAGGT |
| | | GAAGTTCAACTGGTACGTGGACGGCGTGGAGGT |
| | | GCACAACGCCAAGACCAAGCCCAGAGAGGAGCA |
| | | GTACAACAGCACCTACAGGGTGGTGTCCGTGCTG |
| | | ACCGTGCTGCACCAGGACTGGCTGAACGGCAAA |
| | | GAATACAAGTGCAAAGTCTCCAACAAGGCCCTGC |
| | | CAGCCCCAATCGAAAAGACAATCAGCAAGGCCAA |
| | | GGGCCAGCCACGGGAGCCCCAGGTGTACACCCT |
| | | GCCCCCCAGCCGGGAGGAGATGACCAAGAACCA |
| | | GGTGTCCCTGACCTGTCTGGTGAAGGGCTTCTAC |
| | | CCCAGCGATATCGCCGTGGAGTGGGAGAGCAAC |
| | | GGCCAGCCCGAGAACAACTACAAGACCACCCCC |
| | | CCAGTGCTGGACAGCGACGGCAGCTTCTTCCTGT |
| | | ACAGCAAGCTGACCGTGGACAAGTCCAGGTGGC |
| | | AGCAGGGCAACGTGTTCAGCTGCAGCGTGATGCA |
| | | CGAGGCCCTGCACAACCACTACACCCAGAAGTCC |
| | | CTGAGCCTGAGCCCCGGCAAG |
| SEQ ID NO: 414 | LCDR1 (KABAT) | SASSSVSYIH |
| SEQ ID NO: 415 | LCDR2 (KABAT) | STSNLAS |
| SEQ ID NO: 416 | LCDR3 (KABAT) | HQWSSYPWT |
| SEQ ID NO: 417 | LCDR1 (CHOTHIA) | SSSVSY |
| SEQ ID NO: 418 | LCDR2 (CHOTHIA) | STS |
| SEQ ID NO: 419 | LCDR3 (CHOTHIA) | WSSYPW |
| SEQ ID NO: 414 | LCDR1 | SASSSVSYIH |
| | (COMBINED) | |
| SEQ ID NO: 415 | LCDR2 | STSNLAS |
| | (COMBINED) | |
| SEQ ID NO: 416 | LCDR3 | HQWSSYPWT |
| | (COMBINED) | |
| SEQ ID NO: 420 | LCDR1 (IMGT) | SSVSY |
| SEQ ID NO: 418 | LCDR2 (IMGT) | STS |
| SEQ ID NO: 416 | LCDR3 (IMGT) | HQWSSYPWT |
| SEQ ID NO: 429 | VL | EIVLTQSPATLSASPGERITLSCSASSSVSYIHWYQQ |
| | | KPGQAPRLLIYSTSNLASGIPARFSGSGSGTFYTLTI |
| | | SSVEPEDAAVYYCHQWSSYPWTFGGGTKLEIK |
| SEQ ID NO: 430 | VL DNA | GAGATCGTGCTGACCCAGTCCCCTGCCACCCTGT |
| | | CTGCCAGCCCTGGCGAGCGGATCACCCTGTCCT |
| | | GCTCCGCCTCCTCCTCCGTGTCCTACATCCACTG |
| | | GTATCAGCAGAAGCCCGGCCAGGCCCCTCGGCT |
| | | GCTGATCTACTCCACCTCCAACCTGGCCTCCGGC |
| | | ATCCCTGCCAGATTCTCCGGCTCTGGCTCCGGCA |
| | | CCTTTTACACCCTGACCATCTCCAGCGTGGAACC |
| | | CGAGGACGCCGCCGTGTACTACTGCCACCAGTG |
| | | GTCCAGCTACCCCTGGACCTTCGGCGGAGGCAC |
| | | CAAGCTGGAAATCAAG |
| SEQ ID NO: 431 | LC | EIVLTQSPATLSASPGERITLSCSASSSVSYIHWYQQ |
| | | KPGQAPRLLIYSTSNLASGIPARFSGSGSGTFYTLTI |
| | | SSVEPEDAAVYYCHQWSSYPWTFGGGTKLEIKRTV |
| | | AAPSVFIFPPSDEQLKSGTASVVCLLNNFYPREAKV |
| | | QWKVDNALQSGNSQESVTEQDSKDSTYSLSSTLTL |
| | | SKADYEKHKVYACEVTHQGLSSPVTKSFNRGEC |
| SEQ ID NO: 432 | LC DNA | GAGATCGTGCTGACCCAGTCCCCTGCCACCCTGT |
| | | CTGCCAGCCCTGGCGAGCGGATCACCCTGTCCT |
| | | GCTCCGCCTCCTCCTCCGTGTCCTACATCCACTG |
| | | GTATCAGCAGAAGCCCGGCCAGGCCCCTCGGCT |
| | | GCTGATCTACTCCACCTCCAACCTGGCCTCCGGC |
| | | ATCCCTGCCAGATTCTCCGGCTCTGGCTCCGGCA |
| | | CCTTTTACACCCTGACCATCTCCAGCGTGGAACC |
| | | CGAGGACGCCGCCGTGTACTACTGCCACCAGTG |
| | | GTCCAGCTACCCCTGGACCTTCGGCGGAGGCAC |
| | | CAAGCTGGAAATCAAGCGTACGGTGGCCGCTCCC |
| | | AGCGTGTTCATCTTCCCCCCCAGCGACGAGCAGC |
| | | TGAAGAGTGGCACCGCCAGCGTGGTGTGCCTGC |
| | | TGAACAACTTCTACCCCCGGGAGGCCAAGGTGCA |
| | | GTGGAAGGTGGACAACGCCCTGCAGAGCGGCAA |
| | | CAGCCAGGAGAGCGTCACCGAGCAGGACAGCAA |
| | | GGACTCCACCTACAGCCTGAGCAGCACCCTGACC |
| | | CTGAGCAAGGCCGACTACGAGAAGCATAAGGTGT |
| | | ACGCCTGCGAGGTGACCCACCAGGGCCTGTCCA |
| | | GCCCCGTGACCAAGAGCTTCAACAGGGGCGAGT |
| | | GC |
| ANTI-HUMAN | | |
| ENTPD2 MAB4 | | |
| SEQ ID NO: 437 | HCDR1 (KABAT) | SGYYWN |
| SEQ ID NO: 438 | HCDR2 (KABAT) | YISYDADNNYNPSLKN |
| SEQ ID NO: 439 | HCDR3 (KABAT) | GYYRYGLGSYRGSYYYVMDY |
| SEQ ID NO: 440 | HCDR1 (CHOTHIA) | GYSITSGY |
| SEQ ID NO: 441 | HCDR2 (CHOTHIA) | SYDAD |
| SEQ ID NO: 439 | HCDR3 (CHOTHIA) | GYYRYGLGSYRGSYYYVMDY |
| SEQ ID NO: 442 | HCDR1 | GYSITSGYYWN |
| | (COMBINED) | |
| SEQ ID NO: 438 | HCDR2 | YISYDADNNYNPSLKN |
| | (COMBINED) | |
| SEQ ID NO: 439 | HCDR3 | GYYRYGLGSYRGSYYYVMDY |
| | (COMBINED) | |
| SEQ ID NO: 443 | HCDR1 (IMGT) | GYSITSGYY |
| SEQ ID NO: 444 | HCDR2 (IMGT) | ISYDADN |
| SEQ ID NO: 445 | HCDR3 (IMGT) | AGGYYRYGLGSYRGSYYYVMDY |
| SEQ ID NO: 446 | VH | QIQLQESGPGLVKPSQTLSLTCTVSGYSITSGYYWN |
| | | WIRQHPGKGLEWIGYISYDADNNYNPSLKNRVTISR |
| | | DTSKNQFSLKLSSVTAADTAVYYCAGGYYRYGLGS |
| | | YRGSYYYVMDYWGQGTTVTVSS |
| SEQ ID NO: 447 | VH DNA | CAGATCCAGCTGCAGGAATCTGGCCCTGGCCTG |
| | | GTGAAACCCTCCCAGACCCTGTCCCTGACCTGCA |
| | | CCGTGTCCGGCTACTCCATCACCTCCGGCTACTA |
| | | CTGGAACTGGATCCGGCAGCACCCCGGCAAGGG |
| | | CCTGGAATGGATCGGCTACATCTCCTACGACGCT |
| | | GACAACAACTACAACCCCAGCCTGAAGAACAGAG |
| | | TGACCATCTCCCGGGACACCTCCAAGAACCAGTT |
| | | CTCCCTGAAGCTGTCCTCCGTGACCGCCGCTGAC |
| | | ACCGCCGTGTACTACTGCGCTGGCGGCTACTACA |
| | | GATACGGCCTGGGCTCCTACCGGGGCTCCTACTA |
| | | CTACGTGATGGACTACTGGGGCCAGGGCACCAC |
| | | CGTGACCGTGTCCTCT |
| SEQ ID NO: 448 | HC | QIQLQESGPGLVKPSQTLSLTCTVSGYSITSGYYWN |
| | | WIRQHPGKGLEWIGYISYDADNNYNPSLKNRVTISR |
| | | DTSKNQFSLKLSSVTAADTAVYYCAGGYYRYGLGS |
| | | YRGSYYYVMDYWGQGTTVTVSSASTKGPSVFPLAP |
| | | SSKSTSGGTAALGCLVKDYFPEPVTVSWNSGALTS |
| | | GVHTFPAVLQSSGLYSLSSVVTVPSSSLGTQTYICN |
| | | VNHKPSNTKVDKRVEPKSCDKTHTCPPCPAPELLG |
| | | GPSVFLFPPKPKDTLMISRTPEVTCVVVDVSHEDPE |
| | | VKFNWYVDGVEVHNAKTKPREEQYNSTYRVVSVLT |
| | | VLHQDWLNGKEYKCKVSNKALPAPIEKTISKAKGQP |
| | | REPQVYTLPPSREEMTKNQVSLTCLVKGFYPSDIAV |
| | | EWESNGQPENNYKTTPPVLDSDGSFFLYSKLTVDK |
| | | SRWQQGNVFSCSVMHEALHNHYTQKSLSLSPGK |
| SEQ ID NO: 449 | HC DNA | CAGATCCAGCTGCAGGAATCTGGCCCTGGCCTG |
| | | GTGAAACCCTCCCAGACCCTGTCCCTGACCTGCA |
| | | CCGTGTCCGGCTACTCCATCACCTCCGGCTACTA |
| | | CTGGAACTGGATCCGGCAGCACCCCGGCAAGGG |
| | | CCTGGAATGGATCGGCTACATCTCCTACGACGCT |
| | | GACAACAACTACAACCCCAGCCTGAAGAACAGAG |
| | | TGACCATCTCCCGGGACACCTCCAAGAACCAGTT |
| | | CTCCCTGAAGCTGTCCTCCGTGACCGCCGCTGAC |
| | | ACCGCCGTGTACTACTGCGCTGGCGGCTACTACA |
| | | GATACGGCCTGGGCTCCTACCGGGGCTCCTACTA |
| | | CTACGTGATGGACTACTGGGGCCAGGGCACCAC |
| | | CGTGACCGTGTCCTCTGCTAGCACCAAGGGCCCA |
| | | AGTGTGTTTCCCCTGGCCCCCAGCAGCAAGTCTA |
| | | CTTCCGGCGGAACTGCTGCCCTGGGTTGCCTGGT |
| | | GAAGGACTACTTCCCCGAGCCCGTGACAGTGTCC |
| | | TGGAACTCTGGGGCTCTGACTTCCGGCGTGCACA |
| | | CCTTCCCCGCCGTGCTGCAGAGCAGCGGCCTGT |
| | | ACAGCCTGAGCAGCGTGGTGACAGTGCCCTCCA |
| | | GCTCTCTGGGAACCCAGACCTATATCTGCAACGT |
| | | GAACCACAAGCCCAGCAACACCAAGGTGGACAAG |
| | | AGAGTGGAGCCCAAGAGCTGCGACAAGACCCAC |
| | | ACCTGCCCCCCCTGCCCAGCTCCAGAACTGCTGG |
| | | GAGGGCCTTCCGTGTTCCTGTTCCCCCCCAAGCC |
| | | CAAGGACACCCTGATGATCAGCAGGACCCCCGA |
| | | GGTGACCTGCGTGGTGGTGGACGTGTCCCACGA |
| | | GGACCCAGAGGTGAAGTTCAACTGGTACGTGGAC |
| | | GGCGTGGAGGTGCACAACGCCAAGACCAAGCCC |
| | | AGAGAGGAGCAGTACAACAGCACCTACAGGGTG |
| | | GTGTCCGTGCTGACCGTGCTGCACCAGGACTGG |
| | | CTGAACGGCAAAGAATACAAGTGCAAAGTCTCCA |
| | | ACAAGGCCCTGCCAGCCCCAATCGAAAAGACAAT |
| | | CAGCAAGGCCAAGGGCCAGCCACGGGAGCCCCA |
| | | GGTGTACACCCTGCCCCCCAGCCGGGAGGAGAT |
| | | GACCAAGAACCAGGTGTCCCTGACCTGTCTGGTG |
| | | AAGGGCTTCTACCCCAGCGATATCGCCGTGGAGT |
| | | GGGAGAGCAACGGCCAGCCCGAGAACAACTACA |
| | | AGACCACCCCCCCAGTGCTGGACAGCGACGGCA |
| | | GCTTCTTCCTGTACAGCAAGCTGACCGTGGACAA |
| | | GTCCAGGTGGCAGCAGGGCAACGTGTTCAGCTG |
| | | CAGCGTGATGCACGAGGCCCTGCACAACCACTAC |
| | | ACCCAGAAGTCCCTGAGCCTGAGCCCCGGCAAG |
| SEQ ID NO: 450 | LCDR1 (KABAT) | KASQSVDYEGHSYMN |
| SEQ ID NO: 451 | LCDR2 (KABAT) | AASNLES |
| SEQ ID NO: 452 | LCDR3 (KABAT) | QQSNEDPPT |
| SEQ ID NO: 453 | LCDR1 (CHOTHIA) | SQSVDYEGHSY |
| SEQ ID NO: 454 | LCDR2 (CHOTHIA) | AAS |
| SEQ ID NO: 455 | LCDR3 (CHOTHIA) | SNEDPP |
| SEQ ID NO: 450 | LCDR1 | KASQSVDYEGHSYMN |
| | (COMBINED) | |
| SEQ ID NO: 451 | LCDR2 | AASNLES |
| | (COMBINED) | |
| SEQ ID NO: 452 | LCDR3 | QQSNEDPPT |
| | (COMBINED) | |
| SEQ ID NO: 456 | LCDR1 (IMGT) | QSVDYEGHSY |
| SEQ ID NO: 454 | LCDR2 (IMGT) | AAS |
| SEQ ID NO: 452 | LCDR3 (IMGT) | QQSNEDPPT |
| SEQ ID NO: 457 | VL | ETVLTQSPATLSVSPGERATISCKASQSVDYEGHSY |
| | | MNWYQQKPGQAPRLLIYAASNLESGIPARFSGSGS |
| | | GTDFTLTISSVEPEDAATYYCQQSNEDPPTFGGGTK |
| | | LEIK |
| SEQ ID NO: 458 | VL DNA | GAGACAGTGCTGACCCAGTCCCCTGCCACCCTGT |
| | | CCGTGTCTCCCGGCGAGAGAGCCACCATCAGCT |
| | | GCAAGGCCTCCCAGTCCGTGGACTACGAAGGCC |
| | | ATTCCTACATGAACTGGTATCAGCAGAAGCCCGG |
| | | CCAGGCCCCTCGGCTGCTGATCTACGCCGCCTC |
| | | CAACCTGGAATCCGGCATCCCTGCCCGGTTCTCC |
| | | GGCTCTGGCTCTGGCACCGACTTCACCCTGACCA |
| | | TCTCCAGCGTGGAACCCGAGGACGCCGCCACCT |
| | | ACTACTGCCAGCAGTCCAACGAGGACCCCCCCAC |
| | | CTTCGGCGGAGGCACCAAGCTGGAAATCAAG |
| SEQ ID NO: 459 | LC | ETVLTQSPATLSVSPGERATISCKASQSVDYEGHSY |
| | | MNWYQQKPGQAPRLLIYAASNLESGIPARFSGSGS |
| | | GTDFTLTISSVEPEDAATYYCQQSNEDPPTFGGGTK |
| | | LEIKRTVAAPSVFIFPPSDEQLKSGTASVVCLLNNFY |
| | | PREAKVQWKVDNALQSGNSQESVTEQDSKDSTYS |
| | | LSSTLTLSKADYEKHKVYACEVTHQGLSSPVTKSFN |
| | | RGEC |
| SEQ ID NO: 460 | LC DNA | GAGACAGTGCTGACCCAGTCCCCTGCCACCCTGT |
| | | CCGTGTCTCCCGGCGAGAGAGCCACCATCAGCT |
| | | GCAAGGCCTCCCAGTCCGTGGACTACGAAGGCC |
| | | ATTCCTACATGAACTGGTATCAGCAGAAGCCCGG |
| | | CCAGGCCCCTCGGCTGCTGATCTACGCCGCCTC |
| | | CAACCTGGAATCCGGCATCCCTGCCCGGTTCTCC |
| | | GGCTCTGGCTCTGGCACCGACTTCACCCTGACCA |
| | | TCTCCAGCGTGGAACCCGAGGACGCCGCCACCT |
| | | ACTACTGCCAGCAGTCCAACGAGGACCCCCCCAC |
| | | CTTCGGCGGAGGCACCAAGCTGGAAATCAAGCGT |
| | | ACGGTGGCCGCTCCCAGCGTGTTCATCTTCCCCC |
| | | CCAGCGACGAGCAGCTGAAGAGTGGCACCGCCA |
| | | GCGTGGTGTGCCTGCTGAACAACTTCTACCCCCG |
| | | GGAGGCCAAGGTGCAGTGGAAGGTGGACAACGC |
| | | CCTGCAGAGCGGCAACAGCCAGGAGAGCGTCAC |
| | | CGAGCAGGACAGCAAGGACTCCACCTACAGCCT |
| | | GAGCAGCACCCTGACCCTGAGCAAGGCCGACTA |
| | | CGAGAAGCATAAGGTGTACGCCTGCGAGGTGAC |
| | | CCACCAGGGCCTGTCCAGCCCCGTGACCAAGAG |
| | | CTTCAACAGGGGCGAGTGC |
| ANTI-HUMAN | | |
| ENTPD2 MAB5 | | |
| SEQ ID NO: 437 | HCDR1 (KABAT) | SGYYWN |
| SEQ ID NO: 438 | HCDR2 (KABAT) | YISYDADNNYNPSLKN |
| SEQ ID NO: 439 | HCDR3 (KABAT) | GYYRYGLGSYRGSYYYVMDY |
| SEQ ID NO: 440 | HCDR1 (CHOTHIA) | GYSITSGY |
| SEQ ID NO: 441 | HCDR2 (CHOTHIA) | SYDAD |
| SEQ ID NO: 439 | HCDR3 (CHOTHIA) | GYYRYGLGSYRGSYYYVMDY |
| SEQ ID NO: 442 | HCDR1 | GYSITSGYYWN |
| | (COMBINED) | |
| SEQ ID NO: 438 | HCDR2 | YISYDADNNYNPSLKN |
| | (COMBINED) | |
| SEQ ID NO: 439 | HCDR3 | GYYRYGLGSYRGSYYYVMDY |
| | (COMBINED) | |
| SEQ ID NO: 443 | HCDR1 (IMGT) | GYSITSGYY |
| SEQ ID NO: 444 | HCDR2 (IMGT) | ISYDADN |
| SEQ ID NO: 445 | HCDR3 (IMGT) | AGGYYRYGLGSYRGSYYYVMDY |
| SEQ ID NO: 446 | VH | QIQLQESGPGLVKPSQTLSLTCTVSGYSITSGYYWN |
| | | WIRQHPGKGLEWIGYISYDADNNYNPSLKNRVTISR |
| | | DTSKNQFSLKLSSVTAADTAVYYCAGGYYRYGLGS |
| | | YRGSYYYVMDYWGQGTTVTVSS |
| SEQ ID NO: 447 | VH DNA | CAGATCCAGCTGCAGGAATCTGGCCCTGGCCTG |
| | | GTGAAACCCTCCCAGACCCTGTCCCTGACCTGCA |
| | | CCGTGTCCGGCTACTCCATCACCTCCGGCTACTA |
| | | CTGGAACTGGATCCGGCAGCACCCCGGCAAGGG |
| | | CCTGGAATGGATCGGCTACATCTCCTACGACGCT |
| | | GACAACAACTACAACCCCAGCCTGAAGAACAGAG |
| | | TGACCATCTCCCGGGACACCTCCAAGAACCAGTT |
| | | CTCCCTGAAGCTGTCCTCCGTGACCGCCGCTGAC |
| | | ACCGCCGTGTACTACTGCGCTGGCGGCTACTACA |
| | | GATACGGCCTGGGCTCCTACCGGGGCTCCTACTA |
| | | CTACGTGATGGACTACTGGGGCCAGGGCACCAC |
| | | CGTGACCGTGTCCTCT |
| SEQ ID NO: 448 | HC | QIQLQESGPGLVKPSQTLSLTCTVSGYSITSGYYWN |
| | | WIRQHPGKGLEWIGYISYDADNNYNPSLKNRVTISR |
| | | DTSKNQFSLKLSSVTAADTAVYYCAGGYYRYGLGS |
| | | YRGSYYYVMDYWGQGTTVTVSSASTKGPSVFPLAP |
| | | SSKSTSGGTAALGCLVKDYFPEPVTVSWNSGALTS |
| | | GVHTFPAVLQSSGLYSLSSVVTVPSSSLGTQTYICN |
| | | VNHKPSNTKVDKRVEPKSCDKTHTCPPCPAPELLG |
| | | GPSVFLFPPKPKDTLMISRTPEVTCVVVDVSHEDPE |
| | | VKFNWYVDGVEVHNAKTKPREEQYNSTYRVVSVLT |
| | | VLHQDWLNGKEYKCKVSNKALPAPIEKTISKAKGQP |
| | | REPQVYTLPPSREEMTKNQVSLTCLVKGFYPSDIAV |
| | | EWESNGQPENNYKTTPPVLDSDGSFFLYSKLTVDK |
| | | SRWQQGNVFSCSVMHEALHNHYTQKSLSLSPGK |
| SEQ ID NO: 449 | HC DNA | CAGATCCAGCTGCAGGAATCTGGCCCTGGCCTG |
| | | GTGAAACCCTCCCAGACCCTGTCCCTGACCTGCA |
| | | CCGTGTCCGGCTACTCCATCACCTCCGGCTACTA |
| | | CTGGAACTGGATCCGGCAGCACCCCGGCAAGGG |
| | | CCTGGAATGGATCGGCTACATCTCCTACGACGCT |
| | | GACAACAACTACAACCCCAGCCTGAAGAACAGAG |
| | | TGACCATCTCCCGGGACACCTCCAAGAACCAGTT |
| | | CTCCCTGAAGCTGTCCTCCGTGACCGCCGCTGAC |
| | | ACCGCCGTGTACTACTGCGCTGGCGGCTACTACA |
| | | GATACGGCCTGGGCTCCTACCGGGGCTCCTACTA |
| | | CTACGTGATGGACTACTGGGGCCAGGGCACCAC |
| | | CGTGACCGTGTCCTCTGCTAGCACCAAGGGCCCA |
| | | AGTGTGTTTCCCCTGGCCCCCAGCAGCAAGTCTA |
| | | CTTCCGGCGGAACTGCTGCCCTGGGTTGCCTGGT |
| | | GAAGGACTACTTCCCCGAGCCCGTGACAGTGTCC |
| | | TGGAACTCTGGGGCTCTGACTTCCGGCGTGCACA |
| | | CCTTCCCCGCCGTGCTGCAGAGCAGCGGCCTGT |
| | | ACAGCCTGAGCAGCGTGGTGACAGTGCCCTCCA |
| | | GCTCTCTGGGAACCCAGACCTATATCTGCAACGT |
| | | GAACCACAAGCCCAGCAACACCAAGGTGGACAAG |
| | | AGAGTGGAGCCCAAGAGCTGCGACAAGACCCAC |
| | | ACCTGCCCCCCCTGCCCAGCTCCAGAACTGCTGG |
| | | GAGGGCCTTCCGTGTTCCTGTTCCCCCCCAAGCC |
| | | CAAGGACACCCTGATGATCAGCAGGACCCCCGA |
| | | GGTGACCTGCGTGGTGGTGGACGTGTCCCACGA |
| | | GGACCCAGAGGTGAAGTTCAACTGGTACGTGGAC |
| | | GGCGTGGAGGTGCACAACGCCAAGACCAAGCCC |
| | | AGAGAGGAGCAGTACAACAGCACCTACAGGGTG |
| | | GTGTCCGTGCTGACCGTGCTGCACCAGGACTGG |
| | | CTGAACGGCAAAGAATACAAGTGCAAAGTCTCCA |
| | | ACAAGGCCCTGCCAGCCCCAATCGAAAAGACAAT |
| | | CAGCAAGGCCAAGGGCCAGCCACGGGAGCCCCA |
| | | GGTGTACACCCTGCCCCCCAGCCGGGAGGAGAT |
| | | GACCAAGAACCAGGTGTCCCTGACCTGTCTGGTG |
| | | AAGGGCTTCTACCCCAGCGATATCGCCGTGGAGT |
| | | GGGAGAGCAACGGCCAGCCCGAGAACAACTACA |
| | | AGACCACCCCCCCAGTGCTGGACAGCGACGGCA |
| | | GCTTCTTCCTGTACAGCAAGCTGACCGTGGACAA |
| | | GTCCAGGTGGCAGCAGGGCAACGTGTTCAGCTG |
| | | CAGCGTGATGCACGAGGCCCTGCACAACCACTAC |
| | | ACCCAGAAGTCCCTGAGCCTGAGCCCCGGCAAG |
| SEQ ID NO: 461 | LCDR1 (KABAT) | KASQSVDYDGNSYMN |
| SEQ ID NO: 451 | LCDR2 (KABAT) | AASNLES |
| SEQ ID NO: 452 | LCDR3 (KABAT) | QQSNEDPPT |
| SEQ ID NO: 462 | LCDR1 (CHOTHIA) | SQSVDYDGNSY |
| SEQ ID NO: 454 | LCDR2 (CHOTHIA) | AAS |
| SEQ ID NO: 455 | LCDR3 (CHOTHIA) | SNEDPP |
| SEQ ID NO: 461 | LCDR1 | KASQSVDYDGNSYMN |
| | (COMBINED) | |
| SEQ ID NO: 451 | LCDR2 | AASNLES |
| | (COMBINED) | |
| SEQ ID NO: 452 | LCDR3 | QQSNEDPPT |
| | (COMBINED) | |
| SEQ ID NO: 463 | LCDR1 (IMGT) | QSVDYDGNSY |
| SEQ ID NO: 454 | LCDR2 (IMGT) | AAS |
| SEQ ID NO: 452 | LCDR3 (IMGT) | QQSNEDPPT |
| SEQ ID NO: 464 | VL | ETVLTQSPATLSVSPGERATISCKASQSVDYDGNSY |
| | | MNWYQQKPGQAPRLLIYAASNLESGIPARFSGSGS |
| | | GTDFTLTISSVEPEDAATYYCQQSNEDPPTFGGGTK |
| | | LEIK |
| SEQ ID NO: 465 | VL DNA | GAGACAGTGCTGACCCAGTCCCCTGCCACCCTGT |
| | | CCGTGTCTCCCGGCGAGAGAGCCACCATCAGCT |
| | | GCAAGGCCTCCCAGTCCGTGGACTACGACGGCA |
| | | ACTCCTACATGAACTGGTATCAGCAGAAGCCCGG |
| | | CCAGGCCCCTCGGCTGCTGATCTACGCCGCCTC |
| | | CAACCTGGAATCCGGCATCCCTGCCCGGTTCTCC |
| | | GGCTCTGGCTCTGGCACCGACTTCACCCTGACCA |
| | | TCTCCAGCGTGGAACCCGAGGACGCCGCCACCT |
| | | ACTACTGCCAGCAGTCCAACGAGGACCCCCCCAC |
| | | CTTCGGCGGAGGCACCAAGCTGGAAATCAAG |
| SEQ ID NO: 466 | LC | ETVLTQSPATLSVSPGERATISCKASQSVDYDGNSY |
| | | MNWYQQKPGQAPRLLIYAASNLESGIPARFSGSGS |
| | | GTDFTLTISSVEPEDAATYYCQQSNEDPPTFGGGTK |
| | | LEIKRTVAAPSVFIFPPSDEQLKSGTASVVCLLNNFY |
| | | PREAKVQWKVDNALQSGNSQESVTEQDSKDSTYS |
| | | LSSTLTLSKADYEKHKVYACEVTHQGLSSPVTKSFN |
| | | RGEC |
| SEQ ID NO: 467 | LC DNA | GAGACAGTGCTGACCCAGTCCCCTGCCACCCTGT |
| | | CCGTGTCTCCCGGCGAGAGAGCCACCATCAGCT |
| | | GCAAGGCCTCCCAGTCCGTGGACTACGACGGCA |
| | | ACTCCTACATGAACTGGTATCAGCAGAAGCCCGG |
| | | CCAGGCCCCTCGGCTGCTGATCTACGCCGCCTC |
| | | CAACCTGGAATCCGGCATCCCTGCCCGGTTCTCC |
| | | GGCTCTGGCTCTGGCACCGACTTCACCCTGACCA |
| | | TCTCCAGCGTGGAACCCGAGGACGCCGCCACCT |
| | | ACTACTGCCAGCAGTCCAACGAGGACCCCCCCAC |
| | | CTTCGGCGGAGGCACCAAGCTGGAAATCAAGCGT |
| | | ACGGTGGCCGCTCCCAGCGTGTTCATCTTCCCCC |
| | | CCAGCGACGAGCAGCTGAAGAGTGGCACCGCCA |
| | | GCGTGGTGTGCCTGCTGAACAACTTCTACCCCCG |
| | | GGAGGCCAAGGTGCAGTGGAAGGTGGACAACGC |
| | | CCTGCAGAGCGGCAACAGCCAGGAGAGCGTCAC |
| | | CGAGCAGGACAGCAAGGACTCCACCTACAGCCT |
| | | GAGCAGCACCCTGACCCTGAGCAAGGCCGACTA |
| | | CGAGAAGCATAAGGTGTACGCCTGCGAGGTGAC |
| | | CCACCAGGGCCTGTCCAGCCCCGTGACCAAGAG |
| | | CTTCAACAGGGGCGAGTGC |

In some embodiments, the anti-human ENTPD2 antibody or antibody fragment (e.g., an antigen binding fragment) comprises a VH domain having an amino acid sequence of any VH domain described in Table 9. Other suitable anti-human ENTPD2 antibodies or antibody fragments (e.g., antigen binding fragments) can include amino acids that have been mutated, yet have at least 80, 85, 90, 95, 96, 97, 98, or 99 percent identity in the VH domain with the VH regions depicted in the sequences described in Table 9. The present disclosure in certain embodiments also provides antibodies or antibody fragments (e.g., antigen binding fragments) that specifically bind to human ENTPD2, wherein the antibodies or antibody fragments (e.g., antigen binding fragments) comprise a VH CDR having an amino acid sequence of any one of the VH CDRs listed in Table 9. In particular embodiments, the invention provides antibodies or antibody fragments (e.g., antigen binding fragments) that specifically bind to human ENTPD2, comprising (or alternatively, consist of) one, two, three, four, five or more VH CDRs having an amino acid sequence of any one of the VH CDRs listed in Table 9.

In some embodiments, the anti-human ENTPD2 antibody or antibody fragment (e.g., antigen binding fragment) comprises a VL domain having an amino acid sequence of any VL domain described in Table 9. Other suitable anti-human ENTPD2 antibodies or antibody fragments (e.g., antigen binding fragments) can include amino acids that have been mutated, yet have at least 80, 85, 90, 95, 96, 97, 98, or 99 percent identity in the VL domain with the VL regions depicted in the sequences described in Table 9. The present disclosure also provides antibodies or antibody fragments (e.g., antigen binding fragments) that specifically bind to human ENTPD2, the antibodies or antibody fragments (e.g., antigen binding fragments) comprise a VL CDR having an amino acid sequence of any one of the VL CDRs listed in Table 9. In particular, the invention provides antibodies or antibody fragments (e.g., antigen binding fragments) that specifically bind to human ENTPD2, which comprise (or alternatively, consist of) one, two, three or more VL CDRs having an amino acid sequence of any one of the VL CDRs listed in Table 9.

Other anti-human ENTPD2 antibodies or antibody fragments (e.g., antigen binding fragment) disclosed herein include amino acids that have been mutated, yet have at least 80, 85, 90, 95, 96, 97, 98, or 99 percent identity in the CDR regions with the CDR regions depicted in the sequences described in Table 9. In some embodiments, it includes mutant amino acid sequences wherein no more than 1, 2, 3, 4 or 5 amino acids have been mutated in the CDR regions when compared with the CDR regions depicted in the sequence described in Table 9.

Also provided herein are nucleic acid sequences that encode VH, VL, full length heavy chain, and full length light chain of antibodies and antigen binding fragments thereof that specifically bind to human ENTPD2, e.g., the nucleic acid sequences in Table 9. Such nucleic acid sequences can be optimized for expression in mammalian cells.

Other anti-human ENTPD2 antibodies disclosed herein include those where the amino acids or nucleic acids encoding the amino acids have been mutated, yet have at least 80, 85, 90 95, 96, 97, 98, or 99 percent identity to the sequences described in Table 9. In some embodiments, antibodies or antigen binding fragments thereof include mutant amino acid sequences wherein no more than 1, 2, 3, 4 or 5 amino acids have been mutated in the variable regions when compared with the variable regions depicted in the sequence described in Table 9, while retaining substantially the same therapeutic activity.

Since each provided antibody binds to human ENTPD2, the VH, VL, full length light chain, and full length heavy chain sequences (amino acid sequences and the nucleotide sequences encoding the amino acid sequences) can be “mixed and matched” to create other ENTPD2-binding antibodies disclosed herein. Such “mixed and matched” ENTPD2-binding antibodies can be tested using binding assays known in the art (e.g., ELISAs, assays described in the Exemplification). When chains are mixed and matched, a VH sequence from a particular VH/VL pairing should be replaced with a structurally similar VH sequence. A full length heavy chain sequence from a particular full length heavy chain/full length light chain pairing should be replaced with a structurally similar full length heavy chain sequence. A VL sequence from a particular VH/VL pairing should be replaced with a structurally similar VL sequence. A full length light chain sequence from a particular full length heavy chain/full length light chain pairing should be replaced with a structurally similar full length light chain sequence.

Accordingly, in one embodiment, the invention provides an isolated monoclonal antibody or antigen binding fragment thereof having: a heavy chain variable region (VH) comprising an amino acid sequence selected from any one of SEQ ID NOs: 410, 425, 433, 446; and a light chain variable region (VL) comprising an amino acid sequence selected from any one of SEQ ID NOs: 421, 429, 457, 464; wherein the antibody specifically binds to human ENTPD2.

In another embodiment, the invention provides (i) an isolated monoclonal antibody having: a full length heavy chain (HC) comprising an amino acid sequence selected from any one of SEQ ID NOs: 412, 427, 435, 448; and a full length light chain (LC) comprising an amino acid sequence selected from any one of SEQ ID NOs: 423, 431, 459, 466; or (ii) a functional protein comprising an antigen binding portion thereof.

In another embodiment, the present disclosure provides human ENTPD2-binding antibodies or antibody fragments thereof that comprise the heavy chain CDR1, CDR2 and CDR3 and light chain CDR1, CDR2 and CDR3 as described in Table 9, or combinations thereof. The amino acid sequences of the VH CDR1s of the antibodies are shown in SEQ ID NOs: 401, 404, 406, 407, 437, 440, 442, 443. The amino acid sequences of the VH CDR2s of the antibodies and are shown in SEQ ID NOs: 402, 405, 408, 438, 441, 444. The amino acid sequences of the VH CDR3s of the antibodies are shown in SEQ ID NO: 403, 409, 439, 445. The amino acid sequences of the VL CDR1s of the antibodies are shown in SEQ ID NOs: 414, 417, 420, 450, 453, 456, 461, 462, 463. The amino acid sequences of the VL CDR2s of the antibodies are shown in SEQ ID NOs: 415, 418, 451, 454. The amino acid sequences of the VL CDR3s of the antibodies are shown in SEQ ID NOs: 416, 419, 452, 455.

Given that each of the antibodies binds human ENTPD2 and that antigen-binding specificity is provided primarily by the CDR1, CDR2 and CDR3 regions, the VH CDR1, CDR2 and CDR3 sequences and VL CDR1, CDR2 and CDR3 sequences can be “mixed and matched” (i.e., CDRs from different antibodies can be mixed and matched), although each antibody must contain a VH CDR1, CDR2 and CDR3 and a VL CDR1, CDR2 and CDR3 to create other human ENTPD2-binding antibodies disclosed herein. Such “mixed and matched” ENTPD2-binding antibodies can be tested using the binding assays known in the art and those described in the Examples (e.g., ELISAs). When VH CDR sequences are mixed and matched, the CDR1, CDR2 and/or CDR3 sequence from a particular VH sequence should be replaced with a structurally similar CDR sequence(s). Likewise, when VL CDR sequences are mixed and matched, the CDR1, CDR2 and/or CDR3 sequence from a particular VL sequence should be replaced with a structurally similar CDR sequence(s). It will be readily apparent to the ordinarily skilled artisan that novel VH and VL sequences can be created by substituting one or more VH and/or VL CDR region sequences with structurally similar sequences from CDR sequences shown herein for monoclonal antibodies of the present disclosure.

Accordingly, the present disclosure provides an isolated monoclonal antibody or antigen binding region thereof comprising a heavy chain CDR1 comprising an amino acid sequence selected from the group consisting of SEQ ID NOs: 401, 404, 406, 407, 437, 440, 442, 443; a heavy chain CDR2 comprising an amino acid sequence selected from the group consisting of SEQ ID NOs: 402, 405, 408, 438, 441, 444; a heavy chain CDR3 comprising an amino acid sequence selected from the group consisting of SEQ ID NO: 403, 409, 439, 445; a light chain CDR1 comprising an amino acid sequence selected from the group consisting of SEQ ID NOs: 414, 417, 420, 450, 453, 456, 461, 462, 463; a light chain CDR2 comprising an amino acid sequence selected from the group consisting of SEQ ID NOs: 415, 418, 451, 454; and a light chain CDR3 comprising an amino acid sequence selected from the group consisting of SEQ ID NOs: 416, 419, 452, 455; wherein the antibody specifically binds human ENTPD2.

In certain embodiments, an antibody that specifically binds to human ENTPD2 is an antibody or antibody fragment (e.g., antigen binding fragment) that is described in Table 9.

In some embodiments, the antibody or antigen binding region thereof that specifically binds to human ENTPD2 comprises a heavy chain complementary determining region 1 (HCDR1) comprising the amino acid sequence of SEQ ID NO: 401, 404, 406, or 407; a heavy chain complementary determining region 2 (HCDR2) comprising the amino acid sequence of SEQ ID NO: 402, 405, or 408; a heavy chain complementary determining region 3 (HCDR3) comprising the amino acid sequence of SEQ ID NO: 403 or 409; a light chain complementary determining region 1 (LCDR1) comprising the amino acid sequence of SEQ ID NO: 414, 417, or 420; a light chain complementary determining region 2 (LCDR2) comprising the amino acid sequence of SEQ ID NO:415 or 418; and a light chain complementary determining region 3 (LCDR3) comprising the amino acid sequence of SEQ ID NO: 416 or 419.

In some embodiments, the antibody or antigen binding region thereof that specifically binds to human ENTPD2 comprises an HCDR1 comprising the amino acid sequence of SEQ ID NO: 401; an HCDR2 comprising the amino acid sequence of SEQ ID NO: 402; an HCDR3 comprising the amino acid sequence of SEQ ID NO: 403; an LCDR1 comprising the amino acid sequence of SEQ ID NO: 414; an LCDR2 comprising the amino acid sequence of SEQ ID NO: 415; and an LCDR3 comprising the amino acid sequence of SEQ ID NO: 416.

In some embodiments, the antibody or antigen binding region thereof that specifically binds to human ENTPD2 comprises an HCDR1 comprising the amino acid sequence of SEQ ID NO: 404; an HCDR2 comprising the amino acid sequence of SEQ ID NO: 405; an HCDR3 comprising the amino acid sequence of SEQ ID NO: 403; an LCDR1 comprising the amino acid sequence of SEQ ID NO: 417; an LCDR2 comprising the amino acid sequence of SEQ ID NO: 418; and an LCDR3 comprising the amino acid sequence of SEQ ID NO: 419.

In some embodiments, the antibody or antigen binding region thereof that specifically binds to human ENTPD2 comprises an HCDR1 comprising the amino acid sequence of SEQ ID NO: 406; an HCDR2 comprising the amino acid sequence of SEQ ID NO: 402; an HCDR3 comprising the amino acid sequence of SEQ ID NO: 403; an LCDR1 comprising the amino acid sequence of SEQ ID NO: 414; an LCDR2 comprising the amino acid sequence of SEQ ID NO: 415; and an LCDR3 comprising the amino acid sequence of SEQ ID NO: 416.

In some embodiments, the antibody or antigen binding region thereof that specifically binds to human ENTPD2 comprises an HCDR1 comprising the amino acid sequence of SEQ ID NO: 407; an HCDR2 comprising the amino acid sequence of SEQ ID NO: 408; an HCDR3 comprising the amino acid sequence of SEQ ID NO: 409; an LCDR1 comprising the amino acid sequence of SEQ ID NO: 420; an LCDR2 comprising the amino acid sequence of SEQ ID NO: 418; and an LCDR3 comprising the amino acid sequence of SEQ ID NO: 416.

In some embodiments, the antibody or antigen binding region thereof that specifically binds to human ENTPD2 comprises a HCDR1 comprising the amino acid sequence of SEQ ID NO: 437, 440, 442 or 443; a HCDR2 comprising the amino acid sequence of SEQ ID NO: 438, 441, or 444; a HCDR3 comprising the amino acid sequence of SEQ ID NO: 439 or 445; a LCDR1 comprising the amino acid sequence of SEQ ID NO: 450, 453 or 456; a LCDR2 comprising the amino acid sequence of SEQ ID NO: 451 or 454; and a LCDR3 comprising the amino acid sequence of SEQ ID NO: 452 or 455.

In some embodiments, the antibody or antigen binding region thereof that specifically binds to human ENTPD2 comprises an HCDR1 comprising the amino acid sequence of SEQ ID NO: 437; an HCDR2 comprising the amino acid sequence of SEQ ID NO: 438; an HCDR3 comprising the amino acid sequence of SEQ ID NO: 439; an LCDR1 comprising the amino acid sequence of SEQ ID NO: 450; an LCDR2 comprising the amino acid sequence of SEQ ID NO: 451; and an LCDR3 comprising the amino acid sequence of SEQ ID NO: 452.

In some embodiments, the antibody or antigen binding region thereof that specifically binds to human ENTPD2 comprises an HCDR1 comprising the amino acid sequence of SEQ ID NO: 440; an HCDR2 comprising the amino acid sequence of SEQ ID NO: 441; an HCDR3 comprising the amino acid sequence of SEQ ID NO: 439; an LCDR1 comprising the amino acid sequence of SEQ ID NO: 453; an LCDR2 comprising the amino acid sequence of SEQ ID NO: 454; and an LCDR3 comprising the amino acid sequence of SEQ ID NO: 455.

In some embodiments, the antibody or antigen binding region thereof that specifically binds to human ENTPD2 comprises an HCDR1 comprising the amino acid sequence of SEQ ID NO: 442; an HCDR2 comprising the amino acid sequence of SEQ ID NO: 438; an HCDR3 comprising the amino acid sequence of SEQ ID NO: 439; an LCDR1 comprising the amino acid sequence of SEQ ID NO: 450; an LCDR2 comprising the amino acid sequence of SEQ ID NO: 451; and an LCDR3 comprising the amino acid sequence of SEQ ID NO: 452.

In some embodiments, the antibody or antigen binding region thereof that specifically binds to human ENTPD2 comprises an HCDR1 comprising the amino acid sequence of SEQ ID NO: 443; an HCDR2 comprising the amino acid sequence of SEQ ID NO: 444; an HCDR3 comprising the amino acid sequence of SEQ ID NO: 445; an LCDR1 comprising the amino acid sequence of SEQ ID NO: 456; an LCDR2 comprising the amino acid sequence of SEQ ID NO: 454; and an LCDR3 comprising the amino acid sequence of SEQ ID NO: 452.

In some embodiments, the antibody or antigen binding region thereof that specifically binds to human ENTPD2 comprises a HCDR1 comprising the amino acid sequence of SEQ ID NO: 437, 440, 442 or 443; a HCDR2 comprising the amino acid sequence of SEQ ID NO: 438, 441, or 444; a HCDR3 comprising the amino acid sequence of SEQ ID NO: 439 or 445; a LCDR1 comprising the amino acid sequence of SEQ ID NO: 461, 462, or 463; a LCDR2 comprising the amino acid sequence of SEQ ID NO: 451 or 454; and a LCDR3 comprising the amino acid sequence of SEQ ID NO: 452 or 455.

In some embodiments, the antibody or antigen binding region thereof that specifically binds to human ENTPD2 comprises an HCDR1 comprising the amino acid sequence of SEQ ID NO: 437; an HCDR2 comprising the amino acid sequence of SEQ ID NO: 438; an HCDR3 comprising the amino acid sequence of SEQ ID NO: 439; an LCDR1 comprising the amino acid sequence of SEQ ID NO: 461; an LCDR2 comprising the amino acid sequence of SEQ ID NO: 451; and an LCDR3 comprising the amino acid sequence of SEQ ID NO: 452.

In some embodiments, the antibody or antigen binding region thereof that specifically binds to human ENTPD2 comprises an HCDR1 comprising the amino acid sequence of SEQ ID NO: 440; an HCDR2 comprising the amino acid sequence of SEQ ID NO: 441; an HCDR3 comprising the amino acid sequence of SEQ ID NO: 439; an LCDR1 comprising the amino acid sequence of SEQ ID NO: 462; an LCDR2 comprising the amino acid sequence of SEQ ID NO: 454; and an LCDR3 comprising the amino acid sequence of SEQ ID NO: 455.

In some embodiments, the antibody or antigen binding region thereof that specifically binds to human ENTPD2 comprises an HCDR1 comprising the amino acid sequence of SEQ ID NO: 442; an HCDR2 comprising the amino acid sequence of SEQ ID NO: 438; an HCDR3 comprising the amino acid sequence of SEQ ID NO: 439; an LCDR1 comprising the amino acid sequence of SEQ ID NO: 461; an LCDR2 comprising the amino acid sequence of SEQ ID NO: 451; and an LCDR3 comprising the amino acid sequence of SEQ ID NO: 452.

In some embodiments, the antibody or antigen binding region thereof that specifically binds to human ENTPD2 comprises an HCDR1 comprising the amino acid sequence of SEQ ID NO: 443; an HCDR2 comprising the amino acid sequence of SEQ ID NO: 444; an HCDR3 comprising the amino acid sequence of SEQ ID NO: 445; an LCDR1 comprising the amino acid sequence of SEQ ID NO: 463; an LCDR2 comprising the amino acid sequence of SEQ ID NO: 454; and an LCDR3 comprising the amino acid sequence of SEQ ID NO: 452.

In some embodiments, the antibody or antigen binding region thereof that specifically binds to human ENTPD2 comprises a heavy chain variable region (VH) comprising the amino acid sequence of SEQ ID NO: 410 (or a sequence at least about 90%, 95%, 99% or more identical thereto, and/or having one, two, three or more substitutions, insertions, deletions, or modifications), and a light chain variable region (VL) comprising the amino acid sequence of SEQ ID NO: 421 (or a sequence at least about 90%, 95%, 99% or more identical thereto, and/or having one, two, three or more substitutions, insertions, deletions, or modifications).

In some embodiments, the antibody or antigen binding region thereof that specifically binds to human ENTPD2 comprises a heavy chain variable region (VH) comprising the amino acid sequence of SEQ ID NO: 425 (or a sequence at least about 90%, 95%, 99% or more identical thereto, and/or having one, two, three or more substitutions, insertions, deletions, or modifications), and a light chain variable region (VL) comprising the amino acid sequence of SEQ ID NO: 429 (or a sequence at least about 90%, 95%, 99% or more identical thereto, and/or having one, two, three or more substitutions, insertions, deletions, or modifications).

In some embodiments, the antibody or antigen binding region thereof that specifically binds to human ENTPD2 comprises a heavy chain variable region (VH) comprising the amino acid sequence of SEQ ID NO: 433 (or a sequence at least about 90%, 95%, 99% or more identical thereto, and/or having one, two, three or more substitutions, insertions, deletions, or modifications), and a light chain variable region (VL) comprising the amino acid sequence of SEQ ID NO: 429 (or a sequence at least about 90%, 95%, 99% or more identical thereto, and/or having one, two, three or more substitutions, insertions, deletions, or modifications).

In some embodiments, the antibody or antigen binding region thereof that specifically binds to human ENTPD2 comprises a heavy chain variable region (VH) comprising the amino acid sequence of SEQ ID NO: 446 (or a sequence at least about 90%, 95%, 99% or more identical thereto, and/or having one, two, three or more substitutions, insertions, deletions, or modifications), and a light chain variable region (VL) comprising the amino acid sequence of SEQ ID NO: 457 (or a sequence at least about 90%, 95%, 99% or more identical thereto, and/or having one, two, three or more substitutions, insertions, deletions, or modifications).

In some embodiments, the antibody or antigen binding region thereof that specifically binds to human ENTPD2 comprises a heavy chain variable region (VH) comprising the amino acid sequence of SEQ ID NO: 446 (or a sequence at least about 90%, 95%, 99% or more identical thereto, and/or having one, two, three or more substitutions, insertions, deletions, or modifications), and a light chain variable region (VL) comprising the amino acid sequence of SEQ ID NO: 464 (or a sequence at least about 90%, 95%, 99% or more identical thereto, and/or having one, two, three or more substitutions, insertions, deletions, or modifications).

In some embodiments, the antibody that specifically binds to human ENTPD2 comprises a heavy chain comprising the amino acid sequence of SEQ ID NO: 412 (or a sequence at least about 90%, 95%, 99% or more identical thereto, and/or having one, two, three or more substitutions, insertions, deletions, or modifications), and a light chain comprising the amino acid sequence of SEQ ID NO: 423 (or a sequence at least about 90%, 95%, 99% or more identical thereto, and/or having one, two, three or more substitutions, insertions, deletions, or modifications).

In some embodiments, the antibody that specifically binds to human ENTPD2 comprises a heavy chain comprising the amino acid sequence of SEQ ID NO: 427 (or a sequence at least about 90%, 95%, 99% or more identical thereto, and/or having one, two, three or more substitutions, insertions, deletions, or modifications), and a light chain comprising the amino acid sequence of SEQ ID NO: 431 (or a sequence at least about 90%, 95%, 99% or more identical thereto, and/or having one, two, three or more substitutions, insertions, deletions, or modifications).

In some embodiments, the antibody that specifically binds to human ENTPD2 comprises a heavy chain comprising the amino acid sequence of SEQ ID NO: 435 (or a sequence at least about 90%, 95%, 99% or more identical thereto, and/or having one, two, three or more substitutions, insertions, deletions, or modifications), and a light chain comprising the amino acid sequence of SEQ ID NO: 431 (or a sequence at least about 90%, 95%, 99% or more identical thereto, and/or having one, two, three or more substitutions, insertions, deletions, or modifications).

In some embodiments, the antibody that specifically binds to human ENTPD2 comprises a heavy chain comprising the amino acid sequence of SEQ ID NO: 448 (or a sequence at least about 90%, 95%, 99% or more identical thereto, and/or having one, two, three or more substitutions, insertions, deletions, or modifications), and a light chain comprising the amino acid sequence of SEQ ID NO: 459 (or a sequence at least about 90%, 95%, 99% or more identical thereto, and/or having one, two, three or more substitutions, insertions, deletions, or modifications).

In some embodiments, the antibody that specifically binds to human ENTPD2 comprises a heavy chain comprising the amino acid sequence of SEQ ID NO: 448 (or a sequence at least about 90%, 95%, 99% or more identical thereto, and/or having one, two, three or more substitutions, insertions, deletions, or modifications), and a light chain comprising the amino acid sequence of SEQ ID NO: 466 (or a sequence at least about 90%, 95%, 99% or more identical thereto, and/or having one, two, three or more substitutions, insertions, deletions, or modifications).

In some embodiments, the present invention provides an antibody or antigen-binding fragment thereof, which bind to human ENTPD2 protein with a dissociation constant (KD) of less than 10 nM, e.g., a KD of less than 9 nM, less than 8 nM, less than 7 nM, less than 6 nM, less than 5 nM, less than 4 nM, less than 3 nM, less than 2 nM, less than 1 nM, e.g., as measured by Biacore. In some embodiments, the antibodies or antigen-binding fragments provided herein bind to human ENTPD2 protein with a dissociation constant (KD) of less than 5 nM, e.g., as measured by Biacore. In some embodiments, the antibodies or antigen-binding fragments provided herein bind to human ENTPD2 protein with a dissociation constant (KD) of less than 3 nM, e.g., as measured by Biacore. In some embodiments, the antibodies or antigen-binding fragments provided herein bind to human ENTPD2 protein with a dissociation constant (KD) of less than 1 nM, e.g., as measured by Biacore. In some embodiments, the dissociation constant of the antibodies or antigen binding fragments thereof described herein to human ENTPD2 is measured by Biacore at 25° C.

Provided herein are also antibodies or antigen binding fragments thereof that specifically bind to an epitope in human ENTPD2, wherein the epitope comprises at least one (e.g., at least two, at least three, at least four, at least five, at least six, at least seven, at least eight, at least nine, at least ten, at least eleven, at least twelve, at least thirteen, at least fourteen, at least fifteen, at least twenty) of the following residues: His50, Asp76, Pro78, Gly79, Gly80, Tyr85, Asp87, Asn88, Gly91, Gln94, Ser95, Gly98, Glu101, Gln102, Gln105, Asp106, Arg245, Thr272, Gln273, Leu275, Asp278, Arg298, Ala347, Ala350, Thr351, Arg392, Ala393, Arg394, or Tyr398. In some embodiments, such antibodies or antigen binding fragments include, but are not limited to, MAb1, MAb2, and MAb3 as disclosed in Table 9.

Provided herein are also antibodies or antigen binding fragments thereof that specifically bind to an epitope in human ENTPD2, wherein the epitope comprises at least one (e.g., at least two, at least three, at least four, at least five, at least six, at least seven, at least eight, at least nine, at least ten, at least eleven, at least twelve, at least thirteen, at least fourteen, at least fifteen, at least twenty) of the following residues: Gly79, Gln250, Leu253, Trp266, Arg268, Gly269, Phe270, Ser271, Thr272, Gln273, Val274, Leu275, Asp278, Arg298, Ser300, Ser302, Gly303, Thr380, Trp381, Ala382, Gly390, Gln391, Arg392, Ala393, Arg394, or Asp397. In some embodiments, such antibodies or antigen binding fragments include, but are not limited to, MAb4, and MAb5 as disclosed in Table 9.

Once a desired epitope on an antigen is determined, it is possible to generate antibodies to that epitope, e.g., using the techniques described in the present invention. Alternatively, during the discovery process, the generation and characterization of antibodies may elucidate information about desirable epitopes. From this information, it is then possible to competitively screen antibodies for binding to the same epitope. An approach to achieve this is to conduct cross-competition studies to find antibodies that competitively bind with one another, e.g., the antibodies compete for binding to the antigen. A high throughput process for “binning” antibodies based upon their cross-competition is described in International Patent Application No. WO 2003/48731. As will be appreciated by one of skill in the art, practically anything to which an antibody can specifically bind could be an epitope. An epitope can comprises those residues to which the antibody binds.

Generally, antibodies specific for a particular target antigen will preferentially recognize an epitope on the target antigen in a complex mixture of proteins and/or macromolecules.

Regions of a given polypeptide that include an epitope can be identified using any number of epitope mapping techniques, well known in the art. See, e.g., Epitope Mapping Protocols in Methods in Molecular Biology, Vol. 66 (Glenn E. Morris, Ed., 1996, Humana Press, Totowa, N.J). For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al., (1984) Proc. Natl. Acad. Sci. USA 8:3998-4002; Geysen et al., (1985) Proc. Natl. Acad. Sci. USA 82:78-182; Geysen et al., (1986) Mol. Immunol. 23:709-715. Similarly, conformational epitopes are readily identified by determining spatial conformation of amino acids such as by, e.g., x-ray crystallography and two-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al., (1981) Proc. Natl. Acad. Sci USA 78:3824-3828; for determining antigenicity profiles, and the Kyte-Doolittle technique, Kyte et al., (1982) J. Mol. Biol. 157:105-132; for hydropathy plots.

The antibody molecule can be a polyclonal or a monoclonal antibody. A monoclonal antibody can be made by hybridoma technology or by methods that do not use hybridoma technology (e.g., recombinant methods). In some embodiments, the antibody can be recombinantly produced, e.g., produced by phage display or by combinatorial methods.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982).

In one embodiment, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody.

An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric and/or humanized antibodies, can be engineered to minimize the immune response by a human patient to antibodies produced in non-human subjects or derived from the expression of non-human antibody genes. Chimeric antibodies comprise a non-human animal antibody variable region and a human antibody constant region. Such antibodies retain the epitope binding specificity of the original monoclonal antibody, but may be less immunogenic when administered to humans, and therefore more likely to be tolerated by the patient. For example, one or all (e.g., one, two, or three) of the variable regions of the light chain(s) and/or one or all (e.g., one, two, or three) of the variable regions the heavy chain(s) of a mouse antibody (e.g., a mouse monoclonal antibody) can each be joined to a human constant region, such as, without limitation an IgG1 human constant region. Chimeric monoclonal antibodies can be produced by recombinant DNA techniques known in the art. For example, a gene encoding the constant region of a non-human antibody molecule can be substituted with a gene encoding a human constant region (see Robinson et al., PCT Patent Publication PCT/US86/02269; Akira, et al., European Patent Application 184,187; or Taniguchi, M., European Patent Application 171,496). In addition, other suitable techniques that can be used to generate chimeric antibodies are described, for example, in U.S. Pat. Nos. 4,816,567; 4,978,775; 4,975,369; and 4,816,397.

A chimeric antibody can be further “humanized” by replacing portions of the variable region not involved in antigen binding with equivalent portions from human variable regions. Humanized antibodies comprise one or more human framework regions in the variable region together with non-human (e.g., mouse, rat, or hamster) complementarity-determining regions (CDRs) of the heavy and/or light chain. In some embodiments, a humanized antibody comprises sequences that are entirely human except for the CDR regions. Humanized antibodies are typically less immunogenic to humans, relative to non-humanized antibodies, and thus offer therapeutic benefits in certain situations. Humanized ENTPD2 antibodies can be generated using methods known in the art. See for example, Hwang et al., Methods 36:35, 2005; Queen et al., Proc. Natl. Acad. Sci. U.S.A. 86:10029-10033, 1989; Jones et al., Nature 321:522-25, 1986; Riechmann et al., Nature 332:323-27, 1988; Verhoeyen et al., Science 239:1534-36, 1988; Orlandi et al., Proc. Natl. Acad. Sci. U.S.A. 86:3833-3837, 1989; U.S. Pat. Nos. 5,225,539; 5,530,101; 5,585,089; 5,693,761; 5,693,762; and 6,180,370; and WO 90/07861.

Human ENTPD2 antibodies can be generated using methods that are known in the art. For example, the humaneering technology used to converting non-human antibodies into engineered human antibodies. U.S. Patent Publication No. 20050008625 describes an in vivo method for replacing a nonhuman antibody variable region with a human variable region in an antibody while maintaining the same or providing better binding characteristics relative to that of the nonhuman antibody. The method relies on epitope guided replacement of variable regions of a non-human reference antibody with a fully human antibody. The resulting human antibody is generally structurally unrelated to the reference nonhuman antibody, but binds to the same epitope on the same antigen as the reference antibody. Briefly, the serial epitope-guided complementarity replacement approach is enabled by setting up a competition in cells between a “competitor” and a library of diverse hybrids of the reference antibody (“test antibodies”) for binding to limiting amounts of antigen in the presence of a reporter system which responds to the binding of test antibody to antigen. The competitor can be the reference antibody or derivative thereof such as a single-chain Fv fragment. The competitor can also be a natural or artificial ligand of the antigen which binds to the same epitope as the reference antibody. The only requirements of the competitor are that it binds to the same epitope as the reference antibody, and that it competes with the reference antibody for antigen binding. The test antibodies have one antigen-binding V-region in common from the nonhuman reference antibody, and the other V-region selected at random from a diverse source such as a repertoire library of human antibodies. The common V-region from the reference antibody serves as a guide, positioning the test antibodies on the same epitope on the antigen, and in the same orientation, so that selection is biased toward the highest antigen-binding fidelity to the reference antibody.

Many types of reporter system can be used to detect desired interactions between test antibodies and antigen. For example, complementing reporter fragments may be linked to antigen and test antibody, respectively, so that reporter activation by fragment complementation only occurs when the test antibody binds to the antigen. When the test antibody- and antigen-reporter fragment fusions are co-expressed with a competitor, reporter activation becomes dependent on the ability of the test antibody to compete with the competitor, which is proportional to the affinity of the test antibody for the antigen. Other reporter systems that can be used include the reactivator of an auto-inhibited reporter reactivation system (RAIR) as disclosed in U.S. patent application Ser. No. 10/208,730 (Publication No. 20030198971), or competitive activation system disclosed in U.S. patent application Ser. No. 10/076,845 (Publication No. 20030157579).

With the serial epitope-guided complementarity replacement system, selection is made to identify cells expresses a single test antibody along with the competitor, antigen, and reporter components. In these cells, each test antibody competes one-on-one with the competitor for binding to a limiting amount of antigen. Activity of the reporter is proportional to the amount of antigen bound to the test antibody, which in turn is proportional to the affinity of the test antibody for the antigen and the stability of the test antibody. Test antibodies are initially selected on the basis of their activity relative to that of the reference antibody when expressed as the test antibody. The result of the first round of selection is a set of “hybrid” antibodies, each of which is comprised of the same non-human V-region from the reference antibody and a human V-region from the library, and each of which binds to the same epitope on the antigen as the reference antibody. One of more of the hybrid antibodies selected in the first round will have an affinity for the antigen comparable to or higher than that of the reference antibody.

In the second V-region replacement step, the human V-regions selected in the first step are used as guide for the selection of human replacements for the remaining non-human reference antibody V-region with a diverse library of cognate human V-regions. The hybrid antibodies selected in the first round may also be used as competitors for the second round of selection. The result of the second round of selection is a set of fully human antibodies which differ structurally from the reference antibody, but which compete with the reference antibody for binding to the same antigen. Some of the selected human antibodies bind to the same epitope on the same antigen as the reference antibody. Among these selected human antibodies, one or more binds to the same epitope with an affinity which is comparable to or higher than that of the reference antibody.

Using a mouse or chimeric ENTPD2 antibody, human antibodies that bind to human ENTPD2 with the same binding specificity and the same or better binding affinity can be generated. In addition, such human ENTPD2 antibodies can also be commercially obtained from companies which customarily produce human antibodies, e.g., KaloBios, Inc. (Mountain View, Calif.).

In some embodiments, the present invention provides an antibody or antigen-binding fragment thereof that bind to human ENTPD2 protein and modulates one or more ENTPD2 activities/functions, e.g., inhibiting the enzymatic actitivies of human ENTPD2, e.g., by at least 40%, at least 50%, at least 60%, at least 70%, at least 80%, or at least 90%. In some embodiments, the enzymatic activity of human ENTPD2 is measured using an in vitro FRET assay which measures the hydrolysis of ATP to ADP by either recombinant ENTPD2 or ENTPD2 expressed on the surface of cells.

In some embodiments, the anti-human ENTPD2 antibodies or antigen binding fragments thereof described herein inhibit ENTPD2's ability of hydrolysis of adenosine triphosphate (ATP). In some embodiments, ENTPD2's ability of hydrolysis of ATP is measured using an in vitro FRET assay which measures the hydrolysis of ATP to ADP by either recombinant ENTPD2 or ENTPD2 expressed on the surface of cells.

In some embodiments, the anti-human ENTPD2 antibodies or antigen binding fragments thereof described herein interfere with ATP binding to ENTPD2 or trap ATP within the catalytic domain of ENTPD2. In some embodiments, interference with ATP binding to ENTPD2 or trapping ATP within the catalytic domain of ENTPD2 is measured using an in vitro FRET assay which measures the hydrolysis of ATP to ADP by either recombinant ENTPD2 or ENTPD2 expressed on the surface of cells.

### Pharmaceutical Compositions, Kits and Administration

In an embodiment of the antibody for use according to the invention or method according to the invention, the antibody molecule that binds to human CD73 or human ENTPD2 is in the form of a pharmaceutical composition. Compositions, e.g., pharmaceutically acceptable compositions, which include an anti-CD73 antibody molecule or an anti-ENTPD2 antibody molecule described herein, may be formulated together with a pharmaceutically acceptable carrier. As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g. by injection or infusion).

The compositions set out herein may be in a variety of forms. These include, for example, liquid, semi-solid and solid dosage forms, such as liquid solutions (e.g., injectable and infusible solutions), dispersions or suspensions, liposomes, and suppositories. A suitable form depends on the intended mode of administration and therapeutic application. Typical suitable compositions are in the form of injectable or infusible solutions. One suitable mode of administration is parenteral (e.g., intravenous, subcutaneous, intraperitoneal, intramuscular). In some embodiments, the antibody molecule is administered by intravenous infusion or injection. In certain embodiments, the antibody is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibody molecules can be administered by a variety of methods. Several are known in the art, and for many therapeutic applications, an appropriate route/mode of administration is intravenous injection or infusion. In an embodiment, the antibody molecules can be administered by intravenous infusion at a rate of more than 20 mg/min, e.g., 20-40 mg/min. In an embodiment, the antibody molecules can be administered by intravenous infusion at a rate of greater than or equal to 40 mg/min to reach a dose of about 35 to 440 mg/m2, about 70 to 310 mg/m2, or about 110 to 130 mg/m2. In an embodiment, the antibody molecules can be administered by intravenous infusion at a rate of less than 10 mg/min, e.g., less than or equal to 5 mg/min to reach a dose of about 1 to 100 mg/m2, about 5 to 50 mg/m2, about 7 to 25 mg/m2, or about 10 mg/m2. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In an aspect of the invention, there is provided an antibody molecule that binds to human CD73 or human ENTPD2 for use in treating a cancer in a subject or in a method of treating cancer in a subject, whereby the antibody molecule is administered in a step-up dosing regime such that in the main phase the antibody molecule dose is administered at a main dose amount according to a main dosing period with a main dosing period frequency, which is preceded by an initial phase in which said antibody molecule is administered at a higher dosing period frequency than the main dosing period frequency and is administered via fractionated dose amounts of the main dose amount, wherein in the initial phase within a time period equal to the main dosing period the fractionated dose amounts summed together shall not exceed the amount of the main phase dose amount. The fractionated doses can be for example ⅙, ⅓, ½, ⅔ of the main dose amount. In an embodiment of the antibody for use according to the invention or method according to the invention, in the initial phase within a time period equal to the main dosing period, the fractionated dose amounts summed together equal the main dose amount.

In some embodiments, an anti-CD73 antibody molecule or an anti-ENTPD2 antibody molecule disclosed herein is administered in the main phase by injection (e.g., subcutaneously or intravenously) at a dose (e.g., a flat dose) of about 60 mg to 2400 mg, e.g., about 100 mg to 2400 mg, about 100 mg to 2200 mg, about 100 mg to 2000 mg, about 100 mg to 1800 mg, about 100 mg to 1600 mg, about 100 mg to 1400 mg, about 100 mg to 1200 mg, about 100 mg to 1000 mg, about 100 mg to 800 mg, about 100 mg to 600 mg, about 100 mg to 400 mg, about 100 mg to 200 mg. In some embodiments, an anti-CD73 antibody molecule or an anti-ENTPD2 antibody molecule disclosed herein is administered in the main phase by injection (e.g., subcutaneously or intravenously) at a dose (e.g., a flat dose) of about 100 mg, about 150 mg, 200 mg, about 250 mg, about 300 mg, about 350 mg, about 400 mg, about 450 mg, about 500 mg, about 550 mg, 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, and about 2400 mg. The dosing schedule (e.g., flat dosing schedule) in the main phase can vary from e.g., once a week to once every 2, 3, or 4 weeks. In one embodiment, an anti-CD73 antibody molecule or an anti-ENTPD2 antibody molecule disclosed herein is administered in the main phase at a dose from about 100 mg to 1200, once every two weeks. In one embodiment, an anti-CD73 antibody molecule disclosed herein is administered in the main phase at a dose of at least about 600 mg once every two weeks. In one embodiment, an anti-ENTPD2 antibody molecule disclosed herein is administered in the main phase at a dose of at least about 300 mg once every two weeks.

In certain embodiments, in the main phase the antibody molecule is administered, e.g., intravenously, at a dose of about 200 mg, about 400 mg, about 600 mg, about 800 mg, about 1000 mg, about 1200 mg, about 2400 mg, about 3000 mg, or about 3600 mg, e.g., QW, Q2W, or Q4W. In certain embodiments, the antibody molecule is administered, e.g., intravenously, at a dose of about 200 mg Q2W. In certain embodiments, the antibody molecule is administered, e.g., intravenously, at a dose of about 300 mg Q2W. In certain embodiments, the antibody molecule is administered, e.g., intravenously, at a dose of about 400 mg Q2W. In certain embodiments, the antibody molecule is administered, e.g., intravenously, at a dose of about 600 mg Q2W. In certain embodiments, the antibody molecule is administered, e.g., intravenously, at a dose of about 800 mg Q2W. In certain embodiments, the antibody molecule is administered, e.g., intravenously, at a dose of about 1000 mg Q2W. In certain embodiments, the antibody molecule is administered, e.g., intravenously, at a dose of about 1200 mg Q2W. In certain embodiments, the antibody molecule is administered, e.g., intravenously, at a dose of about 2400 mg Q2W. In certain embodiments, the antibody molecule is administered, e.g., intravenously, at a dose of about 3000 mg Q2W. In certain embodiments, the antibody molecule is administered, e.g., intravenously, at a dose of about 3600 mg Q2W.

In an embodiment of the antibody for use according to the invention or method according to the invention, the main dose amount of the antibody molecule that binds to human CD73 is 600 mg. In an embodiment the main dose frequency is Q2W.

In an embodiment of the antibody for use according to the invention or method according to the invention, the main dose amount of the antibody molecule that binds to human ENTPD2 is 300 mg. In an embodiment the main dose frequency is Q2W.

In an embodiment of the antibody for use according to the invention or method according to the invention, in the initial phase the antibody molecule that binds to human CD73 is administered at a frequency of QW.

In an embodiment of the antibody for use according to the invention or method according to the invention, in the initial phase the antibody molecule that binds to human ENTPD2 is administered at a frequency of QW or Q2W.

In an embodiment of the antibody for use according to the invention or method according to the invention, the initial phase of the dosing of the anti-CD73 or anti-ENTPD2 antibody (or both) is two weeks.

In an embodiment of the antibody for use according to the invention or method according to the invention, in the initial phase the fractionated dose amounts that are administered within a time period equal to the main dosing period when summed together equal the main dose amount. In an embodiment these initial phase doses are administered at a lower amount followed by an intermediate amount of the main phase dose amount. The lower amount is lower than the main phase dose amount and the intermediate dose amount is a value between these two amounts. In an embodiment, in the initial phase the fractionated dose amounts are about 200 mg and about 400 mg and are administered within two weeks. In another embodiment, in the initial phase the fractionated dose amounts are about 100 mg and about 500 mg and are administered within two weeks. In an alternative embodiment, in the initial phase the fractionated dose amounts administered within a time period equal to the main dosing period summed together equal the main dose amount and wherein the fractionated doses amounts are equal amounts, e.g. about 300 mg and about 300 mg. The amounts in these embodiments refer to the amount of the anti-CD73 antibody.

In an embodiment the anti-CD73 antibody is administered in a step-up dosing regime such that the anti-CD73 antibody is administered in the initial phase of two weeks once at about 200 mg in the first week and once at about 400 mg in the second week, followed by the main phase with about 600 mg administered Q2W.

For example, the antibody molecule that binds to human CD73 may be administered in the initial phase on day 1 at about 200 mg and on day 8 at about 400 mg, followed by the main phase beginning on day 15 with about 600 mg, and continuing thereafter with about 600 mg administered Q2W.

In some embodiments, the antibody molecule that binds to human ENTPD2 may be administered in the initial phase on day 1 at about 100 mg, followed by the main phase beginning on day 15 with about 300 mg, and continuing thereafter with about 300 mg administered Q2W.

In some embodiments, the antibody molecule that binds to human ENTPD2 may be administered in the initial phase on day 1 at about 100 mg and on day 8 at about 100 mg, followed by the main phase beginning on day 15 with about 300 mg, and continuing thereafter with about 300 mg administered Q2W.

In one embodiment, an anti-CD73 antibody molecule or an anti-ENTPD2 antibody molecule disclosed herein is administered, e.g., by infusion, over a period of 30 minutes, a period of 1 hour, or a period of up to 2 hours. In one embodiment, an anti-CD73 antibody molecule or an anti-ENTPD2 antibody molecule disclosed herein is administered, e.g., by infusion, over a period of 1 to 2 hours.

The pharmaceutical compositions useful in the dosage regime of the invention may include a “therapeutically effective amount” or a “prophylactically effective amount” of an antibody molecule used in the dosage regime of the invention. A “therapeutically effective amount” refers to an amount effective, at dosages and for periods of time necessary, to achieve the desired therapeutic result. A therapeutically effective amount of the modified antibody or antibody fragment may vary according to factors such as the disease state, age, sex, and weight of the individual, and the ability of the antibody or antibody portion to elicit a desired response in the individual. A therapeutically effective amount is also one in which any toxic or detrimental effects of the antibody molecule are outweighed by the therapeutically beneficial effects. A “therapeutically effective dosage” preferably inhibits a measurable parameter by at least about 20%, more preferably by at least about 40%, even more preferably by at least about 60%, and still more preferably by at least about 80% relative to untreated subjects. The measurable parameter may be, e.g., tumor growth rate or pathogen growth rate. The ability of an antibody molecule to inhibit a measurable parameter can be evaluated in an animal model system predictive of efficacy in the corresponding human disease. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also described herein is a kit comprising an antibody molecule described herein. The kit can include one or more other elements including: instructions for use; other reagents, e.g., a label, a therapeutic agent, or an agent useful for chelating, or otherwise coupling, an antibody to a label or therapeutic agent, or a radioprotective composition; devices or other materials for preparing the antibody molecule for administration; pharmaceutically acceptable carriers; and devices or other materials for administration to a subject.

### Therapeutic Uses

As used herein, the term “subject” is intended to include human and non-human animals. In one embodiment, the subject is a human subject, e.g., a human patient having a disorder or condition characterized by abnormal CD73 or ENTPD2 functioning. The term “non-human animals” includes mammals and non-mammals, such as non-human primates. In one embodiment, the subject is a human. In one embodiment, the subject is a human patient in need of enhancement of an immune response. In one embodiment, the subject is immunocompromised, e.g., the subject is undergoing, or has undergone a chemotherapeutic or radiation therapy. Alternatively, or in combination, the subject is, or is at risk of being, immunocompromised as a result of an infection. The methods and compositions described herein are suitable for treating human patients having a disorder that can be treated by augmenting the T-cell mediated immune response. For example, the methods and compositions described herein can enhance a number of immune activities. In one embodiment, the subject has increased number or activity of tumour-infiltrating T lymphocytes (TILs).

### Cancer

The dosage regime according to the invention in one embodiment relates to treatment of a subject in vivo using an anti-CD73 antibody molecule or an anti-ENTPD2 antibody molecule such that growth of cancerous tumors is inhibited or reduced. An anti-CD73 antibody may be used alone to inhibit the growth of cancerous tumors. Alternatively, an anti-CD73 antibody molecule or an anti-ENTPD2 antibody may be used in combination with one or more of: a standard of care treatment (e.g., for cancers), another antibody molecule, an immunomodulator (e.g., an activator of a costimulatory molecule or an inhibitor of a coinhibitory molecule); a vaccine, e.g., a therapeutic cancer vaccine; or other forms of cell therapy, as described below.

Accordingly, in one embodiment, the dosage regime according to the invention provides a method of inhibiting growth of tumor cells in a subject, comprising administering to the subject a therapeutically effective amount of an anti-CD73 antibody molecule or an anti-ENTPD2 antibody molecule described herein, according to the dosage regime as described herein.

In one embodiment, the methods are suitable for the treatment of cancer in vivo. When antibodies to CD73 antibody molecule or an anti-ENTPD2 are administered in combination with one or more agents, the combination can be administered in either order or simultaneously according to the dosage regime as described herein.

### Types of Cancer

In another aspect of the dosage regime according to the invention, a method of treating a subject, e.g., reducing or ameliorating, a hyperproliferative condition or disorder (e.g., a cancer), e.g., solid tumor, a hematological cancer, soft tissue tumor, or a metastatic lesion, in a subject is provided. The method includes administering to the subject one or more anti-CD73 antibody molecule or an anti-ENTPD2 antibody molecules described herein, alone or in combination with other agents or therapeutic modalities.

As used herein, the term “cancer” is meant to include all types of cancerous growths or oncogenic processes, metastatic tissues or malignantly transformed cells, tissues, or organs, irrespective of histopathologic type or stage of invasiveness. Examples of cancerous disorders include, but are not limited to, solid tumors, hematological cancers, soft tissue tumors, and metastatic lesions. Examples of solid tumors include malignancies, e.g., sarcomas, and carcinomas (including adenocarcinomas and squamous cell carcinomas), of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. Squamous cell carcinomas include malignancies, e.g., in the lung, esophagus, skin, head and neck region, oral cavity, anus, and cervix. In one embodiment, the cancer is a melanoma, e.g., an advanced stage melanoma. Metastatic lesions of the aforementioned cancers can also be treated or prevented using the methods and compositions of the dosage regimes of the invention.

Exemplary cancers whose growth can be inhibited using the dosage regimes according to the invention include cancers typically responsive to immunotherapy. Non-limiting examples of preferred cancers for treatment include melanoma (e.g., metastatic malignant melanoma), renal cancer (e.g., clear cell carcinoma), prostate cancer (e.g., hormone refractory prostate adenocarcinoma), breast cancer, colon cancer and lung cancer (e.g., non-small cell lung cancer). In an embodiment of the dosage regimes of the invention the cancer to be treated is non-small cell lung cancer, pancreatic ductal adenocarcinoma, triple-negative breast cancer, microsatellite stable (MSS) colorectal cancer, metastatic castration resistant prostate cancer, ovarian cancer or renal cell carcinoma. Additionally, refractory or recurrent malignancies can be treated using the antibody molecules described herein. Metastatic castration resistant prostate cancer is also known as hormone refractory prostate adenocarcinoma or androgen-independent prostate cancer.

Examples of other cancers that can be treated include bone cancer, pancreatic cancer, skin cancer, cancer of the head or neck, cutaneous or intraocular malignant melanoma, uterine cancer, ovarian cancer, rectal cancer, anal cancer, gastro-esophageal, stomach cancer, testicular cancer, carcinoma of the fallopian tubes, carcinoma of the endometrium, carcinoma of the cervix, carcinoma of the vagina, carcinoma of the vulva, Merkel cell cancer, Hodgkin lymphoma, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, multiple myeloma, myelodisplastic syndromes, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos (e.g., mesothelioma), and combinations of said cancers.

In some embodiments, the therapies here can be used to treat a patient that has (or is identified as having) a cancer associated with an infection, e.g., a viral or bacterial infection. Exemplary cancers include cervical cancer, anal cancer, HPV-associated head and neck squamous cell cancer, HPV-associated esophageal papillomas, HHV6-associated lymphomas, EBV-associated lymphomas (including Burkitt lymphoma), Gastric MALT lymphoma, other infection-associated MALT lymphomas, HCC, and Kaposi's sarcoma.

In other embodiments, the cancer is a hematological malignancy or cancer including but is not limited to a leukemia or a lymphoma. For example, the anti-CD73 antibody molecule or an anti-ENTPD2 antibody molecule can be used to treat cancers and malignancies including, but not limited to, e.g., acute leukemias including but not limited to, e.g., B-cell acute lymphoid leukemia (“BALL”), T-cell acute lymphoid leukemia (“TALL”), acute lymphoid leukemia (ALL); one or more chronic leukemias including but not limited to, e.g., chronic myelogenous leukemia (CML), chronic lymphocytic leukemia (CLL); additional hematologic cancers or hematologic conditions including, but not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like.

In one embodiment, the cancer is chosen from a lung cancer (e.g., a non-small cell lung cancer (NSCLC) (e.g., a NSCLC with squamous and/or non-squamous histology, or a NSCLC adenocarcinoma)), a melanoma (e.g., an advanced melanoma), a renal cancer (e.g., a renal cell carcinoma, e.g., clear cell renal cell carcinoma), a liver cancer, a myeloma (e.g., a multiple myeloma), a prostate cancer, a breast cancer (e.g., a breast cancer that does not express one, two or all of estrogen receptor, progesterone receptor, or Her2/neu, e.g., a triple negative breast cancer), a colorectal cancer (e.g., microsatellite stable (MSS) colorectal cancer), ovarian cancer, a cholangiocarcinoma (intrahepatic or extrahepatic), a pancreatic cancer, a head and neck cancer (e.g., head and neck squamous cell carcinoma (HNSCC), anal cancer, gastro-esophageal cancer, thyroid cancer, cervical cancer, a lymphoproliferative disease (e.g., a post-transplant lymphoproliferative disease) or a hematological cancer, T-cell lymphoma, a non-Hogdkin's lymphoma, or a leukemia (e.g., a myeloid leukemia).

In one embodiment, the cancer is chosen from lung cancer (e.g., non-small cell lung cancer), pancreas cancer (e.g., pancreatic ductal adenocarcinoma), breast cancer (e.g., triple-negative breast cancer), melanoma, head and neck cancer (e.g., squamous head and neck cancer), colorectal cancer (e.g., microsatellite stable (MSS) colorectal cancer), ovarian cancer, metastatic castration resistant prostate cancer or renal cancer (e.g., renal cell carcinoma).

In one embodiment, the cancer is chosen from bladder cancer, leukemia, lymphoma, glioma, glioblastoma, ovarian cancer, thyroid cancer, esophageal cancer, prostate cancer, uterine/cervical cancer, testicular cancer, esophageal cancer, gastrointestinal cancer, colon cancer, kidney cancer, stomach cancer, germ cell cancer, bone cancer, liver cancer, skin cancer, neoplasm of the central nervous system, myeloma, sarcoma, and virus-related cancer.

In one embodiment, the cancer is chosen from a colon cancer (e.g., colorectal cancer (CRC) or colorectal adenocarcinoma), gastric cancer (e.g., stomach adenocarcinoma, gastric carcinoma), esophageal cancer (e.g. esophageal squamous cell carcinoma (ESCC), esophageal gastric junction (EGJ) cancer), lung cancer (e.g., small cell lung cancer), breast cancer (e.g., breast adenocarcinoma) or ovarian cancer.

In one embodiment, the cancer is colorectal cancer (CRC) or colorectal adenocarcinoma. In another embodiment the cancer is MSS colorectal cancer (CRC).

In another embodiment, the cancer is esophageal cancer.

In a further embodiment, the cancer is gastric cancer.

In yet another embodiment, the cancer is esophageal gastric junction (EGJ) cancer.

In yet another embodiment, the cancer is esophageal squamous cell carcinoma (ESCC).

In a further embodiment, the cancer is cholangiocarcinoma. The cholangiocarcinoma may be intrahepatic or extrahepatic.

In another embodiment, the cancer is pancreatic cancer.

Combination of Anti-CD73 Antibody Molecules and/or Anti-ENTPD2 Antibody Molecules

In the dosage regimes according to the invention, the anti-CD73 antibody molecule or anti-ENTPD2 antibody molecules can be used in combination with other therapies, and/or each other. For example, the combination therapy can include a composition comprising the anti-CD73 antibody molecule or anti-ENTPD2 antibodies as described herein co-formulated with, and/or co-administered with, one or more additional therapeutic agents, e.g., one or more anti-cancer agents, cytotoxic or cytostatic agents, hormone treatment, vaccines, and/or other immunotherapies. In some embodiments, the anti-CD73 antibody molecules or anti-ENTPD2 antibody molecules as described herein are co-formulated and/or co-administered with each other. In other embodiments, the anti-CD73 antibody molecules or anti-ENTPD2 antibody molecules are administered in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies.

By “in combination with,” it is not intended to imply that the therapy or the therapeutic agents must be administered at the same time and/or formulated for delivery together, although these methods of delivery are within the scope described herein. The anti-CD73 antibody molecules or anti-ENTPD2 antibody molecules can be administered concurrently with, prior to, or subsequent to, one or more other additional therapies or therapeutic agents. The anti-CD73 antibody molecule or anti-ENTPD2 antibody molecule and the other agent or therapeutic protocol can be administered in any order. In general, each agent will be administered at a dose and/or on a time schedule determined for that agent. It will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

### Exemplary Adenosine A2A Receptor Antagonists

In certain embodiments of the dosage regime according to the invention, the anti-CD73 antibody molecules and/or anti-ENTPD2 antibody molecules described herein are administered in combination with an adenosine A2A receptor (A2AR) antagonist. Exemplary A2AR antagonists include, e.g., PBF509 (Palobiofarma/Novartis), also known as NIR178, CP1444/V81444 (Corvus/Genentech), AZD4635/HTL-1071 (AstraZeneca/Heptares), Vipadenant (Redox/Juno), GBV-2034 (Globavir), AB928 (Arcus Biosciences), Theophylline, Istradefylline (Kyowa Hakko Kogyo), Tozadenant/SYN-115 (Acorda), KW-6356 (Kyowa Hakko Kogyo), ST-4206 (Leadiant Biosciences), and Preladenant/SCH 420814 (Merck/Schering).

In certain embodiments, the A2AR antagonist is PBF509. PBF509 is also known as NIR178. PBF509 and other A2AR antagonists are disclosed in U.S. Pat. No. 8,796,284 and WO 2017/025918, herein incorporated by reference in their entirety. PBF509 refers to 5-bromo-2,6-di-(1H-pyrazol-1-yl)pyrimidine-4-amine with the following structure:

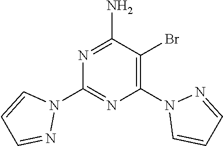 [SMILES: Nc1nc(-n2cccn2)nc(-n2cccn2)c1Br]

In certain embodiments, the A2AR antagonist is CP1444/V81444. CPI-444 and other A2AR antagonists are disclosed in WO 2009/156737, herein incorporated by reference in its entirety. In certain embodiments, the A2AR antagonist is (S)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine. In certain embodiments, the A2AR antagonist is (R)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine, or racemate thereof. In certain embodiments, the A2AR antagonist is 7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine. In certain embodiments, the A2AR antagonist has the following structure:

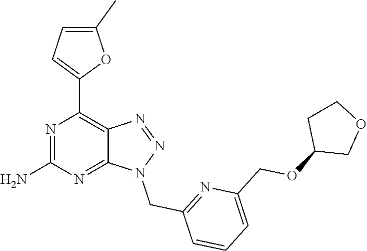 [SMILES: Cc1ccc(-c2nc(N)nc3c2nnn3Cc2cccc(CO[C@H]3CCOC3)n2)o1]

In certain embodiments, the A2AR antagonist is AZD4635/HTL-1071. A2AR antagonists are disclosed in WO 2011/095625, herein incorporated by reference in its entirety. In certain embodiments, the A2AR antagonist is 6-(2-chloro-6-methylpyridin-4-yl)-5-(4-fluorophenyl)-1,2,4-triazin-3-amine. In certain embodiments, the A2AR antagonist has the following structure:

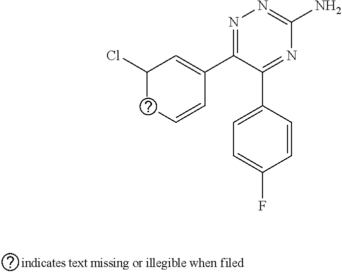 [SMILES: Nc1nnc(C2=CC(Cl)CC=C2)c(-c2ccc(F)cc2)n1]

In certain embodiments, the A2AR antagonist is ST-4206 (Leadiant Biosciences). In certain embodiments, the A2AR antagonist is an A2AR antagonist described in U.S. Pat. No. 9,133,197, herein incorporated by reference in its entirety. In certain embodiments, the A2AR antagonist has the following structure:

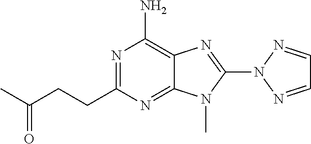 [SMILES: CC(=O)CCc1nc(N)c2nc(-n3nccn3)n(C)c2n1]

In certain embodiments, the A2AR antagonist is an A2AR antagonist described in U.S. Pat. Nos. 8,114,845, 9,029,393, US20170015758, or US20160129108, herein incorporated by reference in their entirety.

In certain embodiments, the A2AR antagonist is istradefylline (CAS Registry Number: 155270-99-8). Istradefylline is also known as KW-6002 or 8-[(E)-2-(3,4-dimethoxyphenyl)vinyl]-1,3-diethyl-7-methyl-3,7-dihydro-1H-purine-2,6-dione. Istradefylline is disclosed, e.g., in LeWitt et al. (2008) Annals of Neurology 63 (3): 295-302).

In certain embodiments, the A2aR antagonist is tozadenant (Biotie). Tozadenant is also known as SYN115 or 4-hydroxy-N-(4-methoxy-7-morpholin-4-yl-1,3-benzothiazol-2-yl)-4-methylpiperidine-1-carboxamide. Tozadenant blocks the effect of endogenous adenosine at the A2a receptors, resulting in the potentiation of the effect of dopamine at the D2 receptor and inhibition of the effect of glutamate at the mGluR5 receptor. In some embodiments, the A2aR antagonist is preladenant (CAS Registry Number: 377727-87-2). Preladenant is also known as SCH 420814 or 2-(2-Furanyl)-7-[2-[4-[4-(2-methoxyethoxy)phenyl]-1-piperazinyl]ethyl]7H-pyrazolo[4,3-e][1,2,4]triazolo[1,5-c]pyrimidine-5-amine. Preladenant was developed as a drug that acted as a potent and selective antagonist at the adenosine A2A receptor.

In certain embodiments, the A2aR antagonist is vipadenan. Vipadenan is also known as BIIB014, V2006, or 3-[(4-amino-3-methylphenyl)methyl]-7-(furan-2-yl)triazolo[4,5-d]pyrimidin-5-amine.

Other exemplary A2aR antagonists include, e.g., ATL-444, MSX-3, SCH-58261, SCH-412,348, SCH-442,416, VER-6623, VER-6947, VER-7835, CGS-15943, or ZM-241,385.

### Exemplary PD-1 Inhibitors

In certain embodiments of the dosage regime of the invention, the anti-CD73 antibody molecules and/or anti-ENTPD2 antibody molecules described herein are administered in combination with a PD-1 inhibitor. The PD-1 inhibitor may be an antibody, an antigen binding fragment thereof, an immunoadhesin, a fusion protein, or an oligopeptide. In some embodiments, the PD-1 inhibitor is chosen from Spartalizumab (PDR001) (Novartis), Nivolumab (Bristol-Myers Squibb), Pembrolizumab (Merck & Co), Pidilizumab (CureTech), MED10680 (Medimmune), REGN2810 (Regeneron), TSR-042 (Tesaro), PF-06801591 (Pfizer), tislelizumab (BGB-A317, Beigene), BGB-108 (Beigene), INCSHR1210 (Incyte), or AMP-224 (Amplimmune).

### Exemplary Anti-PD-1 Antibody Molecules

In one embodiment of the dosage regime of the invention, the PD-1 inhibitor is an anti-PD-1 antibody molecule. In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety.

In one embodiment, the anti-PD-1 antibody molecule comprises at least one, two, three, four, five or six complementarity determining regions (CDRs) (or collectively all of the CDRs) from a heavy and light chain variable region comprising an amino acid sequence shown in Table 5 (e.g., from the heavy and light chain variable region sequences of BAP049-Clone-E or BAP049-Clone-B disclosed in Table 5), or encoded by a nucleotide sequence shown in Table 5. In some embodiments, the CDRs are according to the Kabat definition (e.g., as set out in Table 5). In some embodiments, the CDRs are according to the Chothia definition (e.g., as set out in Table 5). In some embodiments, the CDRs are according to the combined CDR definitions of both Kabat and Chothia (e.g., as set out in Table 5). In one embodiment, the combination of Kabat and Chothia CDR of VH CDR1 comprises the amino acid sequence GYTFTTYWMH (SEQ ID NO: 541). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 5, or encoded by a nucleotide sequence shown in Table 5.

In one embodiment, the anti-PD-1 antibody molecule comprises a heavy chain variable region (VH) comprising a VHCDR1 amino acid sequence of SEQ ID NO: 501, a VHCDR2 amino acid sequence of SEQ ID NO: 502, and a VHCDR3 amino acid sequence of SEQ ID NO: 503; and a light chain variable region (VL) comprising a VLCDR1 amino acid sequence of SEQ ID NO: 510, a VLCDR2 amino acid sequence of SEQ ID NO: 511, and a VLCDR3 amino acid sequence of SEQ ID NO: 512, each disclosed in Table 5.

In one embodiment, the antibody molecule comprises a VH comprising a VHCDR1 encoded by the nucleotide sequence of SEQ ID NO: 524, a VHCDR2 encoded by the nucleotide sequence of SEQ ID NO: 525, and a VHCDR3 encoded by the nucleotide sequence of SEQ ID NO: 526; and a VL comprising a VLCDR1 encoded by the nucleotide sequence of SEQ ID NO: 529, a VLCDR2 encoded by the nucleotide sequence of SEQ ID NO: 530, and a VLCDR3 encoded by the nucleotide sequence of SEQ ID NO: 531, each disclosed in Table 5.

In one embodiment, the anti-PD-1 antibody molecule comprises a VH comprising the amino acid sequence of SEQ ID NO: 506, or an amino acid sequence having at least about 85%, 90%, 95%, or 99% sequence identity or higher to SEQ ID NO: 506. In one embodiment, the anti-PD-1 antibody molecule comprises a VL comprising the amino acid sequence of SEQ ID NO: 520, or an amino acid sequence having at least about 85%, 90%, 95%, or 99% sequence identity or higher to SEQ ID NO: 520. In one embodiment, the anti-PD-1 antibody molecule comprises a VL comprising the amino acid sequence of SEQ ID NO: 516, or an amino acid sequence having at least about 85%, 90%, 95%, or 99% sequence identity or higher to SEQ ID NO: 516. In one embodiment, the anti-PD-1 antibody molecule comprises a VH comprising the amino acid sequence of SEQ ID NO: 506 and a VL comprising the amino acid sequence of SEQ ID NO: 520. In one embodiment, the anti-PD-1 antibody molecule comprises a VH comprising the amino acid sequence of SEQ ID NO: 506 and a VL comprising the amino acid sequence of SEQ ID NO: 516.

In one embodiment, the antibody molecule comprises a VH encoded by the nucleotide sequence of SEQ ID NO: 507, or a nucleotide sequence having at least about 85%, 90%, 95%, or 99% sequence identity or higher to SEQ ID NO: 507. In one embodiment, the antibody molecule comprises a VL encoded by the nucleotide sequence of SEQ ID NO: 521 or 517, or a nucleotide sequence having at least about 85%, 90%, 95%, or 99% sequence identity or higher to SEQ ID NO: 521 or 517. In one embodiment, the antibody molecule comprises a VH encoded by the nucleotide sequence of SEQ ID NO: 507 and a VL encoded by the nucleotide sequence of SEQ ID NO: 521 or 517.

In one embodiment, the anti-PD-1 antibody molecule comprises a heavy chain comprising the amino acid sequence of SEQ ID NO: 508, or an amino acid sequence having at least about 85%, 90%, 95%, or 99% sequence identity or higher to SEQ ID NO: 508. In one embodiment, the anti-PD-1 antibody molecule comprises a light chain comprising the amino acid sequence of SEQ ID NO: 522, or an amino acid sequence having at least about 85%, 90%, 95%, or 99% sequence identity or higher to SEQ ID NO: 522. In one embodiment, the anti-PD-1 antibody molecule comprises a light chain comprising the amino acid sequence of SEQ ID NO: 518, or an amino acid sequence having at least about 85%, 90%, 95%, or 99% sequence identity or higher to SEQ ID NO: 518. In one embodiment, the anti-PD-1 antibody molecule comprises a heavy chain comprising the amino acid sequence of SEQ ID NO: 508 and a light chain comprising the amino acid sequence of SEQ ID NO: 522. In one embodiment, the anti-PD-1 antibody molecule comprises a heavy chain comprising the amino acid sequence of SEQ ID NO: 508 and a light chain comprising the amino acid sequence of SEQ ID NO: 518.

In one embodiment, the antibody molecule comprises a heavy chain encoded by the nucleotide sequence of SEQ ID NO: 509, or a nucleotide sequence having at least about 85%, 90%, 95%, or 99% sequence identity or higher to SEQ ID NO: 509. In one embodiment, the antibody molecule comprises a light chain encoded by the nucleotide sequence of SEQ ID NO: 523 or 519, or a nucleotide sequence having at least about 85%, 90%, 95%, or 99% sequence identity or higher to SEQ ID NO: 523 or 519. In one embodiment, the antibody molecule comprises a heavy chain encoded by the nucleotide sequence of SEQ ID NO: 509 and a light chain encoded by the nucleotide sequence of SEQ ID NO: 523 or 519.

The antibody molecules described herein can be made by vectors, host cells, and methods described in US 2015/0210769, incorporated by reference in its entirety.

TABLE 5
Amino acid and nucleotide sequences of exemplary anti-PD-1 antibody molecules

| | | |
|---|---|---|
| BAP049-Clone-B HC | | |
| SEQ ID NO: 501 | HCDR1 | TYWMH |
| (Kabat) | | |
| SEQ ID NO: 502 | HCDR2 | NIYPGTGGSNFDEKFKN |
| (Kabat) | | |
| SEQ ID NO: 503 | HCDR3 | WTTGTGAY |
| (Kabat) | | |
| SEQ ID NO: 504 | HCDR1 | GYTFTTY |
| (Chothia) | | |
| SEQ ID NO: 505 | HCDR2 | YPGTGG |
| (Chothia) | | |
| SEQ ID NO: 503 | HCDR3 | WTTGTGAY |
| (Chothia) | | |
| SEQ ID NO: 506 | VH | EVQLVQSGAEVKKPGESLRISCKGSGYTFTTYWMHWVRQ |
| | | ATGQGLEWMGNIYPGTGGSNFDEKFKNRVTITADKSTSTA |
| | | YMELSSLRSEDTAVYYCTRWTTGTGAYWGQGTTVTVSS |
| SEQ ID NO: 507 | DNA | GAGGTGCAGCTGGTGCAGTCAGGCGCCGAAGTGAAGAA |
| | VH | GCCCGGCGAGTCACTGAGAATTAGCTGTAAAGGTTCAG |
| | | GCTACACCTTCACTACCTACTGGATGCACTGGGTCCGCC |
| | | AGGCTACCGGTCAAGGCCTCGAGTGGATGGGTAATATC |
| | | TACCCCGGCACCGGCGGCTCTAACTTCGACGAGAAGTT |
| | | TAAGAATAGAGTGACTATCACCGCCGATAAGTCTACTAG |
| | | CACCGCCTATATGGAACTGTCTAGCCTGAGATCAGAGGA |
| | | CACCGCCGTCTACTACTGCACTAGGTGGACTACCGGCA |
| | | CAGGCGCCTACTGGGGTCAAGGCACTACCGTGACCGTG |
| | | TCTAGC |
| SEQ ID NO: 508 | Heavy | EVQLVQSGAEVKKPGESLRISCKGSGYTFTTYWMHWVRQ |
| | chain | ATGQGLEWMGNIYPGTGGSNFDEKFKNRVTITADKSTSTA |
| | | YMELSSLRSEDTAVYYCTRWTTGTGAYWGQGTTVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTYT |
| | | CNVDHKPSNTKVDKRVESKYGPPCPPCPAPEFLGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQV |
| | | SLTCLVKGFYPSDIAVEWESNGQPENNYKTTPPVLDSDGS |
| | | FFLYSRLTVDKSRWQEGNVFSCSVMHEALHNHYTQKSLSL |
| | | SLG |
| SEQ ID NO: 509 | DNA | GAGGTGCAGCTGGTGCAGTCAGGCGCCGAAGTGAAGAA |
| | heavy | GCCCGGCGAGTCACTGAGAATTAGCTGTAAAGGTTCAG |
| | chain | GCTACACCTTCACTACCTACTGGATGCACTGGGTCCGCC |
| | | AGGCTACCGGTCAAGGCCTCGAGTGGATGGGTAATATC |
| | | TACCCCGGCACCGGCGGCTCTAACTTCGACGAGAAGTT |
| | | TAAGAATAGAGTGACTATCACCGCCGATAAGTCTACTAG |
| | | CACCGCCTATATGGAACTGTCTAGCCTGAGATCAGAGGA |
| | | CACCGCCGTCTACTACTGCACTAGGTGGACTACCGGCA |
| | | CAGGCGCCTACTGGGGTCAAGGCACTACCGTGACCGTG |
| | | TCTAGCGCTAGCACTAAGGGCCCGTCCGTGTTCCCCCT |
| | | GGCACCTTGTAGCCGGAGCACTAGCGAATCCACCGCTG |
| | | CCCTCGGCTGCCTGGTCAAGGATTACTTCCCGGAGCCC |
| | | GTGACCGTGTCCTGGAACAGCGGAGCCCTGACCTCCGG |
| | | AGTGCACACCTTCCCCGCTGTGCTGCAGAGCTCCGGGC |
| | | TGTACTCGCTGTCGTCGGTGGTCACGGTGCCTTCATCTA |
| | | GCCTGGGTACCAAGACCTACACTTGCAACGTGGACCAC |
| | | AAGCCTTCCAACACTAAGGTGGACAAGCGCGTCGAATC |
| | | GAAGTACGGCCCACCGTGCCCGCCTTGTCCCGCGCCG |
| | | GAGTTCCTCGGCGGTCCCTCGGTCTTTCTGTTCCCACC |
| | | GAAGCCCAAGGACACTTTGATGATTTCCCGCACCCCTGA |
| | | AGTGACATGCGTGGTCGTGGACGTGTCACAGGAAGATC |
| | | CGGAGGTGCAGTTCAATTGGTACGTGGATGGCGTCGAG |
| | | GTGCACAACGCCAAAACCAAGCCGAGGGAGGAGCAGTT |
| | | CAACTCCACTTACCGCGTCGTGTCCGTGCTGACGGTGC |
| | | TGCATCAGGACTGGCTGAACGGGAAGGAGTACAAGTGC |
| | | AAAGTGTCCAACAAGGGACTTCCTAGCTCAATCGAAAAG |
| | | ACCATCTCGAAAGCCAAGGGACAGCCCCGGGAACCCCA |
| | | AGTGTATACCCTGCCACCGAGCCAGGAAGAAATGACTAA |
| | | GAACCAAGTCTCATTGACTTGCCTTGTGAAGGGCTTCTA |
| | | CCCATCGGATATCGCCGTGGAATGGGAGTCCAACGGCC |
| | | AGCCGGAAAACAACTACAAGACCACCCCTCCGGTGCTG |
| | | GACTCAGACGGATCCTTCTTCCTCTACTCGCGGCTGACC |
| | | GTGGATAAGAGCAGATGGCAGGAGGGAAATGTGTTCAG |
| | | CTGTTCTGTGATGCATGAAGCCCTGCACAACCACTACAC |
| | | TCAGAAGTCCCTGTCCCTCTCCCTGGGA |
| BAP049-Clone-B LC | | |
| SEQ ID NO: 510 | LCDR1 | KSSQSLLDSGNQKNFLT |
| (Kabat) | | |
| SEQ ID NO: 511 | LCDR2 | WASTRES |
| (Kabat) | | |
| SEQ ID NO: 512 | LCDR3 | QNDYSYPYT |
| (Kabat) | | |
| SEQ ID NO: 513 | LCDR1 | SQSLLDSGNQKNF |
| (Chothia) | | |
| SEQ ID NO: 514 | LCDR2 | WAS |
| (Chothia) | | |
| SEQ ID NO: 515 | LCDR3 | DYSYPY |
| (Chothia) | | |
| SEQ ID NO: 516 | VL | EIVLTQSPATLSLSPGERATLSCKSSQSLLDSGNQKNFLTW |
| | | YQQKPGKAPKLLIYWASTRESGVPSRFSGSGSGTDFTFTIS |
| | | SLQPEDIATYYCONDYSYPYTFGQGTKVEIK |
| SEQ ID NO: 517 | DNA | GAGATCGTCCTGACTCAGTCACCCGCTACCCTGAGCCT |
| | VL | GAGCCCTGGCGAGCGGGCTACACTGAGCTGTAAATCTA |
| | | GTCAGTCACTGCTGGATAGCGGTAATCAGAAGAACTTCC |
| | | TGACCTGGTATCAGCAGAAGCCCGGTAAAGCCCCTAAG |
| | | CTGCTGATCTACTGGGCCTCTACTAGAGAATCAGGCGTG |
| | | CCCTCTAGGTTTAGCGGTAGCGGTAGTGGCACCGACTT |
| | | CACCTTCACTATCTCTAGCCTGCAGCCCGAGGATATCGC |
| | | TACCTACTACTGTCAGAACGACTATAGCTACCCCTACAC |
| | | CTTCGGTCAAGGCACTAAGGTCGAGATTAAG |
| SEQ ID NO: 518 | Light | EIVLTQSPATLSLSPGERATLSCKSSQSLLDSGNQKNFLTW |
| | chain | YQQKPGKAPKLLIYWASTRESGVPSRFSGSGSGTDFTFTIS |
| | DNA | SLQPEDIATYYCQNDYSYPYTFGQGTKVEIKRTVAAPSVFIF |
| | | PPSDEQLKSGTASVVCLLNNFYPREAKVQWKVDNALQSG |
| | | NSQESVTEQDSKDSTYSLSSTLTLSKADYEKHKVYACEVT |
| | | HQGLSSPVTKSFNRGEC |
| SEQ ID NO: 519 | light | GAGATCGTCCTGACTCAGTCACCCGCTACCCTGAGCCT |
| | chain | GAGCCCTGGCGAGCGGGCTACACTGAGCTGTAAATCTA |
| | | GTCAGTCACTGCTGGATAGCGGTAATCAGAAGAACTTCC |
| | | TGACCTGGTATCAGCAGAAGCCCGGTAAAGCCCCTAAG |
| | | CTGCTGATCTACTGGGCCTCTACTAGAGAATCAGGCGTG |
| | | CCCTCTAGGTTTAGCGGTAGCGGTAGTGGCACCGACTT |
| | | CACCTTCACTATCTCTAGCCTGCAGCCCGAGGATATCGC |
| | | TACCTACTACTGTCAGAACGACTATAGCTACCCCTACAC |
| | | CTTCGGTCAAGGCACTAAGGTCGAGATTAAGCGTACGG |
| | | TGGCCGCTCCCAGCGTGTTCATCTTCCCCCCCAGCGAC |
| | | GAGCAGCTGAAGAGCGGCACCGCCAGCGTGGTGTGCC |
| | | TGCTGAACAACTTCTACCCCCGGGAGGCCAAGGTGCAG |
| | | TGGAAGGTGGACAACGCCCTGCAGAGCGGCAACAGCCA |
| | | GGAGAGCGTCACCGAGCAGGACAGCAAGGACTCCACCT |
| | | ACAGCCTGAGCAGCACCCTGACCCTGAGCAAGGCCGAC |
| | | TACGAGAAGCATAAGGTGTACGCCTGCGAGGTGACCCA |
| | | CCAGGGCCTGTCCAGCCCCGTGACCAAGAGCTTCAACA |
| | | GGGGCGAGTGC |
| BAP049-Clone-E HC | | |
| SEQ ID NO: 501 | HCDR1 | TYWMH |
| (Kabat) | | |
| SEQ ID NO: 502 | HCDR2 | NIYPGTGGSNFDEKFKN |
| (Kabat) | | |
| SEQ ID NO: 503 | HCDR3 | WTTGTGAY |
| (Kabat) | | |
| SEQ ID NO: 504 | HCDR1 | GYTFTTY |
| (Chothia) | | |
| SEQ ID NO: 505 | HCDR2 | YPGTGG |
| (Chothia) | | |
| SEQ ID NO: 503 | HCDR3 | WTTGTGAY |
| (Chothia) | | |
| SEQ ID NO: 506 | VH | EVQLVQSGAEVKKPGESLRISCKGSGYTFTTYWMHWVRQ |
| | | ATGQGLEWMGNIYPGTGGSNFDEKFKNRVTITADKSTSTA |
| | | YMELSSLRSEDTAVYYCTRWTTGTGAYWGQGTTVTVSS |
| SEQ ID NO: 507 | DNA | GAGGTGCAGCTGGTGCAGTCAGGCGCCGAAGTGAAGAA |
| | VH | GCCCGGCGAGTCACTGAGAATTAGCTGTAAAGGTTCAG |
| | | GCTACACCTTCACTACCTACTGGATGCACTGGGTCCGCC |
| | | AGGCTACCGGTCAAGGCCTCGAGTGGATGGGTAATATC |
| | | TACCCCGGCACCGGCGGCTCTAACTTCGACGAGAAGTT |
| | | TAAGAATAGAGTGACTATCACCGCCGATAAGTCTACTAG |
| | | CACCGCCTATATGGAACTGTCTAGCCTGAGATCAGAGGA |
| | | CACCGCCGTCTACTACTGCACTAGGTGGACTACCGGCA |
| | | CAGGCGCCTACTGGGGTCAAGGCACTACCGTGACCGTG |
| | | TCTAGC |
| SEQ ID NO: 508 | Heavy | EVQLVQSGAEVKKPGESLRISCKGSGYTFTTYWMHWVRQ |
| | chain | ATGQGLEWMGNIYPGTGGSNFDEKFKNRVTITADKSTSTA |
| | | YMELSSLRSEDTAVYYCTRWTTGTGAYWGQGTTVTVSSA |
| | | STKGPSVFPLAPCSRSTSESTAALGCLVKDYFPEPVTVSW |
| | | NSGALTSGVHTFPAVLQSSGLYSLSSVVTVPSSSLGTKTYT |
| | | CNVDHKPSNTKVDKRVESKYGPPCPPCPAPEFLGGPSVFL |
| | | FPPKPKDTLMISRTPEVTCVVVDVSQEDPEVQFNWYVDGV |
| | | EVHNAKTKPREEQFNSTYRVVSVLTVLHQDWLNGKEYKCK |
| | | VSNKGLPSSIEKTISKAKGQPREPQVYTLPPSQEEMTKNQV |
| | | SLTCLVKGFYPSDIAVEWESNGQPENNYKTTPPVLDSDGS |
| | | FFLYSRLTVDKSRWQEGNVFSCSVMHEALHNHYTQKSLSL |
| | | SLG |
| SEQ ID NO: 509 | DNA | GAGGTGCAGCTGGTGCAGTCAGGCGCCGAAGTGAAGAA |
| | heavy | GCCCGGCGAGTCACTGAGAATTAGCTGTAAAGGTTCAG |
| | chain | GCTACACCTTCACTACCTACTGGATGCACTGGGTCCGCC |
| | | AGGCTACCGGTCAAGGCCTCGAGTGGATGGGTAATATC |
| | | TACCCCGGCACCGGCGGCTCTAACTTCGACGAGAAGTT |
| | | TAAGAATAGAGTGACTATCACCGCCGATAAGTCTACTAG |
| | | CACCGCCTATATGGAACTGTCTAGCCTGAGATCAGAGGA |
| | | CACCGCCGTCTACTACTGCACTAGGTGGACTACCGGCA |
| | | CAGGCGCCTACTGGGGTCAAGGCACTACCGTGACCGTG |
| | | TCTAGCGCTAGCACTAAGGGCCCGTCCGTGTTCCCCCT |
| | | GGCACCTTGTAGCCGGAGCACTAGCGAATCCACCGCTG |
| | | CCCTCGGCTGCCTGGTCAAGGATTACTTCCCGGAGCCC |
| | | GTGACCGTGTCCTGGAACAGCGGAGCCCTGACCTCCGG |
| | | AGTGCACACCTTCCCCGCTGTGCTGCAGAGCTCCGGGC |
| | | TGTACTCGCTGTCGTCGGTGGTCACGGTGCCTTCATCTA |
| | | GCCTGGGTACCAAGACCTACACTTGCAACGTGGACCAC |
| | | AAGCCTTCCAACACTAAGGTGGACAAGCGCGTCGAATC |
| | | GAAGTACGGCCCACCGTGCCCGCCTTGTCCCGCGCCG |
| | | GAGTTCCTCGGCGGTCCCTCGGTCTTTCTGTTCCCACC |
| | | GAAGCCCAAGGACACTTTGATGATTTCCCGCACCCCTGA |
| | | AGTGACATGCGTGGTCGTGGACGTGTCACAGGAAGATC |
| | | CGGAGGTGCAGTTCAATTGGTACGTGGATGGCGTCGAG |
| | | GTGCACAACGCCAAAACCAAGCCGAGGGAGGAGCAGTT |
| | | CAACTCCACTTACCGCGTCGTGTCCGTGCTGACGGTGC |
| | | TGCATCAGGACTGGCTGAACGGGAAGGAGTACAAGTGC |
| | | AAAGTGTCCAACAAGGGACTTCCTAGCTCAATCGAAAAG |
| | | ACCATCTCGAAAGCCAAGGGACAGCCCCGGGAACCCCA |
| | | AGTGTATACCCTGCCACCGAGCCAGGAAGAAATGACTAA |
| | | GAACCAAGTCTCATTGACTTGCCTTGTGAAGGGCTTCTA |
| | | CCCATCGGATATCGCCGTGGAATGGGAGTCCAACGGCC |
| | | AGCCGGAAAACAACTACAAGACCACCCCTCCGGTGCTG |
| | | GACTCAGACGGATCCTTCTTCCTCTACTCGCGGCTGACC |
| | | GTGGATAAGAGCAGATGGCAGGAGGGAAATGTGTTCAG |
| | | CTGTTCTGTGATGCATGAAGCCCTGCACAACCACTACAC |
| | | TCAGAAGTCCCTGTCCCTCTCCCTGGGA |
| BAP049-Clone-E LC | | |
| SEQ ID NO: 510 | LCDR1 | KSSQSLLDSGNQKNFLT |
| (Kabat) | | |
| SEQ ID NO: 511 | LCDR2 | WASTRES |
| (Kabat) | | |
| SEQ ID NO: 512 | LCDR3 | QNDYSYPYT |
| (Kabat) | | |
| SEQ ID NO: 513 | LCDR1 | SQSLLDSGNQKNF |
| (Chothia) | | |
| SEQ ID NO: 514 | LCDR2 | WAS |
| (Chothia) | | |
| SEQ ID NO: 515 | LCDR3 | DYSYPY |
| (Chothia) | | |
| SEQ ID NO: 520 | VL | EIVLTQSPATLSLSPGERATLSCKSSQSLLDSGNQKNFLTW |
| | | YQQKPGQAPRLLIYWASTRESGVPSRFSGSGSGTDFTFTI |
| | | SSLEAEDAATYYCONDYSYPYTFGQGTKVEIK |
| SEQ ID NO: 521 | DNA | GAGATCGTCCTGACTCAGTCACCCGCTACCCTGAGCCT |
| | VL | GAGCCCTGGCGAGCGGGCTACACTGAGCTGTAAATCTA |
| | | GTCAGTCACTGCTGGATAGCGGTAATCAGAAGAACTTCC |
| | | TGACCTGGTATCAGCAGAAGCCCGGTCAAGCCCCTAGA |
| | | CTGCTGATCTACTGGGCCTCTACTAGAGAATCAGGCGTG |
| | | CCCTCTAGGTTTAGCGGTAGCGGTAGTGGCACCGACTT |
| | | CACCTTCACTATCTCTAGCCTGGAAGCCGAGGACGCCG |
| | | CTACCTACTACTGTCAGAACGACTATAGCTACCCCTACA |
| | | CCTTCGGTCAAGGCACTAAGGTCGAGATTAAG |
| SEQ ID NO: 522 | Light | EIVLTQSPATLSLSPGERATLSCKSSQSLLDSGNQKNFLTW |
| | chain | YQQKPGQAPRLLIYWASTRESGVPSRFSGSGSGTDFTFTI |
| | | SSLEAEDAATYYCONDYSYPYTFGQGTKVEIKRTVAAPSVF |
| | | IFPPSDEQLKSGTASVVCLLNNFYPREAKVQWKVDNALQS |
| | | GNSQESVTEQDSKDSTYSLSSTLTLSKADYEKHKVYACEV |
| | | THQGLSSPVTKSFNRGEC |
| SEQ ID NO: 523 | DNA | GAGATCGTCCTGACTCAGTCACCCGCTACCCTGAGCCT |
| | light | GAGCCCTGGCGAGCGGGCTACACTGAGCTGTAAATCTA |
| | chain | GTCAGTCACTGCTGGATAGCGGTAATCAGAAGAACTTCC |
| | | TGACCTGGTATCAGCAGAAGCCCGGTCAAGCCCCTAGA |
| | | CTGCTGATCTACTGGGCCTCTACTAGAGAATCAGGCGTG |
| | | CCCTCTAGGTTTAGCGGTAGCGGTAGTGGCACCGACTT |
| | | CACCTTCACTATCTCTAGCCTGGAAGCCGAGGACGCCG |
| | | CTACCTACTACTGTCAGAACGACTATAGCTACCCCTACA |
| | | CCTTCGGTCAAGGCACTAAGGTCGAGATTAAGCGTACG |
| | | GTGGCCGCTCCCAGCGTGTTCATCTTCCCCCCCAGCGA |
| | | CGAGCAGCTGAAGAGCGGCACCGCCAGCGTGGTGTGC |
| | | CTGCTGAACAACTTCTACCCCCGGGAGGCCAAGGTGCA |
| | | GTGGAAGGTGGACAACGCCCTGCAGAGCGGCAACAGC |
| | | CAGGAGAGCGTCACCGAGCAGGACAGCAAGGACTCCAC |
| | | CTACAGCCTGAGCAGCACCCTGACCCTGAGCAAGGCCG |
| | | ACTACGAGAAGCATAAGGTGTACGCCTGCGAGGTGACC |
| | | CACCAGGGCCTGTCCAGCCCCGTGACCAAGAGCTTCAA |
| | | CAGGGGCGAGTGC |
| BAP049-Clone-B HC | | |
| SEQ ID NO: 524 | HCDR1 | ACCTACTGGATGCAC |
| (Kabat) | | |
| SEQ ID NO: 525 | HCDR2 | AATATCTACCCCGGCACCGGCGGCTCTAACTTCGACGA |
| (Kabat) | | GAAGTTTAAGAAT |
| SEQ ID NO: 526 | HCDR3 | TGGACTACCGGCACAGGCGCCTAC |
| (Kabat) | | |
| SEQ ID NO: 527 | HCDR1 | GGCTACACCTTCACTACCTAC |
| (Chothia) | | |
| SEQ ID NO: 528 | HCDR2 | TACCCCGGCACCGGCGGC |
| (Chothia) | | |
| SEQ ID NO: 526 | HCDR3 | TGGACTACCGGCACAGGCGCCTAC |
| (Chothia) | | |
| BAP049-Clone-B LC | | |
| SEQ ID NO: 529 | LCDR1 | AAATCTAGTCAGTCACTGCTGGATAGCGGTAATCAGAAG |
| (Kabat) | | AACTTCCTGACC |
| SEQ ID NO: 530 | LCDR2 | TGGGCCTCTACTAGAGAATCA |
| (Kabat) | | |
| SEQ ID NO: 531 | LCDR3 | CAGAACGACTATAGCTACCCCTACACC |
| (Kabat) | | |
| SEQ ID NO: 532 | LCDR1 | AGTCAGTCACTGCTGGATAGCGGTAATCAGAAGAACTTC |
| (Chothia) | | |
| SEQ ID NO: 533 | LCDR2 | TGGGCCTCT |
| (Chothia) | | |
| SEQ ID NO: 534 | LCDR3 | GACTATAGCTACCCCTAC |
| (Chothia) | | |
| BAP049-Clone-E HC | | |
| SEQ ID NO: 524 | HCDR1 | ACCTACTGGATGCAC |
| (Kabat) | | |
| SEQ ID NO: 525 | HCDR2 | AATATCTACCCCGGCACCGGCGGCTCTAACTTCGACGA |
| (Kabat) | | GAAGTTTAAGAAT |
| SEQ ID NO: 526 | HCDR3 | TGGACTACCGGCACAGGCGCCTAC |
| (Kabat) | | |
| SEQ ID NO: 527 | HCDR1 | GGCTACACCTTCACTACCTAC |
| (Chothia) | | |
| SEQ ID NO: 528 | HCDR2 | TACCCCGGCACCGGCGGC |
| (Chothia) | | |
| SEQ ID NO: 526 | HCDR3 | TGGACTACCGGCACAGGCGCCTAC |
| (Chothia) | | |
| BAP049-Clone-E LC | | |
| SEQ ID NO: 529 | LCDR1 | AAATCTAGTCAGTCACTGCTGGATAGCGGTAATCAGAAG |
| (Kabat) | | AACTTCCTGACC |
| SEQ ID NO: 530 | LCDR2 | TGGGCCTCTACTAGAGAATCA |
| (Kabat) | | |
| SEQ ID NO: 531 | LCDR3 | CAGAACGACTATAGCTACCCCTACACC |
| (Kabat) | | |
| SEQ ID NO: 532 | LCDR1 | AGTCAGTCACTGCTGGATAGCGGTAATCAGAAGAACTTC |
| (Chothia) | | |
| SEQ ID NO: 533 | LCDR2 | TGGGCCTCT |
| (Chothia) | | |
| SEQ ID NO: 534 | LCDR3 | GACTATAGCTACCCCTAC |
| (Chothia) | | |

### Other Exemplary PD-1 Inhibitors

In one embodiment, the anti-PD-1 antibody molecule is Nivolumab (Bristol-Myers Squibb), also known as MDX-1106, MDX-1106-04, ONO-4538, BMS-936558, or OPDIVO®. Nivolumab (clone 5C4) and other anti-PD-1 antibodies are disclosed in U.S. Pat. No. 8,008,449 and WO 2006/121168, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Nivolumab, e.g., as disclosed in Table 6.

In one embodiment, the anti-PD-1 antibody molecule is Pembrolizumab (Merck & Co), also known as Lambrolizumab, MK-3475, MK03475, SCH-900475, or KEYTRUDA®. Pembrolizumab and other anti-PD-1 antibodies are disclosed in Hamid, O. et al. (2013) New England Journal of Medicine 369 (2): 134-44, U.S. Pat. No. 8,354,509, and WO 2009/114335, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pembrolizumab, e.g., as disclosed in Table 6.

In one embodiment, the anti-PD-1 antibody molecule is Pidilizumab (CureTech), also known as CT-011. Pidilizumab and other anti-PD-1 antibodies are disclosed in Rosenblatt, J. et al. (2011) J Immunotherapy 34(5): 409-18, U.S. Pat. Nos. 7,695,715, 7,332,582, and 8,686,119, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pidilizumab, e.g., as disclosed in Table 6.

In one embodiment, the anti-PD-1 antibody molecule is MED10680 (Medimmune), also known as AMP-514. MED10680 and other anti-PD-1 antibodies are disclosed in U.S. Pat. No. 9,205,148 and WO 2012/145493, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of MED10680.

In one embodiment, the anti-PD-1 antibody molecule is REGN2810 (Regeneron). In one embodiment, the anti-PD-1 antibody molecule comprises one or more of the CDR sequences (or collectively all of the CDR sequences), the heavy chain or light chain variable region sequence, or the heavy chain or light chain sequence of REGN2810.

In one embodiment, the anti-PD-1 antibody molecule is PF-06801591 (Pfizer). In one embodiment, the anti-PD-1 antibody molecule comprises one or more of the CDR sequences (or collectively all of the CDR sequences), the heavy chain or light chain variable region sequence, or the heavy chain or light chain sequence of PF-06801591.

In one embodiment, the anti-PD-1 antibody molecule is tislelizumab (BGB-A317) or BGB-108 (Beigene). In one embodiment, the anti-PD-1 antibody molecule comprises one or more of the CDR sequences (or collectively all of the CDR sequences), the heavy chain or light chain variable region sequence, or the heavy chain or light chain sequence of tislelizumab (BGB-A317) or BGB-108.

In one embodiment, the anti-PD-1 antibody molecule is INCSHR1210 (Incyte), also known as INCSHR01210 or SHR-1210. In one embodiment, the anti-PD-1 antibody molecule comprises one or more of the CDR sequences (or collectively all of the CDR sequences), the heavy chain or light chain variable region sequence, or the heavy chain or light chain sequence of INCSHR1210.

In one embodiment, the anti-PD-1 antibody molecule is TSR-042 (Tesaro), also known as ANB011. In one embodiment, the anti-PD-1 antibody molecule comprises one or more of the CDR sequences (or collectively all of the CDR sequences), the heavy chain or light chain variable region sequence, or the heavy chain or light chain sequence of TSR-042.

Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signaling pathway, e.g., as described in U.S. Pat. No. 8,907,053, incorporated by reference in its entirety. In one embodiment, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In one embodiment, the PD-1 inhibitor is AMP-224 (B7-DCIg (Amplimmune), e.g., disclosed in WO 2010/027827 and WO 2011/066342, incorporated by reference in their entirety).

TABLE 6
Amino acid sequences of other exemplary anti-PD-1 antibody molecules
Nivolumab

| | | |
|---|---|---|
| SEQ ID NO: | Heavy | QVQLVESGGGVVQPGRSLRLDCKASGITFSNSGMHWVRQAPGKG |
| 535 | chain | LEWVAVIWYDGSKRYYADSVKGRFTISRDNSKNTLFLQMNSLRAED |
| | | TAVYYCATNDDYWGQGTLVTVSSASTKGPSVFPLAPCSRSTSEST |
| | | AALGCLVKDYFPEPVTVSWNSGALTSGVHTFPAVLQSSGLYSLSSV |
| | | VTVPSSSLGTKTYTCNVDHKPSNTKVDKRVESKYGPPCPPCPAPE |
| | | FLGGPSVFLFPPKPKDTLMISRTPEVTCVVVDVSQEDPEVQFNWYV |
| | | DGVEVHNAKTKPREEQFNSTYRVVSVLTVLHQDWLNGKEYKCKVS |
| | | NKGLPSSIEKTISKAKGQPREPQVYTLPPSQEEMTKNQVSLTCLVK |
| | | GFYPSDIAVEWESNGQPENNYKTTPPVLDSDGSFFLYSRLTVDKSR |
| | | WQEGNVFSCSVMHEALHNHYTQKSLSLSLGK |
| SEQ ID NO: | Light | EIVLTQSPATLSLSPGERATLSCRASQSVSSYLAWYQQKPGQAPRL |
| 536 | chain | LIYDASNRATGIPARFSGSGSGTDFTLTISSLEPEDFAVYYCQQSSN |
| | | WPRTFGQGTKVEIKRTVAAPSVFIFPPSDEQLKSGTASVVCLLNNF |
| | | YPREAKVQWKVDNALQSGNSQESVTEQDSKDSTYSLSSTLTLSKA |
| | | DYEKHKVYACEVTHQGLSSPVTKSFNRGEC |

Pembrolizumab

| | | |
|---|---|---|
| SEQ ID NO: | Heavy | QVQLVQSGVEVKKPGASVKVSCKASGYTFTNYYMYWVRQAPGQG |
| 537 | chain | LEWMGGINPSNGGTNFNEKFKNRVTLTTDSSTTTAYMELKSLQFD |
| | | DTAVYYCARRDYRFDMGFDYWGQGTTVTVSSASTKGPSVFPLAP |
| | | CSRSTSESTAALGCLVKDYFPEPVTVSWNSGALTSGVHTFPAVLQS |
| | | SGLYSLSSVVTVPSSSLGTKTYTCNVDHKPSNTKVDKRVESKYGPP |
| | | CPPCPAPEFLGGPSVFLFPPKPKDTLMISRTPEVTCVVVDVSQEDP |
| | | EVQFNWYVDGVEVHNAKTKPREEQFNSTYRVVSVLTVLHQDWLN |
| | | GKEYKCKVSNKGLPSSIEKTISKAKGQPREPQVYTLPPSQEEMTKN |
| | | QVSLTCLVKGFYPSDIAVEWESNGQPENNYKTTPPVLDSDGSFFLY |
| | | SRLTVDKSRWQEGNVFSCSVMHEALHNHYTQKSLSLSLGK |
| SEQ ID NO: | Light | EIVLTQSPATLSLSPGERATLSCRASKGVSTSGYSYLHWYQQKPGQ |
| 538 | chain | APRLLIYLASYLESGVPARFSGSGSGTDFTLTISSLEPEDFAVYYCQ |
| | | HSRDLPLTFGGGTKVEIKRTVAAPSVFIFPPSDEQLKSGTASVVCLL |
| | | NNFYPREAKVQWKVDNALQSGNSQESVTEQDSKDSTYSLSSTLTL |
| | | SKADYEKHKVYACEVTHQGLSSPVTKSFNRGEC |

Pidilizumab

| | | |
|---|---|---|
| SEQ ID NO: | Heavy | QVQLVQSGSELKKPGASVKISCKASGYTFTNYGMNWVRQAPGQG |
| 539 | chain | LQWMGWINTDSGESTYAEEFKGRFVFSLDTSVNTAYLQITSLTAED |
| | | TGMYFCVRVGYDALDYWGQGTLVTVSSASTKGPSVFPLAPSSKST |
| | | SGGTAALGCLVKDYFPEPVTVSWNSGALTSGVHTFPAVLQSSGLY |
| | | SLSSVVTVPSSSLGTQTYICNVNHKPSNTKVDKRVEPKSCDKTHTC |
| | | PPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNG |
| | | KEYKCKVSNKALPAPIEKTISKAKGQPREPQVYTLPPSREEMTKNQ |
| | | VSLTCLVKGFYPSDIAVEWESNGQPENNYKTTPPVLDSDGSFFLYS |
| | | KLTVDKSRWQQGNVFSCSVMHEALHNHYTQKSLSLSPGK |
| SEQ ID NO: | Light | EIVLTQSPSSLSASVGDRVTITCSARSSVSYMHWFQQKPGKAPKLW |
| 540 | chain | IYRTSNLASGVPSRFSGSGSGTSYCLTINSLQPEDFATYYCQQRSS |
| | | FPLTFGGGTKLEIKRTVAAPSVFIFPPSDEQLKSGTASVVCLLNNFY |
| | | PREAKVQWKVDNALQSGNSQESVTEQDSKDSTYSLSSTLTLSKAD |
| | | YEKHKVYACEVTHQGLSSPVTKSFNRGEC |

Tislelizumab

| | | |
|---|---|---|
| SEQ ID NO: | Heavy | QVQLQESGPGLVKPSETLSLTCTVSGFSLTSYGVHWIRQPPGKGLE |
| 542 | chain | WIGVIYADGSTNYNPSLKSRVTISKDTSKNQVSLKLSSVTAADTAVY |
| | | YCARAYGNYWYIDVWGQGTTVTVSSASTKGPSVFPLAPCSRSTSE |
| | | STAALGCLVKDYFPEPVTVSWNSGALTSGVHTFPAVLQSSGLYSLS |
| | | SVVTVPSSSLGTKTYTCNVDHKPSNTKVDKRVESKYGPPCPPCPA |
| | | PPVAGGPSVFLFPPKPKDTLMISRTPEVTCVVVAVSQEDPEVQFNW |
| | | YVDGVEVHNAKTKPREEQFNSTYRVVSVLTVVHQDWLNGKEYKCK |
| | | VSNKGLPSSIEKTISKAKGQPREPQVYTLPPSQEEMTKNQVSLTCL |
| | | VKGFYPSDIAVEWESNGQPENNYKTTPPVLDSDGSFFLYSKLTVDK |
| | | SRWQEGNVFSCSVMHEALHNHYTQKSLSLSLGK |
| SEQ ID NO: | Light | DIVMTQSPDSLAVSLGERATINCKSSESVSNDVAWYQQKPGQPPK |
| 543 | chain | LLINYAFHRFTGVPDRFSGSGYGTDFTLTISSLQAEDVAVYYCHQAY |
| | | SSPYTFGQGTKLEIKRTVAAPSVFIFPPSDEQLKSGTASVVCLLNNF |
| | | YPREAKVQWKVDNALQSGNSQESVTEQDSKDSTYSLSSTLTLSKA |
| | | DYEKHKVYACEVTHQGLSSPVTKSFNRGEC |

### Exemplary PD-L1 Inhibitors

In certain embodiments of the dosage regime of the invention, the anti-CD73 antibody molecules and/or anti-ENTPD2 antibody molecules described herein are administered in combination with a PD-L1 inhibitor. The PD-L1 inhibitor may be an antibody, an antigen binding fragment thereof, an immunoadhesin, a fusion protein, or an oligopeptide. In some embodiments, the PD-L1 inhibitor is chosen from FAZ053 (Novartis), Atezolizumab (Genentech/Roche), Avelumab (Merck Serono and Pfizer), Durvalumab (MedImmune/AstraZeneca), or BMS-936559 (Bristol-Myers Squibb).

### Exemplary Anti-PD-L1 Antibody Molecules

In one embodiment of the dosage regime of the invention, the PD-L1 inhibitor is an anti-PD-L1 antibody molecule. In one embodiment, the PD-L1 inhibitor is an anti-PD-L1 antibody molecule as disclosed in US 2016/0108123, published on Apr. 21, 2016, entitled “Antibody Molecules to PD-L1 and Uses Thereof,” incorporated by reference in its entirety.

In one embodiment, the anti-PD-L1 antibody molecule comprises at least one, two, three, four, five or six complementarity determining regions (CDRs) (or collectively all of the CDRs) from a heavy and light chain variable region comprising an amino acid sequence shown in Table 7 (e.g., from the heavy and light chain variable region sequences of BAP058-Clone O or BAP058-Clone N disclosed in Table 7), or encoded by a nucleotide sequence shown in Table 7. In some embodiments, the CDRs are according to the Kabat definition (e.g., as set out in Table 7). In some embodiments, the CDRs are according to the Chothia definition (e.g., as set out in Table 7). In some embodiments, the CDRs are according to the combined CDR definitions of both Kabat and Chothia (e.g., as set out in Table 7). In one embodiment, the combination of Kabat and Chothia CDR of VH CDR1 comprises the amino acid sequence GYTFTSYWMY (SEQ ID NO: 647). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 7, or encoded by a nucleotide sequence shown in Table 7.

In one embodiment, the anti-PD-L1 antibody molecule comprises a heavy chain variable region (VH) comprising a VHCDR1 amino acid sequence of SEQ ID NO: 601, a VHCDR2 amino acid sequence of SEQ ID NO: 602, and a VHCDR3 amino acid sequence of SEQ ID NO: 603; and a light chain variable region (VL) comprising a VLCDR1 amino acid sequence of SEQ ID NO: 609, a VLCDR2 amino acid sequence of SEQ ID NO: 610, and a VLCDR3 amino acid sequence of SEQ ID NO: 611, each disclosed in Table 7.

In one embodiment, the anti-PD-L1 antibody molecule comprises a VH comprising a VHCDR1 encoded by the nucleotide sequence of SEQ ID NO: 628, a VHCDR2 encoded by the nucleotide sequence of SEQ ID NO: 629, and a VHCDR3 encoded by the nucleotide sequence of SEQ ID NO: 630; and a VL comprising a VLCDR1 encoded by the nucleotide sequence of SEQ ID NO: 633, a VLCDR2 encoded by the nucleotide sequence of SEQ ID NO: 634, and a VLCDR3 encoded by the nucleotide sequence of SEQ ID NO: 635, each disclosed in Table 7.

In one embodiment, the anti-PD-L1 antibody molecule comprises a VH comprising the amino acid sequence of SEQ ID NO: 606, or an amino acid sequence having at least about 85%, 90%, 95%, or 99% sequence identity to SEQ ID NO: 606. In one embodiment, the anti-PD-L1 antibody molecule comprises a VL comprising the amino acid sequence of SEQ ID NO: 616, or an amino acid sequence having at least about 85%, 90%, 95%, or 99% sequence identity or higher to SEQ ID NO: 616. In one embodiment, the anti-PD-L1 antibody molecule comprises a VH comprising the amino acid sequence of SEQ ID NO: 620, or an amino acid sequence having at least about 85%, 90%, 95%, or 99% sequence identity to SEQ ID NO: 620. In one embodiment, the anti-PD-L1 antibody molecule comprises a VL comprising the amino acid sequence of SEQ ID NO: 624, or an amino acid sequence having at least about 85%, 90%, 95%, or 99% sequence identity to SEQ ID NO: 624. In one embodiment, the anti-PD-L1 antibody molecule comprises a VH comprising the amino acid sequence of SEQ ID NO: 606 and a VL comprising the amino acid sequence of SEQ ID NO: 616. In one embodiment, the anti-PD-L1 antibody molecule comprises a VH comprising the amino acid sequence of SEQ ID NO: 620 and a VL comprising the amino acid sequence of SEQ ID NO: 624.

In one embodiment, the antibody molecule comprises a VH encoded by the nucleotide sequence of SEQ ID NO: 607, or a nucleotide sequence having at least about 85%, 90%, 95%, or 99% sequence identity to SEQ ID NO: 607. In one embodiment, the antibody molecule comprises a VL encoded by the nucleotide sequence of SEQ ID NO: 617, or a nucleotide sequence having at least about 85%, 90%, 95%, or 99% sequence identity to SEQ ID NO: 617. In one embodiment, the antibody molecule comprises a VH encoded by the nucleotide sequence of SEQ ID NO: 621, or a nucleotide sequence having at least about 85%, 90%, 95%, or 99% sequence identity to SEQ ID NO: 621. In one embodiment, the antibody molecule comprises a VL encoded by the nucleotide sequence of SEQ ID NO: 625, or a nucleotide sequence having at least about 85%, 90%, 95%, or 99% sequence identity to SEQ ID NO: 625. In one embodiment, the antibody molecule comprises a VH encoded by the nucleotide sequence of SEQ ID NO: 607 and a VL encoded by the nucleotide sequence of SEQ ID NO: 617. In one embodiment, the antibody molecule comprises a VH encoded by the nucleotide sequence of SEQ ID NO: 621 and a VL encoded by the nucleotide sequence of SEQ ID NO: 625.

In one embodiment, the anti-PD-L1 antibody molecule comprises a heavy chain comprising the amino acid sequence of SEQ ID NO: 608, or an amino acid sequence having at least about 85%, 90%, 95%, or 99% sequence identity to SEQ ID NO: 608. In one embodiment, the anti-PD-L1 antibody molecule comprises a light chain comprising the amino acid sequence of SEQ ID NO: 618, or an amino acid sequence having at least about 85%, 90%, 95%, or 99% sequence identity to SEQ ID NO: 618. In one embodiment, the anti-PD-L1 antibody molecule comprises a heavy chain comprising the amino acid sequence of SEQ ID NO: 622, or an amino acid sequence having at least about 85%, 90%, 95%, or 99% sequence identity to SEQ ID NO: 622. In one embodiment, the anti-PD-L1 antibody molecule comprises a light chain comprising the amino acid sequence of SEQ ID NO: 626, or an amino acid sequence having at least about 85%, 90%, 95%, or 99% sequence identity to SEQ ID NO: 626. In one embodiment, the anti-PD-L1 antibody molecule comprises a heavy chain comprising the amino acid sequence of SEQ ID NO: 608 and a light chain comprising the amino acid sequence of SEQ ID NO: 618. In one embodiment, the anti-PD-L1 antibody molecule comprises a heavy chain comprising the amino acid sequence of SEQ ID NO: 622 and a light chain comprising the amino acid sequence of SEQ ID NO: 626.

In one embodiment, the antibody molecule comprises a heavy chain encoded by the nucleotide sequence of SEQ ID NO: 615, or a nucleotide sequence having at least about 85%, 90%, 95%, or 99% sequence identity to SEQ ID NO: 615. In one embodiment, the antibody molecule comprises a light chain encoded by the nucleotide sequence of SEQ ID NO: 619, or a nucleotide sequence having at least about 85%, 90%, 95%, or 99% sequence identity to SEQ ID NO: 619. In one embodiment, the antibody molecule comprises a heavy chain encoded by the nucleotide sequence of SEQ ID NO: 623, or a nucleotide sequence having at least about 85%, 90%, 95%, or 99% sequence identity to SEQ ID NO: 623. In one embodiment, the antibody molecule comprises a light chain encoded by the nucleotide sequence of SEQ ID NO: 627, or a nucleotide sequence having at least about 85%, 90%, 95%, or 99% sequence identity to SEQ ID NO: 627. In one embodiment, the antibody molecule comprises a heavy chain encoded by the nucleotide sequence of SEQ ID NO: 615 and a light chain encoded by the nucleotide sequence of SEQ ID NO: 619. In one embodiment, the antibody molecule comprises a heavy chain encoded by the nucleotide sequence of SEQ ID NO: 623 and a light chain encoded by the nucleotide sequence of SEQ ID NO: 627.

The antibody molecules described herein can be made by vectors, host cells, and methods described in US 2016/0108123, incorporated by reference in its entirety.

TABLE 7
Amino acid and nucleotide sequences of exemplary anti-PD-L1 antibody molecules
BAP058-Clone O HC

| | | |
|---|---|---|
| SEQ ID NO: 601 | HCDR1 | SYWMY |
| (Kabat) | | |
| SEQ ID NO: 602 | HCDR2 | RIDPNSGSTKYNEKFKN |
| (Kabat) | | |
| SEQ ID NO: 603 | HCDR3 | DYRKGLYAMDY |
| (Kabat) | | |
| SEQ ID NO: 604 | HCDR1 | GYTFTSY |
| (Chothia) | | |
| SEQ ID NO: 605 | HCDR2 | DPNSGS |
| (Chothia) | | |
| SEQ ID NO: 603 | HCDR3 | DYRKGLYAMDY |
| (Chothia) | | |
| SEQ ID NO: 606 | VH | EVQLVQSGAEVKKPGATVKISCKVSGYTFTSYWMYWVRQ |
| | | ARGQRLEWIGRIDPNSGSTKYNEKFKNRFTISRDNSKNTL |
| | | YLQMNSLRAEDTAVYYCARDYRKGLYAMDYWGQGTTVT |
| | | VSS |
| SEQ ID NO: 607 | DNA VH | GAAGTGCAGCTGGTGCAGTCAGGCGCCGAAGTGAAGA |
| | | AACCCGGCGCTACCGTGAAGATTAGCTGTAAAGTCTCA |
| | | GGCTACACCTTCACTAGCTACTGGATGTACTGGGTCCG |
| | | ACAGGCTAGAGGGCAAAGACTGGAGTGGATCGGTAGA |
| | | ATCGACCCTAATAGCGGCTCTACTAAGTATAACGAGAA |
| | | GTTTAAGAATAGGTTCACTATTAGTAGGGATAACTCTAA |
| | | GAACACCCTGTACCTGCAGATGAATAGCCTGAGAGCCG |
| | | AGGACACCGCCGTCTACTACTGCGCTAGAGACTATAGA |
| | | AAGGGCCTGTACGCTATGGACTACTGGGGTCAAGGCA |
| | | CTACCGTGACCGTGTCTTCA |
| SEQ ID NO: 608 | Heavy | EVQLVQSGAEVKKPGATVKISCKVSGYTFTSYWMYWVRQ |
| | chain | ARGQRLEWIGRIDPNSGSTKYNEKFKNRFTISRDNSKNTL |
| | | YLQMNSLRAEDTAVYYCARDYRKGLYAMDYWGQGTTVT |
| | | VSSASTKGPSVFPLAPCSRSTSESTAALGCLVKDYFPEPV |
| | | TVSWNSGALTSGVHTFPAVLQSSGLYSLSSVVTVPSSSLG |
| | | TKTYTCNVDHKPSNTKVDKRVESKYGPPCPPCPAPEFLG |
| | | GPSVFLFPPKPKDTLMISRTPEVTCVVVDVSQEDPEVQFN |
| | | WYVDGVEVHNAKTKPREEQFNSTYRVVSVLTVLHQDWL |
| | | NGKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLG |
| SEQ ID NO: 615 | DNA | GAAGTGCAGCTGGTGCAGTCAGGCGCCGAAGTGAAGA |
| | heavy | AACCCGGCGCTACCGTGAAGATTAGCTGTAAAGTCTCA |
| | chain | GGCTACACCTTCACTAGCTACTGGATGTACTGGGTCCG |
| | | ACAGGCTAGAGGGCAAAGACTGGAGTGGATCGGTAGA |
| | | ATCGACCCTAATAGCGGCTCTACTAAGTATAACGAGAA |
| | | GTTTAAGAATAGGTTCACTATTAGTAGGGATAACTCTAA |
| | | GAACACCCTGTACCTGCAGATGAATAGCCTGAGAGCCG |
| | | AGGACACCGCCGTCTACTACTGCGCTAGAGACTATAGA |
| | | AAGGGCCTGTACGCTATGGACTACTGGGGTCAAGGCA |
| | | CTACCGTGACCGTGTCTTCAGCTAGCACTAAGGGCCCG |
| | | TCCGTGTTCCCCCTGGCACCTTGTAGCCGGAGCACTAG |
| | | CGAATCCACCGCTGCCCTCGGCTGCCTGGTCAAGGATT |
| | | ACTTCCCGGAGCCCGTGACCGTGTCCTGGAACAGCGG |
| | | AGCCCTGACCTCCGGAGTGCACACCTTCCCCGCTGTG |
| | | CTGCAGAGCTCCGGGCTGTACTCGCTGTCGTCGGTGG |
| | | TCACGGTGCCTTCATCTAGCCTGGGTACCAAGACCTAC |
| | | ACTTGCAACGTGGACCACAAGCCTTCCAACACTAAGGT |
| | | GGACAAGCGCGTCGAATCGAAGTACGGCCCACCGTGC |
| | | CCGCCTTGTCCCGCGCCGGAGTTCCTCGGCGGTCCCT |
| | | CGGTCTTTCTGTTCCCACCGAAGCCCAAGGACACTTTG |
| | | ATGATTTCCCGCACCCCTGAAGTGACATGCGTGGTCGT |
| | | GGACGTGTCACAGGAAGATCCGGAGGTGCAGTTCAATT |
| | | GGTACGTGGATGGCGTCGAGGTGCACAACGCCAAAAC |
| | | CAAGCCGAGGGAGGAGCAGTTCAACTCCACTTACCGC |
| | | GTCGTGTCCGTGCTGACGGTGCTGCATCAGGACTGGC |
| | | TGAACGGGAAGGAGTACAAGTGCAAAGTGTCCAACAAG |
| | | GGACTTCCTAGCTCAATCGAAAAGACCATCTCGAAAGC |
| | | CAAGGGACAGCCCCGGGAACCCCAAGTGTATACCCTG |
| | | CCACCGAGCCAGGAAGAAATGACTAAGAACCAAGTCTC |
| | | ATTGACTTGCCTTGTGAAGGGCTTCTACCCATCGGATAT |
| | | CGCCGTGGAATGGGAGTCCAACGGCCAGCCGGAAAAC |
| | | AACTACAAGACCACCCCTCCGGTGCTGGACTCAGACGG |
| | | ATCCTTCTTCCTCTACTCGCGGCTGACCGTGGATAAGA |
| | | GCAGATGGCAGGAGGGAAATGTGTTCAGCTGTTCTGTG |
| | | ATGCATGAAGCCCTGCACAACCACTACACTCAGAAGTC |
| | | CCTGTCCCTCTCCCTGGGA |

BAP058-Clone O LC

| | | |
|---|---|---|
| SEQ ID NO: 609 | LCDR1 | KASQDVGTAVA |
| (Kabat) | | |
| SEQ ID NO: 610 | LCDR2 | WASTRHT |
| (Kabat) | | |
| SEQ ID NO: | LCDR3 | QQYNSYPLT |
| 611(Kabat) | | |
| SEQ ID NO: 612 | LCDR1 | SQDVGTA |
| (Chothia) | | |
| SEQ ID NO: 613 | LCDR2 | WAS |
| (Chothia) | | |
| SEQ ID NO: 614 | LCDR3 | YNSYPL |
| (Chothia) | | |
| SEQ ID NO: 616 | VL | AIQLTQSPSSLSASVGDRVTITCKASQDVGTAVAWYLQKP |
| | | GQSPQLLIYWASTRHTGVPSRFSGSGSGTDFTFTISSLEA |
| | | EDAATYYCQQYNSYPLTFGQGTKVEIK |
| SEQ ID NO: 617 | DNA VL | GCTATTCAGCTGACTCAGTCACCTAGTAGCCTGAGCGC |
| | | TAGTGTGGGCGATAGAGTGACTATCACCTGTAAAGCCT |
| | | CTCAGGACGTGGGCACCGCCGTGGCCTGGTATCTGCA |
| | | GAAGCCTGGTCAATCACCTCAGCTGCTGATCTACTGGG |
| | | CCTCTACTAGACACACCGGCGTGCCCTCTAGGTTTAGC |
| | | GGTAGCGGTAGTGGCACCGACTTCACCTTCACTATCTC |
| | | TTCACTGGAAGCCGAGGACGCCGCTACCTACTACTGTC |
| | | AGCAGTATAATAGCTACCCCCTGACCTTCGGTCAAGGC |
| | | ACTAAGGTCGAGATTAAG |
| SEQ ID NO: 618 | Light | AIQLTQSPSSLSASVGDRVTITCKASQDVGTAVAWYLQKP |
| | chain | GQSPQLLIYWASTRHTGVPSRFSGSGSGTDFTFTISSLEA |
| | | EDAATYYCQQYNSYPLTFGQGTKVEIKRTVAAPSVFIFPP |
| | | SDEQLKSGTASVVCLLNNFYPREAKVQWKVDNALQSGNS |
| | | QESVTEQDSKDSTYSLSSTLTLSKADYEKHKVYACEVTHQ |
| | | GLSSPVTKSFNRGEC |
| SEQ ID NO: 619 | DNA light | GCTATTCAGCTGACTCAGTCACCTAGTAGCCTGAGCGC |
| | chain | TAGTGTGGGCGATAGAGTGACTATCACCTGTAAAGCCT |
| | | CTCAGGACGTGGGCACCGCCGTGGCCTGGTATCTGCA |
| | | GAAGCCTGGTCAATCACCTCAGCTGCTGATCTACTGGG |
| | | CCTCTACTAGACACACCGGCGTGCCCTCTAGGTTTAGC |
| | | GGTAGCGGTAGTGGCACCGACTTCACCTTCACTATCTC |
| | | TTCACTGGAAGCCGAGGACGCCGCTACCTACTACTGTC |
| | | ACTAAGGTCGAGATTAAGCGTACGGTGGCCGCTCCCAG |
| | | CGTGTTCATCTTCCCCCCCAGCGACGAGCAGCTGAAGA |
| | | GCGGCACCGCCAGCGTGGTGTGCCTGCTGAACAACTT |
| | | CTACCCCCGGGAGGCCAAGGTGCAGTGGAAGGTGGAC |
| | | AACGCCCTGCAGAGCGGCAACAGCCAGGAGAGCGTCA |
| | | CCGAGCAGGACAGCAAGGACTCCACCTACAGCCTGAG |
| | | CAGCACCCTGACCCTGAGCAAGGCCGACTACGAGAAG |
| | | CATAAGGTGTACGCCTGCGAGGTGACCCACCAGGGCC |
| | | TGTCCAGCCCCGTGACCAAGAGCTTCAACAGGGGCGA |
| | | GTGC |

BAP058-Clone N HC

| | | |
|---|---|---|
| SEQ ID NO: 601 | HCDR1 | SYWMY |
| (Kabat) | | |
| SEQ ID NO: 602 | HCDR2 | RIDPNSGSTKYNEKFKN |
| (Kabat) | | |
| SEQ ID NO: 603 | HCDR3 | DYRKGLYAMDY |
| (Kabat) | | |
| SEQ ID NO: 604 | HCDR1 | GYTFTSY |
| (Chothia) | | |
| SEQ ID NO: 605 | HCDR2 | DPNSGS |
| (Chothia) | | |
| SEQ ID NO: 603 | HCDR3 | DYRKGLYAMDY |
| (Chothia) | | |
| SEQ ID NO: 620 | VH | EVQLVQSGAEVKKPGATVKISCKVSGYTFTSYWMYWVRQ |
| | | ATGQGLEWMGRIDPNSGSTKYNEKFKNRVTITADKSTSTA |
| | | YMELSSLRSEDTAVYYCARDYRKGLYAMDYWGQGTTVT |
| | | VSS |
| SEQ ID NO: 621 | DNA VH | GAAGTGCAGCTGGTGCAGTCAGGCGCCGAAGTGAAGA |
| | | AACCCGGCGCTACCGTGAAGATTAGCTGTAAAGTCTCA |
| | | GGCTACACCTTCACTAGCTACTGGATGTACTGGGTCCG |
| | | ACAGGCTACCGGTCAAGGCCTGGAGTGGATGGGTAGA |
| | | ATCGACCCTAATAGCGGCTCTACTAAGTATAACGAGAA |
| | | GTTTAAGAATAGAGTGACTATCACCGCCGATAAGTCTAC |
| | | TAGCACCGCCTATATGGAACTGTCTAGCCTGAGATCAG |
| | | AGGACACCGCCGTCTACTACTGCGCTAGAGACTATAGA |
| | | AAGGGCCTGTACGCTATGGACTACTGGGGTCAAGGCA |
| | | CTACCGTGACCGTGTCTTCA |
| SEQ ID NO: 622 | Heavy | EVQLVQSGAEVKKPGATVKISCKVSGYTFTSYWMYWVRQ |
| | chain | ATGQGLEWMGRIDPNSGSTKYNEKFKNRVTITADKSTSTA |
| | | YMELSSLRSEDTAVYYCARDYRKGLYAMDYWGQGTTVT |
| | | VSSASTKGPSVFPLAPCSRSTSESTAALGCLVKDYFPEPV |
| | | TVSWNSGALTSGVHTFPAVLQSSGLYSLSSVVTVPSSSLG |
| | | TKTYTCNVDHKPSNTKVDKRVESKYGPPCPPCPAPEFLG |
| | | GPSVFLFPPKPKDTLMISRTPEVTCVVVDVSQEDPEVQFN |
| | | WYVDGVEVHNAKTKPREEQFNSTYRVVSVLTVLHQDWL |
| | | NGKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLG |
| SEQ ID NO: 623 | DNA | GAAGTGCAGCTGGTGCAGTCAGGCGCCGAAGTGAAGA |
| | heavy | AACCCGGCGCTACCGTGAAGATTAGCTGTAAAGTCTCA |
| | chain | GGCTACACCTTCACTAGCTACTGGATGTACTGGGTCCG |
| | | ACAGGCTACCGGTCAAGGCCTGGAGTGGATGGGTAGA |
| | | ATCGACCCTAATAGCGGCTCTACTAAGTATAACGAGAA |
| | | GTTTAAGAATAGAGTGACTATCACCGCCGATAAGTCTAC |
| | | TAGCACCGCCTATATGGAACTGTCTAGCCTGAGATCAG |
| | | AGGACACCGCCGTCTACTACTGCGCTAGAGACTATAGA |
| | | AAGGGCCTGTACGCTATGGACTACTGGGGTCAAGGCA |
| | | CTACCGTGACCGTGTCTTCAGCTAGCACTAAGGGCCCG |
| | | TCCGTGTTCCCCCTGGCACCTTGTAGCCGGAGCACTAG |
| | | CGAATCCACCGCTGCCCTCGGCTGCCTGGTCAAGGATT |
| | | ACTTCCCGGAGCCCGTGACCGTGTCCTGGAACAGCGG |
| | | AGCCCTGACCTCCGGAGTGCACACCTTCCCCGCTGTG |
| | | CTGCAGAGCTCCGGGCTGTACTCGCTGTCGTCGGTGG |
| | | TCACGGTGCCTTCATCTAGCCTGGGTACCAAGACCTAC |
| | | ACTTGCAACGTGGACCACAAGCCTTCCAACACTAAGGT |
| | | GGACAAGCGCGTCGAATCGAAGTACGGCCCACCGTGC |
| | | CCGCCTTGTCCCGCGCCGGAGTTCCTCGGCGGTCCCT |
| | | CGGTCTTTCTGTTCCCACCGAAGCCCAAGGACACTTTG |
| | | ATGATTTCCCGCACCCCTGAAGTGACATGCGTGGTCGT |
| | | GGACGTGTCACAGGAAGATCCGGAGGTGCAGTTCAATT |
| | | GGTACGTGGATGGCGTCGAGGTGCACAACGCCAAAAC |
| | | CAAGCCGAGGGAGGAGCAGTTCAACTCCACTTACCGC |
| | | GTCGTGTCCGTGCTGACGGTGCTGCATCAGGACTGGC |
| | | TGAACGGGAAGGAGTACAAGTGCAAAGTGTCCAACAAG |
| | | GGACTTCCTAGCTCAATCGAAAAGACCATCTCGAAAGC |
| | | CAAGGGACAGCCCCGGGAACCCCAAGTGTATACCCTG |
| | | CCACCGAGCCAGGAAGAAATGACTAAGAACCAAGTCTC |
| | | ATTGACTTGCCTTGTGAAGGGCTTCTACCCATCGGATAT |
| | | CGCCGTGGAATGGGAGTCCAACGGCCAGCCGGAAAAC |
| | | AACTACAAGACCACCCCTCCGGTGCTGGACTCAGACGG |
| | | ATCCTTCTTCCTCTACTCGCGGCTGACCGTGGATAAGA |
| | | GCAGATGGCAGGAGGGAAATGTGTTCAGCTGTTCTGTG |
| | | ATGCATGAAGCCCTGCACAACCACTACACTCAGAAGTC |
| | | CCTGTCCCTCTCCCTGGGA |

BAP058-Clone N LC

| | | |
|---|---|---|
| SEQ ID NO: 609 | LCDR1 | KASQDVGTAVA |
| (Kabat) | | |
| SEQ ID NO: 610 | LCDR2 | WASTRHT |
| (Kabat) | | |
| SEQ ID NO: | LCDR3 | QQYNSYPLT |
| 611(Kabat) | | |
| SEQ ID NO: 612 | LCDR1 | SQDVGTA |
| (Chothia) | | |
| SEQ ID NO: 613 | LCDR2 | WAS |
| (Chothia) | | |
| SEQ ID NO: 614 | LCDR3 | YNSYPL |
| (Chothia) | | |
| SEQ ID NO: 624 | VL | DVVMTQSPLSLPVTLGQPASISCKASQDVGTAVAWYQQK |
| | | PGQAPRLLIYWASTRHTGVPSRFSGSGSGTEFTLTISSLQ |
| | | PDDFATYYCQQYNSYPLTFGQGTKVEIK |
| SEQ ID NO: 625 | DNA VL | GACGTCGTGATGACTCAGTCACCCCTGAGCCTGCCCGT |
| | | GACCCTGGGGCAGCCCGCCTCTATTAGCTGTAAAGCCT |
| | | CTCAGGACGTGGGCACCGCCGTGGCCTGGTATCAGCA |
| | | GAAGCCAGGGCAAGCCCCTAGACTGCTGATCTACTGG |
| | | GCCTCTACTAGACACACCGGCGTGCCCTCTAGGTTTAG |
| | | CGGTAGCGGTAGTGGCACCGAGTTCACCCTGACTATCT |
| | | CTTCACTGCAGCCCGACGACTTCGCTACCTACTACTGT |
| | | CAGCAGTATAATAGCTACCCCCTGACCTTCGGTCAAGG |
| | | CACTAAGGTCGAGATTAAG |
| SEQ ID NO: 626 | Light | DVVMTQSPLSLPVTLGQPASISCKASQDVGTAVAWYQQK |
| | chain | PGQAPRLLIYWASTRHTGVPSRFSGSGSGTEFTLTISSLQ |
| | | PDDFATYYCQQYNSYPLTFGQGTKVEIKRTVAAPSVFIFP |
| | | PSDEQLKSGTASVVCLLNNFYPREAKVQWKVDNALQSGN |
| | | SQESVTEQDSKDSTYSLSSTLTLSKADYEKHKVYACEVTH |
| | | QGLSSPVTKSFNRGEC |
| SEQ ID NO: 627 | DNA light | GACGTCGTGATGACTCAGTCACCCCTGAGCCTGCCCGT |
| | chain | GACCCTGGGGCAGCCCGCCTCTATTAGCTGTAAAGCCT |
| | | CTCAGGACGTGGGCACCGCCGTGGCCTGGTATCAGCA |
| | | GAAGCCAGGGCAAGCCCCTAGACTGCTGATCTACTGG |
| | | GCCTCTACTAGACACACCGGCGTGCCCTCTAGGTTTAG |
| | | CGGTAGCGGTAGTGGCACCGAGTTCACCCTGACTATCT |
| | | CTTCACTGCAGCCCGACGACTTCGCTACCTACTACTGT |
| | | CAGCAGTATAATAGCTACCCCCTGACCTTCGGTCAAGG |
| | | CACTAAGGTCGAGATTAAGCGTACGGTGGCCGCTCCCA |
| | | GCGTGTTCATCTTCCCCCCCAGCGACGAGCAGCTGAAG |
| | | AGCGGCACCGCCAGCGTGGTGTGCCTGCTGAACAACT |
| | | TCTACCCCCGGGAGGCCAAGGTGCAGTGGAAGGTGGA |
| | | CAACGCCCTGCAGAGCGGCAACAGCCAGGAGAGCGTC |
| | | ACCGAGCAGGACAGCAAGGACTCCACCTACAGCCTGA |
| | | GCAGCACCCTGACCCTGAGCAAGGCCGACTACGAGAA |
| | | GCATAAGGTGTACGCCTGCGAGGTGACCCACCAGGGC |
| | | CTGTCCAGCCCCGTGACCAAGAGCTTCAACAGGGGCG |
| | | AGTGC |

BAP058-Clone O HC

| | | |
|---|---|---|
| SEQ ID NO: 628 | HCDR1 | agctactggatgtac |
| (Kabat) | | |
| SEQ ID NO: 629 | HCDR2 | agaatcgaccctaatagcggctctactaagtataacgagaagtttaagaat |
| (Kabat) | | |
| SEQ ID NO: 630 | HCDR3 | gactatagaaagggcctgtacgctatggactac |
| (Kabat) | | |
| SEQ ID NO: 631 | HCDR1 | ggctacaccttcactagctac |
| (Chothia) | | |
| SEQ ID NO: 632 | HCDR2 | gaccctaatagcggctct |
| (Chothia) | | |
| SEQ ID NO: 630 | HCDR3 | gactatagaaagggcctgtacgctatggactac |
| (Chothia) | | |

BAP058-Clone O LC

| | | |
|---|---|---|
| SEQ ID NO: 633 | LCDR1 | aaagcctctcaggacgtgggcaccgccgtggcc |
| (Kabat) | | |
| SEQ ID NO: 634 | LCDR2 | tgggcctctactagacacacc |
| (Kabat) | | |
| SEQ ID NO: 635 | LCDR3 | cagcagtataatagctaccccctgacc |
| (Kabat) | | |
| SEQ ID NO: 636 | LCDR1 | tctcaggacgtgggcaccgcc |
| (Chothia) | | |
| SEQ ID NO: 637 | LCDR2 | tgggcctct |
| (Chothia) | | |
| SEQ ID NO: 638 | LCDR3 | tataatagctaccccctg |
| (Chothia) | | |

BAP058-Clone N HC

| | | |
|---|---|---|
| SEQ ID NO: 628 | HCDR1 | agctactggatgtac |
| (Kabat) | | |
| SEQ ID NO: 629 | HCDR2 | agaatcgaccctaatagcggctctactaagtataacgagaagtttaagaat |
| (Kabat) | | |
| SEQ ID NO: 630 | HCDR3 | gactatagaaagggcctgtacgctatggactac |
| (Kabat) | | |
| SEQ ID NO: 631 | HCDR1 | ggctacaccttcactagctac |
| (Chothia) | | |
| SEQ ID NO: 632 | HCDR2 | gaccctaatagcggctct |
| (Chothia) | | |
| SEQ ID NO: 630 | HCDR3 | gactatagaaagggcctgtacgctatggactac |
| (Chothia) | | |

BAP058-Clone N LC

| | | |
|---|---|---|
| SEQ ID NO: 633 | LCDR1 | aaagcctctcaggacgtgggcaccgccgtggcc |
| (Kabat) | | |
| SEQ ID NO: 634 | LCDR2 | tgggcctctactagacacacc |
| (Kabat) | | |
| SEQ ID NO: 635 | LCDR3 | cagcagtataatagctaccccctgacc |
| (Kabat) | | |
| SEQ ID NO: 636 | LCDR1 | tctcaggacgtgggcaccgcc |
| (Chothia) | | |
| SEQ ID NO: 637 | LCDR2 | tgggcctct |
| (Chothia) | | |
| SEQ ID NO: 638 | LCDR3 | tataatagctaccccctg |
| (Chothia) | | |

### Other Exemplary PD-L1 Inhibitors

In one embodiment, the anti-PD-L1 antibody molecule is Atezolizumab (Genentech/Roche), also known as MPDL3280A, RG7446, R05541267, YW243.55.S70, or TECENTRIQ™. Atezolizumab and other anti-PD-L1 antibodies are disclosed in U.S. Pat. No. 8,217,149, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Atezolizuma, e.g., as disclosed in Table 8.

In one embodiment, the anti-PD-L1 antibody molecule is Avelumab (Merck Serono and Pfizer), also known as MSB0010718C. Avelumab and other anti-PD-L1 antibodies are disclosed in WO 2013/079174, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Avelumab, e.g., as disclosed in Table 8.

In one embodiment, the anti-PD-L1 antibody molecule is Durvalumab (MedImmune/AstraZeneca), also known as MED14736. Durvalumab and other anti-PD-L1 antibodies are disclosed in U.S. Pat. No. 8,779,108, incorporated by reference in its entirety. In one embodiment, the anti-PD-L1 antibody molecule comprises one or more of the CDR sequences (or collectively all of the CDR sequences), the heavy chain or light chain variable region sequence, or the heavy chain or light chain sequence of Durvalumab, e.g., as disclosed in Table 8.

In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entirety. In one embodiment, the anti-PD-L1 antibody molecule comprises one or more of the CDR sequences (or collectively all of the CDR sequences), the heavy chain or light chain variable region sequence, or the heavy chain or light chain sequence of BMS-936559, e.g., as disclosed in Table 8.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entirety.

In one embodiment, the anti-PD-L1 antibody is an antibody that competes for binding with, and/or binds to the same epitope on PD-L1 as, one of the anti-PD-L1 antibodies described herein.

TABLE 8
Amino acid sequences of other exemplary anti-PD-L1 antibody molecules
Atezolizumab

| | | |
|---|---|---|
| SEQ ID NO: | Heavy | EVQLVESGGGLVQPGGSLRLSCAASGFTFSDSWIHWVRQAPGKGL |
| 639 | chain | EWVAWISPYGGSTYYADSVKGRFTISADTSKNTAYLQMNSLRAEDT |
| | | AVYYCARRHWPGGFDYWGQGTLVTVSSASTKGPSVFPLAPSSKST |
| | | SGGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKF |
| | | NWYVDGVEVHNAKTKPREEQYASTYRVVSVLTVLHQDWLNGKEYK |
| | | CKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDK |
| | | SRWQQGNVFSCSVMHEALHNHYTQKSLSLSPGK |
| SEQ ID NO: | Light | DIQMTQSPSSLSASVGDRVTITCRASQDVSTAVAWYQQKPGKAPKL |
| 640 | chain | LIYSASFLYSGVPSRFSGSGSGTDFTLTISSLQPEDFATYYCQQYLYH |
| | | PATFGQGTKVEIKRTVAAPSVFIFPPSDEQLKSGTASVVCLLNNFYP |
| | | REAKVQWKVDNALQSGNSQESVTEQDSKDSTYSLSSTLTLSKADYE |
| | | KHKVYACEVTHQGLSSPVTKSFNRGEC |

Avelumab

| | | |
|---|---|---|
| SEQ ID NO: | Heavy | EVQLLESGGGLVQPGGSLRLSCAASGFTFSSYIMMWVRQAPGKGL |
| 641 | chain | EWVSSIYPSGGITFYADTVKGRFTISRDNSKNTLYLQMNSLRAEDTA |
| | | VYYCARIKLGTVTTVDYWGQGTLVTVSSASTKGPSVFPLAPSSKSTS |
| | | GGTAALGCLVKDYFPEPVTVSWNSGALTSGVHTFPAVLQSSGLYSL |
| | | SSVVVTVPSSSLGTQTYICNVNHKPSNTKVDKKVEPKSCDKTHTCPP |
| | | CPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYK |
| | | CKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDK |
| | | SRWQQGNVFSCSVMHEALHNHYTQKSLSLSPGK |
| SEQ ID NO: | Light | QSALTQPASVSGSPGQSITISCTGTSSDVGGYNYVSWYQQHPGKAP |
| 642 | chain | KLMIYDVSNRPSGVSNRFSGSKSGNTASLTISGLQAEDEADYYCSSY |
| | | TSSSTRVFGTGTKVTVLGQPKANPTVTLFPPSSEELQANKATLVCLIS |
| | | DFYPGAVTVAWKADGSPVKAGVETTKPSKQSNNKYAASSYLSLTPE |
| | | QWKSHRSYSCQVTHEGSTVEKTVAPTECS |

Durvalumab

| | | |
|---|---|---|
| SEQ ID NO: | Heavy | EVQLVESGGGLVQPGGSLRLSCAASGFTFSRYWMSWVRQAPGKG |
| 643 | chain | LEWVANIKQDGSEKYYVDSVKGRFTISRDNAKNSLYLQMNSLRAED |
| | | TAVYYCAREGGWFGELAFDYWGQGTLVTVSSASTKGPSVFPLAPS |
| | | SKSTSGGTAALGCLVKDYFPEPVTVSWNSGALTSGVHTFPAVLQSS |
| | | GLYSLSSVVTVPSSSLGTQTYICNVNHKPSNTKVDKRVEPKSCDKTH |
| | | TCPPCPAPEFEGGPSVFLFPPKPKDTLMISRTPEVTCVVVDVSHEDP |
| | | EVKFNWYVDGVEVHNAKTKPREEQYNSTYRVVSVLTVLHQDWLNG |
| | | KEYKCKVSNKALPASIEKTISKAKGQPREPQVYTLPPSREEMTKNQV |
| | | SLTCLVKGFYPSDIAVEWESNGQPENNYKTTPPVLDSDGSFFLYSKL |
| | | TVDKSRWQQGNVFSCSVMHEALHNHYTQKSLSLSPGK |
| SEQ ID NO: | Light | EIVLTQSPGTLSLSPGERATLSCRASQRVSSSYLAWYQQKPGQAPR |
| 644 | chain | LLIYDASSRATGIPDRFSGSGSGTDFTLTISRLEPEDFAVYYCQQYGS |
| | | LPWTFGQGTKVEIKRTVAAPSVFIFPPSDEQLKSGTASVVCLLNNFY |
| | | PREAKVQWKVDNALQSGNSQESVTEQDSKDSTYSLSSTLTLSKADY |
| | | EKHKVYACEVTHQGLSSPVTKSFNRGEC |

BMS-936559

| | | |
|---|---|---|
| SEQ ID NO: | VH | QVQLVQSGAEVKKPGSSVKVSCKTSGDTFSTYAISWVRQAPGQGL |
| 645 | | EWMGGIIPIFGKAHYAQKFQGRVTITADESTSTAYMELSSLRSEDTA |
| | | VYFCARKFHFVSGSPFGMDVWGQGTTVTVSS |
| SEQ ID NO: | VL | EIVLTQSPATLSLSPGERATLSCRASQSVSSYLAWYQQKPGQAPRLL |
| 646 | | IYDASNRATGIPARFSGSGSGTDFTLTISSLEPEDFAVYYCQQRSNW |
| | | PTFGQGTKVEIK |

### Exemplary ENTPD2 Inhibitors

In one embodiment of the dosage regime according to the invention, the additional therapeutic agent is an ENTPD2 agent. In an embodiment the ENTPD2 agent is an ENTPD2 inhibitor.

The ENTPD2 inhibitor may be an antibody, an immunoadhesin, a fusion protein, or an oligopeptide. In one embodiment, the ENTPD2 agent or ENTPD2 inhibitor is an ENTPD2 antibody molecule. In one embodiment of the dosage regime of the invention, the ENTPD2 inhibitor is an ENTPD2 antibody molecule as described in WO2019229658, published on 5 Dec. 2019, entitled “ENTPD2 Antibodies, Combination Therapies, and Methods of Using the Antibodies and Combination Therapies”, which is incorporated by reference in its entirety.

In one embodiment of the dosage regime according to the invention, the additional therapeutic agent is an anti-ENTPD2 antibody molecule which comprises at least one, two, three, four, five or six complementarity determining regions (CDRs) (or collectively all of the CDRs) from a heavy and light chain variable region comprising an amino acid sequence shown in Table 9 (e.g., from the heavy and light chain variable region sequences of ANTI-HUMAN ENTPD2 MAB1, ANTI-HUMAN ENTPD2 MAB2, ANTI-HUMAN ENTPD2 MAB3, ANTI-HUMAN ENTPD2 MAB4 or ANTI-HUMAN ENTPD2 MAB5 disclosed in Table 9), or encoded by a nucleotide sequence shown in Table 9. In some embodiments, the CDRs are according to the Kabat definition (e.g., as set out in Table 9). In some embodiments, the CDRs are according to the Chothia definition (e.g., as set out in Table 9). In some embodiments, the CDRs are according to the combined CDR definitions of both Kabat and Chothia (e.g., as set out in Table 9). In one embodiment, one or more of the CDRs (or collectively all of the CDRs) have one, two, three, four, five, six or more changes, e.g., amino acid substitutions (e.g., conservative amino acid substitutions) or deletions, relative to an amino acid sequence shown in Table 9, or encoded by a nucleotide sequence shown in Table 9.

In one embodiment, the anti-ENTPD2 antibody molecule comprises a heavy chain variable region (VH) comprising a VHCDR1 amino acid sequence of SEQ ID NO: 401, a VHCDR2 amino acid sequence of SEQ ID NO: 402, and a VHCDR3 amino acid sequence of SEQ ID NO: 403; and a light chain variable region (VL) comprising a VLCDR1 amino acid sequence of SEQ ID NO: 414, a VLCDR2 amino acid sequence of SEQ ID NO: 415, and a VLCDR3 amino acid sequence of SEQ ID NO: 416 each disclosed in Table 9.

In one embodiment, the anti-ENTPD2 antibody molecule comprises a VH comprising the amino acid sequence of SEQ ID NO: 410, or an amino acid sequence having at least about 85%, 90%, 95%, or 99% sequence identity or higher to SEQ ID NO: 410. In one embodiment, the anti-ENTPD2 antibody molecule comprises a VL comprising the amino acid sequence of SEQ ID NO: 421, or an amino acid sequence having at least about 85%, 90%, 95%, or 99% sequence identity or higher to SEQ ID NO: 421. In one embodiment, the anti-ENTPD2 antibody molecule comprises a VH comprising the amino acid sequence of SEQ ID NO: 410 and a VL comprising the amino acid sequence of SEQ ID NO: 421.

In one embodiment, the anti-ENTPD2 antibody molecule comprises a heavy chain comprising the amino acid sequence of SEQ ID NO: 412, or an amino acid sequence having at least about 85%, 90%, 95%, or 99% sequence identity or higher to SEQ ID NO: 412. In one embodiment, the anti-ENTPD2 antibody molecule comprises a light chain comprising the amino acid sequence of SEQ ID NO: 423, or an amino acid sequence having at least about 85%, 90%, 95%, or 99% sequence identity or higher to SEQ ID NO: 423. In one embodiment, the anti-ENTPD2 antibody molecule comprises a heavy chain comprising the amino acid sequence of SEQ ID NO: 412 and a light chain comprising the amino acid sequence of SEQ ID NO: 423.

The antibody molecules described herein can be made by vectors, host cells, and methods described in WO2019229658, incorporated by reference in its entirety.

### Exemplary TGF-β Inhibitors

In certain embodiments of the dosage regime according to the invention, the additional therapeutic agent is a transforming growth factor beta (TGF-β) inhibitor. In some embodiments, the combination is used to treat a cancer, e.g., a cancer described herein.

TGF-β belongs to a large family of structurally-related cytokines including, e.g., bone morphogenetic proteins (BMPs), growth and differentiation factors, activins and inhibins. In some embodiments, the TGF-β inhibitors described herein can bind and/or inhibit one or more isoforms of TGF-β (e.g., one, two, or all of TGF-β 1, TGF-β 2, or TGF-β 3).

In some embodiments, the TGF-β inhibitor is fresolimumab (CAS Registry Number: 948564-73-6). Fresolimumab is also known as GC1008. Fresolimumab is a human monoclonal antibody that binds to and inhibits TGF-beta isoforms 1, 2 and 3.

The heavy chain of fresolimumab has the amino acid sequence of:

(SEQ ID NO: 687)
QVQLVQSGAEVKKPGSSVKVSCKASGYTFSSNVISWVRQAPGQGLEWMGG
VIPIVDIANYAQRFKGRVTITADESTSTTYMELSSLRSEDTAVYYCASTL
GLVLDAMDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPSCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK.

The light chain of fresolimumab has the amino acid sequence of:

(SEQ ID NO: 688)
ETVLTQSPGTLSLSPGERATLSCRASQSLGSSYLAWYQQKPGQAPRLLIY
GASSRAPGIPDRFSGSGSGTDFTLTISRLEPEDFAVYYCQQYADSPITFG
QGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

Fresolimumab is disclosed, e.g., in WO 2006/086469, U.S. Pat. Nos. 8,383,780, and 8,591,901.
In some embodiments, the TGF-β inhibitor is XOMA 089. XOMA 089 is also known as XPA.42.089. XOMA 089 is a fully human monoclonal antibody that binds and neutralizes TGF-beta 1 and 2 ligands.

The heavy chain variable region of XOMA 089 has the amino acid sequence of:

(SEQ ID NO: 689)
QVQLVQSGAEVKKPGSSVKVSCKASGGTFSSYAISWVRQAPGQGLEWMGG
IIPIFGTANYAQKFQGRVTITADESTSTAYMELSSLRSEDTAVYYCARGL
WEVRALPSVYWGQGTLVTVSS
(disclosed as SEQ ID NO: 6 in WO 2012/167143).

The light chain variable region of XOMA 089 has the amino acid sequence of:

(SEQ ID NO: 690)
SYELTQPPSVSVAPGQTARITCGANDIGSKSVHWYQQKAGQAPVLVVSED
IIRPSGIPERISGSNSGNTATLTISRVEAGDEADYYCQVWDRDSDQYVFG
TGTKVTVLG
(disclosed as SEQ ID NO: 8 in WO 2012/167143).

In certain embodiments, the dosage regime includes an inhibitor of ENTPD2 (e.g., an anti-ENTPD2 antibody molecule described herein) and an inhibitor of CD73 (e.g., an anti-CD73 antibody molecule described herein) and a TGF-β inhibitor (e.g., a TGF-β inhibitor described herein).

In one embodiment, the dosage regime includes a TGF-β inhibitor, XOMA 089, or a compound disclosed in PCT Publication No. WO 2012/167143, and an inhibitor of ENTPD2 (e.g., an anti-ENTPD2 antibody described herein) and an inhibitor of CD73 (e.g., an anti-CD73 antibody described herein).

In one embodiment, the TGF-β inhibitor, XOMA 089, or a compound disclosed in PCT Publication No. WO 2012/167143, is administered in a dosage regime with an inhibitor of ENTPD2 (e.g., an anti-ENTPD2 antibody described herein) and an inhibitor of CD73 (e.g., an anti-CD73 antibody molecule) to treat a cancer, wherein the cancer is MSS colorectal cancer (CRC), cholangiocarcinoma (intrahepatic or extrahepatic), pancreatic cancer, esophageal cancer, esophageal gastric junction (EGJ) cancer, or gastric cancer.

### Triptans

In an embodiment of the antibody for use according to the invention or method according to the invention, a triptan is administered with one or more doses of the antibody molecule that binds to human CD73 or human ENTPD2. The triptan may be administered for example prior to, or concurrently with, the anti-CD73 antibody or anti-ENTPD2 antibody. Triptans are a family of tryptamine based drugs that act as agonists for serotonin 5-HT_{IB} and 5-HT_{ID} receptors at blood vessels and nerve endings in the brain. In an embodiment the triptan is selected from Almotriptan (Axert), Eletriptan (Relpax), Frovatriptan (Frova), Naratriptan (Amerge), Rizatriptan (Maxalt), Sumatriptan (Imitrex), Zolmitriptan (Zomig), Lasmiditan (Reyvow), which may be combined with an additional agent, such as Sumatriptan combined with naproxen sodium (Treximet). In a preferred embodiment the triptan is Sumatriptan or Zolmitriptan. The triptan may for example be administered on Day 1, prior to the anti-CD73 antibody infusion on that day. The triptan may be used according to the standard dosage amounts for the particular triptan, e.g. sumatriptan tablet 25 mg, 50 mg, or 100 mg.

### EXAMPLES

The Examples below are set forth to aid in the understanding of the inventions but are not intended to, and should not be construed to limit its scope in any way.

### Example 1: Generation and Characterization of Anti-CD73 Antibodies

Selection and Optimization of Anti-CD73 Antibodies from Synthetic Yeast Antibody Libraries

Anti-CD73 monoclonal antibodies representing five distinct epitope bins were selected from eight naïve human synthetic yeast libraries using methods described below.

### Materials and Methods

Antigens were biotinylated using the EZ-Link Sulfo-NHS-Biotinylation Kit from Pierce. Goat F(ab′)₂ anti-human kappa-FITC (LC-FITC), ExtrAvidin-PE (EA-PE) and Streptavidin-AF633 (SA-633) were obtained from Southern Biotech, Sigma, and Molecular Probes, respectively. Streptavidin MicroBeads and MACS LC separation columns were purchased from Miltenyi Biotec. Goat anti-human IgG-PE (Human-PE) was obtained from Southern Biotech.

### Primary Discovery

Eight naïve human synthetic yeast libraries each of ˜10⁹ diversity were propagated as previously described (see, e.g., Y. Xu et al, Addressing polyspecificity of antibodies selected from an in vitro yeast presentation system: a FACS-based, high-throughput selection and analytical tool. PEDS 26.10, 663-70 (2013); WO2009036379; WO2010105256; and WO2012009568, incorporated by reference herein in their entireties). For the first two rounds of selection, a magnetic bead sorting technique utilizing the Miltenyi MACS system was performed, as previously described (see, e.g., Siegel et al, High efficiency recovery and epitope-specific sorting of an scFv yeast display library. J Immunol Methods 286(1-2), 141-153 (2004), herein incorporated by reference in its entirety). Briefly, yeast cells (˜10¹⁰ cells/library) were incubated with 3 ml of 100 nM biotinylated antigen for 30 min at 30° C. in wash buffer (phosphate-buffered saline (PBS)/01% bovine serum albumin (BSA)). After washing once with 40 ml ice-cold wash buffer, the cell pellet was resuspended in 20 mL wash buffer, and Streptavidin MicroBeads (500 μl) were added to the yeast and incubated for 15 min at 4° C. Next, the yeast cells were pelleted, resuspended in 20 mL wash buffer, and loaded onto a Miltenyi LS column. After the 20 mL was loaded, the column was washed 3 times with 3 ml wash buffer. The column was then removed from the magnetic field, and the yeast cells were eluted with 5 mL of growth media and then grown overnight. The following rounds of selection were performed using flow cytometry. Approximately 2×10⁷ yeast cells were pelleted, washed three times with wash buffer, and incubated at 30° C. with either decreasing concentrations of biotinylated antigen (100 to 1 nM) under equilibrium conditions, 30 nM biotinylated antigens of different species in order to obtain species cross-reactivity, or with a poly-specificity depletion reagent (PSR) to remove non-specific antibodies from the selection. For the PSR depletion, the libraries were incubated with a 1:10 dilution of biotinylated PSR reagent as previously described (see, e.g., Y. Xu et al, Addressing polyspecificity of antibodies selected from an in vitro yeast presentation system: a FACS-based, high-throughput selection and analytical tool. PEDS 26.10, 663-70 (2013), herein incorporated by reference in its entirety). Yeast cells were then washed twice with wash buffer and stained with LC-FITC (diluted 1:100) and either SA-633 (diluted 1:500) or EAPE (diluted 1:50) secondary reagents for 15 min at 4° C. After washing twice with wash buffer, the cell pellets were resuspended in 0.3 mL wash buffer and transferred to strainer-capped sort tubes. Sorting was performed using a FACS ARIA sorter (BD Biosciences) and sort gates were determined to select for antibodies with desired characteristics. Selection rounds were repeated until a population with all of the desired characteristics was obtained. After the final round of sorting, yeast cells were plated and individual colonies were picked for characterization.

Light chain diversification protocol was used during the primary discovery phase for further discovery and improvement of antibodies.

Light chain batch diversification protocol: Heavy chain plasmids from a naïve selection output were extracted from the yeast via smash and grab, propagated in and subsequently purified from E. coli, and transformed into a light chain library with a diversity of 5×10⁶. Selections were performed with one round of MACS and four rounds of FACS employing the same conditions as the naïve discovery.

### Antibody Optimization

Optimization of antibodies was performed by introducing diversities into the heavy chain and light chain variable regions as described below.

CDRH1 and CDRH2 selection: The CDRH3 of a single antibody was recombined into a premade library with CDRH1 and CDRH2 variants of a diversity of 1×10⁸ and selections were performed with one round of MACS and four rounds of FACS as described in the naïve discovery. In the different FACS rounds the libraries were looked at for PSR binding, species cross-reactivity, and affinity pressure by titration or parental Fab pre-complexing, and sorting was performed in order to obtain a population with the desired characteristics.

### Antibody Production and Purification

Yeast clones were grown to saturation and then induced for 48 h at 30° C. with shaking. After induction, yeast cells were pelleted and the supernatants were harvested for purification. IgGs were purified using a Protein A column and eluted with acetic acid, pH 2.0. Fab fragments were generated by papain digestion and purified over KappaSelect (GE Healthcare LifeSciences).

### ForteBio K_{D} Measurements

ForteBio affinity measurements were performed on an Octet RED384 generally as previously described (see, e.g., Estep et al, High throughput solution-based measurement of antibody-antigen affinity and epitope binning. Mabs 5(2), 270-278 (2013), herein incorporated by reference in its entirety). Briefly, ForteBio affinity measurements were performed by loading IgGs on-line onto AHQ sensors. Sensors were equilibrated off-line in assay buffer for 30 min and then monitored on-line for 60 seconds for baseline establishment. Sensors with loaded IgGs were exposed to 100 nM antigen for 3 minutes, and afterwards were transferred to assay buffer for 3 min for off-rate measurement. All kinetics were analyzed using the 1:1 binding model. Antigens used were:
Human CD73-His: Recombinant Human 5′-Nucleotidase/CD73 Protein, CF from R&D Systems Cat: 5795-EN
Mouse CD73-His: Recombinant Mouse 5′-Nucleotidase/CD73 Protein, CF from R&D Systems Cat: 4488-EN
Cynomolgus CD73-His: Cynomolgus CD73/NT5E Protein (His Tag) from Sino Biological Cat: 90192-C08H-50

### ForteBio Epitope Binning/Ligand Blocking

Epitope binning/ligand blocking was performed using a standard sandwich format cross-blocking assay. Control anti-target IgG was loaded onto AHQ sensors and unoccupied Fc-binding sites on the sensor were blocked with an irrelevant human IgG1 antibody. The sensors were then exposed to 100 nM target antigen followed by a second anti-target antibody or ligand. Additional binding by the second antibody or ligand after antigen association indicates an unoccupied epitope (non-competitor), while no binding indicates epitope blocking (competitor or ligand blocking).

### MSD-SET Kinetic Assay

Equilibrium affinity measurements performed as previously described (Estep et al., 2013). Solution equilibrium titrations (SET) were performed in PBS+0.1% IgG-Free BSA (PBSF) with antigen held constant at 10-100 pM and incubated with 3- to 5-fold serial dilutions of antibody starting at 5-100 nM (experimental condition is sample dependent). Antibodies (20 nM in PBS) were coated onto standard bind MSD-ECL plates overnight at 4° C. or at room temperature for 30 min. Plates were then blocked for 30 min with shaking at 700 rpm, followed by three washes with wash buffer (PBSF+0.05% Tween 20). SET samples were applied and incubated on the plates for 150s with shaking at 700 rpm followed by one wash. Antigen captured on a plate was detected with 250 ng/mL sulfotag-labeled streptavidin in PBSF by incubation on the plate for 3 min. The plates were washed three times with wash buffer and then read on the MSD Sector Imager 2400 instrument using 1× Read Buffer T with surfactant. The percent free antigen was plotted as a function of titrated antibody in Prism and fit to a quadratic equation to extract the K_{D}. To improve throughput, liquid handling robots were used throughout MSD-SET experiments, including SET sample preparation.

### Cell Binding Analysis

Approximately 100,000 cells overexpressing the antigen were washed with wash buffer and incubated with 100 μl 100 nM IgG for 5 minutes at room temperature. Cells were then washed twice with wash buffer and incubated with 100 μl of 1:100 Human-PE for 15 minutes on ice. Cells were then washed twice with wash buffer and analyzed on a FACS Canto II analyzer (BD Biosciences).

### Results

Yeast cells expressing a library of human antibodies on their surface were screened for binding to human CD73. Two antibodies from epitope bin 4, 918 and 930, bound well to CD73 and inhibited the enzymatic activity of CD73 (data not shown). These two antibodies were subjected to affinity maturation which produced two lineages of related antibodies, referred to as lineage 1 and lineage 3, respectively (Table 10). These anti-CD73 antibodies were expressed in three different formats: IgG1 antibodies (referred to as .C constructs, e.g., 350.C), IgG4 antibodies comprising an S228P mutation in the Fc region (IgG4 S228P, referred to as .A constructs, e.g., 350.A), or IgG4 antibodies comprising S228P and L235E mutations in the Fc region (IgG4 S228P/L235E, referred to as .B constructs, e.g., 350.B), numbered according to Eu numbering. The sequences of these antibodies are disclosed in Table 1. For the antibody 350.A, two lots of antibodies were produced, referred to hereafter as 350.A1 and 350.A2.

TABLE 10
Two lineages of anti-CD73 antibodies

| | | IgG1 | IgG4 S228P | IgG4 S228P/L235E |
|---|---|---|---|---|
| | Antibody | format | format | format |
| Lineage 1 | | | | |
| Parent | 918 | 918.C | 918.A | 918.B |
| Child | 350 | 350.C | 350.A1, 350.A2 | 350.B |
| Child | 356 | 356.C | 356.A | 356.B |
| Child | 358 | 358.C | 358.A | 358.B |
| Lineage 3 | | | | |
| Parent | 930 | 930.C | 930.A | 930.B |
| Child | 373 | 373.C | 373.A | 373.B |
| Child | 374 | 374.C | 374.A | 374.B |
| Child | 376 | 376.C | 376.A | 376.B |
| Child | 377 | 377.C | 377.A | 377.B |
| Child | 379 | 379.C | 379.A | 379.B |

All the anti-CD73 antibodies tested bind to human and cynomolgus CD73. The Lineage 1 antibodies also bind to murine CD73. Table 11 provides Kd values of these antibodies measured using Octet as described above.

TABLE 11
Affinities of anti-CD73 antibodies

| | Human ForteBio | Cyno ForteBio | Murine ForteBio |
|---|---|---|---|
| | Whole IgG | Whole IgG | Whole IgG |
| mAb | Kd (M) | Kd (M) | Kd (M) |
| 918.C | 1.53E−09 | Not Determined | 8.88E−09 |
| 350.C | 2.48E−10* | 6.35E−10 | 4.09E−10* |
| 356.C | 2.43E−10* | 6.93E−10 | 4.01E−10 |
| 358.C | 3.17E−10 | 8.14E−10 | 3.51E−09 |
| 930.C | 1.83E−09 | Not Determined | Not Bound |
| 373.C | 6.21E−10 | 9.14E−10 | Not Bound |
| 374.C | 3.58E−10 | 1.03E−10 | Not Bound |
| 376.C | 3.01E−10 | Not Determined | Not Bound |
| 377.C | 5.72E−10 | 1.42E−09 | Not Bound |
| 379.C | 5.50E−10 | 1.62E−09 | Not Bound |

*Value approaching upper limit for Kd measurement

Next, using epitope binning/ligand blocking studies, it was shown that the parental antibody 918 competed for binding to CD73 with the progeny antibodies 350, 356, and 358. Similarly, the parental antibody 930 competed for binding to CD73 with the progeny antibodies 373, 374, 376, 377, and 379. Both 918 and 930 were shown to compete with an internal reference anti-CD73 antibody, suggesting that these antibodies share the same epitope bin.

### Fab and Antibody Affinity Measurement Using Surface Plasmon Resonance

Fabs of mAbs 350 and 373 were generated by engineering a stop between the two proline residues above the core hinge region of the heavy chain of 350 and 373. Both were expressed in Expi293F (ThermoFisher) cells and purified using CaptureSelect IgG CH1 Affinity resin (ThermoFisher).

Biacore was used to measure cross-species affinity for the Fab materials of mAbs 350 and 373. Proteins used were as follows: recombinant human CD73 (R&D Systems 5795-EN); recombinant cynomolgus monkey CD73 (Sino Biological 90912-C08H); recombinant mouse CD73 (R&D Systems 4488-EN); and recombinant rat CD73 (Sino Biological 80375-R08H). Anti-human Fab (GE Healthcare Life Sciences) was immobilized on all 4 flow cells (Fc) on a CM5 chip (GE). Fabs 350 and 373 were captured on Fc2 and Fc4, at ˜20 RU. 0.01 nM to 90 nM CD73 (3-fold dilution series) was flown over all 4 Fcs. All samples were diluted in running buffer HBS-EP+(pH 7.4, 0.01 M HEPES, 150 mM NaCl, 3 mM EDTA and 0.05% (v/v) P20).

Shown in Table 12 are results for Kd (M) affinity for cross-species binding of 350 and 373 Fabs.

TABLE 12
Affinities of anti-CD73 Fabs

| Fab | Antigen | K_{d} (M) |
|---|---|---|
| Fab 350 | hCD73 | ≤1E−10 |
| Fab 350 | cCD73 | ≤1E−10 |
| Fab 350 | mCD73 | 1.728E−8 |
| Fab 350 | rCD73 | 2.829E−8 |
| Fab 373 | hCD73 | 1.304E−8 |
| Fab 373 | cCD73 | 9.465E−9 |
| Fab 373 | mCD73 | No Binding |
| Fab 373 | rCD73 | No Binding |

In a separate study, the affinity of the full-length antibody 373.A or Fab fragments of 373.A to human, cynomolgus monkey, mouse and rat CD73 was determined using an anti-histidine (His) antibody capture Biacore method utilizing surface plasmon resonance (SPR). The anti-His Ab was directly immobilized onto a CM5 chip surface by amine coupling. The His-tagged human CD73/His, cynomolgus monkey CD73/His, mouse CD73/His or rat CD73/His was flowed over and captured at a desired resonance unit (RU) for an Rmax of 20. Antibody analyte concentrations in serial dilutions of IgG or Fab were flowed over at 60 μL/min. The sensorgrams were analyzed using the manufacturer's software for a 1:1 binding model. Binding to mouse CD73/His protein and rat CD73/His protein was undetectable for 373.A and 373.A Fab, demonstrating that 373.A is not rodent cross-reactive.

Affinities were established for human and cynomolgus monkey CD73 with both 373.A and 373.A Fab. Hydrogen-deuterium mass spectrometry and size exclusion chromatography studies support a model of conformational locking of the CD73-dimer by 373.A into the open-open (inactive-inactive) conformation, supporting a 1:1 bidentate binding of 1 Ab:1 CD73 dimer. Therefore, given that 1:1 bidentate binding will favor avidity, the whole Ab affinities were used rather than Fab measurements. The full length antibody 373.A binds recombinant human CD73 with a Kd of 0.991±0.267 nM and cross-reacts with recombinant cynomolgus monkey CD73 with a Kd of 0.068±0.009 nM as determined by Biacore kinetic binding studies.

### Whole Blood Target Engagement by Anti-CD73 Antibodies

Whole blood target engagement was assessed by flow cytometry using whole blood from healthy human donors. Briefly, biotinylated antibodies were incubated with whole blood for 30 minutes prior to red blood cell lysis and fixation. Fixed cells were stained for CD3 and CD8 to identify CD8+ T cells, and streptavidin-APC to detect biotin. After staining, the cell were washed and subjected to flow cytometry analysis.

Dose dependent binding, as measured by median fluorescence intensity (MFI) of APC signal, was observed for the anti-CD73 antibodies tested (data not shown). A biotinylated isotype control antibody did not show binding to CD8+ T cells.

### CD73 Target Occupancy on Human Whole Blood Samples

Conceptual demonstration of the target occupancy (TO) of CD73 on human whole blood samples was performed by treating donor blood ex vivo with unlabeled 373.A. A titration of 373.A or DNP-IgG4sm isotype control from 10 μg/mL to 0.17 ng/mL was performed. Samples from the two donors that were treated with unlabeled 373.A at higher doses (10 μg/mL to ˜0.1 μg/mL) prevented biotinylated 373.A from binding to the cells, reducing the geometric mean fluorescence intensity (gMFI) values to a plateau at the background level of fluorescence (˜550 gMFI for both donors). This is indicative of full CD73 target occupancy. In contrast, gMFI values for cells that were pretreated with lower amounts of unlabeled 373.A (0.17 ng/mL and 0.51 ng/mL) were similar to samples that were pretreated with DNP-IgG4sm isotype control (˜1600 gMFI for donor 1 and ˜2200 gMFI for donor 2). Isotype control treated samples mimicked blood that had zero target occupancy.

### Inhibition of the Enzymatic Activity of Soluble Recombinant CD73

5′ ectonucleotidase CD73 is the rate limiting step in the conversion of AMP to adenosine. The ability of anti-CD73 antibodies to inhibit the enzymatic activity of CD73 was measured using a malachite green phosphate assay. Briefly, 25 ng/ml recombinant human CD73 was incubated with a dose titration of the substrate adenosine monophosphate (AMP) (0-500 μM) with buffer alone, or in the presence of an isotype control antibody at 1 μg/ml or the anti-CD73 antibody 350.C at 1, 0.3, or 0.1 μg/ml. Release of inorganic phosphate (Pi) was measured using a malachite green phosphate assay kit (Enzo Life Sciences, Catalog #BML-AK111).

The control antibody at the tested concentration had no effect on the Michaelis constant (Km) of recombinant human CD73. In contrast, the anti-CD73 antibody 350.C caused dose-dependent reduction of Vmax on Km curves (data not shown), indicating that the antibody 350.C is a non-competitive inhibitor of human CD73.

Next, the anti-CD73 antibodies 350, 356, 373, and 374, expressed in either the .A or .B format, were tested for their ability to inhibit the enzymatic activity of recombinant human and cynomolgus monkey CD73 using a similar malachite green phosphate assay as described above. In brief, anti-CD73 antibodies were incubated for 10 minutes with 25 ng/ml recombinant human or cynomolgus CD73 in the presence of 25 μM AMP. Release of inorganic phosphate (Pi) was measured using a malachite green phosphate assay kit (Enzo Life Sciences, Catalog #BML-AK111). Normalized percent inhibition (% INH) was determined using time zero control as 100% INH and no antibody control as 0% INH.

All the anti-CD73 antibodies tested inhibited the enzymatic activity of soluble recombinant human and cynomolgus CD73 (data not shown).

### Inhibition of the Enzymatic Activity of Soluble Endogenous CD73

Further, the enzyme inhibition activity of anti-CD73 antibodies was tested against soluble endogenous CD73, for example, CD73 shed from the cell surface.

In a first study, anti-CD73 antibodies 350 and 373, expressed in either the .A or .B format, or isotype control antibodies were incubated for 240 minutes with MDA-MB-231 (a human breast cancer cell line) conditioned serum free media in the presence of 100 μM AMP. Disappearance of AMP was measured by a modified Cell Titer Glo (CTG) assay (Promega, Cat #G9242/3). AMP inhibits the luciferase signal in the CTG kit. The luciferase signal increases as the added AMP is enzymatically consumed by CD73. Normalized percent inhibition (% INH) was determined using time zero control as 100% INH and no antibody control as 0% INH.

The anti-CD73 antibodies dose-dependently inhibited the enzymatic activity of CD73 shed from the breast cancer cell line MDA-MB-231 (data not shown).

In a second study, anti-CD73 antibodies 350, 356, 358, 373, 374, 377, and 379, all expressed in the .B format, were incubated for 60 minutes with diluted (12.5% v:v in PBS) serum from a pancreatic cancer patient in the presence of 100 μM AMP. Similar to the first study, disappearance of AMP was measured by the modified Cell Titer Glo (CTG) assay and normalized percent inhibition (% INH) was determined using time zero control as 100% INH and no antibody control as 0% INH.

Anti-CD73 antibodies also inhibited CD73 enzymatic activity in the serum from the pancreatic cancer patient in a dose-dependent manner (data not shown).

Inhibition of the Enzymatic Activity of CD73 Expressed on the Cell Surface

First, a malachite green phosphate assay was used to examine the ability of anti-CD73 antibodies 350, 356, 358, 373, 374, 377, and 379 (all in the .B format) to inhibit CD73 expressed on a breast cancer cell line MDA-MB-231. Briefly, antibodies were incubated for 180 minutes with cells in the presence of 100 μM AMP. Release of inorganic phosphate from AMP was measured using a malachite green phosphate assay kit (Enzo Life Sciences, Catalog #BML-AK111). Normalized percent inhibition (% INH) was determined using time zero control as 100% INH and no antibody control as 0% INH.

All the anti-CD73 antibodies tested inhibited CD73 enzymatic activity expressed on the surface of the breast cancer cell line MDA-MB-231 (data not shown).

Next, since the Lineage 1 antibodies cross-react with mouse CD73 whereas the Lineage 3 antibodies do not, antibodies from both lineages were tested against CD73 expressed on the surface of a human or murine breast cancer cell line. Anti-CD73 antibodies were incubated for 240 minutes with a human breast cancer cell line MDA-MB-231 or a murine breast cancer cell line 4T1 in the presence of 100 μM AMP. Disappearance of AMP was measured by the modified Cell Titer Glo (CTG) assay described above and normalized percent inhibition (% INH) was determined using time zero control as 100% INH and no antibody control as 0% INH.

Consistent with their binding profiles, the Lineage 1 antibodies 918, 350, 356, and 358 inhibited both human and murine CD73, whereas the Lineage 3 antibodies 930, 373, 374, 376, 377, and 379 inhibited human, but not murine, CD73 (data not shown).

Furthermore, two modified Cell Titer Glo (CTG) assays were conducted to test the enzyme inhibition activity of anti-CD73 antibodies against CD73 expressed on a human breast cancer cell line MDA-MB-231 or a human ovarian cancer cell line SKOV3. In both studies, 1000 ng/ml anti-CD73 antibodies were incubated for 240 minutes with 20,000 cells/ml cells in the presence of 100 μM AMP at 37° C. Disappearance of AMP was measured by the modified Cell Titer Glo (CTG) assay described above and normalized percent inhibition (% INH) was determined using time zero control as 100% INH and no antibody control as 0% INH.

In both studies, all the anti-CD73 antibodies tested were able to inhibit surface CD73 expressed on the human breast cancer cell line MDA-MB-231 or the human ovarian cancer cell line SKOV3 (data not shown)

Next, a similar Cell Titer Glo (CTG) assay was performed to examine the ability of anti-CD73 antibodies to inhibit human CD73 expressed on a HEK 293 cell line. Briefly, a HEK 293 cell line was engineered to stably overexpress human CD73 and incubated with anti-CD73 antibodies for 150 minutes in the presence of 100 μM AMP. Disappearance of AMP was measured by the modified Cell Titer Glo (CTG) assay described above and normalized percent inhibition (% INH) was determined using time zero control as 100% INH and no antibody control as 0% INH.

The anti-CD73 antibodies 350, 356, 373, and 374, in the .A or .B format, inhibited membrane-bound human CD73 in a dose-dependent manner (data not shown).

In addition, the enzyme inhibition activity of anti-CD73 antibodies were also examined using human PBMCs. In brief, primary human PBMCs were isolated from two separate donors and incubated with anti-CD73 antibodies for 480 minutes in the presence of 25 μM AMP. Disappearance of AMP was measured by the modified Cell Titer Glo (CTG) assay described above and normalized percent inhibition (% INH) was determined using time zero control as 100% INH and no antibody control as 0% INH.

The anti-CD73 antibodies tested inhibited the enzymatic activity of CD73 expressed on primary human PBMCs from both donors (data not shown)

Restoration of CD4+ and CD8+ T Cell Proliferation in the Presence of AMP

Next, anti-CD73 antibodies were tested for their ability to relieve AMP-mediated inhibition of CD4+ T cells. Briefly, CD4+ T cells were isolated from healthy human donor pooled Peripheral Blood Mononuclear Cells (PBMC). Prior to stimulation with anti-CD3/28 beads in the presence of 800 μM AMP, CD4+ T cells were stained with CellTrace Violet (CTV) (Thermo Fisher Scientific, Cat #C34557) to track cell division. On day 4, proliferation was determined by CTV dilution using flow cytometry. Cells stained with CTV lose approximately half of their fluorescence signal as measured on the flow cytometer with each division. Proliferation index was calculated as a measure of the level of T cell division for each condition where 100 represents maximal proliferation and 0 represents no proliferation.

The anti-CD73 antibodies tested were able to restore CD4+ T cell proliferation in the presence of AMP (data not shown).

Inhibition of the Enzymatic Activity of CD73 In Vivo

Furthermore, anti-CD73 antibodies were examined for their enzyme inhibition activity in vivo. Athymic nude, female mice (6-8 weeks of age) were implanted with high CD73-expressing MDA-MB231 breast cancer cell line (ATCC HTB-26) at 10×106 cells/mouse/200 microl. Five mice per group were randomized when tumors were 200 mm3 and treated intraperitoneally with either 20 or 200 microg/mouse of control polyclonal human IgG or a panel of anti-CD73 mAbs. The antibodies tested are the anti-CD73 antibodies 350, 356, 373, and 374, expressed in either the .A or .B format.

Plasma was collected three days post-dose at a ratio of one portion of plasma into five portions of methanol by volume. The methanol quenched samples were stored at −80° C. before use, at which time the samples were centrifuged. The precipitations were discarded and the supernatants were transferred to new Eppendorf tubes. Stock solutions of internal standards (IS, C-13 labeled adenosine and N-15 labeled Inosine, Cambridge Isotope Laboratories, MA) were added to the final concentration of 50 nM. The prepared samples were then analyzed using an LC/MS system of API-6500 QTrap (AB Sciex, US) coupled with a Shimadzu LC pump (LC-20AD) and a CTC auto sampler with DLW wash. For each sample, 5 μL was injected and separated using a SeQuant ZIC-pHILIC column (5 μm, 150×2.1 mm, Millipore, MA) maintained at 40° C. A binary gradient was used for the elution, where mobile phase B is 100% acetonitrile with no additives and mobile phase A is 12 mM ammonium formate and 12 mM formic acid in 1:1 (v/v) mix of water and acetonitrile. The elution was programed as (0, 85, 0.6), (0.5, 85, 0.4), (2, 10, 0.4), (4.5, 10, 0.4), (5, 85, 0.4), (5.5, 85, 0.6), where values in the parentheses are time in minutes, percent of mobile phase B and flow rate in mL/min in order. Adenosine and C13-Adenosine were monitored from 0.5 to 4.5 minutes at ESI positive mode and mass transitions 268->136 and 273->136 respectively. Inosine and N15-Inosine were monitored from 0.5 to 4.5 minutes at ESI negative mode and mass transitions 267->135 and 271->139 respectively. Results were reported as nM adenosine or inosine.

All the anti-CD73 antibodies tested effectively reduced the accumulation of adenosine and inosine in the serum of immunocompromised mice implanted with the high CD73-expressing MDA-MB231 breast cancer cell line (data not shown).

### Example 2: A Phase I/Ib Study of the Anti-CD73 Antibody 373.A as a Single Agent and in Combination with BAP049-Clone-E and/or PBF509 in Patients with Advanced Malignancies

During the last decade, immunotherapies that target different immune checkpoints (e.g., PD-1, PD-L1 and CTLA-4) have shown efficacy in a numbers of cancer indications. However, while some patients achieve objective and long lasting responses to checkpoint blockade, the majority of patients show modest or no clinical benefit, indicating that tumors use alternative immunosuppressive mechanisms to achieve immune escape (Allard et al., Clin Cancer Res. 2013; 19(20):5626-35; Vesely et al., Annu Rev Immunol 2011; 29:235-271). Thus, concomitant blockade of multiple immune suppressive pathways may be required to induce clinically meaningful responses in a larger number of patients.

Over the past years, adenosine generation and signaling have emerged as potential therapeutic targets in cancer treatment. Adenosine creates an immunosuppressive tumor microenvironment by reducing the cytotoxic anti-tumor immune response, enhancing the proliferation and polarization of immune suppressive cells, and by increasing neovascularization (Young et al., Cancer Discovery 2014; 4(8):879-88). Preclinical data demonstrate that CD73 blockade can significantly delay primary tumor growth and inhibit the development of lung metastases in an immune-competent syngeneic mouse model (Stagg et al 2010). Similar results were observed in a study where genetic deletion of A2aR in the host resulted in rejection of the established immunogenic tumors in A2aR deficient mice with no rejection seen in control wild type mice (Ohta et al., PNAS 2006; 103(35):13132-37).

A phase I/Ib, open-label, multi-center study has been designed to evaluate the safety, tolerability, preliminary anti-tumor activity, pharmacokinetics (PK) and pharmacodynamics (PD) of the anti-CD73 antibody 373.A as a single agent and in combination with the A2aR antagonist PBF509 (NIR178) and/or the anti-PD-1 antibody BAP049-Clone-E (Spartalizumab) in patients with advanced malignancies.

The sequence of the anti-CD73 antibody 373.A is given in Table 1—see the antibody designated 373. It has an IgG4 format with S228P mutation. The heavy chain of anti-CD73 antibody 373.A is 373.A (SEQ ID NO: 46, wherein X is K) and for convenience this anti-CD73 antibody with this heavy chain sequence will be referred to as anti-CD73 antibody 373.A.

The primary objectives are to characterize the safety and tolerability, and to determine the recommended dose (RD) for anti-CD73 antibody 373.A as a single agent and in combination with PBF509 and/or BAP049-Clone-E. The secondary objectives are to assess the preliminary anti-tumor activity and PK of anti-CD73 antibody 373.A as a single agent and in combination with PBF509 and/or BAP049-Clone-E, assess the immunogenicity of anti-CD73 antibody 373.A and BAP049-Clone-E, and to characterize changes in the immune infiltrate in tumors following treatment, e.g., change from baseline in tumor infiltrating lymphocytes (TILs), tumor associated macrophages (TAMs), CD8+ T-cells, and PDL-1 expression.

BAP049-Clone-E (Spartalizumab) is a high-affinity, ligand-blocking, humanized anti-programmed death-1 (PD-1) IgG4 antibody that blocks the binding of PD-L1 and PD-L2 to PD-1. BAP049-Clone-E is being tested in a phase I/II study in advanced malignancies. The sequence of BAP049-Clone-E is disclosed in Table 5.

PBF509 (NIR178), a new, non-xanthine-based compound, is a potent oral adenosine A2aR antagonist.

Two ongoing Phase I/Ib and Phase II studies evaluate PBF509 as a single agent and/or in combination with BAP049-Clone-E in patients with advanced non-small cell lung cancer (NSCLC) and solid tumors and non-Hodgkin lymphoma, respectively.

This I/Ib study will initially enroll adult patients with advanced malignancies that have progressed or are intolerant to standard therapy in indications where moderate to high CD73 expression has been associated with poorer outcome, indicating adenosine-mediated immune escape (Wu et al., Journal of Surgical Oncology 2012, 106(2): 130-137; Gaudreau et al., Oncoimmunology; 2016, 5(5): e1127496; Inoue et al., Oncotarget.; 2017, 8(5):8738-8751). These indications include non-small cell lung cancer (NSCLC), triple negative breast cancer (TNBC), pancreatic cancer (PDAC), renal cell carcinoma (RCC), ovarian cancer, micro-satellite stable (MSS) colorectal cancer and metastatic castration resistant prostate cancer (mCRPC), though additional indications may be enrolled based on emerging clinical data (e.g. efficacy data or proven pathway activation).

The study consists of two parts: (1) a dose escalation part for single agent anti-CD73 antibody 373.A, doublet combinations anti-CD73 antibody 373.A/PBF509 and anti-CD73 antibody 373.A/BAP049-Clone-E, or a triplet combination anti-CD73 antibody 373.A/PBF509/BAP049-Clone-E, which leads to declaration of recommended doses (RDs) for each treatment, and (2) a dose expansion part where patients will be treated at the RDs for single agent, doublet combinations, and triplet combination. The escalation part will enroll patients with advanced NSCLC, TNBC, PDAC, RCC, ovarian cancer, and colorectal cancer (MSS); and there is no restriction on the number of prior treatments. The expansion part will enroll patients with advanced malignancies having received up to 3 lines of prior treatment.

In the expansion part, patients in each indication will be equally randomized to the combination treatment arms. Randomization will be performed per indication, and further stratified within certain indications by prior PD-1/PD-L1 treatment (naïve or resistant).

### Dose and Regimen Selection

### Anti-CD73 Antibody 373.A Monotherapy

The starting dose of 60 mg flat dose anti-CD73 antibody 373.A, administered intravenously every 2 weeks (Q2W), was selected based upon preclinical safety, tolerability, and PK data observed in cynomolgus monkey as well as published case histories of CD73 deficient patients.

The 60 mg dose is considered to be a minimally pharmacological active dose (mPAD) as it is predicted to provide (1) approximately 20 hrs of >90% CD8+ T-cell CD73 occupancy, (2) approximately 22 hrs of >90% adenosine inhibition, (3) approximately 17 hrs of >90% imputed overall CD73 occupancy.

Based on modeling of TK data from the cynomolgus monkey Toxicology studies, ex-vivo CD8+ T cell CD73 occupancy data and in-vitro data on inhibition of adenosine formation, a dose ≥1200 mg Q2W is predicted to achieve >90% target occupancy on CD8+ T cells throughout the dosing interval and a dose ≥600 mg Q2W is predicted to achieve >90% inhibition of adenosine production.

The dose of anti-CD73 antibody 373.A will be escalated in sequential cohorts, guided by a Bayesian Logistic Regression model (BLRM) coupled with overdose control (EWOC) criterion, until a maximum tolerated dose (MTD) or recommended dose (RD) for expansion is identified. Preclinical data and modeling suggest that there may be a high antigen sink and that high doses (e.g. >1200 mg Q2W) may be needed to achieve continuous target occupancy throughout the dose interval. Dose escalation will primarily be performed with a Q2W regimen. However, if this regimen shows rapid anti-CD73 antibody 373.A elimination and lack of target saturation within the dosing interval, a more frequent QW regimen may be tested. If on the other hand, a Q4W regimen is predicted to have no rapid elimination within the dosing interval, Q4W regimen may be explored instead.

For the step-up dosing schedule, anti-CD73 antibody 373.A will initially be administered on a weekly schedule (QW) on C1 D1 and C1D8, then at C1D15 onwards, anti-CD73 antibody 373.A will be administered on a bi-weekly schedule (Q2W).

### Anti-CD73 Antibody 373.A/PBF509 Combination

The maximum starting doses for the anti-CD73 antibody 373.A/PBF509 doublet combination will be 200 mg Q2W anti-CD73 antibody 373.A and 80 mg BID PBF509.

200 mg Q2W anti-CD73 antibody 373.A is a low dose of anti-CD73 antibody 373.A that is predicted to achieve ˜2.3 days of >90% target occupancy on CD8+ T cells. The 200 mg Q2W anti-CD73 antibody 373.A dose is 16% of the 1200 mg Q2W dose that is anticipated to achieve >90% CD8+ T cell target occupancy throughout the dosing interval.

A dose-escalation approach of anti-CD73 antibody 373.A and PBF509 will be undertaken in order to determine the appropriate dose of each drug in combination, guided by Bayesian Logistic Regression modeling (BLRM) coupled with overdose control (EWOC) principle criteria.

### Anti-CD73 Antibody 373.A/BAP049-Clone-E Combination

The maximum starting dose for the 373.A/BAP049-Clone-E doublet combination will be 200 mg Q2W anti-CD73 antibody 373.A and 400 mg Q4W BAP049-Clone-E.

The rationale for 200 mg Q2W anti-CD73 antibody 373.A has been described above. The 200 mg Q2W anti-CD73 antibody 373.A will be combined with the RD for BAP049-Clone-E which is 400 mg Q4W, which has been shown to be safe and efficacious.

Anti-CD73 antibody 373.A dose level will be escalated sequentially with a fixed dose of BAP049-Clone-E, guided by Bayesian Logistic Regression modeling (BLRM) coupled with overdose control (EWOC) principle criteria.

### 373.A/BAP049-Clone-E/PBF509 Combination

The maximum starting dose for the anti-CD73 antibody 373.A/BAP049-Clone-E/PBF509 triplet combination will be 200 mg Q2W anti-CD73 antibody 373.A, 400 mg Q4W BAP049-Clone-E and 80 mg BID PBF509.

A dose-escalation approach for anti-CD73 antibody 373.A/BAP049-Clone-E/PBF509 with a fixed dose of BAP049-Clone-E will be undertaken in order to determine the appropriate dose of anti-CD73 antibody 373.A and PBF509 in the triplet combination, guided by a Bayesian Logistic Regression model (BLRM) coupled with overdose control (EWOC) criterion.

The anti-CD73 antibody 373.A (100 mg powder for solution for infusion) will be administered intravenously as a 1 hr infusion (up to 2 hours if clinically indicated). BAP049-Clone-E (100 mg powder for solution for infusion) will be administered intravenously as a 30 minute infusion (up to 2 hours, if clinically indicated). When given in combination, anti-CD73 antibody 373.A and BAP049-Clone-E may be administered on the same day using separate infusion materials (bag, lines, filters) for each infusion. The same access site may be used for both infusions. Anti-CD73 antibody 373.A may be infused first followed by a 30 minute break before infusing BAP049-Clone-E. PBF509 (40 mg and/or 80 mg and/or 160 mg capsule for oral use) may be taken orally twice daily (BID) continuously. On the visits where anti-CD73 antibody 373.A and/or BAP049-Clone-E will be administered, the dose of PBF509 may be taken first followed by the anti-CD73 antibody 373.A infusion. A break between PBF509 administration and the anti-CD73 antibody 373.A infusion is not required.

Tables 13-16 describe the starting dose and the dose levels that may be evaluated during this trial. Patients treated with anti-CD73 antibody 373.A single agent or anti-CD73 antibody 373.A in combination with BAP049-Clone-E and/or PBF509 will begin study treatment on Cycle 1 Day 1. Each cycle will consist of 28 days. BAP049-Clone-E Q4W will be administered on Day 1 of a cycle. PBF509 BID will be taken every day of a cycle.

In the step-up dosing schedule of anti-CD73 antibody 373.A, an initial Cycle 1 day 1 dose of anti-CD73 antibody 373.A will be followed by a Cycle 1 day 8 dose of anti-CD73 antibody 373.A, followed by Q2W anti-CD73 antibody 373.A starting day 15. The sum of the first two doses of anti-CD73 antibody 373.A (day 1 and day 8) shall not exceed the dose level of anti-CD73 antibody 373.A being administered Q2W, as single agent or in combination with BAP049-Clone-E and/or PBF509. Cohorts with anti-CD73 antibody 373.A step-up dosing will be explored in dose levels that have been previously tested with anti-CD73 antibody 373.A continuous dosing.

TABLE 13
Provisional dose levels for anti-CD73 antibody 373.A

| | Proposed | Increment |
|---|---|---|
| | dosing | from previous |
| Dose level | regimen* | dose |
| −2** | 6 mg Q2W | −233% |
| −1** | 20 mg Q2W | −200% |
| 1 | 60 mg Q2W | (starting dose) |
| 2 | 200 mg Q2W | 233% |
| 3 | 600 mg Q2W | 200% |
| 4 | 1200 mg Q2W | 100% |
| 5 | 2400 mg Q2W | 100% |
| 6 | 3600 mg Q2W | 50% |

*It is possible for additional and/or intermediate dose levels to be added during the course of the study Cohorts may be added at any dose level below the MTD in order to better understand safety, PK or PD.
**Dose level −1 and −2 represent treatment doses for patients requiring a dose reduction from the starting dose level. No dose reduction below dose level −2 is permitted for this study.

TABLE 14
Provisional dose levels for anti-CD73 antibody
373.A in combination with PBF509

| | Proposed dose | Increment from | | Increment |
|---|---|---|---|---|
| | anti-CD73 | previous dose | Proposed | from previous |
| | antibody | anti-CD73 | dose | dose |
| Dose level | 373.A* | antibody 373.A | PBF509* | PBF509 |
| −2** | 20 mg Q2W | −200% | 40 mg BID | 0% |
| −1** | 60 mg Q2W | −233% | 40 mg BID | −100% |
| 1 | 200 mg Q2W | (starting dose) | 80 mg BID | (starting dose) |
| 2 | 200 mg Q2W | 0% | 160 mg BID | 100% |
| 3 | 600 mg Q2W | 200% | 160 mg BID | 0% |
| 4 | 1200 mg Q2W | 100% | 160 mg BID | 0% |
| 5 | 2400 mg Q2W | 100% | 160 mg BID | 0% |
| 6 | 2400 mg Q2W | 100% | 320 mg BID | 100% |
| 7 | 2400 mg Q2W | | 480 mg BID | |
| 8 | 2400 mg Q2W | | 620 mg BID | |
| 9 | 3600 mg Q2W | | 320 mg BID | |
| 10 | 3600 mg Q2W | | 480 mg BID | |
| 11 | 3600 mg Q2W | | 620 mg BID | |

*It is possible for additional and/or intermediate dose levels to be added during the course of the study Cohorts may be added at any dose level below the MTD in order to better understand safety, PK or PD.
**Dose level −1 and −2 represent treatment doses for patients requiring a dose reduction from the starting dose level. No dose reduction below dose level −2 is permitted for this study.

TABLE 15
Provisional dose levels for anti-CD73 antibody
373.A in combination with BAP049-Clone-E

| | Proposed dose | | Increment from | Proposed | Increment from |
|---|---|---|---|---|---|
| | anti-CD73 | | previous dose | dose | previous dose |
| | antibody | | anti-CD73 | BAP049- | BAP049- |
| Dose level | 373.A* | | antibody 373.A | Clone-E | Clone-E |
| −2** | 20 | mg Q2W | −200% | 400 mg Q4w | 0% |
| −1** | 60 | mg Q2W | −233% | 400 mg Q4w | 0% |
| 1 | 200 | mg Q2W | (starting dose) | 400 mg Q4w | (starting dose) |
| 2 | 600 | mg Q2W | 200% | 400 mg Q4w | 0% |
| 3 | 1200 | mg Q2W | 100% | 400 mg Q4w | 0% |
| 4 | 2400 | mg Q2W | 100% | 400 mg Q4w | 0% |
| 5 | 3600 | mg Q2W | 50% | 400 mg Q4W | 0% |

*It is possible for additional and/or intermediate dose levels to be added during the course of the study Cohorts may be added at any dose level below the MTD in order to better understand safety, PK or PD.
**Dose level −1 and −2 represent treatment doses for patients requiring a dose reduction from the starting dose level. No dose reduction below dose level −2 is permitted for this study.

TABLE 16
Provisional dose levels for PBF509 in combination with anti-CD73
antibody 373.A and BAP049-Clone-E

| | | Increment | | Increment | | |
|---|---|---|---|---|---|---|
| | Proposed | from previous | | from | | Increment |
| | dose anti- | dose anti- | Proposed | previous | | from |
| | CD73 | CD73 | dose | dose | Proposed | previous |
| Dose | antibody | antibody | BAP049- | BAP049- | dose | dose |
| level | 373.A* | 373.A | Clone-E | Clone-E | PBF509* | PBF509 |
| −2** | 20 mg Q2W | −200% | 400 mg Q4W | 0% | 40 mg BID | 0% |
| −1** | 60 mg Q2W | −233% | 400 mg Q4W | 0% | 40 mg BID | −100% |
| 1 | 200 mg Q2W | (starting dose) | 400 mg Q4W | (starting dose) | 80 mg BID | (starting dose) |
| 2 | 200 mg Q2W | 0% | 400 mg Q4W | 0% | 160 mg BID | 100% |
| 3 | 600 mg Q2W | 200% | 400 mg Q4W | 0% | 160 mg BID | 0% |
| 4 | 1200 mg Q2W | 100% | 400 mg Q4W | 0% | 160 mg BID | 0% |
| 5 | 2400 mg Q2W | 100% | 400 mg Q4W | 0% | 160 mg BID | 0% |
| 6 | 2400 mg Q2W | | 400 mg Q4W | | 320 mg BID | |
| 7 | 2400 mg Q2W | | 400 mg Q4W | | 480 mg BID | |
| 8 | 2400 mg Q2W | | 400 mg Q4W | | 620 mg BID | |
| 9 | 3600 mg Q2W | | 400 mg Q4W | | 320 mg BID | |
| 10 | 3600 mg Q2W | | 400 mg Q4W | | 480 mg BID | |
| 11 | 3600 mg Q2W | | 400 mg Q4W | | 620 mg BID | |

*It is possible for additional and/or intermediate dose levels to be added during the course of the study Cohorts may be added at any dose level below the MTD in order to better understand safety, PK or PD.
**Dose level −1 and −2 represent treatment doses for patients requiring a dose reduction from the starting dose level. No dose reduction below dose level −2 is permitted for this study.

### Example 3: Step Up Dosing Regime

It was observed that most patients (>90%) that experienced any grade headache had the event after the first dose of anti-CD73 antibody 373.A. The maximum concentration (Cmax) of anti-CD73 antibody 373.A after the first dose was explored to establish the relationship with the probability to experience headaches by using logistic regression analysis. Statistically significant relationships were found between anti-CD73 antibody 373.A Cmax after the first dose and the probabilities to experience any grade headache event. When the Cmax of anti-CD73 antibody 373.A was below 100 g/ml, the probability to experience Grade 2 or higher headache events was estimated to be below 25% and the probability to experience Grade 3 or higher was estimated to be less than 10%.

The population PK model was developed to simulate the ranges of Cmax following the administration of varying anti-CD73 antibody 373.A dose levels. It was shown that most patients would have Cmax less than 100 g/ml with 200 mg anti-CD73 antibody 373.A dose in cycle 1 indicating the use of 200 mg would significantly lower the rate of headaches. Since the headaches were a first dose effect and were resolved within 48 hours, additional dose of 400 mg in the second week (Cycle 1 Day 8) was proposed to maintain the dose intensity over two weeks comparable to 600 mg Q2W regimen. The population PK simulations showed that the splitting anti-CD73 antibody 373.A dose of 600 mg into 200 mg (Week 1)+400 mg (Week 2) would have similar exposure (i.e., AUC) to 600 mg Q2W with ˜3 times lower Cmax in week 1.

### Example 4. A Phase I/Ib, Open-Label, Multi-Center, Study of Anti-ENTPD2 mAb1 as a Single Agent and in Combination with Spartalizumab, an Anti-CD73 Ab and NIR178 in Patients with Advanced Solid Tumors

### Study Design

This study is a FIH, open-label, phase I/Ib, multi-center study which consists of a dose escalation part of anti-ENTPD2 mAb1 as a single agent and in combination with Spartalizumab, an anti-CD73 Ab or NIR178, followed by an expansion part. Moreover, an optional triple combination may be considered after evaluation of all safety and efficacy data and the MTD/RD for the doublet combinations have been determined. Enrollment will be limited to subjects with MSS CRC, cholangiocarcinoma, pancreatic cancer, esophageal, EGJ or gastric cancer.

During the escalation part, the first dose for the first two subjects treated at an untested dose level of anti-ENTPD2 mAb1, either as single agent, or in combination with Spartalizumab or NIR178 or an anti-CD73 Ab will be staggered by 48 hours. The dose escalation of the combination may start after 3 dose levels of anti-ENTPD2 mAb1 single agent have been determined to be safe and tolerable. Once the RD or MTD of anti-ENTPD2 mAb1 as single agent and anti-ENTPD2 mAb1 in combination with Spartalizumab, an anti-CD73 Ab or NIR178 are determined, the corresponding expansion part(s) may commence.

The “Priming dose” is designated as either only the C1 D1 (S2) or the C1D1 and the C1D8 dose (S3). In both cases, the Priming dose(s) will be lower than the Experimental dose. The “Experimental dose” is designated as the dose to be given at C1D15 and beyond on a Q2W schedule. In S3, the initial C1D8 Priming dose will be equal to the C1 D1 Priming dose and may be further escalated based on tolerability. The main dose escalation regimen will focus on exploring the Experimental dose using the BLRM with EWOC criteria. However, in case the Priming dose at C1 D1 and/or C1D8 needs to be modified, a separate BLRM will be used.

### Study Treatment

For this study, the terms “investigational drug” or “study drug” refer to anti-ENTPD2 mAb1, an anti-CD73 Ab, Spartalizumab (PDR001), and NIR178. The study treatment is defined as anti-ENTPD2 mAb1 alone or in combination with Spartalizumab (PDR001), an anti-CD73 Ab or NIR178.

All dosages prescribed and dispensed to subjects and all dose changes during the study must be recorded in the appropriate dosage administration record eCRF.

Investigational and Control Drugs

TABLE 18
Investigational drug

| Investigational | | | | | | |
|---|---|---|---|---|---|---|
| Drug | | | Frequency | | | |
| (Name and | Pharmaceutical | | and or | Route of | Supply | Sponsor |
| Strength) | Dosage Form | Dose | regimen | Administration | Type | (global) |
| anti-ENTPD2 | Concentrate for | 100 mg | Q2W or | Intravenous use | Open label | Sponsor |
| mAb1 150 mg/1 mL | Solution for | starting | Q4W | | bulk supply; | (global) |
| | infusion | dose | | | vials | |
| Spartalizumab | Concentrate for | 400 mg | Q4W | Intravenous use | Open label | Sponsor |
| (PDR001) | solution for | | | | bulk supply; | (global) |
| 100mg/4mL | infusion | | | | vials | |
| NIR178 | Capsule | 160 mg | BID | Oral use | Open label | Sponsor |
| 40 mg, 80 mg, | | starting | | | bulk supply; | (global) |
| 160 mg | | dose | | | vials | |
| Anti-CD73 Ab | Powder for | 400 mg | Q2W | Intravenous use | Open label | Sponsor |
| 100 mg | solution for | starting | | | bulk supply; | (global) |
| | infusion | dose | | | vials | |

### Starting Doses

Prior to the first subject treated and following incorporation of all additional relevant data into the model and a clinical review of all available data, a lower starting dose may be recommended. The starting dose will satisfy the EWOC criterion.

Anti-ENTPD2 mAb1 Single Agent Treatment:
The starting dose for anti-ENTPD2 mAb1 in this FIH study will be 100 mg Q2W and is based upon the anti-ENTPD2 mAb1 preclinical data.

### Combination Treatments:

Anti-ENTPD2 mAb1 in combination with Spartalizumab: Spartalizumab at the RP2D (400 mg Q4W)
Anti-ENTPD2 mAb1 in combination with NIR178: NIR178 160 mg BID (RP2D established in combination with Spartalizumab and NIR178 MTD established at 480 mg BID)
Anti-ENTPD2 mAb1 in combination with an anti-CD73 Ab: The provisional starting dose for the anti-CD73 Ab will be 100 mg Q2W.
For all the combination dose escalations, the provisional starting Experimental dose of anti-ENTPD2 mAb1 is selected as 300 mg Q2W regardless of the dosing schedule. In practice, at the time of the initiation of the respective combination dose escalations, a lower starting dose may be selected upon the review of all available safety data from respective single agents to ensure that the EWOC criteria is satisfied by the combination dose based on available DLT data from the respective single agents.

Note that the starting Experimental dose for anti-ENTPD2 mAb1 in the combination treatments will not exceed the highest tolerable dose during the single agent dose escalation that satisfies the EWOC criteria as per the BHLRM/BLRM.

The starting Priming dose in the combination treatments will be selected based on the Priming dose from anti-ENTPD2 mAb1 single agent. In case that the Priming dose(s) needs to be escalated during the combination dose escalation, the following conditions must be satisfied:
the cumulative exposure, in the first 14 days, used to select the Priming dose(s) must satisfy the EWOC criteria as per the BLRM used to guide the Priming dose selection;
the Experimental dose must satisfy the EWOC criteria as per the BLRM used to guide the Experimental dose selection for anti-ENTPD2 mAb1 single agent.

### Provisional Dose Levels

The provisional dose levels shown in the tables below will be used for anti-ENTPD2 mAb1 alone and in combination. Prior to any dose being escalated, all information from previous doses will be evaluated, decisions will be based on a synthesis of all relevant data available from all dose levels. The recommended dose for the next cohort of subjects will be guided by BLRM/BHLRM with EWOC principle.

Table 19 describes the starting dose and the provisional dose levels for anti-ENTPD2 mAb1 monotherapy that may be evaluated during this trial. In Schedule 1, anti-ENTPD2 mAb1 will be administered bi-weekly (Q2W) or every 4 weeks (Q4W) without a Priming dose. The Q4W dosing regimen may be tested without a Priming dose if a less frequent dosing regimen is supported by emerging data from the study. The two additional dosing schedules outlined in Schedule 2 and Schedule 3 incorporate one or more Priming doses which are lower than the Experimental dose administered on C1D15.

Schedule 2 (S2) incorporates a single Priming dose at C1 D1, with the Experimental dose beginning on C1D15, and continues with that dose on a Q2W regimen. The initial Priming dose will be fixed at anti-ENTPD2 mAb1 100 mg. Subsequently, this may be adjusted based on emerging safety/tolerability, PK and/or PD data. The C1 D1 dose will not exceed the dose level satisfying the EWOC principle by the BLRM defined for Priming dose modification. The provisional starting Experimental dose is set to 300 mg.

Schedule 3 (S3) incorporates two Priming doses, the first at C1 D1 and the second at C1D8, with the Experimental dose beginning at C1D15, and continues with that dose on a Q2W regimen. The initial Priming dose at C1D8 will be the same as the Priming dose at C1 D1. Subsequently, this may be adjusted based on emerging safety/tolerability, PK and/or PD data and if further immune tolerance prior to the first Experimental dose administration is thought to be required. The starting Experimental dose for Schedule 3 will not be higher than the highest Experimental dose in Schedule 2 that satisfies the EWOC criteria as per the BLRM. The cumulative dose given within the first 14 days of cycle 1 will not exceed the dose satisfying the EWOC principle by the BLRM defined for Priming dose modification. In addition, the C1D8 dose will be chosen such that the cumulative dose C1 D1 plus C1D8 does not increase beyond 100% of a previously tested cumulative dose that has been deemed to be well-tolerated. The same principle applies to any higher C1D8 dose.

The proposed provisional Experimental dose levels for Cycle 1 Day 15 anti-ENTPD2 mAb1 are outlined in Table 19, Table 20, Table 21, Table 22, and Table 23.

The anti-ENTPD2 mAb1 dosing strategies described above may be evaluated for single agent anti-ENTPD2 mAb1, anti-ENTPD2 mAb1 in combination with Spartalizumab (See 21), anti-ENTPD2 mAb1 in combination with NIR178 (See Table 22) and anti-ENTPD2 mAb1 in combination with an anti-CD73 Ab (See Table 23).

TABLE 19
Provisional dose levels for anti-ENTPD2 mAb1 Schedule 1 (S1)

| | Proposed dose for | Increment from |
|---|---|---|
| Dose level | anti-ENTPD2 mAb1* | previous dose |
| −2 | 10 mg Q2W | −67% |
| −1 | 30 mg Q2W | −70% |
| 1 | 100 mg Q2W | (starting dose) |
| 2 | 200 mg Q2W | 100% |
| 3 | 300 mg Q2W | 50% |
| 4 | 600 mg Q2W | 100% |
| 5 | 1200 mg Q2W | 100% |
| 6 | 2400 mg Q2W | 100% |

*It is possible for additional and/or intermediate/lower (dose levels −1, −2) dose levels to be added during the course of the study. Enrichment cohorts may be added at any dose level below the MTD in order to better understand safety, PK or PD.

TABLE 20
Provisional dose levels for anti-ENTPD2 mAb1
Schedule 2 and Schedule 3 (S2 and S3)

| | Proposed | Proposed | |
|---|---|---|---|
| | Priming | Experimental dose | Increment from |
| Dose level | dose(s)* | anti-ENTPD2 mAb1** | previous dose |
| −2 | 50 mg | 150 mg Q2W | −50% in both Priming |
| | | | and Experimental |
| | | | dose |
| −1 | 50 mg | 300 mg Q2W | −50% reduction in |
| | | | Priming dose |
| 1 | 100 mg | 300 mg Q2W | (starting dose) |
| 2 | 100 mg | 600 mg Q2W | 100% |
| 3 | 100 mg | 1200 mg Q2W | 100% |
| 4 | 100 mg | 2400 mg Q2W | 100% |

*For S2, the Priming dose of 100 mg anti-ENTPD2 mAb1 will be administered on Cycle 1 Day 1 only. If deemed necessary and based on safety and tolerability the Priming dose may be modified; For S3, an additional Priming dose will be administered on Cycle 1 Day 8, and will initially be the same as the Cycle 1 Day 1 dose. Subsequently, it may be escalated such that the cumulative dose Cycle 1 Day 1 plus Cycle 1 Day 8 is not >100% of the cumulative dose previously evaluated to be tolerable.
**The starting Experimental dose of anti-ENTPD2 mAb1 for the Q2W regimen as presented in this Table 20 is provisional and will commence on Cycle 1 Day 15. It will not exceed a 100% increase from the previous Experimental dose tested and proven safe. Table 21 describes the provisional dose levels for anti-ENTPD2 mAb1 in combination with Spartalizumab that may be evaluated during this trial. There is no dose reduction for Spartalizumab.

TABLE 21
Provisional dose levels for anti-ENTPD2
mAb1 in combination with Spartalizumab

| | Proposed | Increment from | | Increment |
|---|---|---|---|---|
| | Experimental | previous dose | Proposed | from previous |
| | dose anti- | anti-ENTPD2 | dose | dose |
| Dose level* | ENTPD2 mAb1 ** | mAb1 | Spartalizumab | Spartalizumab |
| −1 | 150 mg Q2W | −50% | 400 mg Q4W | N/A |
| 1 | 300 mg Q2W | (starting dose) | 400 mg Q4W | N/A |
| 2 | 600 mg Q2W | 100% | 400 mg Q4W | N/A |
| 3 | 1200 mg Q2W | 100% | 400 mg Q4W | N/A |
| 4 | 2400 mg Q2W | 100% | 400 mg Q4W | N/A |

*It is possible for additional and/or intermediate/lower (dose level −1) dose levels/schedule to be added during the course of the study. Enrichment cohorts may be added at any dose level below the MTD in order to better understand safety, PK or PD.
** The starting dose of anti-ENTPD2 mAb1 for the combination as presented in this table is provisional. A lower starting dose may be selected upon the review of all available safety data from both single agents and to ensure that the EWOC criteria is satisfied by the combination dose based on available DLT data from both single agents.

Table 22 describes the provisional dose levels for anti-ENTPD2 mAb1 in combination with NIR178 that may be evaluated during this trial.

TABLE 22
Provisional dose levels for anti-ENTPD2 mAb1 in combination with NIR178

| | Proposed | | | | |
|---|---|---|---|---|---|
| | Experimental | Increment from | | | |
| | dose anti- | previous dose | | | Increment |
| | ENTPD2 | anti-ENTPD2 | Proposed dose | | from previous |
| Dose level* | mAb1 ** | mAb1 | NIR178 ** | | dose NIR178 |
| −1 | 150 mg Q2W | −100% | 80 | mg BID | −50% |
| 1 | 300 mg Q2W | (starting dose) | 160 | mg BID | (starting dose) |
| 2 | 600 mg Q2W | 100% | 160 | mg BID | 0% |
| 3 | 1200 mg Q2W | 100% | 160 | mg BID*** | 0% |

*It is possible for additional and/or intermediate/lower (dose level −1) dose levels/schedule to be added during the course of the study. Enrichment cohorts may be added at any dose level below the MTD in order to better understand safety, PK or PD.
** The starting dose of anti-ENTPD2 mAb1 for the combination as presented in this table is provisional. A lower starting dose may be selected upon the review of all available safety data from both single agents and to ensure that the EWOC criteria is satisfied by the combination dose based on available DLT data from both single agents.
***Incremental doses for NIR178 (up to 480 mg BID) may be considered on the basis of PK/PD and safety data.

Table 23 describes the provisional dose levels for anti-ENTPD2 mAb1 in combination with an anti-CD73 Ab that may be evaluated during the trial.

TABLE 23
Provisional dose levels for anti-ENTPD2
mAb1 and an anti-CD73 Ab in combination

| | Proposed | Increment from | | |
|---|---|---|---|---|
| | Experimental | previous dose | Proposed dose | Increment from |
| | dose anti- | anti-ENTPD2 | anti-CD73 | previous dose |
| Dose level* | ENTPD2 mAb1 ** | mAb1 | Ab ** | anti-CD73 Ab |
| −1 | 150 mg Q2W | −50% | 60 mg Q2W | −40% |
| 1 | 300 mg Q2W | (starting dose) | 100 mg Q2W | (starting dose) |
| 2 | 600 mg Q2W | 100% | 100 mg Q2W | 0% |
| 3 | 600 mg Q2W | 0% | 200 mg Q2W | 100% |
| 4 | 1200 mg Q2W | 100% | 200 mg Q2W | 0% |
| 5 | 1200 mg Q2W | 0% | 400 mg Q2W | 100% |

*It is possible for additional and/or intermediate/lower (−1 dose level) dose levels/schedules to be added during the course of the study. Enrichment cohorts may be added at any dose level below the MTD in order to better understand safety, PK or PD.
** The starting dose of anti-ENTPD2 mAb1 for the combination as presented in this table is provisional. A lower starting dose may be selected upon the review of all available safety data from both single agents and to ensure that the EWOC criteria is satisfied by the combination dose based on available DLT data from both single agents.

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. An antibody molecule that binds to human CD73 for use in treating a cancer in a subject, whereby the antibody molecule is administered in a step-up dosing regime such that in the main phase the antibody molecule dose is administered at a main dose amount according to a main dosing period with a main dosing period frequency, which is preceded by an initial phase in which said antibody molecule is administered at a higher dosing period frequency than the main dosing period frequency and is administered via fractionated dose amounts of the main dose amount, wherein in the initial phase within a time period equal to the main dosing period the fractionated dose amounts summed together shall not exceed the amount of the main phase dose amount, wherein the antibody molecule comprises (i) a heavy chain variable region (VH) comprising a heavy chain complementarity determining region 1 (VHCDR1) amino acid sequence of SEQ ID NO: 38 a VHCDR2 amino acid sequence of SEQ ID NO: 36, and a VHCDR3 amino acid sequence of SEQ ID NO: 37; and (ii) a light chain variable region (VL) comprising a light chain complementarity determining region 1 (VLCDR1) amino acid sequence of SEQ ID NO: 48, a VLCDR2 amino acid sequence of SEQ ID NO: 49, and a VLCDR3 amino acid sequence of SEQ ID NO: 50.

2. A method of treating cancer in a subject comprising administering to the subject an antibody molecule that binds to human CD73 in an amount effective to treat the cancer, whereby the antibody molecule is administered in a step-up dosing regime such that in the main phase the antibody molecule dose is administered at a main dose amount according to a main dosing period with a main dosing period frequency, which is preceded by an initial phase in which said antibody molecule is administered at a higher dosing period frequency than the main dosing period frequency and is administered via fractionated dose amounts of the main dose amount, wherein in the initial phase within a time period equal to the main dosing period the fractionated dose amounts summed together shall not exceed the amount of the main phase dose amount, wherein the antibody molecule comprises (i) a heavy chain variable region (VH) comprising a heavy chain complementarity determining region 1 (VHCDR1) amino acid sequence of SEQ ID NO: 38, a VHCDR2 amino acid sequence of SEQ ID NO: 36, and a VHCDR3 amino acid sequence of SEQ ID NO: 37; and (ii) a light chain variable region (VL) comprising a light chain complementarity determining region 1 (VLCDR1) amino acid sequence of SEQ ID NO: 48, a VLCDR2 amino acid sequence of SEQ ID NO: 49, and a VLCDR3 amino acid sequence of SEQ ID NO: 50.

3. The antibody for use of claim 1 or method of claim 2, wherein the antibody molecule that binds to human CD73 comprises a heavy chain variable region comprising the amino acid sequence of SEQ ID NO: 44, or an amino acid sequence having at least about 85%, 90%, 95%, or 99% sequence identity to SEQ ID NO: 44 and/or a light chain variable region comprising the amino acid sequence of SEQ ID NO: 55, or an amino acid sequence having at least about 85%, 90%, 95%, or 99% sequence identity to SEQ ID NO: 55.

4. The antibody for use of any one of claim 1 or 3 or method of any one of claim 2 or 3, wherein the antibody molecule that binds to human CD73 comprises a heavy chain comprising the amino acid sequence of SEQ ID NO: 46, or an amino acid sequence having at least about 85%, 90%, 95%, or 99% sequence identity to SEQ ID NO: 46, wherein X in SEQ ID NO: 46 is K and/or a light chain comprising the amino acid sequence of SEQ ID NO: 57, or an amino acid sequence having at least about 85%, 90%, 95%, or 99% sequence identity to SEQ ID NO: 57.

5. The antibody for use of any one of claim 1 or 3 to 4 or method of any one of claims 2 to 4, wherein the antibody molecule that binds to human CD73 comprises a heavy chain constant region of IgG4 and a light chain constant region of kappa.

6. The antibody for use of any one of claim 1 or 3 to 5 or method of any one of claims 2 to 5, wherein the antibody molecule that binds to human CD73 comprises
i) a human IgG4 heavy chain constant region with a mutation at position 228 according to EU numbering, or
ii) a human IgG4 heavy chain constant region with a Serine to Proline mutation at position 228 according to EU numbering, or
iii) a heavy chain constant region comprising the amino acid sequence of SEQ ID NO: 92 or 93.

7. The antibody for use of any one of claim 1 or 3 to 6 or method of any one of claims 2 to 6, wherein in the initial phase within a time period equal to the main dosing period the fractionated dose amounts summed together equal the main dose amount.

8. The antibody for use of any one of claim 1 or 3 to 7 or method of any one of claims 2 to 7, such that the initial phase is used to prevent or reduce the incidence or severity of headaches and/or migraines.

9. The antibody for use of any one of claim 1 or 3 to 8 or method of any one of claims 2 to 8, wherein the main dose amount is 600 mg and/or the main dose frequency is Q2W.

10. The antibody for use of any one of claim 1 or 3 to 9 or method of any one of claims 2 to 9, wherein in the initial phase the antibody molecule is administered at a frequency of QW.

11. The antibody for use of any one of claim 1 or 3 to 10 or method of any one of claims 2 to 10, wherein in the initial phase the fractionated dose amounts that are administered within a time period equal to the main dosing period when summed together equal the main dose amount and are administered at a lower amount followed by an intermediate amount of the main phase dose amount.

12. The antibody for use of any one of claim 1 or 3 to 11 or method of any one of claims 2 to 11, wherein in the initial phase the fractionated dose amounts are about 200 mg and about 400 mg and are administered within two weeks.

13. The antibody for use of any one of claim 1 or 3 to 12 or method of any one of claims 2 to 12, whereby the antibody molecule that binds to human CD73 is administered in the initial phase on day 1 at about 200 mg and on day 8 at about 400 mg, followed by the main phase beginning on day 15 with about 600 mg, and continuing thereafter with about 600 mg administered Q2W.

14. The antibody for use of any one of claim 1 or 3 to 13 or method of any one of claims 2 to 13, whereby the antibody molecule is administered by IV infusion over 1 to 2 hours at the dosing period frequency according to the respective phase.

15. The antibody for use of any one of claim 1 or 3 to 14 or method of any one of claims 2 to 14, wherein the antibody molecule that binds to human CD73 is administered in combination with one or more therapeutic agents or procedures.

16. The antibody for use of any one of claim 1 or 3 to 15 or method of any one of claims 2 to 15, wherein the antibody molecule that binds to human CD73 is administered in combination with a triptan.

17. The antibody for use of claim 16 or method of claim 16, wherein the triptan is selected from Almotriptan, Eletriptan, Frovatriptan, Naratriptan, Rizatriptan, Sumatriptan, Zolmitriptan, Lasmiditan, optionally which may be combined with an additional agent, such as sumatriptan combined with naproxen sodium.

18. The antibody for use of any one of claims 15 to 17 or method of any one of claims 15 to 17 wherein the antibody molecule that binds to human CD73 is administered in combination with a PD-1 inhibitor.

19. The antibody for use of claim 18 or method of claim 18, wherein the PD-1 inhibitor is selected from the group consisting of Spartalizumab, Nivolumab, Pembrolizumab, Pidilizumab, MED10680, REGN2810, TSR-042, PF-06801591, and AMP-224, preferably Spartalizumab.

20. The antibody for use of any one of claims 18 to 19 or method of any one of claims 18 to 19, wherein the PD-1 inhibitor is an anti-PD-1 antibody molecule, wherein the anti-PD-1 antibody molecule is administered at a dose of about 400 mg Q4W.

21. The antibody for use of any one of claims 15 to 20 or method of any one of claims 15 to 20, wherein the antibody molecule that binds to human CD73 is administered in combination with an adenosine A2AR antagonist.

22. The antibody for use of claim 21 or method of claim 21, wherein
(i) the adenosine A2AR antagonist is selected from the group consisting of PBF509, CP1444, AZD4635, Vipadenant, GBV-2034, and AB928; or
(ii) the adenosine A2AR antagonist is selected from the group consisting of 5-bromo-2,6-di-(1H-pyrazol-1-yl)pyrimidine-4-amine; (S)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine; (R)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine, or racemate thereof; 7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine; and 6-(2-chloro-6-methylpyridin-4-yl)-5-(4-fluorophenyl)-1,2,4-triazin-3-amine;
preferably wherein the adenosine A2AR antagonist is PBF509.

23. The antibody for use of any one of claims 21 to 22 or method of any one of claims 21 to 22, wherein the adenosine A2AR antagonist is administered at a dose of about 80 mg, about 160 mg, 240 mg or about 320 mg, preferably wherein the adenosine A2AR antagonist is administered at a dose of about 240 mg twice a day (BID).

24. The antibody for use of any one of claims 15 to 23 or method of any one of claims 15 to 23, wherein the antibody molecule that binds to human CD73 is administered in combination with an anti-human ENTPD2 antibody.

25. The antibody for use of claim 24 or method of claim 24, wherein the antibody molecule that binds to human CD73 is administered in combination with an anti-human ENTPD2 antibody comprising a VH comprising a VHCDR1 amino acid sequence of SEQ ID NO: 401, a VHCDR2 amino acid sequence of SEQ ID NO: 402, and a VHCDR3 amino acid sequence of SEQ ID NO: 403; and a VL comprising a VLCDR1 amino acid sequence of SEQ ID NO: 414, a VLCDR2 amino acid sequence of SEQ ID NO: 415, and a VLCDR3 amino acid sequence of SEQ ID NO: 416.

26. The antibody for use of claim 24 or 25 or method of claim 24 or 25, wherein the antibody molecule that binds to human CD73 is administered in combination with an anti-human ENTPD2 antibody that comprises a VH with a sequence of SEQ ID NO: 410 and VL with a sequence of SEQ ID NO: 421.

27. The antibody for use of any one of claims 24 to 26 or method of any one of claims 24 to 26, wherein the antibody molecule that binds to human CD73 is administered in combination with an anti-human ENTPD2 antibody that comprises a heavy chain with a sequence of SEQ ID NO: 412 and a light chain with a sequence of SEQ ID NO: 423.

28. The antibody for use of any one of claim 1 or 3 to 27 or method of any one of claims 2 to 27, wherein the antibody molecule that binds to human CD73 is administered in the initial phase at about 200 mg QW, then about 400 mg QW, followed by the main phase at about 600 mg Q2W, in combination with Spartalizumab administered at about 400 mg Q4W and PBF509 administered at about 240 mg BID.

29. The antibody for use of any one of claim 1 or 3 to 28 or method of any one of claims 2 to 28, wherein the cancer is chosen from non-small cell lung cancer, pancreatic ductal adenocarcinoma, triple-negative breast cancer, microsatellite stable (MSS) colorectal cancer, metastatic castration resistant prostate cancer, ovarian cancer or renal cell carcinoma.

30. The antibody for use of any one of claim 1 or 3 to 29 or method of any one of claims 2 to 29, wherein the antibody molecule that binds to human CD73 is in the form of a pharmaceutical composition comprising the antibody molecule as defined in any one of claims 1 to 6 and a pharmaceutically acceptable carrier, excipient or stabilizer.

31. An antibody molecule that binds to human ENTPD2 for use in treating a cancer in a subject,
whereby the antibody molecule is administered in a step-up dosing regime such that in the main phase the antibody molecule dose is administered at a main dose amount according to a main dosing period with a main dosing period frequency, which is preceded by an initial phase in which said antibody molecule is administered at an equal or higher dosing period frequency than the main dosing period frequency and is administered via fractionated dose amounts of the main dose amount,
wherein in the initial phase within a time period equal to the main dosing period the fractionated dose amounts summed together shall not exceed the amount of the main phase dose amount.

32. A method of treating cancer in a subject comprising administering to the subject an antibody molecule that binds to human ENTPD2 in an amount effective to treat the cancer,
whereby the antibody molecule is administered in a step-up dosing regime such that in the main phase the antibody molecule dose is administered at a main dose amount according to a main dosing period with a main dosing period frequency, which is preceded by an initial phase in which said antibody molecule is administered at an equal or higher dosing period frequency than the main dosing period frequency and is administered via fractionated dose amounts of the main dose amount,
wherein in the initial phase within a time period equal to the main dosing period the fractionated dose amounts summed together shall not exceed the amount of the main phase dose amount,

33. The antibody for use of claim 31 or method of claim 32, wherein the antibody molecule comprises:
(i) a heavy chain variable region (VH) comprising a heavy chain complementarity determining region 1 (VHCDR1) amino acid sequence of SEQ ID NO: 401, a VHCDR2 amino acid sequence of SEQ ID NO: 402, and a VHCDR3 amino acid sequence of SEQ ID NO: 403; and
(ii) a light chain variable region (VL) comprising a light chain complementarity determining region 1 (VLCDR1) amino acid sequence of SEQ ID NO: 414, a VLCDR2 amino acid sequence of SEQ ID NO: 415, and a VLCDR3 amino acid sequence of SEQ ID NO: 416.

34. The antibody for use of claim 31 or 33 or method of claim 32 or 33, wherein the initial phase is used to prevent or reduce the incidence or severity of cytokine release syndrome (CRS).

35. The antibody for use of any one of claim 31 or 33-34 or method of any one of claims 32-34, wherein the main dose amount is 300 mg, 600 mg, 1200 mg, or 2400 mg, and/or the main dose frequency is Q2W.

36. The antibody for use of any one of claim 31 or 33-35 or method of any one of claims 32-35, wherein in the initial phase the antibody molecule is administered at a frequency of QW or Q2W.

37. The antibody for use of any one of claim 31 or 33-36 or method of any one of claims 32-36, wherein in the initial phase the fractionated dose amount is 100 mg.

38. The antibody for use of any one of claim 31 or 33-37 or method of any one of claims 32-37, wherein in the initial phase the antibody molecule is administered once or twice within two weeks.

39. The antibody for use of any one of claim 31 or 33-38 or method of any one of claims 32-38, whereby the antibody molecule is administered in the initial phase on day 1 at about 100 mg, followed by the main phase beginning on day 15 with about 300 mg, and continuing thereafter with about 300 mg administered Q2W.

40. The antibody for use of any one of claim 31 or 33-38 or method of any one of claims 32-38, whereby the antibody molecule is administered in the initial phase on day 1 at about 100 mg and on day 8 at about 100 mg, followed by the main phase beginning on day 15 with about 300 mg, and continuing thereafter with about 300 mg administered Q2W.

41. The antibody for use of any one of claim 31 or 33-40 or method of any one of claims 32-40, whereby the antibody molecule is administered to the subject intravenously as a 1 hr infusion (up to 2 hours if clinically indicated).

42. The antibody for use of any one of claim 31 or 33-41 or method of any one of claims 32-41, wherein the antibody molecule is administered in combination with one or more therapeutic agents or procedures.

43. The antibody for use or method of claim 42, wherein the antibody molecule is administered in combination with a PD-1 inhibitor.

44. The antibody for use or method of claim 43, wherein the PD-1 inhibitor is selected from the group consisting of Tislelizumab, Spartalizumab, Nivolumab, Pembrolizumab, Pidilizumab, MED10680, REGN2810, TSR-042, PF-06801591, and AMP-224.

45. The antibody for use or method of claim 43 or 44, wherein the PD-1 inhibitor is an anti-PD-1 antibody molecule, wherein the anti-PD-1 antibody molecule is administered at a dose of about 400 mg Q4W.

46. The antibody for use or method of claim 42, wherein the antibody molecule is administered in combination with an adenosine A2AR antagonist.

47. The antibody for use or method of claim 46, wherein
(i) the adenosine A2AR antagonist is selected from the group consisting of PBF509, CP1444, AZD4635, Vipadenant, GBV-2034, and AB928; or
(ii) the adenosine A2AR antagonist is selected from the group consisting of 5-bromo-2,6-di-(1H-pyrazol-1-yl)pyrimidine-4-amine; (S)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine; (R)-7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine, or racemate thereof; 7-(5-methylfuran-2-yl)-3-((6-(((tetrahydrofuran-3-yl)oxy)methyl)pyridin-2-yl)methyl)-3H-[1,2,3]triazolo[4,5-d]pyrimidin-5-amine; and 6-(2-chloro-6-methylpyridin-4-yl)-5-(4-fluorophenyl)-1,2,4-triazin-3-amine;
preferably wherein the adenosine A2AR antagonist is PBF509.

48. The antibody for use or method of claim 46 or 47, wherein the adenosine A2AR antagonist is administered at a dose of about 80 mg, about 160 mg, 240 mg or about 320 mg, preferably wherein the adenosine A2AR antagonist is administered at a dose of about 160 mg twice a day (BID).

49. The antibody for use or method of claim 42, wherein the antibody molecule is administered in combination with an anti-human CD73 antibody.

50. The antibody for use or method of claim 49, wherein the anti-human CD73 antibody comprises: (i) a heavy chain variable region (VH) comprising a heavy chain complementarity determining region 1 (VHCDR1) amino acid sequence of SEQ ID NO: 38, a VHCDR2 amino acid sequence of SEQ ID NO: 36, and a VHCDR3 amino acid sequence of SEQ ID NO: 37; and (ii) a light chain variable region (VL) comprising a light chain complementarity determining region 1 (VLCDR1) amino acid sequence of SEQ ID NO: 48, a VLCDR2 amino acid sequence of SEQ ID NO: 49, and a VLCDR3 amino acid sequence of SEQ ID NO: 50.

51. The antibody for use or method of claim 49, wherein the anti-human CD73 antibody comprises: a heavy chain variable region comprising the amino acid sequence of SEQ ID NO: 44, or an amino acid sequence having at least about 85%, 90%, 95%, or 99% sequence identity to SEQ ID NO: 44 and/or a light chain variable region comprising the amino acid sequence of SEQ ID NO: 55, or an amino acid sequence having at least about 85%, 90%, 95%, or 99% sequence identity to SEQ ID NO: 55.

52. The antibody for use or method of claim 49, wherein the anti-human CD73 antibody comprises: a heavy chain comprising the amino acid sequence of SEQ ID NO: 46, or an amino acid sequence having at least about 85%, 90%, 95%, or 99% sequence identity to SEQ ID NO: 46, wherein X in SEQ ID NO: 46 is K and/or a light chain comprising the amino acid sequence of SEQ ID NO: 57, or an amino acid sequence having at least about 85%, 90%, 95%, or 99% sequence identity to SEQ ID NO: 57.

53. The antibody for use of any one of claim 31 or 33-52 or method of any one of claims 32-52, wherein the cancer is MSS colorectal cancer (CRC), cholangiocarcinoma (intrahepatic or extrahepatic), pancreatic cancer, esophageal cancer, esophageal gastric junction (EGJ) cancer, or gastric cancer.

54. The antibody for use of any one of claim 31 or 33-53 or method of any one of claims 32-53, wherein the antibody molecule that binds to human ENTPD2 is in the form of a pharmaceutical composition comprising the antibody molecule as defined in claim 33 and a pharmaceutically acceptable carrier, excipient or stabilizer.

55. The antibody for use of any one of claim 31 or 33-54 or method of any one of claims 32-54, wherein the antibody molecule comprises:
a heavy chain variable region (VH) comprising SEQ ID NO: 410 or a sequence at least about 95% or more identical thereto, and a light chain variable region (VL) comprising SEQ ID NO: 421 or a sequence at least about 95% or more identical thereto.

56. The antibody for use of any one of claim 31 or 33-54 or method of any one of claims 32-54, wherein the antibody molecule comprises:
a heavy chain comprising SEQ ID NO: 412 or a sequence at least about 95% or more identical thereto, and a light chain comprising SEQ ID NO: 423 or a sequence at least about 95% or more identical thereto.


SEQUENCE LISTING (926 sequences)

SEQ ID NO: 1 - protein, 10 aa, Artificial Sequence
  source: /note=“Description of Artificial Sequence: Synthetic peptide”
GGSISGRYWS

SEQ ID NO: 2 - protein, 16 aa, Artificial Sequence
  source: /note=“Description of Artificial Sequence: Synthetic peptide”
YIYGTGSTNYNPSLKS

SEQ ID NO: 3 - protein, 13 aa, Artificial Sequence
  source: /note=“Description of Artificial Sequence: Synthetic peptide”
ESQESPYNNWFDP

SEQ ID NO: 4 - protein, 5 aa, Artificial Sequence
  source: /note=“Description of Artificial Sequence: Synthetic peptide”
GRYWS

SEQ ID NO: 5 - protein, 7 aa, Artificial Sequence
  source: /note=“Description of Artificial Sequence: Synthetic peptide”
GGSISGR

SEQ ID NO: 6 - protein, 5 aa, Artificial Sequence
  source: /note=“Description of Artificial Sequence: Synthetic peptide”
YGTGS

SEQ ID NO: 7 - protein, 8 aa, Artificial Sequence
  source: /note=“Description of Artificial Sequence: Synthetic peptide”
GGSISGRY

SEQ ID NO: 8 - protein, 7 aa, Artificial Sequence
  source: /note=“Description of Artificial Sequence: Synthetic peptide”
IYGTGST

SEQ ID NO: 9 - protein, 15 aa, Artificial Sequence
  source: /note=“Description of Artificial Sequence: Synthetic peptide”
ARESQESPYNNWFDP

SEQ ID NO: 10 - protein, 121 aa, Artificial Sequence
  source: /note=“Description of Artificial Sequence: Synthetic polypeptide”
QVQLQESGPGLVKPSETLSLTCTVSGGSISGRYWSWIRQPPGKGLEWIGYIYGTGSTNYNPSLKSRVTISVDTSKNQFSLKLSSVTAADTAVYYCARESQESPYNNWFDPWGQGTLVTVSS

SEQ ID NO: 11 - DNA, 363 bp, Artificial Sequence
  source: /note=“Description of Artificial Sequence: Synthetic polynucleotide”
caagtgcagctgcaggaatctggccctggcctggtcaagccctccgagacactgtccctgacctgcaccgtgtccggcggctccatctccggccggtactggtcttggatccggcagcctcccggcaagggcctggaatggatcggctacatctacggcaccggctccaccaactacaaccccagcctgaagtccagagtgaccatctccgtggacacctccaagaaccagttctccctgaagctgtcctccgtgaccgccgctgacaccgccgtgtactactgcgccagagagtcccaggaatccccttacaacaattggttcgacccctggggccagggcaccctggtcaccgtgtcctct

SEQ ID NO: 12 - protein, 448 aa, Artificial Sequence
  source: /note=“Description of Artificial Sequence: Synthetic polypeptide”
  VARIANT (448)..(448): /replace=“ ”
  SITE (1)..(448): /note=“Variant residues given in the sequence have no preference with respect to those in the annotations for variant positions”
QVQLQESGPGLVKPSETLSLTCTVSGGSISGRYWSWIRQPPGKGLEWIGYIYGTGSTNYNPSLKSRVTISVDTSKNQFSLKLSSVTAADTAVYYCARESQESPYNNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - DNA, 1344 bp, Artificial Sequence
  source: /note=“Description of Artificial Sequence: Synthetic polynucleotide”
  variation (1342)..(1342): /replace=“ ”
  variation (1343)..(1343): /replace=“ ”
  variation (1344)..(1344): /replace=“ ”
  misc_feature (1)..(1344): /note=“Variant nucleotides given in the sequence have no preference with respect to those in the annotations for variant positions”
caagtgcagctgcaggaatctggccctggcctggtcaagccctccgagacactgtccctgacctgcaccgtgtccggcggctccatctccggccggtactggtcttggatccggcagcctcccggcaagggcctggaatggatcggctacatctacggcaccggctccaccaactacaaccccagcctgaagtccagagtgaccatctccgtggacacctccaagaaccagttctccctgaagctgtcctccgtgaccgccgctgacaccgccgtgtactactgcgccagagagtcccaggaatccccttacaacaattggttcgacccctggggccagggcaccctggtcaccgtgtcctctgcttccaccaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgccctccttgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggcaag

SEQ ID NO: 14 - protein, 11 aa, Artificial Sequence
  source: /note=“Description of Artificial Sequence: Synthetic peptide”
RASQGISSWLA

SEQ ID NO: 15 - protein, 7 aa, Artificial Sequence
  source: /note=“Description of Artificial Sequence: Synthetic peptide”
AASSLQS

SEQ ID NO: 16 - protein, 9 aa, Artificial Sequence
  source: /note=“Description of Artificial Sequence: Synthetic peptide”
QQGNSFPRT

SEQ ID NO: 17 - protein, 7 aa, Artificial Sequence
  source: /note=“Description of Artificial Sequence: Synthetic peptide”
SQGISSW

SEQ ID NO: 18 - protein, 3 aa, Artificial Sequence
  source: /note=“Description of Artificial Sequence: Synthetic peptide”
AAS

SEQ ID NO: 19 - protein, 6 aa, Artificial Sequence
  source: /note=“Description of Artificial Sequence: Synthetic peptide”
GNSFPR

SEQ ID NO: 20 - protein, 6 aa, Artificial Sequence
  source: /note=“Description of Artificial Sequence: Synthetic peptide”
QGISSW

SEQ ID NO: 21 - protein, 107 aa, Artificial Sequence
  source: /note=“Description of Artificial Sequence: Synthetic polypeptide”
DIQMTQSPSSVSASVGDRVTITCRASQGISSWLAWYQQKPGKAPKLLIYAASSLQSGVPSRFSGSGSGTDFTLTISSLQPEDFATYYCQQGNSFPRTFGGGTKVEIK

SEQ ID NO: 22 - DNA, 321 bp, Artificial Sequence
  source: /note=“Description of Artificial Sequence: Synthetic polynucleotide”
gacatccagatgacccagagcccctcctccgtgtccgcctccgtgggcgacagagtgaccatcacctgtcgggcctcccagggcatctccagctggctggcctggtatcagcagaagcccggcaaggcccctaagctgctgatctacgccgcctccagcctgcagtccggcgtgccctccagattctccggctctggctccggcaccgacttcaccctgaccatcagctccctgcagcccgaggacttcgccacctactactgccagcagggcaactccttccctcggaccttcggcggaggcaccaaagtggaaatcaag

SEQ ID NO: 23 - protein, 214 aa, Artificial Sequence
  source: /note=“Description of Artificial Sequence: Synthetic polypeptide”
DIQMTQSPSSVSASVGDRVTITCRASQGISSWLAWYQQKPGKAPKLLIYAASSLQSGVPSRFSGSGSGTDFTLTISSLQPEDFATYYCQQGNSFPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - DNA, 642 bp, Artificial Sequence
  source: /note=“Description of Artificial Sequence: Synthetic polynucleotide”
gacatccagatgacccagagcccctcctccgtgtccgcctccgtgggcgacagagtgaccatcacctgtcgggcctcccagggcatctccagctggctggcctggtatcagcagaagcccggcaaggcccctaagctgctgatctacgccgcctccagcctgcagtccggcgtgccctccagattctccggctctggctccggcaccgacttcaccctgaccatcagctccctgcagcccgaggacttcgccacctactactgccagcagggcaactccttccctcggaccttcggcggaggcaccaaagtggaaatcaagcgtacggtggccgctccctccgtgttcatcttcccaccctccgacgagcagctgaagtccggcaccgcctccgtcgtgtgcctgctgaacaacttctaccctcgcgaggccaaagtgcagtggaaagtggacaacgccctgcagtccggcaactcccaggaatccgtcaccgagcaggactccaaggacagcacctactccctgtcctccaccctgaccctgtccaaggccgactacgagaagcacaaagtgtacgcctgcgaagtgacccaccagggcctgtccagccccgtgaccaagtccttcaaccggggcgagtgc

SEQ ID NO: 25 - protein, 10 aa, Artificial Sequence
  source: /note=“Description of Artificial Sequence: Synthetic peptide”
GGSIEGRYWS

SEQ ID NO: 26 - protein, 16 aa, Artificial Sequence
  source: /note=“Description of Artificial Sequence: Synthetic peptide”
YIYGSGSTKYNPSLKS

SEQ ID NO: 27 - protein, 7 aa, Artificial Sequence
  source: /note=“Description of Artificial Sequence: Synthetic peptide”
GGSIEGR

SEQ ID NO: 28 - protein, 5 aa, Artificial Sequence
  source: /note=“Description of Artificial Sequence: Synthetic peptide”
YGSGS

SEQ ID NO: 29 - protein, 8 aa, Artificial Sequence
  source: /note=“Description of Artificial Sequence: Synthetic peptide”
GGSIEGRY

SEQ ID NO: 30 - protein, 7 aa, Artificial Sequence
  source: /note=“Description of Artificial Sequence: Synthetic peptide”
IYGSGST

SEQ ID NO: 31 - protein, 121 aa, Artificial Sequence
  source: /note=“Description of Artificial Sequence: Synthetic polypeptide”
QVQLQESGPGLVKPSETLSLTCTVSGGSIEGRYWSWIRQPPGKGLEWIGYIYGSGSTKYNPSLKSRVTISVDTSKNQFSLKLSSVTAADTAVYYCARESQESPYNNWFDPWGQGTLVTVSS

SEQ ID NO: 32 - DNA, 363 bp, Artificial Sequence
  source: /note=“Description of Artificial Sequence: Synthetic polynucleotide”
caagtgcagctgcaggaatctggccctggcctggtcaagccctccgagacactgtccctgacctgcaccgtgtccggcggctctatcgagggccggtactggtcctggatccggcagcctcctggcaagggcctggaatggatcggctacatctacggctccggctccaccaagtacaaccccagcctgaagtccagagtgaccatctccgtggacacctccaagaaccagttctccctgaagctgtcctccgtgaccgccgctgacaccgccgtgtactactgcgccagagagtcccaggaatccccttacaacaattggttcgacccctggggccagggcaccctggtcaccgtgtcctct

SEQ ID NO: 33 - protein, 448 aa, Artificial Sequence
  source: /note=“Description of Artificial Sequence: Synthetic polypeptide”
  VARIANT (448)..(448): /replace=“ ”
  SITE (1)..(448): /note=“Variant residues given in the sequence have no preference with respect to those in the annotations for variant positions”
QVQLQESGPGLVKPSETLSLTCTVSGGSIEGRYWSWIRQPPGKGLEWIGYIYGSGSTKYNPSLKSRVTISVDTSKNQFSLKLSSVTAADTAVYYCARESQESPYNNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - DNA, 1344 bp, Artificial Sequence
  source: /note=“Description of Artificial Sequence: Synthetic polynucleotide”
  variation (1342)..(1342): /replace=“ ”
  variation (1343)..(1343): /replace=“ ”
  variation (1344)..(1344): /replace=“ ”
  misc_feature (1)..(1344): /note=“Variant nucleotides given in the sequence have no preference with respect to those in the annotations for variant positions”
caagtgcagctgcaggaatctggccctggcctggtcaagccctccgagacactgtccctgacctgcaccgtgtccggcggctctatcgagggccggtactggtcctggatccggcagcctcctggcaagggcctggaatggatcggctacatctacggctccggctccaccaagtacaaccccagcctgaagtccagagtgaccatctccgtggacacctccaagaaccagttctccctgaagctgtcctccgtgaccgccgctgacaccgccgtgtactactgcgccagagagtcccaggaatccccttacaacaattggttcgacccctggggccagggcaccctggtcaccgtgtcctctgcctccaccaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgccctccttgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggcaag

SEQ ID NO: 35 - protein, 10 aa, Artificial Sequence
  source: /note=“Description of Artificial Sequence: Synthetic peptide”
GFTFHRYAMS

SEQ ID NO: 36 - protein, 17 aa, Artificial Sequence
  source: /note=“Description of Artificial Sequence: Synthetic peptide”
AISGSGMNTYYADSVKG

SEQ ID NO: 37 - protein, 15 aa, Artificial Sequence
  source: /note=“Description of Artificial Sequence: Synthetic peptide”
GGLYGSGSYLSDFDL

SEQ ID NO: 38 - protein, 5 aa, Artificial Sequence
  source: /note=“Description of Artificial Sequence: Synthetic peptide”
RYAMS

SEQ ID NO: 39 - protein, 7 aa, Artificial Sequence
  source: /note=“Description of Artificial Sequence: Synthetic peptide”
GFTFHRY

SEQ ID NO: 40 - protein, 6 aa, Artificial Sequence
  source: /note=“Description of Artificial Sequence: Synthetic peptide”
SGSGMN

SEQ ID NO: 41 - protein, 8 aa, Artificial Sequence
  source: /note=“Description of Artificial Sequence: Synthetic peptide”
GFTFHRYA

SEQ ID NO: 42 - protein, 8 aa, Artificial Sequence
  source: /note=“Description of Artificial Sequence: Synthetic peptide”
ISGSGMNT

SEQ ID NO: 43 - protein, 17 aa, Artificial Sequence
  source: /note=“Description of Artificial Sequence: Synthetic peptide”
ARGGLYGSGSYLSDFDL

SEQ ID NO: 44 - protein, 124 aa, Artificial Sequence
  source: /note=“Description of Artificial Sequence: Synthetic polypeptide”
EVQLLESGGGLVQPGGSLRLSCAASGFTFHRYAMSWVRQAPGKGLEWVSAISGSGMNTYYADSVKGRFTISRDNSKNTLYLQMNSLRAEDTAVYYCARGGLYGSGSYLSDFDLWGRGTLVTVSS

SEQ ID NO: 45 - DNA, 372 bp, Artificial Sequence
  source: /note=“Description of Artificial Sequence: Synthetic polynucleotide”
gaagtgcagctgctggaatctggcggcggactggtgcagcctggcggctccctgagactgtcttgcgccgcctccggcttcaccttccacagatacgccatgtcctgggtccgacaggcccctggcaagggcctggagtgggtgtccgccatctccggctccggcatgaacacctactacgccgactccgtgaagggccggttcaccatctcccgggacaactccaagaacaccctgtacctgcagatgaactccctgcgggccgaggacaccgccgtgtactactgtgccagaggcggcctgtacggctccggctcctacctgtccgacttcgacctgtggggcagaggcaccctggtcaccgtgtcctcc

SEQ ID NO: 46 - protein, 451 aa, Artificial Sequence
  source: /note=“Description of Artificial Sequence: Synthetic polypeptide”
  VARIANT (451)..(451): /replace=“ ”
  SITE (1)..(451): /note=“Variant residues given in the sequence have no preference with respect to those in the annotations for variant positions”
EVQLLESGGGLVQPGGSLRLSCAASGFTFHRYAMSWVRQAPGKGLEWVSAISGSGMNTYYADSVKGRFTISRDNSKNTLYLQMNSLRAEDTAVYYCARGGLYGSGSYLSDFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - DNA, 1353 bp, Artificial Sequence
  source: /note=“Description of Artificial Sequence: Synthetic polynucleotide”
  variation (1351)..(1351): /replace=“ ”
  variation (1352)..(1352): /replace=“ ”
  variation (1353)..(1353): /replace=“ ”
  misc_feature (1)..(1353): /note=“Variant nucleotides given in the sequence have no preference with respect to those in the annotations for variant positions”
gaagtgcagctgctggaatctggcggcggactggtgcagcctggcggctccctgagactgtcttgcgccgcctccggcttcaccttccacagatacgccatgtcctgggtccgacaggcccctggcaagggcctggagtgggtgtccgccatctccggctccggcatgaacacctactacgccgactccgtgaagggccggttcaccatctcccgggacaactccaagaacaccctgtacctgcagatgaactccctgcgggccgaggacaccgccgtgtactactgtgccagaggcggcctgtacggctccggctcctacctgtccgacttcgacctgtggggcagaggcaccctggtcaccgtgtcctccgcctccacaaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgccctccttgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggcaag

SEQ ID NO: 48 - protein, 11 aa, Artificial Sequence
  source: /note=“Description of Artificial Sequence: Synthetic peptide”
RASQSVGSNLA

SEQ ID NO: 49 - protein, 7 aa, Artificial Sequence
  source: /note=“Description of Artificial Sequence: Synthetic peptide”
GASTRAT

SEQ ID NO: 50 - protein, 9 aa, Artificial Sequence
  source: /note=“Description of Artificial Sequence: Synthetic peptide”
QQHNAFPYT

SEQ ID NO: 51 - protein, 7 aa, Artificial Sequence
  source: /note=“Description of Artificial Sequence: Synthetic peptide”
SQSVGSN

SEQ ID NO: 52 - protein, 3 aa, Artificial Sequence
  source: /note=“Description of Artificial Sequence: Synthetic peptide”
GAS

SEQ ID NO: 53 - protein, 6 aa, Artificial Sequence
  source: /note=“Description of Artificial Sequence: Synthetic peptide”
HNAFPY

SEQ ID NO: 54 - protein, 6 aa, Artificial Sequence
  source: /note=“Description of Artificial Sequence: Synthetic peptide”
QSVGSN

SEQ ID NO: 55 - protein, 107 aa, Artificial Sequence
  source: /note=“Description of Artificial Sequence: Synthetic polypeptide”
EIVLTQSPATLSVSPGERATLSCRASQSVGSNLAWYQQKPGQAPRLLIYGASTRATGIPARFSGSGSGTEFTLTISSLQSEDFAVYYCQQHNAFPYTFGGGTKVEIK

SEQ ID NO: 56 - DNA, 321 bp, Artificial Sequence
  source: /note=“Description of Artificial Sequence: Synthetic polynucleotide”
gagatcgtgctgacacagtcccctgccaccctgtctgtgtctcccggcgagagagccaccctgagctgcagagcctcccagtccgtgggctccaacctggcctggtatcagcagaagcccggccaggcccctcggctgctgatctacggcgcctctaccagagccaccggcatccctgccagattctccggctctggctccggcaccgagttcaccctgaccatctccagcctgcagtccgaggacttcgccgtgtactactgccagcagcacaacgccttcccttacaccttcggcggaggcaccaaagtggaaatcaag

SEQ ID NO: 57 - protein, 214 aa, Artificial Sequence
  source: /note=“Description of Artificial Sequence: Synthetic polypeptide”
EIVLTQSPATLSVSPGERATLSCRASQSVGSNLAWYQQKPGQAPRLLIYGASTRATGIPARFSGSGSGTEFTLTISSLQSEDFAVYYCQQHNAFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - DNA, 642 bp, Artificial Sequence
  source: /note=“Description of Artificial Sequence: Synthetic polynucleotide”
gagatcgtgctgacacagtcccctgccaccctgtctgtgtctcccggcgagagagccaccctgagctgcagagcctcccagtccgtgggctccaacctggcctggtatcagcagaagcccggccaggcccctcggctgctgatctacggcgcctctaccagagccaccggcatccctgccagattctccggctctggctccggcaccgagttcaccctgaccatctccagcctgcagtccgaggacttcgccgtgtactactgccagcagcacaacgccttcccttacaccttcggcggaggcaccaaagtggaaatcaagcgtacggtggccgctccctccgtgttcatcttcccaccctccgacgagcagctgaagtccggcaccgcctccgtcgtgtgcctgctgaacaacttctaccctcgcgaggccaaagtgcagtggaaagtggacaacgccctgcagtccggcaactcccaggaatccgtcaccgagcaggactccaaggacagcacctactccctgtcctccaccctgaccctgtccaaggccgactacgagaagcacaaagtgtacgcctgcgaagtgacccaccagggcctgtccagccccgtgaccaagtccttcaaccggggcgagtgc

SEQ ID NO: 59 - protein, 10 aa, Artificial Sequence
  source: /note=“Description of Artificial Sequence: Synthetic peptide”
GGSIERYYWS

SEQ ID NO: 60 - protein, 16 aa, Artificial Sequence
  source: /note=“Description of Artificial Sequence: Synthetic peptide”
YIYGRGSTNYNPSLKS

SEQ ID NO: 61 - protein, 5 aa, Artificial Sequence
  source: /note=“Description of Artificial Sequence: Synthetic peptide”
RYYWS

SEQ ID NO: 62 - protein, 7 aa, Artificial Sequence
  source: /note=“Description of Artificial Sequence: Synthetic peptide”
GGSIERY

SEQ ID NO: 63 - protein, 5 aa, Artificial Sequence
  source: /note=“Description of Artificial Sequence: Synthetic peptide”
YGRGS

SEQ ID NO: 64 - protein, 8 aa, Artificial Sequence
  source: /note=“Description of Artificial Sequence: Synthetic peptide”
GGSIERYY

SEQ ID NO: 65 - protein, 7 aa, Artificial Sequence
  source: /note=“Description of Artificial Sequence: Synthetic peptide”
IYGRGST

SEQ ID NO: 66 - protein, 121 aa, Artificial Sequence
  source: /note=“Description of Artificial Sequence: Synthetic polypeptide”
QVQLQESGPGLVKPSETLSLTCTVSGGSIERYYWSWIRQPPGKGLEWIGYIYGRGSTNYNPSLKSRVTISVDTSKNQFSLKLSSVTAADTAVYYCARESQESPYNNWFDPWGQGTLVTVSS

SEQ ID NO: 67 - DNA, 363 bp, Artificial Sequence
  source: /note=“Description of Artificial Sequence: Synthetic polynucleotide”
caagtgcagctgcaggaatctggccctggcctggtcaagccctccgagacactgtccctgacctgcaccgtgtccggcggctccatcgagcggtactactggtcctggatccggcagcctcccggcaagggcctggaatggatcggctacatctacggcagaggctccaccaactacaaccccagcctgaagtccagagtgaccatctccgtggacacctccaagaaccagttctccctgaagctgtcctccgtgaccgccgctgacaccgccgtgtactactgcgccagagagtcccaggaatccccttacaacaattggttcgacccctggggccagggcaccctggtcaccgtgtcctct

SEQ ID NO: 68 - protein, 448 aa, Artificial Sequence
  source: /note=“Description of Artificial Sequence: Synthetic polypeptide”
  VARIANT (448)..(448): /replace=“ ”
  SITE (1)..(448): /note=“Variant residues given in the sequence have no preference with respect to those in the annotations for variant positions”
QVQLQESGPGLVKPSETLSLTCTVSGGSIERYYWSWIRQPPGKGLEWIGYIYGRGSTNYNPSLKSRVTISVDTSKNQFSLKLSSVTAADTAVYYCARESQESPYNNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 69 - DNA, 1344 bp, Artificial Sequence
  source: /note=“Description of Artificial Sequence: Synthetic polynucleotide”
  variation (1342)..(1342): /replace=“ ”
  variation (1343)..(1343): /replace=“ ”
  variation (1344)..(1344): /replace=“ ”
  misc_feature (1)..(1344): /note=“Variant nucleotides given in the sequence have no preference with respect to those in the annotations for variant positions”
caagtgcagctgcaggaatctggccctggcctggtcaagccctccgagacactgtccctgacctgcaccgtgtccggcggctccatcgagcggtactactggtcctggatccggcagcctcccggcaagggcctggaatggatcggctacatctacggcagaggctccaccaactacaaccccagcctgaagtccagagtgaccatctccgtggacacctccaagaaccagttctccctgaagctgtcctccgtgaccgccgctgacaccgccgtgtactactgcgccagagagtcccaggaatccccttacaacaattggttcgacccctggggccagggcaccctggtcaccgtgtcctctgcctccaccaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgccctccttgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggcaag

SEQ ID NO: 70 - protein, 10 aa, Artificial Sequence
  source: /note=“Description of Artificial Sequence: Synthetic peptide”
GFTFSYNAMS

SEQ ID NO: 71 - protein, 17 aa, Artificial Sequence
  source: /note=“Description of Artificial Sequence: Synthetic peptide”
SISGTGGSTYYADSVKG

SEQ ID NO: 72 - protein, 5 aa, Artificial Sequence
  source: /note=“Description of Artificial Sequence: Synthetic peptide”
YNAMS

SEQ ID NO: 73 - protein, 7 aa, Artificial Sequence
  source: /note=“Description of Artificial Sequence: Synthetic peptide”
GFTFSYN

SEQ ID NO: 74 - protein, 6 aa, Artificial Sequence
  source: /note=“Description of Artificial Sequence: Synthetic peptide”
SGTGGS

SEQ ID NO: 75 - protein, 8 aa, Artificial Sequence
  source: /note=“Description of Artificial Sequence: Synthetic peptide”
GFTFSYNA

SEQ ID NO: 76 - protein, 8 aa, Artificial Sequence
  source: /note=“Description of Artificial Sequence: Synthetic peptide”
ISGTGGST

SEQ ID NO: 77 - protein, 124 aa, Artificial Sequence
  source: /note=“Description of Artificial Sequence: Synthetic polypeptide”
EVQLLESGGGLVQPGGSLRLSCAASGFTFSYNAMSWVRQAPGKGLEWVSSISGTGGSTYYADSVKGRFTISRDNSKNTLYLQMNSLRAEDTAVYYCARGGLYGSGSYLSDFDLWGRGTLVTVSS

SEQ ID NO: 78 - DNA, 372 bp, Artificial Sequence
  source: /note=“Description of Artificial Sequence: Synthetic polynucleotide”
gaagtgcagctgctggaatctggcggcggactggtgcagcctggcggctccctgagactgtcttgcgccgcctccggcttcaccttctcctacaacgccatgtcctgggtccgacaggcccctggcaagggcctggagtgggtgtcctccatctccggcaccggcggctccacctactacgccgactctgtgaagggccggttcaccatctcccgggacaactccaagaacaccctgtacctgcagatgaactccctgcgggccgaggacaccgccgtgtactactgtgccagaggcggcctgtacggctccggctcctacctgtccgacttcgacctgtggggcagaggcaccctggtcaccgtgtcctcc

SEQ ID NO: 79 - protein, 451 aa, Artificial Sequence
  source: /note=“Description of Artificial Sequence: Synthetic polypeptide”
  VARIANT (451)..(451): /replace=“ ”
  SITE (1)..(451): /note=“Variant residues given in the sequence have no preference with respect to those in the annotations for variant positions”
EVQLLESGGGLVQPGGSLRLSCAASGFTFSYNAMSWVRQAPGKGLEWVSSISGTGGSTYYADSVKGRFTISRDNSKNTLYLQMNSLRAEDTAVYYCARGGLYGSGSYLSDFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 80 - DNA, 1353 bp, Artificial Sequence
  source: /note=“Description of Artificial Sequence: Synthetic polynucleotide”
  variation (1351)..(1351): /replace=“ ”
  variation (1352)..(1352): /replace=“ ”
  variation (1353)..(1353): /replace=“ ”
  misc_feature (1)..(1353): /note=“Variant nucleotides given in the sequence have no preference with respect to those in the annotations for variant positions”
gaagtgcagctgctggaatctggcggcggactggtgcagcctggcggctccctgagactgtcttgcgccgcctccggcttcaccttctcctacaacgccatgtcctgggtccgacaggcccctggcaagggcctggagtgggtgtcctccatctccggcaccggcggctccacctactacgccgactctgtgaagggccggttcaccatctcccgggacaactccaagaacaccctgtacctgcagatgaactccctgcgggccgaggacaccgccgtgtactactgtgccagaggcggcctgtacggctccggctcctacctgtccgacttcgacctgtggggcagaggcaccctggtcaccgtgtcctccgcctccacaaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgccctccttgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggcaag

SEQ ID NO: 81 - protein, 10 aa, Artificial Sequence
  source: /note=“Description of Artificial Sequence: Synthetic peptide”
GFTFSRYAMS

SEQ ID NO: 82 - protein, 7 aa, Artificial Sequence
  source: /note=“Description of Artificial Sequence: Synthetic peptide”
GFTFSRY

SEQ ID NO: 83 - protein, 8 aa, Artificial Sequence
  source: /note=“Description of Artificial Sequence: Synthetic peptide”
GFTFSRYA

SEQ ID NO: 84 - protein, 124 aa, Artificial Sequence
  source: /note=“Description of Artificial Sequence: Synthetic polypeptide”
EVQLLESGGGLVQPGGSLRLSCAASGFTFSRYAMSWVRQAPGKGLEWVSSISGTGGSTYYADSVKGRFTISRDNSKNTLYLQMNSLRAEDTAVYYCARGGLYGSGSYLSDFDLWGRGTLVTVSS

SEQ ID NO: 85 - DNA, 372 bp, Artificial Sequence
  source: /note=“Description of Artificial Sequence: Synthetic polynucleotide”
gaagtgcagctgctggaatctggcggcggactggtgcagcctggcggctccctgagactgtcttgcgccgcctccggcttcaccttctccagatacgccatgtcctgggtccgacaggcccctggcaagggcctggagtgggtgtcctccatctccggcaccggcggctccacctactacgccgactctgtgaagggccggttcaccatctcccgggacaactccaagaacaccctgtacctgcagatgaactccctgcgggccgaggacaccgccgtgtactactgtgccagaggcggcctgtacggctccggctcctacctgtccgacttcgacctgtggggcagaggcaccctggtcaccgtgtcctcc

SEQ ID NO: 86 - protein, 451 aa, Artificial Sequence
  source: /note=“Description of Artificial Sequence: Synthetic polypeptide”
  VARIANT (451)..(451): /replace=“ ”
  SITE (1)..(451): /note=“Variant residues given in the sequence have no preference with respect to those in the annotations for variant positions”
EVQLLESGGGLVQPGGSLRLSCAASGFTFSRYAMSWVRQAPGKGLEWVSSISGTGGSTYYADSVKGRFTISRDNSKNTLYLQMNSLRAEDTAVYYCARGGLYGSGSYLSDFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 87 - DNA, 1353 bp, Artificial Sequence
  source: /note=“Description of Artificial Sequence: Synthetic polynucleotide”
  variation (1351)..(1351): /replace=“ ”
  variation (1352)..(1352): /replace=“ ”
  variation (1353)..(1353): /replace=“ ”
  misc_feature (1)..(1353): /note=“Variant nucleotides given in the sequence have no preference with respect to those in the annotations for variant positions”
gaagtgcagctgctggaatctggcggcggactggtgcagcctggcggctccctgagactgtcttgcgccgcctccggcttcaccttctccagatacgccatgtcctgggtccgacaggcccctggcaagggcctggagtgggtgtcctccatctccggcaccggcggctccacctactacgccgactctgtgaagggccggttcaccatctcccgggacaactccaagaacaccctgtacctgcagatgaactccctgcgggccgaggacaccgccgtgtactactgtgccagaggcggcctgtacggctccggctcctacctgtccgacttcgacctgtggggcagaggcaccctggtcaccgtgtcctccgcctccacaaagggcccctccgtgttccctctggccccttgctcccggtccacctccgagtctaccgccgctctgggctgcctcgtgaaggactacttccccgagcccgtgaccgtgtcctggaactctggcgccctgacctccggcgtgcacaccttccctgccgtgctgcagtcctccggcctgtactccctgtccagcgtcgtgaccgtgccctcctccagcctgggcaccaagacctacacctgtaacgtggaccacaagccctccaacaccaaagtggacaagcgggtggaatctaagtacggccctccctgccctccttgccctgcccctgagttcctgggcggaccttccgtgttcctgttccctccaaagcccaaggacaccctgatgatctcccggacccctgaagtgacctgcgtggtggtggacgtgtcccaggaagatcccgaagtccagttcaattggtacgtggacggcgtggaagtgcacaacgccaagaccaagcccagagaggaacagttcaactccacctaccgggtggtgtccgtgctgaccgtgctgcaccaggactggctgaacggcaaagagtacaagtgcaaagtgtccaacaagggcctgccctccagcatcgaaaagaccatctccaaggccaagggccagccccgcgagccccaagtgtacaccctgcctcccagccaggaagagatgaccaagaatcaagtgtccctgacttgtctggtcaagggcttctacccctccgatatcgccgtggagtgggagtccaacggccagcccgagaacaactacaagaccacccctcccgtgctggactccgacggctccttcttcctgtactctcggctgaccgtggacaagtcccggtggcaggaaggcaacgtcttctcctgctccgtgatgcacgaggccctgcacaaccactacacccagaagtccctgtccctgtctctgggcaag

SEQ ID NO: 88 - protein, 5 aa, Artificial Sequence
  source: /note=“Description of Artificial Sequence: Synthetic peptide”
  VARIANT (1)..(1): /replace=“Y” or “S”
  VARIANT (2)..(2): /replace=“N”
  SITE (1)..(5): /note=“Variant residues given in the sequence have no preference with respect to those in the annotations for variant positions”
RYAMS

SEQ ID NO: 89 - protein, 17 aa, Artificial Sequence
  source: /note=“Description of Artificial Sequence: Synthetic peptide”
  VARIANT (1)..(1): /replace=“S”
  VARIANT (3)..(3): /replace=“T”
  VARIANT (5)..(5): /replace=“T”
  VARIANT (7)..(7): /replace=“G” or “S”
  VARIANT (8)..(8): /replace=“S” or “L” or “Y”
  SITE (1)..(17): /note=“Variant residues given in the sequence have no preference with respect to those in the annotations for variant positions”
AISGSGMNTYYADSVKG

SEQ ID NO: 90 - protein, 5 aa, Artificial Sequence
  source: /note=“Description of Artificial Sequence: Synthetic peptide”
  VARIANT (1)..(1): /replace=“G” or “S”
  VARIANT (2)..(2): /replace=“R”
  SITE (1)..(5): /note=“Variant residues given in the sequence have no preference with respect to those in the annotations for variant positions”
RYYWS

SEQ ID NO: 91 - protein, 16 aa, Artificial Sequence
  source: /note=“Description of Artificial Sequence: Synthetic peptide”
  VARIANT (4)..(4): /replace=“S”
  VARIANT (5)..(5): /replace=“S” or “T”
  VARIANT (9)..(9): /replace=“K”
  SITE (1)..(16): /note=“Variant residues given in the sequence have no preference with respect to those in the annotations for variant positions”
YIYGRGSTNYNPSLKS

SEQ ID NO: 92 - protein, 327 aa, Artificial Sequence
  source: /note=“Description of Artificial Sequence: Synthetic polypeptide”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 93 - protein, 326 aa, Artificial Sequence
  source: /note=“Description of Artificial Sequence: Synthetic polypeptide”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 94 - protein, 327 aa, Artificial Sequence
  source: /note=“Description of Artificial Sequence: Synthetic polypeptide”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 95 - protein, 326 aa, Artificial Sequence
  source: /note=“Description of Artificial Sequence: Synthetic polypeptide”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 96 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 98 - protein, 330 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 329 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 100 - protein, 330 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 329 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 102 - protein, 330 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 329 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 104 - protein, 107 aa, Homo sapiens [sequence omitted]

SEQ ID NO: 105 - protein, 574 aa, Homo sapiens [sequence omitted]

SEQ ID NO: 106 - protein, 574 aa, Homo sapiens
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFSTGSHCHGSFSLIFLSLWAVIFVLYQ

SEQ ID NO: 107 - protein, 524 aa, Homo sapiens
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFSTGSHCHGSFSLIFLSLWAVIFVLYQ

SEQ ID NO: 108 - protein, 15 aa, Homo sapiens
YLPYKVLPVGDEVVG

SEQ ID NO: 109 - protein, 10 aa, Homo sapiens
KLKTLNVNKI

SEQ ID NO: 110 - protein, 20 aa, Homo sapiens
MINNNLRHADETFWNHVSMC

SEQ ID NO: 111 - protein, 18 aa, Homo sapiens
YQGTIWFTVYKGAEVAHF

SEQ ID NO: 112 - protein, 448 aa, Artificial Sequence
  source: /note=“Description of Artificial Sequence: Synthetic polypeptide”
  VARIANT (448)..(448): /replace=“ ”
  SITE (1)..(448): /note=“Variant residues given in the sequence have no preference with respect to those in the annotations for variant positions”
QVQLQESGPGLVKPSETLSLTCTVSGGSISGRYWSWIRQPPGKGLEWIGYIYGTGSTNYNPSLKSRVTISVDTSKNQFSLKLSSVTAADTAVYYCARESQESPYNNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 113 - protein, 448 aa, Artificial Sequence
  source: /note=“Description of Artificial Sequence: Synthetic polypeptide”
  VARIANT (448)..(448): /replace=“ ”
  SITE (1)..(448): /note=“Variant residues given in the sequence have no preference with respect to those in the annotations for variant positions”
QVQLQESGPGLVKPSETLSLTCTVSGGSIEGRYWSWIRQPPGKGLEWIGYIYGSGSTKYNPSLKSRVTISVDTSKNQFSLKLSSVTAADTAVYYCARESQESPYNNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 114 - protein, 451 aa, Artificial Sequence
  source: /note=“Description of Artificial Sequence: Synthetic polypeptide”
  VARIANT (451)..(451): /replace=“ ”
  SITE (1)..(451): /note=“Variant residues given in the sequence have no preference with respect to those in the annotations for variant positions”
EVQLLESGGGLVQPGGSLRLSCAASGFTFHRYAMSWVRQAPGKGLEWVSAISGSGMNTYYADSVKGRFTISRDNSKNTLYLQMNSLRAEDTAVYYCARGGLYGSGSYLSDFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 115 - protein, 448 aa, Artificial Sequence
  source: /note=“Description of Artificial Sequence: Synthetic polypeptide”
  VARIANT (448)..(448): /replace=“ ”
  SITE (1)..(448): /note=“Variant residues given in the sequence have no preference with respect to those in the annotations for variant positions”
QVQLQESGPGLVKPSETLSLTCTVSGGSIERYYWSWIRQPPGKGLEWIGYIYGRGSTNYNPSLKSRVTISVDTSKNQFSLKLSSVTAADTAVYYCARESQESPYNNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 116 - protein, 451 aa, Artificial Sequence
  source: /note=“Description of Artificial Sequence: Synthetic polypeptide”
  VARIANT (451)..(451): /replace=“ ”
  SITE (1)..(451): /note=“Variant residues given in the sequence have no preference with respect to those in the annotations for variant positions”
EVQLLESGGGLVQPGGSLRLSCAASGFTFSYNAMSWVRQAPGKGLEWVSSISGTGGSTYYADSVKGRFTISRDNSKNTLYLQMNSLRAEDTAVYYCARGGLYGSGSYLSDFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 117 - protein, 451 aa, Artificial Sequence
  source: /note=“Description of Artificial Sequence: Synthetic polypeptide”
  VARIANT (451)..(451): /replace=“ ”
  SITE (1)..(451): /note=“Variant residues given in the sequence have no preference with respect to those in the annotations for variant positions”
EVQLLESGGGLVQPGGSLRLSCAASGFTFSRYAMSWVRQAPGKGLEWVSSISGTGGSTYYADSVKGRFTISRDNSKNTLYLQMNSLRAEDTAVYYCARGGLYGSGSYLSDFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 118 - protein, 13 aa, Homo sapiens
RGNVISSHGNPIL

SEQ ID NO: 119 - protein, 330 aa, Homo sapiens [sequence omitted]

SEQ ID NO: 120 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 121 - protein, 20 aa, Homo sapiens
MINNNLRHTDEMFWNHVSMC

SEQ ID NO: 122 - protein, 7 aa, Artificial Sequence
  source: /note=“Description of Artificial Sequence: Synthetic peptide”
  VARIANT (5)..(5): /replace=“S” or “R” or “K”
  VARIANT (6)..(6): /replace=“Y” or “S”
  VARIANT (7)..(7): /replace=“N”
  SITE (1)..(7): /note=“Variant residues given in the sequence have no preference with respect to those in the annotations for variant positions”
GFTFHRY

SEQ ID NO: 123 - protein, 6 aa, Artificial Sequence
  source: /note=“Description of Artificial Sequence: Synthetic peptide”
  VARIANT (1)..(1): /replace=“T”
  VARIANT (3)..(3): /replace=“T”
  VARIANT (5)..(5): /replace=“G” or “S”
  VARIANT (6)..(6): /replace=“S” or “L” or “Y”
  SITE (1)..(6): /note=“Variant residues given in the sequence have no preference with respect to those in the annotations for variant positions”
SGSGMN

SEQ ID NO: 124 - protein, 7 aa, Artificial Sequence
  source: /note=“Description of Artificial Sequence: Synthetic peptide”
  VARIANT (5)..(5): /replace=“S”
  VARIANT (6)..(6): /replace=“G” or “S”
  VARIANT (7)..(7): /replace=“R”
  SITE (1)..(7): /note=“Variant residues given in the sequence have no preference with respect to those in the annotations for variant positions”
GGSIERY

SEQ ID NO: 125 - protein, 5 aa, Artificial Sequence
  source: /note=“Description of Artificial Sequence: Synthetic peptide”
  VARIANT (2)..(2): /replace=“S”
  VARIANT (3)..(3): /replace=“S” or “T”
  SITE (1)..(5): /note=“Variant residues given in the sequence have no preference with respect to those in the annotations for variant positions”
YGRGS

SEQ ID NO: 126 - protein, 10 aa, Artificial Sequence
  source: /note=“Description of Artificial Sequence: Synthetic peptide”
  VARIANT (5)..(5): /replace=“S” or “R” or “K”
  VARIANT (6)..(6): /replace=“Y” or “S”
  VARIANT (7)..(7): /replace=“N”
  SITE (1)..(10): /note=“Variant residues given in the sequence have no preference with respect to those in the annotations for variant positions”
GFTFHRYAMS

SEQ ID NO: 127 - protein, 10 aa, Artificial Sequence
  source: /note=“Description of Artificial Sequence: Synthetic peptide”
  VARIANT (5)..(5): /replace=“S”
  VARIANT (6)..(6): /replace=“G” or “S”
  VARIANT (7)..(7): /replace=“R”
  SITE (1)..(10): /note=“Variant residues given in the sequence have no preference with respect to those in the annotations for variant positions”
GGSIERYYWS

SEQ ID NO: 128 - protein, 8 aa, Artificial Sequence
  source: /note=“Description of Artificial Sequence: Synthetic peptide”
  VARIANT (5)..(5): /replace=“S” or “R” or “K”
  VARIANT (6)..(6): /replace=“Y” or “S”
  VARIANT (7)..(7): /replace=“N”
  SITE (1)..(8): /note=“Variant residues given in the sequence have no preference with respect to those in the annotations for variant positions”
GFTFHRYA

SEQ ID NO: 129 - protein, 8 aa, Artificial Sequence
  source: /note=“Description of Artificial Sequence: Synthetic peptide”
  VARIANT (2)..(2): /replace=“T”
  VARIANT (4)..(4): /replace=“T”
  VARIANT (6)..(6): /replace=“G” or “S”
  VARIANT (7)..(7): /replace=“S” or “L” or “Y”
  SITE (1)..(8): /note=“Variant residues given in the sequence have no preference with respect to those in the annotations for variant positions”
ISGSGMNT

SEQ ID NO: 130 - protein, 8 aa, Artificial Sequence
  source: /note=“Description of Artificial Sequence: Synthetic peptide”
  VARIANT (5)..(5): /replace=“S”
  VARIANT (6)..(6): /replace=“G” or “S”
  VARIANT (7)..(7): /replace=“R”
  SITE (1)..(8): /note=“Variant residues given in the sequence have no preference with respect to those in the annotations for variant positions”
GGSIERYY

SEQ ID NO: 131 - protein, 7 aa, Artificial Sequence
  source: /note=“Description of Artificial Sequence: Synthetic peptide”
  VARIANT (3)..(3): /replace=“S”
  VARIANT (4)..(4): /replace=“S” or “T”
  SITE (1)..(7): /note=“Variant residues given in the sequence have no preference with respect to those in the annotations for variant positions”
IYGRGST

SEQ ID NO: 132-133 - skipped (2 entries)

SEQ ID NO: 134 - protein, 521 aa, Homo sapiens
WELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHADETFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIK

SEQ ID NO: 135 - protein, 10 aa, Artificial Sequence
  source: /note=“Description of Artificial Sequence: Synthetic peptide”
GFTFRSYAMS

SEQ ID NO: 136 - protein, 17 aa, Artificial Sequence
  source: /note=“Description of Artificial Sequence: Synthetic peptide”
AITGSGGLTYYADSVKG

SEQ ID NO: 137 - protein, 5 aa, Artificial Sequence
  source: /note=“Description of Artificial Sequence: Synthetic peptide”
SYAMS

SEQ ID NO: 138 - protein, 7 aa, Artificial Sequence
  source: /note=“Description of Artificial Sequence: Synthetic peptide”
GFTFRSY

SEQ ID NO: 139 - protein, 6 aa, Artificial Sequence
  source: /note=“Description of Artificial Sequence: Synthetic peptide”
TGSGGL

SEQ ID NO: 140 - protein, 8 aa, Artificial Sequence
  source: /note=“Description of Artificial Sequence: Synthetic peptide”
GFTFRSYA

SEQ ID NO: 141 - protein, 8 aa, Artificial Sequence
  source: /note=“Description of Artificial Sequence: Synthetic peptide”
ITGSGGLT

SEQ ID NO: 142 - protein, 124 aa, Artificial Sequence
  source: /note=“Description of Artificial Sequence: Synthetic polypeptide”
EVQLLESGGGLVQPGGSLRLSCAASGFTFRSYAMSWVRQAPGKGLEWVSAITGSGGLTYYADSVKGRFTISRDNSKNTLYLQMNSLRAEDTAVYYCARGGLYGSGSYLSDFDLWGRGTLVTVSS

SEQ ID NO: 143 - DNA, 372 bp, Artificial Sequence
  source: /note=“Description of Artificial Sequence: Synthetic polynucleotide”
gaggtgcagctgttggagtctgggggaggcttggtacagcctggggggtccctgagactctcctgtgcagcctctggattcacctttcgtagctatgccatgagctgggtccgccaggctccagggaaggggctggagtgggtctcagctattacgggaagtggtggtttgacatactacgcagactccgtgaagggccggttcaccatctccagagacaattccaagaacacgctgtatctgcaaatgaacagcctgagagccgaggacacggcggtgtactactgcgccagaggtggattgtacggaagcggaagctacttgagtgacttcgacctatgggggagaggtaccttggtcaccgtctcctca

SEQ ID NO: 144 - DNA, 321 bp, Artificial Sequence
  source: /note=“Description of Artificial Sequence: Synthetic polynucleotide”
gaaatagtgttgacgcagtctccagccaccctgtctgtgtctccaggggaaagagccaccctctcctgcagggccagtcagagtgttggcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagcacaatgccttcccttacacttttggcggagggaccaaggttgagatcaaa

SEQ ID NO: 145 - protein, 10 aa, Artificial Sequence
  source: /note=“Description of Artificial Sequence: Synthetic peptide”
GFTFKSYAMS

SEQ ID NO: 146 - protein, 17 aa, Artificial Sequence
  source: /note=“Description of Artificial Sequence: Synthetic peptide”
AISGSGSYTYYADSVKG

SEQ ID NO: 147 - protein, 7 aa, Artificial Sequence
  source: /note=“Description of Artificial Sequence: Synthetic peptide”
GFTFKSY

SEQ ID NO: 148 - protein, 6 aa, Artificial Sequence
  source: /note=“Description of Artificial Sequence: Synthetic peptide”
SGSGSY

SEQ ID NO: 149 - protein, 8 aa, Artificial Sequence
  source: /note=“Description of Artificial Sequence: Synthetic peptide”
GFTFKSYA

SEQ ID NO: 150 - protein, 8 aa, Artificial Sequence
  source: /note=“Description of Artificial Sequence: Synthetic peptide”
ISGSGSYT

SEQ ID NO: 151 - protein, 124 aa, Artificial Sequence
  source: /note=“Description of Artificial Sequence: Synthetic polypeptide”
EVQLLESGGGLVQPGGSLRLSCAASGFTFKSYAMSWVRQAPGKGLEWVSAISGSGSYTYYADSVKGRFTISRDNSKNTLYLQMNSLRAEDTAVYYCARGGLYGSGSYLSDFDLWGRGTLVTVSS

SEQ ID NO: 152 - DNA, 372 bp, Artificial Sequence
  source: /note=“Description of Artificial Sequence: Synthetic polynucleotide”
gaggtgcagctgttggagtctgggggaggcttggtacagcctggggggtccctgagactctcctgtgcagcctctggattcacctttaagagctatgccatgagttgggtccgccaggctccagggaaggggctggagtgggtctcagctattagtggaagtggttcgtatacatactacgcagactccgtgaagggccggttcaccatctccagagacaattccaagaacacgctgtatctgcaaatgaacagcctgagagccgaggacacggcggtgtactactgcgccagaggtggattgtacggaagcggaagctacttgagtgacttcgacctatgggggagaggtaccttggtcaccgtctcctca

SEQ ID NO: 153 - protein, 10 aa, Artificial Sequence
  source: /note=“Description of Artificial Sequence: Synthetic peptide”
GFTFSSYAMS

SEQ ID NO: 154 - protein, 17 aa, Artificial Sequence
  source: /note=“Description of Artificial Sequence: Synthetic peptide”
AISGSGGSTYYADSVKG

SEQ ID NO: 155 - protein, 7 aa, Artificial Sequence
  source: /note=“Description of Artificial Sequence: Synthetic peptide”
GFTFSSY

SEQ ID NO: 156 - protein, 6 aa, Artificial Sequence
  source: /note=“Description of Artificial Sequence: Synthetic peptide”
SGSGGS

SEQ ID NO: 157 - protein, 8 aa, Artificial Sequence
  source: /note=“Description of Artificial Sequence: Synthetic peptide”
GFTFSSYA

SEQ ID NO: 158 - protein, 8 aa, Artificial Sequence
  source: /note=“Description of Artificial Sequence: Synthetic peptide”
ISGSGGST

SEQ ID NO: 159 - protein, 124 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGGLYGSGSYLSDFDLWGRGTLVTVSS

SEQ ID NO: 160 - DNA, 372 bp, Artificial Sequence
  source: /note=“Description of Artificial Sequence: Synthetic polynucleotide”
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggcggtgtactactgcgccagaggtggattgtacggaagcggaagctacttgagtgacttcgacctatgggggagaggtaccttggtcaccgtctcctca

SEQ ID NO: 161 - protein, 10 aa, Artificial Sequence
  source: /note=“Description of Artificial Sequence: Synthetic peptide”
GGSISSYYWS

SEQ ID NO: 162 - protein, 16 aa, Artificial Sequence
  source: /note=“Description of Artificial Sequence: Synthetic peptide”
YIYSSGSTNYNPSLKS

SEQ ID NO: 163 - protein, 5 aa, Artificial Sequence
  source: /note=“Description of Artificial Sequence: Synthetic peptide”
SYYWS

SEQ ID NO: 164 - protein, 7 aa, Artificial Sequence
  source: /note=“Description of Artificial Sequence: Synthetic peptide”
GGSISSY

SEQ ID NO: 165 - protein, 5 aa, Artificial Sequence
  source: /note=“Description of Artificial Sequence: Synthetic peptide”
YSSGS

SEQ ID NO: 166 - protein, 8 aa, Artificial Sequence
  source: /note=“Description of Artificial Sequence: Synthetic peptide”
GGSISSYY

SEQ ID NO: 167 - protein, 7 aa, Artificial Sequence
  source: /note=“Description of Artificial Sequence: Synthetic peptide”
IYSSGST

SEQ ID NO: 168 - protein, 121 aa, Artificial Sequence
  source: /note=“Description of Artificial Sequence: Synthetic polypeptide”
QVQLQESGPGLVKPSETLSLTCTVSGGSISSYYWSWIRQPPGKGLEWIGYIYSSGSTNYNPSLKSRVTISVDTSKNQFSLKLSSVTAADTAVYYCARESQESPYNNWFDPWGQGTLVTVSS

SEQ ID NO: 169 - DNA, 363 bp, Artificial Sequence
  source: /note=“Description of Artificial Sequence: Synthetic polynucleotide”
caggtgcagctgcaggagtcgggcccaggactggtgaagccttcggagaccctgtccctcacctgcactgtctctggtggctccatcagtagttactactggagctggatccggcagcccccagggaagggactggagtggattgggtatatctatagtagtgggagcaccaactacaacccctccctcaagagtcgagtcaccatatcagtagacacgtccaagaaccagttctccctgaagctgagttctgtgaccgccgcagacacggcggtgtactactgcgccagagaatctcaggagagtccatacaacaattggttcgacccatggggacagggtacattggtcaccgtctcctca

SEQ ID NO: 170 - DNA, 321 bp, Artificial Sequence
  source: /note=“Description of Artificial Sequence: Synthetic polynucleotide”
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttattactgtcagcagggaaacagtttccctaggacttttggcggagggaccaaggttgagatcaaa

SEQ ID NO: 171 - protein, 527 aa, Artificial Sequence
  source: /note=“Description of Artificial Sequence: Synthetic polypeptide”
WELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHADETFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKHHHHHH

SEQ ID NO: 172-188 - skipped (17 entries)

SEQ ID NO: 189 - protein, 9 aa, Artificial Sequence
  source: /note=“Description of Artificial Sequence: Synthetic peptide”
  VARIANT (4)..(4): /replace=“S” or “R” or “K”
  VARIANT (5)..(5): /replace=“Y” or “S”
  VARIANT (6)..(6): /replace=“N”
  SITE (1)..(9): /note=“Variant residues given in the sequence have no preference with respect to those in the annotations for variant positions”
FTFHRYAMS

SEQ ID NO: 190 - protein, 9 aa, Artificial Sequence
  source: /note=“Description of Artificial Sequence: Synthetic peptide”
FTFHRYAMS

SEQ ID NO: 191 - protein, 9 aa, Artificial Sequence
  source: /note=“Description of Artificial Sequence: Synthetic peptide”
FTFSYNAMS

SEQ ID NO: 192 - protein, 9 aa, Artificial Sequence
  source: /note=“Description of Artificial Sequence: Synthetic peptide”
FTFSRYAMS

SEQ ID NO: 193 - protein, 9 aa, Artificial Sequence
  source: /note=“Description of Artificial Sequence: Synthetic peptide”
FTFRSYAMS

SEQ ID NO: 194 - protein, 9 aa, Artificial Sequence
  source: /note=“Description of Artificial Sequence: Synthetic peptide”
FTFKSYAMS

SEQ ID NO: 195 - protein, 9 aa, Artificial Sequence
  source: /note=“Description of Artificial Sequence: Synthetic peptide”
FTFSSYAMS

SEQ ID NO: 196 - protein, 9 aa, Artificial Sequence
  source: /note=“Description of Artificial Sequence: Synthetic peptide”
  VARIANT (4)..(4): /replace=“S”
  VARIANT (5)..(5): /replace=“G” or “S”
  VARIANT (6)..(6): /replace=“R”
  SITE (1)..(9): /note=“Variant residues given in the sequence have no preference with respect to those in the annotations for variant positions”
GSIERYYWS

SEQ ID NO: 197 - protein, 9 aa, Artificial Sequence
  source: /note=“Description of Artificial Sequence: Synthetic peptide”
GSIERYYWS

SEQ ID NO: 198 - protein, 9 aa, Artificial Sequence
  source: /note=“Description of Artificial Sequence: Synthetic peptide”
GSIEGRYWS

SEQ ID NO: 199 - protein, 9 aa, Artificial Sequence
  source: /note=“Description of Artificial Sequence: Synthetic peptide”
GSISGRYWS

SEQ ID NO: 200 - protein, 9 aa, Artificial Sequence
  source: /note=“Description of Artificial Sequence: Synthetic peptide”
GSISSYYWS

SEQ ID NO: 201 - protein, 17 aa, Artificial Sequence
  source: /note=“Description of Artificial Sequence: Synthetic peptide”
AISGTGISTYYADSVKG

SEQ ID NO: 202 - protein, 6 aa, Artificial Sequence
  source: /note=“Description of Artificial Sequence: Synthetic peptide”
SGTGIS

SEQ ID NO: 203 - protein, 8 aa, Artificial Sequence
  source: /note=“Description of Artificial Sequence: Synthetic peptide”
ISGTGIST

SEQ ID NO: 204 - protein, 124 aa, Artificial Sequence
  source: /note=“Description of Artificial Sequence: Synthetic polypeptide”
EVQLLESGGGLVQPGGSLRLSCAASGFTFSRYAMSWVRQAPGKGLEWVSAISGTGISTYYADSVKGRFTISRDNSKNTLYLQMNSLRAEDTAVYYCARGGLYGSGSYLSDFDLWGRGTLVTVSS

SEQ ID NO: 205 - protein, 11 aa, Artificial Sequence
  source: /note=“Description of Artificial Sequence: Synthetic peptide”
RASQSISSWLA

SEQ ID NO: 206 - protein, 7 aa, Artificial Sequence
  source: /note=“Description of Artificial Sequence: Synthetic peptide”
DASSLES

SEQ ID NO: 207 - protein, 8 aa, Artificial Sequence
  source: /note=“Description of Artificial Sequence: Synthetic peptide”
QQSNTFYT

SEQ ID NO: 208 - protein, 7 aa, Artificial Sequence
  source: /note=“Description of Artificial Sequence: Synthetic peptide”
SQSISSW

SEQ ID NO: 209 - protein, 3 aa, Artificial Sequence
  source: /note=“Description of Artificial Sequence: Synthetic peptide”
DAS

SEQ ID NO: 210-227 - skipped (18 entries)

SEQ ID NO: 228 - protein, 5 aa, Artificial Sequence
  source: /note=“Description of Artificial Sequence: Synthetic peptide”
SNTFY

SEQ ID NO: 229 - protein, 6 aa, Artificial Sequence
  source: /note=“Description of Artificial Sequence: Synthetic peptide”
QSISSW

SEQ ID NO: 230 - protein, 106 aa, Artificial Sequence
  source: /note=“Description of Artificial Sequence: Synthetic polypeptide”
DIQMTQSPSTLSASVGDRVTITCRASQSISSWLAWYQQKPGKAPKLLIYDASSLESGVPSRFSGSGSGTEFTLTISSLQPDDFATYYCQQSNTFYTFGGGTKVEIK

SEQ ID NO: 231 - protein, 17 aa, Artificial Sequence
  source: /note=“Description of Artificial Sequence: Synthetic peptide”
AISGTGLSTYYADSVKG

SEQ ID NO: 232 - protein, 6 aa, Artificial Sequence
  source: /note=“Description of Artificial Sequence: Synthetic peptide”
SGTGLS

SEQ ID NO: 233 - protein, 8 aa, Artificial Sequence
  source: /note=“Description of Artificial Sequence: Synthetic peptide”
ISGTGLST

SEQ ID NO: 234 - protein, 124 aa, Artificial Sequence
  source: /note=“Description of Artificial Sequence: Synthetic polypeptide”
EVQLLESGGGLVQPGGSLRLSCAASGFTFSRYAMSWVRQAPGKGLEWVSAISGTGLSTYYADSVKGRFTISRDNSKNTLYLQMNSLRAEDTAVYYCARGGLYGSGSYLSDFDLWGRGTLVTVSS

SEQ ID NO: 235 - protein, 10 aa, Artificial Sequence
  source: /note=“Description of Artificial Sequence: Synthetic peptide”
GYTFTYYWMH

SEQ ID NO: 236 - protein, 17 aa, Artificial Sequence
  source: /note=“Description of Artificial Sequence: Synthetic peptide”
SINPNSGSTNYAQKFQG

SEQ ID NO: 237 - protein, 18 aa, Artificial Sequence
  source: /note=“Description of Artificial Sequence: Synthetic peptide”
DTGGDKSPLTYYYYGMDV

SEQ ID NO: 238 - protein, 5 aa, Artificial Sequence
  source: /note=“Description of Artificial Sequence: Synthetic peptide”
YYWMH

SEQ ID NO: 239 - protein, 7 aa, Artificial Sequence
  source: /note=“Description of Artificial Sequence: Synthetic peptide”
GYTFTYY

SEQ ID NO: 240 - protein, 6 aa, Artificial Sequence
  source: /note=“Description of Artificial Sequence: Synthetic peptide”
NPNSGS

SEQ ID NO: 241 - protein, 8 aa, Artificial Sequence
  source: /note=“Description of Artificial Sequence: Synthetic peptide”
GYTFTYYW

SEQ ID NO: 242 - protein, 8 aa, Artificial Sequence
  source: /note=“Description of Artificial Sequence: Synthetic peptide”
INPNSGST

SEQ ID NO: 243 - protein, 20 aa, Artificial Sequence
  source: /note=“Description of Artificial Sequence: Synthetic peptide”
ARDTGGDKSPLTYYYYGMDV

SEQ ID NO: 244 - protein, 9 aa, Artificial Sequence
  source: /note=“Description of Artificial Sequence: Synthetic peptide”
YTFTYYWMH

SEQ ID NO: 245 - protein, 127 aa, Artificial Sequence
  source: /note=“Description of Artificial Sequence: Synthetic polypeptide”
QVQLVQSGAEVKKPGASVKVSCKASGYTFTYYWMHWVRQAPGQGLEWMGSINPNSGSTNYAQKFQGRVTMTRDTSISTAYMELSRLRSDDTAVYYCARDTGGDKSPLTYYYYGMDVWGQGTTVTVSS

SEQ ID NO: 246 - protein, 11 aa, Artificial Sequence
  source: /note=“Description of Artificial Sequence: Synthetic peptide”
RASQGISRWLA

SEQ ID NO: 247 - protein, 10 aa, Artificial Sequence
  source: /note=“Description of Artificial Sequence: Synthetic peptide”
QQAIALPPFT

SEQ ID NO: 248 - protein, 7 aa, Artificial Sequence
  source: /note=“Description of Artificial Sequence: Synthetic peptide”
SQGISRW

SEQ ID NO: 249 - protein, 7 aa, Artificial Sequence
  source: /note=“Description of Artificial Sequence: Synthetic peptide”
AIALPPF

SEQ ID NO: 250 - protein, 6 aa, Artificial Sequence
  source: /note=“Description of Artificial Sequence: Synthetic peptide”
QGISRW

SEQ ID NO: 251 - protein, 108 aa, Artificial Sequence
  source: /note=“Description of Artificial Sequence: Synthetic polypeptide”
DIQMTQSPSSVSASVGDRVTITCRASQGISRWLAWYQQKPGKAPKLLIYAASSLQSGVPSRFSGSGSGTDFTLTISSLQPEDFATYYCQQAIALPPFTFGGGTKVEIK

SEQ ID NO: 252 - protein, 10 aa, Artificial Sequence
  source: /note=“Description of Artificial Sequence: Synthetic peptide”
GFTFSSYWMS

SEQ ID NO: 253 - protein, 17 aa, Artificial Sequence
  source: /note=“Description of Artificial Sequence: Synthetic peptide”
NIKQDGSEKYYVDSVKG

SEQ ID NO: 254 - protein, 19 aa, Artificial Sequence
  source: /note=“Description of Artificial Sequence: Synthetic peptide”
APEYYSTTTRLYYYYGMDV

SEQ ID NO: 255 - protein, 5 aa, Artificial Sequence
  source: /note=“Description of Artificial Sequence: Synthetic peptide”
SYWMS

SEQ ID NO: 256 - protein, 6 aa, Artificial Sequence
  source: /note=“Description of Artificial Sequence: Synthetic peptide”
KQDGSE

SEQ ID NO: 257 - protein, 8 aa, Artificial Sequence
  source: /note=“Description of Artificial Sequence: Synthetic peptide”
GFTFSSYW

SEQ ID NO: 258 - protein, 8 aa, Artificial Sequence
  source: /note=“Description of Artificial Sequence: Synthetic peptide”
IKQDGSEK

SEQ ID NO: 259 - protein, 21 aa, Artificial Sequence
  source: /note=“Description of Artificial Sequence: Synthetic peptide”
ARAPEYYSTTTRLYYYYGMDV

SEQ ID NO: 260 - protein, 9 aa, Artificial Sequence
  source: /note=“Description of Artificial Sequence: Synthetic peptide”
FTFSSYWMS

SEQ ID NO: 261 - protein, 128 aa, Artificial Sequence
  source: /note=“Description of Artificial Sequence: Synthetic polypeptide”
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARAPEYYSTTTRLYYYYGMDVWGQGTTVTVSS

SEQ ID NO: 262 - protein, 17 aa, Artificial Sequence
  source: /note=“Description of Artificial Sequence: Synthetic peptide”
KSSQSVLYSSNNKNYLA

SEQ ID NO: 263 - protein, 7 aa, Artificial Sequence
  source: /note=“Description of Artificial Sequence: Synthetic peptide”
WASTRES

SEQ ID NO: 264 - protein, 9 aa, Artificial Sequence
  source: /note=“Description of Artificial Sequence: Synthetic peptide”
QQYDAHPFT

SEQ ID NO: 265 - protein, 13 aa, Artificial Sequence
  source: /note=“Description of Artificial Sequence: Synthetic peptide”
SQSVLYSSNNKNY

SEQ ID NO: 266 - protein, 3 aa, Artificial Sequence
  source: /note=“Description of Artificial Sequence: Synthetic peptide”
WAS

SEQ ID NO: 267 - protein, 6 aa, Artificial Sequence
  source: /note=“Description of Artificial Sequence: Synthetic peptide”
YDAHPF

SEQ ID NO: 268 - protein, 12 aa, Artificial Sequence
  source: /note=“Description of Artificial Sequence: Synthetic peptide”
QSVLYSSNNKNY

SEQ ID NO: 269 - protein, 113 aa, Artificial Sequence
  source: /note=“Description of Artificial Sequence: Synthetic polypeptide”
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYDAHPFTFGGGTKVEIK

SEQ ID NO: 270-286 - skipped (17 entries)

SEQ ID NO: 287 - protein, 10 aa, Artificial Sequence
  source: /note=“Description of Artificial Sequence: Synthetic peptide”
GGTFSSYAIS

SEQ ID NO: 288 - protein, 17 aa, Artificial Sequence
  source: /note=“Description of Artificial Sequence: Synthetic peptide”
GIIPIFGTANYAQKFQG

SEQ ID NO: 289 - protein, 16 aa, Artificial Sequence
  source: /note=“Description of Artificial Sequence: Synthetic peptide”
HYYDYWSGYYTNTGIY

SEQ ID NO: 290 - protein, 5 aa, Artificial Sequence
  source: /note=“Description of Artificial Sequence: Synthetic peptide”
SYAIS

SEQ ID NO: 291 - protein, 7 aa, Artificial Sequence
  source: /note=“Description of Artificial Sequence: Synthetic peptide”
GGTFSSY

SEQ ID NO: 292 - protein, 6 aa, Artificial Sequence
  source: /note=“Description of Artificial Sequence: Synthetic peptide”
IPIFGT

SEQ ID NO: 293 - protein, 8 aa, Artificial Sequence
  source: /note=“Description of Artificial Sequence: Synthetic peptide”
GGTFSSYA

SEQ ID NO: 294 - protein, 8 aa, Artificial Sequence
  source: /note=“Description of Artificial Sequence: Synthetic peptide”
IIPIFGTA

SEQ ID NO: 295 - protein, 18 aa, Artificial Sequence
  source: /note=“Description of Artificial Sequence: Synthetic peptide”
ARHYYDYWSGYYTNTGIY

SEQ ID NO: 296 - protein, 9 aa, Artificial Sequence
  source: /note=“Description of Artificial Sequence: Synthetic peptide”
GTFSSYAIS

SEQ ID NO: 297 - protein, 125 aa, Artificial Sequence
  source: /note=“Description of Artificial Sequence: Synthetic polypeptide”
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARHYYDYWSGYYTNTGIYWGQGTLVTVSS

SEQ ID NO: 298 - protein, 11 aa, Artificial Sequence
  source: /note=“Description of Artificial Sequence: Synthetic peptide”
RASQSVSSNLA

SEQ ID NO: 299 - protein, 9 aa, Artificial Sequence
  source: /note=“Description of Artificial Sequence: Synthetic peptide”
QQSGALPIT

SEQ ID NO: 300 - protein, 7 aa, Artificial Sequence
  source: /note=“Description of Artificial Sequence: Synthetic peptide”
SQSVSSN

SEQ ID NO: 301 - protein, 6 aa, Artificial Sequence
  source: /note=“Description of Artificial Sequence: Synthetic peptide”
SGALPI

SEQ ID NO: 302 - protein, 6 aa, Artificial Sequence
  source: /note=“Description of Artificial Sequence: Synthetic peptide”
QSVSSN

SEQ ID NO: 303 - protein, 107 aa, Artificial Sequence
  source: /note=“Description of Artificial Sequence: Synthetic polypeptide”
EIVMTQSPATLSVSPGERATLSCRASQSVSSNLAWYQQKPGQAPRLLIYGASTRATGIPARFSGSGSGTEFTLTISSLQSEDFAVYYCQQSGALPITFGGGTKVEIK

SEQ ID NO: 304 - protein, 10 aa, Artificial Sequence
  source: /note=“Description of Artificial Sequence: Synthetic peptide”
GGSFLRYYWS

SEQ ID NO: 305 - protein, 16 aa, Artificial Sequence
  source: /note=“Description of Artificial Sequence: Synthetic peptide”
EIDHSGSTNYNPSLKS

SEQ ID NO: 306 - protein, 15 aa, Artificial Sequence
  source: /note=“Description of Artificial Sequence: Synthetic peptide”
GQNYYGSGSADGFDP

SEQ ID NO: 307 - protein, 7 aa, Artificial Sequence
  source: /note=“Description of Artificial Sequence: Synthetic peptide”
GGSFLRY

SEQ ID NO: 308 - protein, 5 aa, Artificial Sequence
  source: /note=“Description of Artificial Sequence: Synthetic peptide”
DHSGS

SEQ ID NO: 309 - protein, 8 aa, Artificial Sequence
  source: /note=“Description of Artificial Sequence: Synthetic peptide”
GGSFLRYY

SEQ ID NO: 310 - protein, 7 aa, Artificial Sequence
  source: /note=“Description of Artificial Sequence: Synthetic peptide”
IDHSGST

SEQ ID NO: 311 - protein, 17 aa, Artificial Sequence
  source: /note=“Description of Artificial Sequence: Synthetic peptide”
ARGQNYYGSGSADGFDP

SEQ ID NO: 312 - protein, 9 aa, Artificial Sequence
  source: /note=“Description of Artificial Sequence: Synthetic peptide”
GSFLRYYWS

SEQ ID NO: 313 - protein, 123 aa, Artificial Sequence
  source: /note=“Description of Artificial Sequence: Synthetic polypeptide”
QVQLQQWGAGLLKPSETLSLTCAVYGGSFLRYYWSWIRQPPGKGLEWIGEIDHSGSTNYNPSLKSRVTISVDTSKNQFSLKLSSVTAADTAVYYCARGQNYYGSGSADGFDPWGQGTLVTVSS

SEQ ID NO: 314 - protein, 9 aa, Artificial Sequence
  source: /note=“Description of Artificial Sequence: Synthetic peptide”
QQANSFPPT

SEQ ID NO: 315 - protein, 6 aa, Artificial Sequence
  source: /note=“Description of Artificial Sequence: Synthetic peptide”
ANSFPP

SEQ ID NO: 316 - protein, 107 aa, Artificial Sequence
  source: /note=“Description of Artificial Sequence: Synthetic polypeptide”
DIQMTQSPSSVSASVGDRVTITCRASQGISSWLAWYQQKPGKAPKLLIYAASSLQSGVPSRFSGSGSGTDFTLTISSLQPEDFATYYCQQANSFPPTFGGGTKVEIK

SEQ ID NO: 317 - protein, 10 aa, Artificial Sequence
  source: /note=“Description of Artificial Sequence: Synthetic peptide”
GGSFSGYYWS

SEQ ID NO: 318 - protein, 16 aa, Artificial Sequence
  source: /note=“Description of Artificial Sequence: Synthetic peptide”
EIDASGSTRYNPSLKS

SEQ ID NO: 319 - protein, 9 aa, Artificial Sequence
  source: /note=“Description of Artificial Sequence: Synthetic peptide”
PLYDAYLDV

SEQ ID NO: 320 - protein, 5 aa, Artificial Sequence
  source: /note=“Description of Artificial Sequence: Synthetic peptide”
GYYWS

SEQ ID NO: 321 - protein, 7 aa, Artificial Sequence
  source: /note=“Description of Artificial Sequence: Synthetic peptide”
GGSFSGY

SEQ ID NO: 322 - protein, 5 aa, Artificial Sequence
  source: /note=“Description of Artificial Sequence: Synthetic peptide”
DASGS

SEQ ID NO: 323 - protein, 8 aa, Artificial Sequence
  source: /note=“Description of Artificial Sequence: Synthetic peptide”
GGSFSGYY

SEQ ID NO: 324 - protein, 7 aa, Artificial Sequence
  source: /note=“Description of Artificial Sequence: Synthetic peptide”
IDASGST

SEQ ID NO: 325 - protein, 11 aa, Artificial Sequence
  source: /note=“Description of Artificial Sequence: Synthetic peptide”
ARPLYDAYLDV

SEQ ID NO: 326 - protein, 9 aa, Artificial Sequence
  source: /note=“Description of Artificial Sequence: Synthetic peptide”
GSFSGYYWS

SEQ ID NO: 327 - protein, 117 aa, Artificial Sequence
  source: /note=“Description of Artificial Sequence: Synthetic polypeptide”
QVQLQQWGAGLLKPSETLSLTCAVYGGSFSGYYWSWIRQPPGKGLEWIGEIDASGSTRYNPSLKSRVTISVDTSKNQFSLKLSSVTAADTAVYYCARPLYDAYLDVWGQGTMVTVSS

SEQ ID NO: 328 - protein, 9 aa, Artificial Sequence
  source: /note=“Description of Artificial Sequence: Synthetic peptide”
QQAPIYPIT

SEQ ID NO: 329 - protein, 6 aa, Artificial Sequence
  source: /note=“Description of Artificial Sequence: Synthetic peptide”
APIYPI

SEQ ID NO: 330 - protein, 107 aa, Artificial Sequence
  source: /note=“Description of Artificial Sequence: Synthetic polypeptide”
DIQMTQSPSSVSASVGDRVTITCRASQGISSWLAWYQQKPGKAPKLLIYAASSLQSGVPSRFSGSGSGTDFTLTISSLQPEDFATYYCQQAPIYPITFGGGTKVEIK

SEQ ID NO: 331 - protein, 225 aa, Artificial Sequence
  source: /note=“Description of Artificial Sequence: Synthetic polypeptide”
QVQLQESGPGLVKPSETLSLTCTVSGGSIERYYWSWIRQPPGKGLEWIGYIYGRGSTNYNPSLKSRVTISVDTSKNQFSLKLSSVTAADTAVYYCARESQESPYNNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGP

SEQ ID NO: 332 - protein, 553 aa, Artificial Sequence
  source: /note=“Description of Artificial Sequence: Synthetic polypeptide”
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHADETFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKHHHHHH

SEQ ID NO: 333-400 - skipped (68 entries)

SEQ ID NO: 401 - protein, 5 aa, Artificial Sequence
  source: /note=“Description of Artificial Sequence: Synthetic peptide”
DYNMD

SEQ ID NO: 402 - protein, 17 aa, Artificial Sequence
  source: /note=“Description of Artificial Sequence: Synthetic peptide”
DINPKYDISTYNQQFKG

SEQ ID NO: 403 - protein, 16 aa, Artificial Sequence
  source: /note=“Description of Artificial Sequence: Synthetic peptide”
RGFFLYYGINYYYFDV

SEQ ID NO: 404 - protein, 7 aa, Artificial Sequence
  source: /note=“Description of Artificial Sequence: Synthetic peptide”
GYTFTDY

SEQ ID NO: 405 - protein, 6 aa, Artificial Sequence
  source: /note=“Description of Artificial Sequence: Synthetic peptide”
NPKYDI

SEQ ID NO: 406 - protein, 10 aa, Artificial Sequence
  source: /note=“Description of Artificial Sequence: Synthetic peptide”
GYTFTDYNMD

SEQ ID NO: 407 - protein, 8 aa, Artificial Sequence
  source: /note=“Description of Artificial Sequence: Synthetic peptide”
GYTFTDYN

SEQ ID NO: 408 - protein, 8 aa, Artificial Sequence
  source: /note=“Description of Artificial Sequence: Synthetic peptide”
INPKYDIS

SEQ ID NO: 409 - protein, 18 aa, Artificial Sequence
  source: /note=“Description of Artificial Sequence: Synthetic peptide”
ARRGFFLYYGINYYYFDV

SEQ ID NO: 410 - protein, 125 aa, Artificial Sequence
  source: /note=“Description of Artificial Sequence: Synthetic polypeptide”
EVQLVQSGAEVKKPGESLKISCKASGYTFTDYNMDWVRQMPGKGLEWMGDINPKYDISTYNQQFKGQVTISADKSIRTAYLQWSSLKASDTAMYYCARRGFFLYYGINYYYFDVWGQGTLVTVSS

SEQ ID NO: 411 - DNA, 375 bp, Artificial Sequence
  source: /note=“Description of Artificial Sequence: Synthetic polynucleotide”
gaagtgcagctggtgcagtctggcgccgaagtgaagaagcctggcgagtccctgaagatctcctgcaaggcctccggctacaccttcaccgactacaacatggactgggtgcgacagatgcccggcaagggcctggaatggatgggcgacatcaaccctaagtacgacatctccacctacaaccagcagttcaagggccaagtgaccatctccgccgacaagtccatccggaccgcctacctgcagtggtcctccctgaaggcctctgacaccgccatgtactactgcgccagacggggcttcttcctgtactacggcatcaactactactacttcgacgtgtggggccagggcaccctcgtgacagtgtcatct

SEQ ID NO: 412 - protein, 455 aa, Artificial Sequence
  source: /note=“Description of Artificial Sequence: Synthetic polypeptide”
EVQLVQSGAEVKKPGESLKISCKASGYTFTDYNMDWVRQMPGKGLEWMGDINPKYDISTYNQQFKGQVTISADKSIRTAYLQWSSLKASDTAMYYCARRGFFLYYGINYYYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 413 - DNA, 1365 bp, Artificial Sequence
  source: /note=“Description of Artificial Sequence: Synthetic polynucleotide”
gaagtgcagctggtgcagtctggcgccgaagtgaagaagcctggcgagtccctgaagatctcctgcaaggcctccggctacaccttcaccgactacaacatggactgggtgcgacagatgcccggcaagggcctggaatggatgggcgacatcaaccctaagtacgacatctccacctacaaccagcagttcaagggccaagtgaccatctccgccgacaagtccatccggaccgcctacctgcagtggtcctccctgaaggcctctgacaccgccatgtactactgcgccagacggggcttcttcctgtactacggcatcaactactactacttcgacgtgtggggccagggcaccctcgtgacagtgtcatctgctagcaccaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 414 - protein, 10 aa, Artificial Sequence
  source: /note=“Description of Artificial Sequence: Synthetic peptide”
SASSSVSYIH

SEQ ID NO: 415 - protein, 7 aa, Artificial Sequence
  source: /note=“Description of Artificial Sequence: Synthetic peptide”
STSNLAS

SEQ ID NO: 416 - protein, 9 aa, Artificial Sequence
  source: /note=“Description of Artificial Sequence: Synthetic peptide”
HQWSSYPWT

SEQ ID NO: 417 - protein, 6 aa, Artificial Sequence
  source: /note=“Description of Artificial Sequence: Synthetic peptide”
SSSVSY

SEQ ID NO: 418 - protein, 3 aa, Artificial Sequence
  source: /note=“Description of Artificial Sequence: Synthetic peptide”
STS

SEQ ID NO: 419 - protein, 6 aa, Artificial Sequence
  source: /note=“Description of Artificial Sequence: Synthetic peptide”
WSSYPW

SEQ ID NO: 420 - protein, 5 aa, Artificial Sequence
  source: /note=“Description of Artificial Sequence: Synthetic peptide”
SSVSY

SEQ ID NO: 421 - protein, 106 aa, Artificial Sequence
  source: /note=“Description of Artificial Sequence: Synthetic polypeptide”
DIQLTQSPSSLSASVGDRVTITCSASSSVSYIHWFQQKPGKAPKLLIYSTSNLASGVPSRFSGSGSGTFFTLTISSLQPEDFATYFCHQWSSYPWTFGQGTKVEIK

SEQ ID NO: 422 - DNA, 318 bp, Artificial Sequence
  source: /note=“Description of Artificial Sequence: Synthetic polynucleotide”
gatatccagctgacccagtccccttccagcctgtctgcctctgtgggcgacagagtgacaattacctgctccgcctcctcctccgtgtcctacatccactggttccagcagaagcccggcaaggcccccaagctgctgatctactccacctccaacctggcctccggcgtgccctctagattctccggctctggctctggcaccttttttaccctgaccatctccagcctgcagcccgaggacttcgccacctacttttgccaccagtggtccagctacccctggacctttggccagggcaccaaggtggaaatcaag

SEQ ID NO: 423 - protein, 213 aa, Artificial Sequence
  source: /note=“Description of Artificial Sequence: Synthetic polypeptide”
DIQLTQSPSSLSASVGDRVTITCSASSSVSYIHWFQQKPGKAPKLLIYSTSNLASGVPSRFSGSGSGTFFTLTISSLQPEDFATYFCHQWSSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 424 - DNA, 639 bp, Artificial Sequence
  source: /note=“Description of Artificial Sequence: Synthetic polynucleotide”
gatatccagctgacccagtccccttccagcctgtctgcctctgtgggcgacagagtgacaattacctgctccgcctcctcctccgtgtcctacatccactggttccagcagaagcccggcaaggcccccaagctgctgatctactccacctccaacctggcctccggcgtgccctctagattctccggctctggctctggcaccttttttaccctgaccatctccagcctgcagcccgaggacttcgccacctacttttgccaccagtggtccagctacccctggacctttggccagggcaccaaggtggaaatcaagcgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 425 - protein, 125 aa, Artificial Sequence
  source: /note=“Description of Artificial Sequence: Synthetic polypeptide”
QVQLVQSGAEVVKPGASVKISCKASGYTFTDYNMDWVKQAPGQRLEWIGDINPKYDISTYNQQFKGKATITVDKSASTAYMELSSLRSEDTAVYYCARRGFFLYYGINYYYFDVWGQGTLVTVSS

SEQ ID NO: 426 - DNA, 375 bp, Artificial Sequence
  source: /note=“Description of Artificial Sequence: Synthetic polynucleotide”
caggtgcagctggtgcagtctggcgccgaggtggtgaaacctggcgcctccgtgaagatctcctgcaaggcctccggctacaccttcaccgactacaacatggactgggtgaaacaggcccctggccagcggctggaatggatcggcgacatcaaccctaagtacgacatctccacctacaaccagcagttcaagggcaaggccaccatcaccgtggacaagtccgcctccaccgcctacatggaactgtcctccctgcggagcgaggacaccgccgtgtactactgcgccagacggggcttcttcctgtactacggcatcaactactactacttcgacgtgtggggccagggcaccctggtgacagtgtcctcc

SEQ ID NO: 427 - protein, 455 aa, Artificial Sequence
  source: /note=“Description of Artificial Sequence: Synthetic polypeptide”
QVQLVQSGAEVVKPGASVKISCKASGYTFTDYNMDWVKQAPGQRLEWIGDINPKYDISTYNQQFKGKATITVDKSASTAYMELSSLRSEDTAVYYCARRGFFLYYGINYYYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 428 - DNA, 1365 bp, Artificial Sequence
  source: /note=“Description of Artificial Sequence: Synthetic polynucleotide”
caggtgcagctggtgcagtctggcgccgaggtggtgaaacctggcgcctccgtgaagatctcctgcaaggcctccggctacaccttcaccgactacaacatggactgggtgaaacaggcccctggccagcggctggaatggatcggcgacatcaaccctaagtacgacatctccacctacaaccagcagttcaagggcaaggccaccatcaccgtggacaagtccgcctccaccgcctacatggaactgtcctccctgcggagcgaggacaccgccgtgtactactgcgccagacggggcttcttcctgtactacggcatcaactactactacttcgacgtgtggggccagggcaccctggtgacagtgtcctccgctagcaccaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 429 - protein, 106 aa, Artificial Sequence
  source: /note=“Description of Artificial Sequence: Synthetic polypeptide”
EIVLTQSPATLSASPGERITLSCSASSSVSYIHWYQQKPGQAPRLLIYSTSNLASGIPARFSGSGSGTFYTLTISSVEPEDAAVYYCHQWSSYPWTFGGGTKLEIK

SEQ ID NO: 430 - DNA, 318 bp, Artificial Sequence
  source: /note=“Description of Artificial Sequence: Synthetic polynucleotide”
gagatcgtgctgacccagtcccctgccaccctgtctgccagccctggcgagcggatcaccctgtcctgctccgcctcctcctccgtgtcctacatccactggtatcagcagaagcccggccaggcccctcggctgctgatctactccacctccaacctggcctccggcatccctgccagattctccggctctggctccggcaccttttacaccctgaccatctccagcgtggaacccgaggacgccgccgtgtactactgccaccagtggtccagctacccctggaccttcggcggaggcaccaagctggaaatcaag

SEQ ID NO: 431 - protein, 213 aa, Artificial Sequence
  source: /note=“Description of Artificial Sequence: Synthetic polypeptide”
EIVLTQSPATLSASPGERITLSCSASSSVSYIHWYQQKPGQAPRLLIYSTSNLASGIPARFSGSGSGTFYTLTISSVEPEDAAVYYCHQWSSYPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 432 - DNA, 639 bp, Artificial Sequence
  source: /note=“Description of Artificial Sequence: Synthetic polynucleotide”
gagatcgtgctgacccagtcccctgccaccctgtctgccagccctggcgagcggatcaccctgtcctgctccgcctcctcctccgtgtcctacatccactggtatcagcagaagcccggccaggcccctcggctgctgatctactccacctccaacctggcctccggcatccctgccagattctccggctctggctccggcaccttttacaccctgaccatctccagcgtggaacccgaggacgccgccgtgtactactgccaccagtggtccagctacccctggaccttcggcggaggcaccaagctggaaatcaagcgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 433 - protein, 125 aa, Artificial Sequence
  source: /note=“Description of Artificial Sequence: Synthetic polypeptide”
QVQLVQSGAEVVKPGASVKISCKASGYTFTDYNMDWVKQAPGQRLEWIGDINPKYDISTYNQQFKGKATITVDTSASTAYMELSSLRSEDTAVYYCARRGFFLYYGINYYYFDVWGQGTLVTVSS

SEQ ID NO: 434 - DNA, 375 bp, Artificial Sequence
  source: /note=“Description of Artificial Sequence: Synthetic polynucleotide”
caggtgcagctggtgcagtctggcgccgaggtggtgaaacctggcgcctccgtgaagatctcctgcaaggcctccggctacaccttcaccgactacaacatggactgggtgaaacaggcccctggccagcggctggaatggatcggcgacatcaaccctaagtacgacatctccacctacaaccagcagttcaagggcaaggccaccatcaccgtggacacctccgcctccaccgcctacatggaactgtcctccctgcggagcgaggacaccgccgtgtactactgcgccagacggggcttcttcctgtactacggcatcaactactactacttcgacgtgtggggccagggcaccctggtgacagtgtcctcc

SEQ ID NO: 435 - protein, 455 aa, Artificial Sequence
  source: /note=“Description of Artificial Sequence: Synthetic polypeptide”
QVQLVQSGAEVVKPGASVKISCKASGYTFTDYNMDWVKQAPGQRLEWIGDINPKYDISTYNQQFKGKATITVDTSASTAYMELSSLRSEDTAVYYCARRGFFLYYGINYYYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 436 - DNA, 1365 bp, Artificial Sequence
  source: /note=“Description of Artificial Sequence: Synthetic polynucleotide”
caggtgcagctggtgcagtctggcgccgaggtggtgaaacctggcgcctccgtgaagatctcctgcaaggcctccggctacaccttcaccgactacaacatggactgggtgaaacaggcccctggccagcggctggaatggatcggcgacatcaaccctaagtacgacatctccacctacaaccagcagttcaagggcaaggccaccatcaccgtggacacctccgcctccaccgcctacatggaactgtcctccctgcggagcgaggacaccgccgtgtactactgcgccagacggggcttcttcctgtactacggcatcaactactactacttcgacgtgtggggccagggcaccctggtgacagtgtcctccgctagcaccaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 437 - protein, 6 aa, Artificial Sequence
  source: /note=“Description of Artificial Sequence: Synthetic peptide”
SGYYWN

SEQ ID NO: 438 - protein, 16 aa, Artificial Sequence
  source: /note=“Description of Artificial Sequence: Synthetic peptide”
YISYDADNNYNPSLKN

SEQ ID NO: 439 - protein, 20 aa, Artificial Sequence
  source: /note=“Description of Artificial Sequence: Synthetic peptide”
GYYRYGLGSYRGSYYYVMDY

SEQ ID NO: 440 - protein, 8 aa, Artificial Sequence
  source: /note=“Description of Artificial Sequence: Synthetic peptide”
GYSITSGY

SEQ ID NO: 441 - protein, 5 aa, Artificial Sequence
  source: /note=“Description of Artificial Sequence: Synthetic peptide”
SYDAD

SEQ ID NO: 442 - protein, 11 aa, Artificial Sequence
  source: /note=“Description of Artificial Sequence: Synthetic peptide”
GYSITSGYYWN

SEQ ID NO: 443 - protein, 9 aa, Artificial Sequence
  source: /note=“Description of Artificial Sequence: Synthetic peptide”
GYSITSGYY

SEQ ID NO: 444 - protein, 7 aa, Artificial Sequence
  source: /note=“Description of Artificial Sequence: Synthetic peptide”
ISYDADN

SEQ ID NO: 445 - protein, 22 aa, Artificial Sequence
  source: /note=“Description of Artificial Sequence: Synthetic peptide”
AGGYYRYGLGSYRGSYYYVMDY

SEQ ID NO: 446 - protein, 129 aa, Artificial Sequence
  source: /note=“Description of Artificial Sequence: Synthetic polypeptide”
QIQLQESGPGLVKPSQTLSLTCTVSGYSITSGYYWNWIRQHPGKGLEWIGYISYDADNNYNPSLKNRVTISRDTSKNQFSLKLSSVTAADTAVYYCAGGYYRYGLGSYRGSYYYVMDYWGQGTTVTVSS

SEQ ID NO: 447 - DNA, 387 bp, Artificial Sequence
  source: /note=“Description of Artificial Sequence: Synthetic polynucleotide”
cagatccagctgcaggaatctggccctggcctggtgaaaccctcccagaccctgtccctgacctgcaccgtgtccggctactccatcacctccggctactactggaactggatccggcagcaccccggcaagggcctggaatggatcggctacatctcctacgacgctgacaacaactacaaccccagcctgaagaacagagtgaccatctcccgggacacctccaagaaccagttctccctgaagctgtcctccgtgaccgccgctgacaccgccgtgtactactgcgctggcggctactacagatacggcctgggctcctaccggggctcctactactacgtgatggactactggggccagggcaccaccgtgaccgtgtcctct

SEQ ID NO: 448 - protein, 459 aa, Artificial Sequence
  source: /note=“Description of Artificial Sequence: Synthetic polypeptide”
QIQLQESGPGLVKPSQTLSLTCTVSGYSITSGYYWNWIRQHPGKGLEWIGYISYDADNNYNPSLKNRVTISRDTSKNQFSLKLSSVTAADTAVYYCAGGYYRYGLGSYRGSYYYVMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 449 - DNA, 1377 bp, Artificial Sequence
  source: /note=“Description of Artificial Sequence: Synthetic polynucleotide”
cagatccagctgcaggaatctggccctggcctggtgaaaccctcccagaccctgtccctgacctgcaccgtgtccggctactccatcacctccggctactactggaactggatccggcagcaccccggcaagggcctggaatggatcggctacatctcctacgacgctgacaacaactacaaccccagcctgaagaacagagtgaccatctcccgggacacctccaagaaccagttctccctgaagctgtcctccgtgaccgccgctgacaccgccgtgtactactgcgctggcggctactacagatacggcctgggctcctaccggggctcctactactacgtgatggactactggggccagggcaccaccgtgaccgtgtcctctgctagcaccaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 450 - protein, 15 aa, Artificial Sequence
  source: /note=“Description of Artificial Sequence: Synthetic peptide”
KASQSVDYEGHSYMN

SEQ ID NO: 451 - protein, 7 aa, Artificial Sequence
  source: /note=“Description of Artificial Sequence: Synthetic peptide”
AASNLES

SEQ ID NO: 452 - protein, 9 aa, Artificial Sequence
  source: /note=“Description of Artificial Sequence: Synthetic peptide”
QQSNEDPPT

SEQ ID NO: 453 - protein, 11 aa, Artificial Sequence
  source: /note=“Description of Artificial Sequence: Synthetic peptide”
SQSVDYEGHSY

SEQ ID NO: 454 - protein, 3 aa, Artificial Sequence
  source: /note=“Description of Artificial Sequence: Synthetic peptide”
AAS

SEQ ID NO: 455 - protein, 6 aa, Artificial Sequence
  source: /note=“Description of Artificial Sequence: Synthetic peptide”
SNEDPP

SEQ ID NO: 456 - protein, 10 aa, Artificial Sequence
  source: /note=“Description of Artificial Sequence: Synthetic peptide”
QSVDYEGHSY

SEQ ID NO: 457 - protein, 111 aa, Artificial Sequence
  source: /note=“Description of Artificial Sequence: Synthetic polypeptide”
ETVLTQSPATLSVSPGERATISCKASQSVDYEGHSYMNWYQQKPGQAPRLLIYAASNLESGIPARFSGSGSGTDFTLTISSVEPEDAATYYCQQSNEDPPTFGGGTKLEIK

SEQ ID NO: 458 - DNA, 333 bp, Artificial Sequence
  source: /note=“Description of Artificial Sequence: Synthetic polynucleotide”
gagacagtgctgacccagtcccctgccaccctgtccgtgtctcccggcgagagagccaccatcagctgcaaggcctcccagtccgtggactacgaaggccattcctacatgaactggtatcagcagaagcccggccaggcccctcggctgctgatctacgccgcctccaacctggaatccggcatccctgcccggttctccggctctggctctggcaccgacttcaccctgaccatctccagcgtggaacccgaggacgccgccacctactactgccagcagtccaacgaggacccccccaccttcggcggaggcaccaagctggaaatcaag

SEQ ID NO: 459 - protein, 218 aa, Artificial Sequence
  source: /note=“Description of Artificial Sequence: Synthetic polypeptide”
ETVLTQSPATLSVSPGERATISCKASQSVDYEGHSYMNWYQQKPGQAPRLLIYAASNLESGIPARFSGSGSGTDFTLTISSVEPEDAATYYCQQSNEDPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 460 - DNA, 654 bp, Artificial Sequence
  source: /note=“Description of Artificial Sequence: Synthetic polynucleotide”
gagacagtgctgacccagtcccctgccaccctgtccgtgtctcccggcgagagagccaccatcagctgcaaggcctcccagtccgtggactacgaaggccattcctacatgaactggtatcagcagaagcccggccaggcccctcggctgctgatctacgccgcctccaacctggaatccggcatccctgcccggttctccggctctggctctggcaccgacttcaccctgaccatctccagcgtggaacccgaggacgccgccacctactactgccagcagtccaacgaggacccccccaccttcggcggaggcaccaagctggaaatcaagcgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 461 - protein, 15 aa, Artificial Sequence
  source: /note=“Description of Artificial Sequence: Synthetic peptide”
KASQSVDYDGNSYMN

SEQ ID NO: 462 - protein, 11 aa, Artificial Sequence
  source: /note=“Description of Artificial Sequence: Synthetic peptide”
SQSVDYDGNSY

SEQ ID NO: 463 - protein, 10 aa, Artificial Sequence
  source: /note=“Description of Artificial Sequence: Synthetic peptide”
QSVDYDGNSY

SEQ ID NO: 464 - protein, 111 aa, Artificial Sequence
  source: /note=“Description of Artificial Sequence: Synthetic polypeptide”
ETVLTQSPATLSVSPGERATISCKASQSVDYDGNSYMNWYQQKPGQAPRLLIYAASNLESGIPARFSGSGSGTDFTLTISSVEPEDAATYYCQQSNEDPPTFGGGTKLEIK

SEQ ID NO: 465 - DNA, 333 bp, Artificial Sequence
  source: /note=“Description of Artificial Sequence: Synthetic polynucleotide”
gagacagtgctgacccagtcccctgccaccctgtccgtgtctcccggcgagagagccaccatcagctgcaaggcctcccagtccgtggactacgacggcaactcctacatgaactggtatcagcagaagcccggccaggcccctcggctgctgatctacgccgcctccaacctggaatccggcatccctgcccggttctccggctctggctctggcaccgacttcaccctgaccatctccagcgtggaacccgaggacgccgccacctactactgccagcagtccaacgaggacccccccaccttcggcggaggcaccaagctggaaatcaag

SEQ ID NO: 466 - protein, 218 aa, Artificial Sequence
  source: /note=“Description of Artificial Sequence: Synthetic polypeptide”
ETVLTQSPATLSVSPGERATISCKASQSVDYDGNSYMNWYQQKPGQAPRLLIYAASNLESGIPARFSGSGSGTDFTLTISSVEPEDAATYYCQQSNEDPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 467 - DNA, 654 bp, Artificial Sequence
  source: /note=“Description of Artificial Sequence: Synthetic polynucleotide”
gagacagtgctgacccagtcccctgccaccctgtccgtgtctcccggcgagagagccaccatcagctgcaaggcctcccagtccgtggactacgacggcaactcctacatgaactggtatcagcagaagcccggccaggcccctcggctgctgatctacgccgcctccaacctggaatccggcatccctgcccggttctccggctctggctctggcaccgacttcaccctgaccatctccagcgtggaacccgaggacgccgccacctactactgccagcagtccaacgaggacccccccaccttcggcggaggcaccaagctggaaatcaagcgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 468-500 - skipped (33 entries)

SEQ ID NO: 501 - protein, 5 aa, Artificial Sequence
  source: /note=“Description of Artificial Sequence: Synthetic peptide”
TYWMH

SEQ ID NO: 502 - protein, 17 aa, Artificial Sequence
  source: /note=“Description of Artificial Sequence: Synthetic peptide”
NIYPGTGGSNFDEKFKN

SEQ ID NO: 503 - protein, 8 aa, Artificial Sequence
  source: /note=“Description of Artificial Sequence: Synthetic peptide”
WTTGTGAY

SEQ ID NO: 504 - protein, 7 aa, Artificial Sequence
  source: /note=“Description of Artificial Sequence: Synthetic peptide”
GYTFTTY

SEQ ID NO: 505 - protein, 6 aa, Artificial Sequence
  source: /note=“Description of Artificial Sequence: Synthetic peptide”
YPGTGG

SEQ ID NO: 506 - protein, 117 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 507 - DNA, 351 bp, Artificial Sequence
  source: /note=“Description of Artificial Sequence: Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagc

SEQ ID NO: 508 - protein, 443 aa, Artificial Sequence
  source: /note=“Description of Artificial Sequence: Synthetic polypeptide”
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 509 - DNA, 1329 bp, Artificial Sequence
  source: /note=“Description of Artificial Sequence: Synthetic polynucleotide”
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 510 - protein, 17 aa, Artificial Sequence
  source: /note=“Description of Artificial Sequence: Synthetic peptide”
KSSQSLLDSGNQKNFLT

SEQ ID NO: 511 - protein, 7 aa, Artificial Sequence
  source: /note=“Description of Artificial Sequence: Synthetic peptide”
WASTRES

SEQ ID NO: 512 - protein, 9 aa, Artificial Sequence
  source: /note=“Description of Artificial Sequence: Synthetic peptide”
QNDYSYPYT

SEQ ID NO: 513 - protein, 13 aa, Artificial Sequence
  source: /note=“Description of Artificial Sequence: Synthetic peptide”
SQSLLDSGNQKNF

SEQ ID NO: 514 - protein, 3 aa, Artificial Sequence
  source: /note=“Description of Artificial Sequence: Synthetic peptide”
WAS

SEQ ID NO: 515 - protein, 6 aa, Artificial Sequence
  source: /note=“Description of Artificial Sequence: Synthetic peptide”
DYSYPY

SEQ ID NO: 516 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 517 - DNA, 339 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaag

SEQ ID NO: 518 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 519 - DNA, 660 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 520 - protein, 113 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 521 - DNA, 339 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaag

SEQ ID NO: 522 - protein, 220 aa, Artificial Sequence
  source: /note=“Description of Artificial Sequence: Synthetic polypeptide”
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 523 - DNA, 660 bp, Artificial Sequence
  source: /note=“Description of Artificial Sequence: Synthetic polynucleotide”
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 524 - DNA, 15 bp, Artificial Sequence
  source: /note=“Description of Artificial Sequence: Synthetic oligonucleotide”
acctactggatgcac

SEQ ID NO: 525 - DNA, 51 bp, Artificial Sequence
  source: /note=“Description of Artificial Sequence: Synthetic oligonucleotide”
aatatctaccccggcaccggcggctctaacttcgacgagaagtttaagaat

SEQ ID NO: 526 - DNA, 24 bp, Artificial Sequence
  source: /note=“Description of Artificial Sequence: Synthetic oligonucleotide”
tggactaccggcacaggcgcctac

SEQ ID NO: 527 - DNA, 21 bp, Artificial Sequence
  source: /note=“Description of Artificial Sequence: Synthetic oligonucleotide”
ggctacaccttcactacctac

SEQ ID NO: 528 - DNA, 18 bp, Artificial Sequence
  source: /note=“Description of Artificial Sequence: Synthetic oligonucleotide”
taccccggcaccggcggc

SEQ ID NO: 529 - DNA, 51 bp, Artificial Sequence
  source: /note=“Description of Artificial Sequence: Synthetic oligonucleotide”
aaatctagtcagtcactgctggatagcggtaatcagaagaacttcctgacc

SEQ ID NO: 530 - DNA, 21 bp, Artificial Sequence
  source: /note=“Description of Artificial Sequence: Synthetic oligonucleotide”
tgggcctctactagagaatca

SEQ ID NO: 531 - DNA, 27 bp, Artificial Sequence
  source: /note=“Description of Artificial Sequence: Synthetic oligonucleotide”
cagaacgactatagctacccctacacc

SEQ ID NO: 532 - DNA, 39 bp, Artificial Sequence
  source: /note=“Description of Artificial Sequence: Synthetic oligonucleotide”
agtcagtcactgctggatagcggtaatcagaagaacttc

SEQ ID NO: 533 - DNA, 9 bp, Artificial Sequence
  source: /note=“Description of Artificial Sequence: Synthetic oligonucleotide”
tgggcctct

SEQ ID NO: 534 - DNA, 18 bp, Artificial Sequence
  source: /note=“Description of Artificial Sequence: Synthetic oligonucleotide”
gactatagctacccctac

SEQ ID NO: 535 - protein, 440 aa, Artificial Sequence
  source: /note=“Description of Artificial Sequence: Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 536 - protein, 214 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 537 - protein, 447 aa, Artificial Sequence
  source: /note=“Description of Artificial Sequence: Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 538 - protein, 218 aa, Artificial Sequence
  source: /note=“Description of Artificial Sequence: Synthetic polypeptide”
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 539 - protein, 447 aa, Artificial Sequence
  source: /note=“Description of Artificial Sequence: Synthetic polypeptide”
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 540 - protein, 213 aa, Artificial Sequence
  source: /note=“Description of Artificial Sequence: Synthetic polypeptide”
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 541 - protein, 10 aa, Artificial Sequence
  source: /note=“Description of Artificial Sequence: Synthetic peptide”
GYTFTTYWMH

SEQ ID NO: 542 - protein, 445 aa, Artificial Sequence
  source: /note=“Description of Artificial Sequence: Synthetic polypeptide”
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 543 - protein, 214 aa, Artificial Sequence
  source: /note=“Description of Artificial Sequence: Synthetic polypeptide”
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 544-600 - skipped (57 entries)

SEQ ID NO: 601 - protein, 5 aa, Artificial Sequence
  source: /note=“Description of Artificial Sequence: Synthetic peptide”
SYWMY

SEQ ID NO: 602 - protein, 17 aa, Artificial Sequence
  source: /note=“Description of Artificial Sequence: Synthetic peptide”
RIDPNSGSTKYNEKFKN

SEQ ID NO: 603 - protein, 11 aa, Artificial Sequence
  source: /note=“Description of Artificial Sequence: Synthetic peptide”
DYRKGLYAMDY

SEQ ID NO: 604 - protein, 7 aa, Artificial Sequence
  source: /note=“Description of Artificial Sequence: Synthetic peptide”
GYTFTSY

SEQ ID NO: 605 - protein, 6 aa, Artificial Sequence
  source: /note=“Description of Artificial Sequence: Synthetic peptide”
DPNSGS

SEQ ID NO: 606 - protein, 120 aa, Artificial Sequence
  source: /note=“Description of Artificial Sequence: Synthetic polypeptide”
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSS

SEQ ID NO: 607 - DNA, 360 bp, Artificial Sequence
  source: /note=“Description of Artificial Sequence: Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctagagggcaaagactggagtggatcggtagaatcgaccctaatagcggctctactaagtataacgagaagtttaagaataggttcactattagtagggataactctaagaacaccctgtacctgcagatgaatagcctgagagccgaggacaccgccgtctactactgcgctagagactatagaaagggcctgtacgctatggactactggggtcaaggcactaccgtgaccgtgtcttca

SEQ ID NO: 608 - protein, 446 aa, Artificial Sequence
  source: /note=“Description of Artificial Sequence: Synthetic polypeptide”
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 609 - protein, 11 aa, Artificial Sequence
  source: /note=“Description of Artificial Sequence: Synthetic peptide”
KASQDVGTAVA

SEQ ID NO: 610 - protein, 7 aa, Artificial Sequence
  source: /note=“Description of Artificial Sequence: Synthetic peptide”
WASTRHT

SEQ ID NO: 611 - protein, 9 aa, Artificial Sequence
  source: /note=“Description of Artificial Sequence: Synthetic peptide”
QQYNSYPLT

SEQ ID NO: 612 - protein, 7 aa, Artificial Sequence
  source: /note=“Description of Artificial Sequence: Synthetic peptide”
SQDVGTA

SEQ ID NO: 613 - protein, 3 aa, Artificial Sequence
  source: /note=“Description of Artificial Sequence: Synthetic peptide”
WAS

SEQ ID NO: 614 - protein, 6 aa, Artificial Sequence
  source: /note=“Description of Artificial Sequence: Synthetic peptide”
YNSYPL

SEQ ID NO: 615 - DNA, 1338 bp, Artificial Sequence
  source: /note=“Description of Artificial Sequence: Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctagagggcaaagactggagtggatcggtagaatcgaccctaatagcggctctactaagtataacgagaagtttaagaataggttcactattagtagggataactctaagaacaccctgtacctgcagatgaatagcctgagagccgaggacaccgccgtctactactgcgctagagactatagaaagggcctgtacgctatggactactggggtcaaggcactaccgtgaccgtgtcttcagctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 616 - protein, 107 aa, Artificial Sequence
  source: /note=“Description of Artificial Sequence: Synthetic polypeptide”
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIK

SEQ ID NO: 617 - DNA, 321 bp, Artificial Sequence
  source: /note=“Description of Artificial Sequence: Synthetic polynucleotide”
gctattcagctgactcagtcacctagtagcctgagcgctagtgtgggcgatagagtgactatcacctgtaaagcctctcaggacgtgggcaccgccgtggcctggtatctgcagaagcctggtcaatcacctcagctgctgatctactgggcctctactagacacaccggcgtgccctctaggtttagcggtagcggtagtggcaccgacttcaccttcactatctcttcactggaagccgaggacgccgctacctactactgtcagcagtataatagctaccccctgaccttcggtcaaggcactaaggtcgagattaag

SEQ ID NO: 618 - protein, 214 aa, Artificial Sequence
  source: /note=“Description of Artificial Sequence: Synthetic polypeptide”
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 619 - DNA, 642 bp, Artificial Sequence
  source: /note=“Description of Artificial Sequence: Synthetic polynucleotide”
gctattcagctgactcagtcacctagtagcctgagcgctagtgtgggcgatagagtgactatcacctgtaaagcctctcaggacgtgggcaccgccgtggcctggtatctgcagaagcctggtcaatcacctcagctgctgatctactgggcctctactagacacaccggcgtgccctctaggtttagcggtagcggtagtggcaccgacttcaccttcactatctcttcactggaagccgaggacgccgctacctactactgtcagcagtataatagctaccccctgaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 620 - protein, 120 aa, Artificial Sequence
  source: /note=“Description of Artificial Sequence: Synthetic polypeptide”
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSS

SEQ ID NO: 621 - DNA, 360 bp, Artificial Sequence
  source: /note=“Description of Artificial Sequence: Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctaccggtcaaggcctggagtggatgggtagaatcgaccctaatagcggctctactaagtataacgagaagtttaagaatagagtgactatcaccgccgataagtctactagcaccgcctatatggaactgtctagcctgagatcagaggacaccgccgtctactactgcgctagagactatagaaagggcctgtacgctatggactactggggtcaaggcactaccgtgaccgtgtcttca

SEQ ID NO: 622 - protein, 446 aa, Artificial Sequence
  source: /note=“Description of Artificial Sequence: Synthetic polypeptide”
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 623 - DNA, 1338 bp, Artificial Sequence
  source: /note=“Description of Artificial Sequence: Synthetic polynucleotide”
gaagtgcagctggtgcagtcaggcgccgaagtgaagaaacccggcgctaccgtgaagattagctgtaaagtctcaggctacaccttcactagctactggatgtactgggtccgacaggctaccggtcaaggcctggagtggatgggtagaatcgaccctaatagcggctctactaagtataacgagaagtttaagaatagagtgactatcaccgccgataagtctactagcaccgcctatatggaactgtctagcctgagatcagaggacaccgccgtctactactgcgctagagactatagaaagggcctgtacgctatggactactggggtcaaggcactaccgtgaccgtgtcttcagctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 624 - protein, 107 aa, Artificial Sequence
  source: /note=“Description of Artificial Sequence: Synthetic polypeptide”
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIK

SEQ ID NO: 625 - DNA, 321 bp, Artificial Sequence
  source: /note=“Description of Artificial Sequence: Synthetic polynucleotide”
gacgtcgtgatgactcagtcacccctgagcctgcccgtgaccctggggcagcccgcctctattagctgtaaagcctctcaggacgtgggcaccgccgtggcctggtatcagcagaagccagggcaagcccctagactgctgatctactgggcctctactagacacaccggcgtgccctctaggtttagcggtagcggtagtggcaccgagttcaccctgactatctcttcactgcagcccgacgacttcgctacctactactgtcagcagtataatagctaccccctgaccttcggtcaaggcactaaggtcgagattaag

SEQ ID NO: 626 - protein, 214 aa, Artificial Sequence
  source: /note=“Description of Artificial Sequence: Synthetic polypeptide”
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 627 - DNA, 642 bp, Artificial Sequence
  source: /note=“Description of Artificial Sequence: Synthetic polynucleotide”
gacgtcgtgatgactcagtcacccctgagcctgcccgtgaccctggggcagcccgcctctattagctgtaaagcctctcaggacgtgggcaccgccgtggcctggtatcagcagaagccagggcaagcccctagactgctgatctactgggcctctactagacacaccggcgtgccctctaggtttagcggtagcggtagtggcaccgagttcaccctgactatctcttcactgcagcccgacgacttcgctacctactactgtcagcagtataatagctaccccctgaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 628 - DNA, 15 bp, Artificial Sequence
  source: /note=“Description of Artificial Sequence: Synthetic oligonucleotide”
agctactggatgtac

SEQ ID NO: 629 - DNA, 51 bp, Artificial Sequence
  source: /note=“Description of Artificial Sequence: Synthetic oligonucleotide”
agaatcgaccctaatagcggctctactaagtataacgagaagtttaagaat

SEQ ID NO: 630 - DNA, 33 bp, Artificial Sequence
  source: /note=“Description of Artificial Sequence: Synthetic oligonucleotide”
gactatagaaagggcctgtacgctatggactac

SEQ ID NO: 631 - DNA, 21 bp, Artificial Sequence
  source: /note=“Description of Artificial Sequence: Synthetic oligonucleotide”
ggctacaccttcactagctac

SEQ ID NO: 632 - DNA, 18 bp, Artificial Sequence
  source: /note=“Description of Artificial Sequence: Synthetic oligonucleotide”
gaccctaatagcggctct

SEQ ID NO: 633 - DNA, 33 bp, Artificial Sequence
  source: /note=“Description of Artificial Sequence: Synthetic oligonucleotide”
aaagcctctcaggacgtgggcaccgccgtggcc

SEQ ID NO: 634 - DNA, 21 bp, Artificial Sequence
  source: /note=“Description of Artificial Sequence: Synthetic oligonucleotide”
tgggcctctactagacacacc

SEQ ID NO: 635 - DNA, 27 bp, Artificial Sequence
  source: /note=“Description of Artificial Sequence: Synthetic oligonucleotide”
cagcagtataatagctaccccctgacc

SEQ ID NO: 636 - DNA, 21 bp, Artificial Sequence
  source: /note=“Description of Artificial Sequence: Synthetic oligonucleotide”
tctcaggacgtgggcaccgcc

SEQ ID NO: 637 - DNA, 9 bp, Artificial Sequence
  source: /note=“Description of Artificial Sequence: Synthetic oligonucleotide”
tgggcctct

SEQ ID NO: 638 - DNA, 18 bp, Artificial Sequence
  source: /note=“Description of Artificial Sequence: Synthetic oligonucleotide”
tataatagctaccccctg

SEQ ID NO: 639 - protein, 448 aa, Artificial Sequence
  source: /note=“Description of Artificial Sequence: Synthetic polypeptide”
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 640 - protein, 214 aa, Artificial Sequence
  source: /note=“Description of Artificial Sequence: Synthetic polypeptide”
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 641 - protein, 450 aa, Artificial Sequence
  source: /note=“Description of Artificial Sequence: Synthetic polypeptide”
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 642 - protein, 216 aa, Artificial Sequence
  source: /note=“Description of Artificial Sequence: Synthetic polypeptide”
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 643 - protein, 451 aa, Artificial Sequence
  source: /note=“Description of Artificial Sequence: Synthetic polypeptide”
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 644 - protein, 215 aa, Artificial Sequence
  source: /note=“Description of Artificial Sequence: Synthetic polypeptide”
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 645 - protein, 123 aa, Artificial Sequence
  source: /note=“Description of Artificial Sequence: Synthetic polypeptide”
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 646 - protein, 106 aa, Artificial Sequence
  source: /note=“Description of Artificial Sequence: Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 647 - protein, 10 aa, Artificial Sequence
  source: /note=“Description of Artificial Sequence: Synthetic peptide”
GYTFTSYWMY

SEQ ID NO: 648-686 - skipped (39 entries)

SEQ ID NO: 687 - protein, 447 aa, Artificial Sequence
  source: /note=“Description of Artificial Sequence: Synthetic polypeptide”
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 688 - protein, 215 aa, Artificial Sequence
  source: /note=“Description of Artificial Sequence: Synthetic polypeptide”
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 689 - protein, 121 aa, Artificial Sequence
  source: /note=“Description of Artificial Sequence: Synthetic polypeptide”
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLWEVRALPSVYWGQGTLVTVSS

SEQ ID NO: 690 - protein, 109 aa, Artificial Sequence
  source: /note=“Description of Artificial Sequence: Synthetic polypeptide”
SYELTQPPSVSVAPGQTARITCGANDIGSKSVHWYQQKAGQAPVLVVSEDIIRPSGIPERISGSNSGNTATLTISRVEAGDEADYYCQVWDRDSDQYVFGTGTKVTVLG

SEQ ID NO: 691-921 - skipped (231 entries)

SEQ ID NO: 922 - protein, 6 aa, Artificial Sequence
  source: /note=“Description of Artificial Sequence: Synthetic 6xHis tag”
HHHHHH

SEQ ID NO: 923 - protein, 495 aa, Homo sapiens
MAGKVRSLLPPLLLAAAGLAGLLLLCVPTRDVREPPALKYGIVLDAGSSHTSMFIYKWPADKENDTGIVGQHSSCDVPGGGISSYADNPSGASQSLVGCLEQALQDVPKERHAGTPLYLGATAGMRLLNLTNPEASTSVLMAVTHTLTQYPFDFRGARILSGQEEGVFGWVTANYLLENFIKYGWVGRWFRPRKGTLGAMDLGGASTQITFETTSPAEDRASEVQLHLYGQHYRVYTHSFLCYGRDQVLQRLLASALQTHGFHPCWPRGFSTQVLLGDVYQSPCTMAQRPQNFNSSARVSLSGSSDPHLCRDLVSGLFSFSSCPFSRCSFNGVFQPPVAGNFVAFSAFFYTVDFLRTSMGLPVATLQQLEAAAVNVCNQTWAQLQARVPGQRARLADYCAGAMFVQQLLSRGYGFDERAFGGVIFQKKAADTAVGWALGYMLNLTNLIPADPPGLRKGTDFSSWVVLLLLFASALLAALVLLLRQVHSAKLPSTI

SEQ ID NO: 924 - DNA, 2156 bp, Homo sapiens
ggctccccgcactctccgggtccacgcatcgtcctcccgcgcgcccgcccgcccatggccgggaaggtgcggtcactgctgccgccgctgctgctggccgccgcgggcctcgccggcctcctactgctgtgcgtccccacccgcgacgtccgggagccgcccgccctcaagtatggcatcgtcctggacgctggttcttcacacacgtccatgtttatctacaagtggccggcagacaaggagaacgacacaggcattgtgggccagcacagctcctgtgatgttccaggtgggggcatctccagctatgcagacaacccttctggggccagccagagtcttgttggatgcctcgaacaggcgcttcaggatgtgcccaaagagagacacgcgggcacacccctctacctgggagccacagcgggtatgcgcctgctcaacctgaccaatccagaggcctcgaccagtgtgctcatggcagtgactcacacactgacccagtacccctttgacttccggggtgcacgcatcctctcgggccaggaagagggggtgtttggctgggtgactgccaactacctgctggagaacttcatcaagtacggctgggtgggccggtggttccggccacggaaggggacactgggggccatggacctggggggtgcctctacccagatcacttttgagacaaccagtccagctgaggacagagccagcgaggtccagctgcatctctacggccagcactaccgagtctacacccacagcttcctctgctatggccgtgaccaggtcctccagaggctgctggccagcgccctccagacccacggcttccacccctgctggccgaggggcttttccacccaagtgctgctcggggatgtgtaccagtcaccatgcaccatggcccagcggccccagaacttcaacagcagtgccagggtcagcctgtcagggagcagtgacccccacctctgccgagatctggtttctgggctcttcagcttctcctcctgccccttctcccgatgctctttcaatggggtcttccagcccccagtggctgggaactttgtggccttctctgccttcttctacactgtggactttttgcggacttcgatggggctgcccgtggccaccctgcagcagctggaggcagccgcagtgaatgtctgcaaccagacctgggctcagctgcaagctcgggtgccagggcaacgggcccgcctggccgactactgcgccggggccatgttcgtgcagcagctgctgagtcgcggctacggcttcgacgagcgcgccttcggcggcgtgatcttccagaagaaggccgcggacactgcagtgggctgggcgctcggctacatgctgaacctgaccaacctgatccccgccgacccgccggggctgcgcaagggcacagacttcagctcctgggtcgtcctcctgctgctcttcgcctccgcgctcctggctgcgcttgtcctgctgctgcgtcaggtgcactccgccaagctgccaagcaccatttaggggccgacgggggcagctgccccatccctcccccaacccctgtatccccaccccgtactcccacccctcccacaacccctgtacctcccacccctgtatccccacccctccacccacccctctcccaacctctctccccgcccctgtatcctgcattcctccacccaccctctatcccccaccgctccaccccaccactgtcttctccatccttccaccccaccctcagcgtctctgcccctaaggcagcccaggaaataggaactgagactctggtacccacaggagcctgggtgggcaaagagcgctcaatccagctccttgaacccctccagcccgcttcagcctgggcatcactgcaggccccgtgctcctcctcctcctcctcagggctgggtctccagagagtggggccttggtcctgagaatcagcccttagaggctccttctgtgtagtctgggtctgtactggggagggtcacagcccacgggctggcagccagcccagcacctacttgtaaaaattttgtaataaaaagtttttcctagagacgtgaaaaaaaaaaaaaaaaaaaaaaaaaaaaaaaaaaaaaaaaaaaaaaaaaaaaaaaaaa

SEQ ID NO: 925 - protein, 472 aa, Homo sapiens
MAGKVRSLLPPLLLAAAGLAGLLLLCVPTRDVREPPALKYGIVLDAGSSHTSMFIYKWPADKENDTGIVGQHSSCDVPGGGISSYADNPSGASQSLVGCLEQALQDVPKERHAGTPLYLGATAGMRLLNLTNPEASTSVLMAVTHTLTQYPFDFRGARILSGQEEGVFGWVTANYLLENFIKYGWVGRWFRPRKGTLGAMDLGGASTQITFETTSPAEDRASEVQLHLYGQHYRVYTHSFLCYGRDQVLQRLLASALQTHGFHPCWPRGFSTQVLLGDVYQSPCTMAQRPQNFNSSARVSLSGSSDPHLCRDLVSGLFSFSSCPFSRCSFNGVFQPPVAGNFVAFSAFFYTVDFLRTSMGLPVATLQQLEAAAVNVCNQTWAQQLLSRGYGFDERAFGGVIFQKKAADTAVGWALGYMLNLTNLIPADPPGLRKGTDFSSWVVLLLLFASALLAALVLLLRQVHSAKLPSTI

SEQ ID NO: 926 - DNA, 2087 bp, Homo sapiens
ggctccccgcactctccgggtccacgcatcgtcctcccgcgcgcccgcccgcccatggccgggaaggtgcggtcactgctgccgccgctgctgctggccgccgcgggcctcgccggcctcctactgctgtgcgtccccacccgcgacgtccgggagccgcccgccctcaagtatggcatcgtcctggacgctggttcttcacacacgtccatgtttatctacaagtggccggcagacaaggagaacgacacaggcattgtgggccagcacagctcctgtgatgttccaggtgggggcatctccagctatgcagacaacccttctggggccagccagagtcttgttggatgcctcgaacaggcgcttcaggatgtgcccaaagagagacacgcgggcacacccctctacctgggagccacagcgggtatgcgcctgctcaacctgaccaatccagaggcctcgaccagtgtgctcatggcagtgactcacacactgacccagtacccctttgacttccggggtgcacgcatcctctcgggccaggaagagggggtgtttggctgggtgactgccaactacctgctggagaacttcatcaagtacggctgggtgggccggtggttccggccacggaaggggacactgggggccatggacctggggggtgcctctacccagatcacttttgagacaaccagtccagctgaggacagagccagcgaggtccagctgcatctctacggccagcactaccgagtctacacccacagcttcctctgctatggccgtgaccaggtcctccagaggctgctggccagcgccctccagacccacggcttccacccctgctggccgaggggcttttccacccaagtgctgctcggggatgtgtaccagtcaccatgcaccatggcccagcggccccagaacttcaacagcagtgccagggtcagcctgtcagggagcagtgacccccacctctgccgagatctggtttctgggctcttcagcttctcctcctgccccttctcccgatgctctttcaatggggtcttccagcccccagtggctgggaactttgtggccttctctgccttcttctacactgtggactttttgcggacttcgatggggctgcccgtggccaccctgcagcagctggaggcagccgcagtgaatgtctgcaaccagacctgggctcagcagctgctgagtcgcggctacggcttcgacgagcgcgccttcggcggcgtgatcttccagaagaaggccgcggacactgcagtgggctgggcgctcggctacatgctgaacctgaccaacctgatccccgccgacccgccggggctgcgcaagggcacagacttcagctcctgggtcgtcctcctgctgctcttcgcctccgcgctcctggctgcgcttgtcctgctgctgcgtcaggtgcactccgccaagctgccaagcaccatttaggggccgacgggggcagctgccccatccctcccccaacccctgtatccccaccccgtactcccacccctcccacaacccctgtacctcccacccctgtatccccacccctccacccacccctctcccaacctctctccccgcccctgtatcctgcattcctccacccaccctctatcccccaccgctccaccccaccactgtcttctccatccttccaccccaccctcagcgtctctgcccctaaggcagcccaggaaataggaactgagactctggtacccacaggagcctgggtgggcaaagagcgctcaatccagctccttgaacccctccagcccgcttcagcctgggcatcactgcaggccccgtgctcctcctcctcctcctcagggctgggtctccagagagtggggccttggtcctgagaatcagcccttagaggctccttctgtgtagtctgggtctgtactggggagggtcacagcccacgggctggcagccagcccagcacctacttgtaaaaattttgtaataaaaagtttttcctagagacgtgaaaaaaaaaaaaaaaaaaaaaaaaaaaaaaaaaaaaaaaaaaaaaaaaaaaaaaaaaa